(19) United States
(12) Patent Application Publication
(10) Pub. No.: US 2023/0114107 A1
(43) Pub. Date: Apr. 13, 2023

(54) COMBINATION ANTI-CANCER THERAPIES WITH INDUCERS OF IRON-DEPENDENT CELLULAR DISASSEMBLY

(71) Applicant: Flagship Pioneering Innovations V, Inc., Cambridge, MA (US)

(72) Inventors: Anthony Michael Barsotti, New York, NY (US); Alexandra Masu Cantley, New York, NY (US); Jason Park, West Newton, MA (US); Darby Rye Schmidt, Arlington, MA (US); Peter Joseph Gough, Wayne, PA (US)

(21) Appl. No.: 17/787,287

(22) PCT Filed: Dec. 17, 2020
(86) PCT No.: PCT/US2020/065669

§ 371 Date: Jun. 17, 2022

Related U.S. Application Data
(60) Provisional application No. 62/949,334, filed on Dec. 17, 2019.

(51) Int. Cl. A61K 31/472; A61K 31/69; A61K 31/635; A61K 35/768; A61K 35/761; A61K 31/444; A61K 31/436; A61K 31/496; A61K 31/506; A61K 31/5025; A61K 31/517; A61K 31/5377; A61K 31/437; A61K 31/4545; A61K 31/519; A61K 31/502; A61K 39/395; A61K 31/4365; A61K 31/4375; A61P 35/00

(52) U.S. Cl. CPC A61K 31/472; A61K 31/69; A61K 31/635; A61K 35/768; A61K 35/761; A61K 31/444; A61K 31/436; A61K 31/496; A61K 31/506; A61K 31/5025; A61K 31/517; A61K 31/5377; A61K 31/437; A61K 31/4545; A61K 31/519; A61K 31/502; A61K 39/3955; A61K 31/4365; A61K 31/4375; A61P 35/00

(57) ABSTRACT

The invention provides methods of treating a cancer in a subject, comprising administering to the subject a combination of (a) an anti-neoplastic agent and (b) an agent that induces iron-dependent cellular disassembly, thereby treating the cancer in the subject. In some embodiments, the cancer is resistant to the anti-neoplastic agent.

### RELATED APPLICATIONS

This application claims priority to U.S. Provisional Application No. 62/949,334, filed on Dec. 17, 2019, the entire contents of which are expressly incorporated herein by reference.

### BACKGROUND

In multicellular organisms, cell death is a critical and active process that is believed to maintain tissue homeostasis and eliminate potentially harmful cells.

### SUMMARY OF THE INVENTION

In certain aspects, the disclosure relate to a method of killing a cancer cell in a subject, comprising contacting the cancer cell, or cells adjacent to the cancer cell, with a combination of (a) an anti-neoplastic agent and (b) an agent that induces iron-dependent cellular disassembly, wherein the cancer cell is resistant to the anti-neoplastic agent, thereby killing the cancer cell. In one embodiment, the anti-neoplastic agent is a cytotoxic agent. In one embodiment, the anti-neoplastic agent is radiotherapy. In one embodiment, the anti-neoplastic agent is an apoptosis inducer. In one embodiment, the apoptosis inducer is selected from the group consisting of ONY-015, INGN201, PS1145, Bortezomib, CCI779, RAD-001 and ABT-199 (Venetoclax). In one embodiment, the antineoplastic agent is selected from the group consisting of Bosutinib, Dasatinib, Imatinib, Nilotinib, Ponatinib, Cetuximab, Panitumumab, Afatinib, Erlotinib, Gefitinib, Dabrafenib, Vemurafenib, Ceritinib, Crizotinib Trametinib, Olaparib, Ado-trastuzumab, emtansine, Lapatinib, Pertuzumab and Trastuzumab. In one embodiment, said contacting induces iron-dependent cellular disassembly of the resistant cancer cell. In one embodiment, said contacting results in an increase in immune response to the resistant cancer cell in the subject.

In one embodiment, the resistant cancer cell exhibits (i) increased expression of a marker selected from the group consisting of HIF1, CD133, CD24, KDM5A/RBP2/Jarid1A, IGFBP3 (IGF-binding protein 3), Stat3, IRF-1, Interferon gamma, type I interferon, pax6, AKT pathway activation, IGF1, EGF, ANGPTL7, PDGFD, FRA1 (FOSL1), FGFR, KIT, IGF1R and DDR1, relative to a cancer cell that is sensitive to the anti-neoplastic agent; or (ii) decreased expression of IGFBP-3 relative to a cancer cell that is sensitive to the anti-neoplastic agent. In one embodiment, the antineoplastic agent and the cancer cell are selected from the antineoplastic agent and corresponding cancer cell listed in Table 4.

In certain aspects, the disclosure relate to a method of killing cancer cells in a subject, comprising contacting the cancer cells, or cells adjacent to the cancer cells, with a combination of (a) an anti-neoplastic agent and (b) an agent that induces iron-dependent cellular disassembly, wherein the method increases the number of cancer cells undergoing iron-dependent cellular disassembly relative to cancer cells treated with the agent that induces iron-dependent cellular disassembly alone. In one embodiment, the anti-neoplastic agent is an apoptosis inducer, in certain embodiments, wherein the anti-neoplastic agent is an apoptosis inducer in the absence of an agent that induces iron-dependent cellular disassembly. In one embodiment, the apoptosis inducer is selected from the group consisting of ONY-015, INGN201, PS1145, Bortezomib, CCI779, RAD-001 and ABT-199 (Venetoclax). In one embodiment, said contacting results in an increase in immune response to the cancer cell in the subject.

In certain aspects, the disclosure relate to a method of treating a cancer in a subject in need thereof, comprising administering to the subject, in combination (a) an anti-neoplastic agent and (b) an agent that induces iron-dependent cellular disassembly, thereby treating the cancer in the subject, wherein the cancer is resistant to the anti-neoplastic agent. In one embodiment, the anti-neoplastic agent is a cytotoxic agent. In one embodiment, the anti-neoplastic agent is an apoptosis inducer. In one embodiment, the antineoplastic agent is selected from the group consisting of Bosutinib, Dasatinib, Imatinib, Nilotinib, Ponatinib, Cetuximab, Panitumumab, Afatinib, Erlotinib, Gefitinib, Dabrafenib, Vemurafenib, Ceritinib, Crizotinib Trametinib, Olaparib, Ado-trastuzumab, emtansine, Lapatinib, Pertuzumab and Trastuzumab. In one embodiment, said administration results in resistant cancer cells undergoing iron-dependent cellular disassembly in the subject. In one embodiment, said administration results in an increase in immune response to the resistant cancer. In one embodiment, said method further comprises administering an immunotherapy to the subject. In one embodiment, the resistant cancer exhibits (i) increased expression of a marker selected from the group consisting of HIF1, CD133, CD24, KDM5A/RBP2/Jarid1A, IGFBP3 (IGF-binding protein 3), Stat3, IRF-1, Interferon gamma, type I interferon, pax6, AKT pathway activation, IGF1, EGF, ANGPTL7, PDGFD, FRA1 (FOSL1), FGFR, KIT, IGF1R and DDR1, relative to a cancer that is sensitive to the anti-neoplastic agent; or (ii) decreased expression of IGFBP-3 relative to a cancer that is sensitive to the anti-neoplastic agent. In one embodiment, the antineoplastic agent and the cancer are selected from the antineoplastic agent and corresponding cancer cell listed in Table 4.

In certain aspects, the disclosure relate to a method of treating a cancer in a subject in need thereof, comprising administering to the subject, in combination (a) an anti-neoplastic agent and (b) an agent that induces iron-dependent cellular disassembly, thereby treating the cancer in the subject, wherein the anti-neoplastic agent is known to induce resistance in the cancer. In one embodiment, the anti-neoplastic agent is a cytotoxic agent. In one embodiment, the anti-neoplastic agent is an apoptosis inducer. In one embodiment, the antineoplastic agent is selected from the group consisting of Bosutinib, Dasatinib, Imatinib, Nilotinib, Ponatinib, Cetuximab, Panitumumab, Afatinib, Erlotinib, Gefitinib, Dabrafenib, Vemurafenib, Ceritinib, Crizotinib Trametinib, Olaparib, Ado-trastuzumab, emtansine, Lapatinib, Pertuzumab and Trastuzumab.

In certain aspects, the disclosure relate to a method of reducing the heterogeneity of a cancer in a subject in need thereof, wherein the cancer comprises cells that are resistant to an anti-neoplastic agent and cells that are sensitive to the anti-neoplastic agent, the method comprising administering to the subject, in combination (a) the anti-neoplastic agent and (b) an agent that induces iron-dependent cellular disassembly, thereby reducing the heterogeneity of the cancer. In one embodiment, the cells that are resistant to the anti-neoplastic agent comprise persister cells. In one embodiment, the subject was previously determined to have elevated levels of the persister cells. In one embodiment, said administration results in reduction of the number of the persister cells in the cancer. In one embodiment, said administration results in preferential killing of the persister cells in the cancer. In one embodiment, the persister cells exhibit (i) increased expression of a marker selected from the group consisting of HIF1, CD133, CD24, KDM5A/RBP2/Jarid1A, IGFBP3 (IGF-binding protein 3), Stat3, IRF-1, Interferon gamma, type I interferon, pax6, AKT pathway activation, IGF1, EGF, ANGPTL7, PDGFD, FRA1 (FOSL1), FGFR, KIT, IGF1R and DDR1, relative to a cancer cell that is sensitive to the anti-neoplastic agent; or (ii) decreased expression of IGFBP-3 relative to a cancer cell that is sensitive to the anti-neoplastic agent.

In some embodiments, the cancer is selected from the group consisting of gastrointestinal stromal tumor (GIST), colorectal cancer (CRC), non-small cell lung cancer (NSCLC), melanoma, ovarian cancer, breast cancer and gastric cancer. In one embodiment, the cells that are resistant to the anti-neoplastic agent comprise cancer stem cells (CSCs). In one embodiment, the cancer further comprises non-CSCs. In one embodiment, the non-CSCs are sensitive to the anti-neoplastic agent. In one embodiment, the subject was previously determined to have elevated levels of the CSCs. In one embodiment, the CSCs are epithelial-mesenchymal transition (EMT) cells. In one embodiment, the non-CSCs are epithelial cells. In one embodiment, said administration results in reduction of the number of the CSCs in the cancer. In one embodiment, said administration results in preferential killing of the CSCs in the cancer. In one embodiment, the CSCs exhibit (i) increased expression of a marker selected from the group consisting of Vimentin (S100A4), Beta-catenin, N-cadherin, Beta6 integrin, Alpha4 integrin, DDR2, FSP1, Alpha-SMA, Beta-Catenin, Laminin 5, FTS-1, Twist, FOXX2, OB-cadherin, Alpha5beta1 integrin, alphaVbeta6 integrin, Syndecan-1, Alpha1 (I) collagen, Alpha1 (III) collagen, Snail1, Snail2, ZEB1, CBF-A/KAP-1 complex, LEF-1, Ets-1 and miR-21, relative to a non-CSC; or (ii) decreased expression of a marker selected from the group consisting of E-cadherin, ZO-1, cytokeratin, Alpha1 (IV) collagen and Laminin 1, relative to a non-CSC. In one embodiment, the cancer is selected from the group consisting of gastrointestinal stromal tumor (GIST), colorectal cancer (CRC), non-small cell lung cancer (NSCLC), melanoma, ovarian cancer, breast cancer and gastric cancer. In one embodiment, the method reduces risk of relapse of the cancer. In one embodiment, the method reduces risk of metastasis of the cancer.

In certain aspects, the disclosure relate to a method of increasing the therapeutic index of an anti-neoplastic agent for treating a cancer in a subject in need thereof, comprising administering to the subject, in combination (a) the anti-neoplastic agent and (b) an agent that induces iron-dependent cellular disassembly, thereby increasing the therapeutic index of the anti-neoplastic agent for treating the cancer in the subject.

In certain aspects, the disclosure relate to a method of treating a cancer in a subject in need thereof, comprising administering to the subject, in combination (a) an anti-neoplastic agent and (b) an agent that induces iron-dependent cellular disassembly, wherein the anti-neoplastic agent is administered at a dose that is lower than an effective dose of the anti-neoplastic agent when administered alone to treat the cancer, thereby treating the cancer in the subject. In one embodiment, the anti-neoplastic agent has a dose limiting effect. In one embodiment, the anti-neoplastic agent is administered at a dose that is at least 5%, 10%, 20%, 30%, 40%, 50%, or 60% less than the effective dose of the anti-neoplastic agent when administered alone to treat the cancer.

In some embodiments of the methods described herein, the cancer has a mesenchymal phenotype. In one embodiment, the cancer exhibits (i) increased expression of a marker selected from the group consisting of Vimentin (S100A4), Beta-catenin, N-cadherin, Beta6 integrin, Alpha4 integrin, DDR2, FSP1, Alpha-SMA, Beta-Catenin, Laminin 5, FTS-1, Twist, FOXX2, OB-cadherin, Alpha5beta1 integrin, alphaVbeta6 integrin, Syndecan-1, Alpha1 (I) collagen, Alpha1 (III) collagen, Snail1, Snail2, ZEB1, CBF-A/KAP-1 complex, LEF-1, Ets-1 and miR-21, relative to a non-CSC; and/or (ii) decreased expression of a marker selected from the group consisting of E-cadherin, ZO-1, cytokeratin, Alpha1 (IV) collagen and Laminin 1, relative to a non-mesenchymal cancer. In one embodiment, the cancer is selected from the group consisting of chronic myeloid leukemia (CML), acute lymphoblastic leukemia (ALL), gastrointestinal stromal tumor (GIST), colorectal cancer (CRC), non-small cell lung cancer (NSCLC), melanoma, ovarian cancer, breast cancer and gastric cancer.

In some embodiments of the methods described herein, the anti-neoplastic agent and the agent that induces iron dependent cellular disassembly are administered to the subject simultaneously. In one embodiment, the anti-neoplastic agent and the agent that induces iron-dependent cellular disassembly are administered to the subject sequentially. In one embodiment, the anti-neoplastic agent and the agent that induces iron-dependent cellular disassembly are administered in an amount that causes a synergistic effect. In one embodiment, the method results in an increased immune response to the cancer. In one embodiment, the increased immune response comprises activation of one or more cells selected from the group consisting of monocytes, pro-inflammatory macrophages, dendritic cells, neutrophils, NK cells and T cells. Wherein the increased immune response comprises an increase in the level or activity of NFκB, IRF or STING in an immune cell. In one embodiment, the immune cell is a THP-1 cell. In one embodiment, the method further comprises administering an immunotherapeutic agent to the subject. In one embodiment, the anti-neoplastic agent, the agent that induces iron-dependent cellular disassembly, and the immunotherapeutic agent are administered in an amount that causes a synergistic effect.

In some embodiments of the methods described herein, the antineoplastic agent is a cytotoxic agent. In one embodiment, the cytotoxic agent is an apoptosis inducer. In one embodiment, the apoptosis inducer is selected from the group consisting of ONY-015, INGN201, PS1145, Bortezomib, CCI779, RAD-001 and ABT-199 (Venetoclax). In one embodiment, the antineoplastic agent is selected from the group consisting of Bosutinib, Dasatinib, Imatinib, Nilotinib, Ponatinib, Cetuximab, Panitumumab, Afatinib, Erlotinib, Gefitinib, Dabrafenib, Vemurafenib, Ceritinib, Crizotinib Trametinib, Olaparib, Ado-trastuzumab, emtansine, Lapatinib, Pertuzumab and Trastuzumab. In one embodiment, the antineoplastic agent is unconjugated. In one embodiment, the antineoplastic agent is conjugated to a targeting moiety. In one embodiment, the targeting moiety is an antibody or antigen-binding fragment thereof. In one embodiment, the agent that induces iron-dependent cellular disassembly is selected from the group consisting of an inhibitor of antiporter system Xc-, an inhibitor of GPX4, and a statin. In one embodiment, the iron-dependent cellular disassembly is ferroptosis. In one embodiment, the inhibitor of antiporter system Xc− is erastin or a derivative or analog thereof.

In some embodiments of the methods described herein, the erastin or derivative or analog thereof has the following formula:

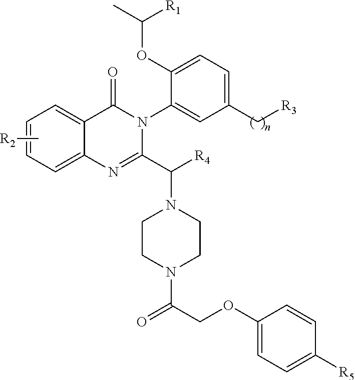

or pharmaceutically acceptable salts or esters thereof, wherein

R₁ is selected from the group consisting of H, C₁₋₄ alkyl, C₁₋₄ alkoxy, hydroxy, and halogen;

R₂ is selected from the group consisting of H, halo, and C₁₋₄ alkyl;

R₃ is selected from the group consisting of H, C₁₋₄ alkyl, C₁₋₄ alkoxy, 5-7 membered heterocycloalkyl, and 5-6 membered heteroaryl;

R₄ is selected from the group consisting of H and C₁₋₄ alkyl;

R₅ is halo;

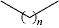

is optionally substituted with ═O; and
n is an integer from 0-4.

In some embodiments, the analog of erastin is PE or IKE.

In some embodiments, the inhibitor of GPX4 is selected from the group consisting of (1S,3R)-RSL3 or a derivative or analog thereof, ML162, DPI compound 7, DPI compound 10, DPI compound 12, DPI compound 13, DPI compound 17, DPI compound 18, DPI compound 19, FIN56, and FINO2.

In some embodiments, the RSL3 derivative or analog is a compound represented by Structural Formula (I):

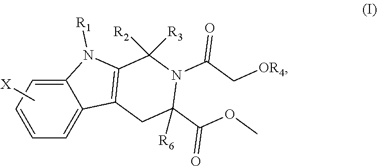

or an enantiomer, optical isomer, diastereomer, N-oxide, crystalline form, hydrate, or pharmaceutically acceptable salt thereof, wherein

R₁, R₂, R₃, and R₆ are independently selected from H, C₁₋₈alkyl, C₁₋₈alkoxy, C₁₋₈aralkyl, 3- to 8-membered carbocyclic, 3- to 8-membered heterocyclic, 3- to 8-membered aryl, or 3- to 8-membered heteroaryl, acyl, alkylsulfonyl, and arylsulfonyl, wherein each alkyl, alkoxy, aralkyl, carbocyclic, heterocyclic, aryl, heteroaryl, acyl, alkylsulfonyl, and arylsulfonyl is optionally substituted with at least one substituent;

R₄ and R₅ are independently selected from H₁ C₁₋₈alkyl, C₁₋₈alkoxy, 3- to 8-membered carbocyclic, 3- to 8-membered heterocyclic, 3- to 8-membered aryl, or 3- to 8-membered heteroaryl, carboxylate, ester, amide, carbohydrate, amino acid, acyl, alkoxy-substituted acyl, alditol, NR⁷R⁸, OC(R⁷)₂COOH, SC(R⁷)₂COOH, NHCHR⁷COOH, COR⁸, CO₂R⁸, sulfate, sulfonamide, sulfoxide, sulfonate, sulfone, thioalkyl, thioester, and thioether, wherein each alkyl, alkoxy, carbocyclic, heterocyclic, aryl, heteroaryl, carboxylate, ester, amide, carbohydrate, amino acid, acyl, alkoxy-substituted acyl, alditol, NR⁷R⁸, OC(R⁷)₂COOH, SC(R⁷)₂COOH, NHCHR⁷COOH, COR⁸, CO₂R⁸, sulfate, sulfonamide, sulfoxide, sulfonate, sulfone, thioalkyl, thioester, and thioether is optionally substituted with at least one substituent;

R⁷ is selected from H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;

R⁸ is selected from H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent; and

X is 0-4 substituents on the ring to which it is attached.

In some embodiments, the RSL3 derivative or analog is a compound represented by Structural Formula (II):

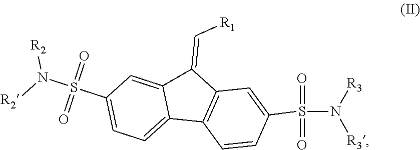

or an N-oxide, crystalline form, hydrate, or pharmaceutically acceptable salt thereof; wherein:

R₁ is selected from the group consisting of H, OH, and —(OCH₂CH₂)ₓOH;

X is an integer from 1 to 6; and

R₂, R₂′, R₃, and R₃′ independently are selected from the group consisting of H, C₃₋₈cycloalkyl, and combinations thereof, or R₂ and R₂′ may be joined together to form a pyridinyl or pyranyl and R₃ and R₃′ may be joined together to form a pyridinyl or pyranyl.

In some embodiments, the RSL3 derivative or analog is a compound represented by Structural Formula (III):

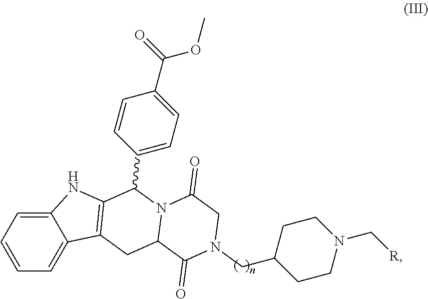

or a stereoisomer thereof, or a pharmaceutically acceptable salt thereof; wherein: n is 2, 3 or 4; and R is a substituted or unsubstituted C₁-C₆ alkyl group, a substituted or unsubstituted C₃-C₁₀ cycloalkyl group, a substituted or unsubstituted C₂-C₈ heterocycloalkyl group, a substituted or unsubstituted C₆-C₁₀ aromatic ring group, or a substituted or unsubstituted C₃-C₈ heteroaryl ring group; wherein the substitution means that one or more hydrogen atoms in each group are substituted by the following groups selected from the group consisting of: halogen, cyano, nitro, hydroxy, C₁-C₆ alkyl, halogenated C₁-C₆ alkyl, C₁-C₆ alkoxy, halogenated C₁-C₆ alkoxy, COOH (carboxy), COOC₁-C₆ alkyl, OCOC₁-C₆ alkyl.

In some embodiments, the RSL3 derivative or analog is a compound represented by Structural Formula (VI):

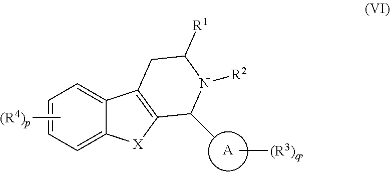

or an enantiomer, optical isomer, diastereomer, N-oxide, crystalline form, hydrate, or pharmaceutically acceptable salt thereof, wherein

ring A is C₄-C₁₀cycloalkyl, heterocyclyl, aryl, or heteroaryl;

X is NR⁵, O or S;

p is 0, 1, 2 or 3;

q is 0, 1, 2 or 3;

R¹ is C₁-C₆alkyl, C₂-C₆alkenyl, C₂-C₆alkynyl, C₁-C₆haloalkyl, C₃-C₁₀cycloalkyl, —CN, —OH, —C(O)OR⁶, —C(O)N(R⁷)₂, —OC(O)R⁶, —S(O)₂R⁸, —S(O)₂N(R⁷)₂, —S(O)N(R⁷)₂, —S(O)R⁸, —NH₂, —NHR⁸, —N(R⁸)₂, —NO₂, —OR⁸, —C₁-C₆alkyl-OH, —C₁-C₆alkyl-OR, or —Si(R¹⁵)₃;

R² is —C(O)R⁹;

each R³ is independently halo, —CN, —OH, —OR, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R, —NR¹²C(O)OR⁸, —OC(O)N(R⁷)₂, —OC(O)R⁸, —C(O)R⁶, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R³ is independently optionally substituted with one to three R¹⁰;

each R⁴ is independently halo, —CN, —OH, —OR, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, —C₁-C₆alkyl, —C₂-C₆alkenyl, —C₂-C₆alkynyl, —C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R⁴ is optionally independently optionally substituted with one to three R¹⁰;

R⁵ is hydrogen or C₁-C₆alkyl;

each R⁶ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁶ is independently further substituted with one to three R¹¹;

each R⁷ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₆cycloalkyl, —C₂-C₆alkenylC₃-C₆cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, —C₂-C₆alkenylheteroaryl, or two R⁷ together with the nitrogen atom to which they are attached, form a 4 to 7 membered heterocyclyl; wherein each R⁷ or ring formed thereby is independently further substituted with one to three R¹¹;

each R⁸ is independently C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁸ is independently further substituted with one to three R¹¹;

R⁹ is —C₁-C₂haloalkyl, —C₂-C₃alkenyl, —C₂-C₃haloalkenyl, C₂alkynyl, or —CH₂OS(O)₂-phenyl, wherein the C₁-C₂alkylhalo and C₂-C₃alkenylhalo are optionally substituted with one or two —CH₃, and the C₂alkynyl and phenyl are optionally substituted with one —CH₃;

each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl, wherein each C₁-C₆alkyl, C₁-C₆haloalkyl, C₂-C₆alkenyl, C₂-C₆alkynyl, C₃-C₁₀cycloalkyl, heterocyclyl, aryl, or heteroaryl of R¹⁰ is optionally independently substituted with one to three R¹¹;

each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl;

each R¹² is independently hydrogen, C₁-C₆alkyl or C₃-C₁₀cycloalkyl;

each R¹³ is independently C₁-C₆alkyl or C₃-C₁₀cycloalkyl; and

each R¹⁵ is independently C₁-C₆alkyl, C₂-C₆alkenyl, aryl, heteroaryl, —C₁-C₆alkylaryl, —C₂-C₆alkenylaryl, —C₁-C₆alkylheteroaryl, and —C₂-C₆alkenylheteroaryl.

In certain embodiments, when X is NR⁵, then R⁹ is C₂alkynyl.

In certain embodiments, when X is NR⁵, and R⁹ is —C₁-C₂haloalkyl, —C₂-C₃alkenyl, —C₂-C₃haloalkenyl, or —CH₂OS(O)₂-phenyl, wherein the C₁-C₂alkylhalo and —C₂-C₃alkenylhalo are optionally substituted with one or two —CH₃, and the phenyl is optionally substituted with —CH₃, then R¹ is other than —C(O)OR⁶ and —C(O)N(R⁷)₂.

In certain embodiments, when X is NR⁵, then (i) R⁹ is C₂alkynyl; or (ii) R⁹ is —C₁-C₂haloalkyl, —C₂-C₃alkenyl, —C₂-C₃haloalkenyl, or —CH₂OS(O)₂-phenyl, wherein the C₁-C₂alkylhalo and —C₂-C₃alkenylhalo are optionally substituted with one or two —CH₃, and the phenyl is optionally substituted with —CH₃, and R¹ is other than —C(O)OR⁶ and —C(O)N(R⁷)₂.

In certain embodiments, when X is NH, R¹ is —C(O)OR⁶, R² is —C(O)CH₂Cl or C(O)CH₂F, q is 1, p is 0, and ring A with the R³ is

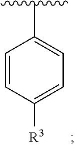

R³; then (i) R³ and R⁶ are not simultaneously —NO₂ and —CH₃, respectively, and (ii) when R⁶ is —CH₃, then R³ is other than H, halo, and —NO₂.

In certain embodiments, when X is NH, R¹ is —C(O)OR⁶, R² is —C(O)CH₂Cl or C(O)CH₂F, q is 1, p is 0, ring A with the R³ is

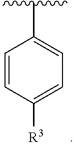

R³, and R³ is —C(O)OR⁶; then both R⁶ are not simultaneously

(i) —CH₃;

(ii) —CH₃ and C₂-C₆alkynyl, respectively; or

(iii) —CH₂CH₃ and —CH₃, respectively.

In certain embodiments, when X is NH, R¹ is —C(O)OCH₃, R² is —C(O)CH₂Cl or —C(O)CH₂F, q is 1, p is 0, and R³ is H; then ring A is other than phenyl.

In certain embodiments, and when X is NH, R¹ is —C(O)N(R⁷)₂, wherein R⁷ are H, R² is —C(O)CH₂Cl or —C(O)CH₂F, q is 0, or 1, p is 0, and ring A is phenyl; then q is not 0, or when q is 1, R³ is other than halo.

In some embodiments, the statin is selected from the group consisting of atorvastatin, fluvastatin, lovastatin, pitavastatin, pravastatin, rosuvastatin, cerivastatin and simvastatin. In one embodiment, the agent that induces iron-dependent cellular disassembly is selected from the group consisting of sorafenib or a derivative or analog thereof, sulfasalazine, glutamate, BSO, DPI2, cisplatin, cysteinase, silica based nanoparticles, CCI4, ferric ammonium citrate, trigonelline and brusatol.

In some embodiments, the agent that induces iron-dependent cellular disassembly has one or more of the following characteristics:

(a) induces iron-dependent cellular disassembly of a target cell in vitro and subsequent activation of an immune response in a co-cultured cell;

(b) induces iron-dependent cellular disassembly of a target cell in vitro and subsequent activation of co-cultured macrophages, e.g., RAW264.7 macrophages;

(c) induces iron-dependent cellular disassembly of a target cell in vitro and subsequent activation of co-cultured monocytes, e.g., THP-1 monocytes;

(d) induces iron-dependent cellular disassembly of a target cell in vitro and subsequent activation of co-cultured bone marrow-derived dendritic cells (BMDCs);

(e) induces iron-dependent cellular disassembly of a target cell in vitro and subsequent increase in levels or activity of NFkB, IRF and/or STING in a co-cultured cell;

(f) induces iron-dependent cellular disassembly of a target cell in vitro and subsequent increase in levels or activity of a pro-immune cytokine in a co-cultured cell; and

(g) induces iron-dependent cellular disassembly of a target cell in vitro and subsequent activation of co-cultured CD4+ cells, CD8+ cells and/or CD3+ cells.

In some embodiments, the agent that induces iron-dependent cellular disassembly is targeted to a cancer cell.

### BRIEF DESCRIPTION OF THE DRAWINGS

FIG. 1A shows HT1080 fibrosarcoma cells treated with various concentrations of erastin. FIG. 1B shows NFkB activity in THP1 monocytes co-cultured with HT1080 cells treated with erastin. Error bars represent standard deviation among three replicates.

FIG. 1C shows HT1080 fibrosarcoma cells treated with DMSO or various concentrations of erastin (ERAS) or the erastin analogs piperazine erastin (PE) or imidazole ketoerastin (IKE). The DMSO control is on the far left. The erastin or erastin analog concentrations increase from left to right and are the same as those shown in FIG. 1A.

FIG. 1D shows NFkB activity in THP1 monocytes co-cultured with HT1080 cells treated with erastin (ERAS) or the erastin analogs piperazine erastin (PE) or imidazole ketoerastin (IKE). The DMSO control is on the far left. The erastin or erastin analog concentrations increase from left to right and are the same as those shown in FIG. 1B. Error bars represent standard deviation among three replicates.

FIG. 2A shows pancreatic cancer cells (PANC1) treated with various concentrations of erastin. FIG. 2B shows NFkB activity in THP1 monocytes co-cultured with PANC1 cells treated with erastin.

FIG. 3A shows renal cell carcinoma cells (Caki-1) treated with various concentrations of erastin. FIG. 3B shows NFkB activity in THP1 monocytes co-cultured with Caki-1 cells treated with erastin.

FIG. 4A shows renal cell carcinoma cells (Caki-1) treated with various concentrations of RSL3. FIG. 4B shows NFkB activity in THP1 monocytes co-cultured with Caki-1 cells treated with RSL3.

FIG. 5A shows Jurkat T cell leukemia cells treated with various concentrations of RSL3. FIG. 5B shows NFkB activity in THP1 monocytes co-cultured with Jurkat cells treated with RSL3.

FIG. 6A shows A20 B-cell leukemia cells treated with various concentrations of RSL3. FIG. 6B shows NFkB activity in THP1 monocytes co-cultured with A20 cells treated with RSL3. FIG. 6C shows IRF activity in THP1 monocytes co-cultured with A20 cells treated with RSL3.

FIG. 7A shows the viability of HT1080 fibrosarcoma cells treated with various concentrations of Erastin alone or in combination with a ferroptosis inhibitor (Ferrostatin-1, Liproxstatin-1 or Trolox).

FIG. 7B shows NFkB activity in THP1 monocytes co-cultured with HT1080 fibrosarcoma cells treated with Erastin alone or in combination with a ferroptosis inhibitor (Ferrostatin-1, Liproxstatin-1 or Trolox).

FIG. 8A shows the viability of HT1080 fibrosarcoma cells treated with various concentrations of Erastin alone or in combination with a ferroptosis inhibitor (Ferrostatin-1, β-Mercaptoethanol, or Deferoxamine).

FIG. 8B shows NFkB activity in THP1 monocytes co-cultured with HT1080 fibrosarcoma cells treated with Erastin alone or in combination with a ferroptosis inhibitor (Ferrostatin-1, β-Mercaptoethanol, or Deferoxamine).

FIG. 9A shows the viability of HT1080 fibrosarcoma cells treated with various concentrations of Erastin in combination with an siRNA control (siControl) or an siRNA directed to the ACSL4 gene (siACSL4).

FIG. 9B shows the viability of H1080 fibrosarcoma cells treated with DMSO or Erastin in combination with an siRNA control (siControl), an siRNA directed to the ACSL4 gene (siACSL4), or an siRNA directed to the CARS gene (siCARS).

FIG. 9C shows the fold change in NFkB activity in THP1 monocytes co-cultured with HT1080 fibrosarcoma cells treated with DMSO or Erastin in combination with an siRNA control (siControl), an siRNA directed to the ACSL4 gene (siACSL4), or an siRNA directed to the CARS gene (siCARS).

FIG. 10A shows the viability of A20 lymphoma cells treated with DMSO or various concentrations of RSL3 alone or in combination with Ferrostatin-1.

FIG. 10B shows NFkB activity in THP1 monocytes co-cultured with A20 lymphoma cells treated with DMSO or various concentrations of RSL3 alone or in combination with Ferrostatin-1.

FIG. 11A shows the viability of A20 lymphoma cells treated with DMSO or various concentrations of ML162 alone or in combination with Ferrostatin-1.

FIG. 11B shows NFkB activity in THP1 monocytes co-cultured with A20 lymphoma cells treated with DMSO or various concentrations of ML162 alone or in combination with Ferrostatin-1.

FIG. 12A shows the viability of A20 lymphoma cells treated with DMSO or various concentrations of ML210 alone or in combination with Ferrostatin-1.

FIG. 12B shows NFkB activity in THP1 monocytes co-cultured with A20 lymphoma cells treated with DMSO or various concentrations of ML210 alone or in combination with Ferrostatin-1.

FIG. 13A shows the viability of Caki-1 renal carcinoma cells treated with DMSO or various concentrations of RSL3 alone or in combination with Ferrostatin-1.

FIG. 13B shows NFkB activity in THP1 monocytes co-cultured with Caki-1 renal carcinoma cells treated with DMSO or various concentrations of RSL3 alone or in combination with Ferrostatin-1.

FIG. 14A shows the viability of Caki-1 renal carcinoma cells treated with DMSO or various concentrations of ML162 alone or in combination with Ferrostatin-1.

FIG. 14B shows NFkB activity in THP1 monocytes co-cultured with Caki-1 renal carcinoma cells treated with DMSO or various concentrations of ML162 alone or in combination with Ferrostatin-1.

FIG. 15A shows HT1080 fibrosarcoma cells treated with various concentrations of erastin, docetaxel or H₂O₂. FIG. 15B shows NFkB activity in THP1 monocytes co-cultured with HT1080 cells treated with erastin, docetaxel or H₂O₂. Error bars represent standard deviation among three replicates.

FIGS. 16A, 16B and 16C show the effects of the ferroptosis inducer RSL3 on human melanoma A375 cell lines with acquired resistance to standard of care therapeutic BRAF inhibitors dabrafenib (A375-DR, FIG. 16A) or vemurafenib (A375-VR, FIG. 16B), or a human breast cancer cell line that is resistant to trastuzumab (BT-474 Clone 5, FIG. 16C). Cellular viability was assessed using the CellTiter-Glo 2.0 Cell Viability Assay. BRAF inhibitor-resistant melanoma cell lines A375-DR and A375-VR, and trastuzumab-resistant BT-474 clone 5 cell line, were all sensitive to RSL3-induced ferroptosis. The error bars represent the standard deviation. The line is the fit curve for a non-linear regression model run to calculate IC₅₀.

FIG. 17 shows the results of a screen of over 100 FDA-approved anticancer drugs and 6 inducers of ferroptosis for their ability to induce innate immune activation, as measured by induction of an NFkB reporter in THP1 monocytes in vitro. Erastin was among the top ten most effective inducers of innate immune signaling, exhibiting a 3.2-fold increase in NFkB activity relative to the baseline. IKE also induced innate immune signaling, exhibiting a 2.4-fold increase in NFkB activity relative to the baseline. The other four inducers of ferroptosis could not be evaluated, since they exhibited toxicity to the THP1 monocyte reporter cell line under the conditions of this assay. The intensity of the color of the band indicates the level of NFkB activation, with greater intensity indicating greater activation. The intensity of color corresponding to a particular fold increase in NFkB activity relative to the baseline is indicated in the box on the right, with the color intensity for 5-fold, 10-fold, 15-fold and 20-fold increases in NFkB activity indicated.

### DETAILED DESCRIPTION

The present disclosure relates to methods of treating cancer comprising administering a combination of (a) an anti-neoplastic agent and (b) an agent that induces iron-dependent cellular disassembly. Applicants have surprisingly shown that induction of iron-dependent cellular disassembly (e.g. ferroptosis) increases immune response as evidenced by increases in NFKB and IRF activity in immune cells. Accordingly, administration of an agent that induces iron-dependent cellular disassembly may be used to treat disorders that would benefit from increased immune activity, such as cancer. In addition, combining an agent that induces iron-dependent cellular disassembly with an antineoplastic agent may be used to kill cancer cells that are resistant to the anti-neoplastic agent.

### I. Definitions

The terms “administer”, “administering” or “administration” include any method of delivery of a pharmaceutical composition or agent into a subject's system or to a particular region in or on a subject.

As used herein, “administering in combination”, “co-administration” or “combination therapy” is understood as administration of two or more active agents using separate formulations or a single pharmaceutical formulation, or consecutive administration in any order such that, there is a time period while both (or all) active agents overlap in exerting their biological activities. It is contemplated herein that one active agent (e.g., an agent that induces iron-dependent cellular disassembly) can improve the activity of a second agent, for example, can sensitize target cells, e.g., cancer cells, to the activities of the second agent. “Administering in combination” does not require that the agents are administered at the same time, at the same frequency, or by the same route of administration. As used herein, “administering in combination”, “co-administration” or “combination therapy” includes administration of an agent that induces iron-dependent cellular disassembly with one or more additional anti-cancer agents, e.g., immune checkpoint modulators. Examples of immune checkpoint modulators are provided herein.

As used herein, an “anti-neoplastic agent” refers to a therapy used for the treatment of cancer. Anti-neoplastic agents include chemotherapeutic agents (e.g. alkylating agents, antimetabolites, anti-tumor antibiotics, topoisomerase inhibitors, mitotic inhibitors and corticosteroids)) and biologic anti-cancer agents (e.g. enzymes and antibodies). In one embodiment, the anti-neoplastic agent does not comprise an immunotherapeutic agent.

A “cancer treatment regimen” or “anti-neoplastic regimen” is a clinically accepted dosing protocol for the treatment of cancer that includes administration of one or more anti-neoplastic agents to a subject in specific amounts on a specific schedule.

“Cellular disassembly” refers to a dynamic process that reorders and disseminates the material within a cell and results in the production and release from the cell of postcellular signaling factors.

“Ferroptosis”, as used herein, refers to a process of regulated cell death that is iron dependent and involves the production of reactive oxygen species.

As used herein, an “immune checkpoint” or “immune checkpoint molecule” is a molecule in the immune system that modulates a signal. An immune checkpoint molecule can be a stimulatory checkpoint molecule, i.e., increase a signal, or inhibitory checkpoint molecule, i.e., decrease a signal. A “stimulatory checkpoint molecule” as used herein is a molecule in the immune system that increases a signal or is co-stimulatory. An “inhibitory checkpoint molecule”, as used herein is a molecule in the immune system that decreases a signal or is co-inhibitory.

As used herein, an “immune checkpoint modulator” is an agent capable of altering the activity of an immune checkpoint in a subject. In certain embodiments, an immune checkpoint modulator alters the function of one or more immune checkpoint molecules including, but not limited to, CD27, CD28, CD40, CD122, OX40, GITR, ICOS, 4-1BB, ADORA2A, B7-H3, B7-H4, BTLA, CTLA-4, IDO, KIR, LAG-3, PD-1, PD-L1, PD-L2, TIM-3, and VISTA. The immune checkpoint modulator may be an agonist or an antagonist of the immune checkpoint. In some embodiments, the immune checkpoint modulator is an immune checkpoint binding protein (e.g., an antibody, antibody Fab fragment, divalent antibody, antibody drug conjugate, scFv, fusion protein, bivalent antibody, or tetravalent antibody). In other embodiments, the immune checkpoint modulator is a small molecule. In a particular embodiment, the immune checkpoint modulator is an anti-PD1, anti-PD-L1, or anti-CTLA-4 binding protein, e.g., antibody or antibody fragment.

An “immunotherapeutic” as used herein refers to a pharmaceutically acceptable compound, composition or therapy that induces or enhances an immune response. Immunotherapeutics include, but are not limited to, immune checkpoint modulators, Toll-like receptor (TLR) agonists, cell-based therapies, cytokines and cancer vaccines.

As used herein, the terms “increasing” (or “activating”) and “decreasing” refer to modulating resulting in, respectively, greater or lesser amounts, function or activity of a parameter relative to a reference. For example, subsequent to administration of a preparation described herein, a parameter (e.g., activation of NFkB, activation of macrophages, size or growth of a tumor) may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the parameter prior to administration. Generally, the metric is measured subsequent to administration at a time that the administration has had the recited effect, e.g., at least one day, one week, one month, 3 months, 6 months, after a treatment regimen has begun. Similarly, pre-clinical parameters (such as activation of NFkB of cells in vitro, and/or reduction in tumor burden of a test mammal, by a preparation described herein) may be increased by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the parameter prior to administration.

As used herein, “oncological disorder” or “cancer” or “neoplasm” refer to all types of cancer or neoplasm found in humans, including, but not limited to: leukemias, lymphomas, melanomas, carcinomas and sarcomas. As used herein, the terms “oncological disorder”, “cancer,” and “neoplasm,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also cancer stem cells, as well as cancer progenitor cells or any cell derived from a cancer cell ancestor. This includes metastasized cancer cells, and in vitro cultures and cell lines derived from cancer cells.

Specific criteria for the staging of cancer are dependent on the specific cancer type based on tumor size, histological characteristics, tumor markers, and other criteria known by those of skill in the art. Generally, cancer stages can be described as follows: (i) Stage 0, Carcinoma in situ; (ii) Stage I, Stage II, and Stage III, wherein higher numbers indicate more extensive disease, including larger tumor size and/or spread of the cancer beyond the organ in which it first developed to nearby lymph nodes and/or tissues or organs adjacent to the location of the primary tumor; and (iii) Stage IV, wherein the cancer has spread to distant tissues or organs.

“Postcellular signaling factors” are molecules and cell fragments produced by a cell undergoing cellular disassembly (e.g., iron-dependent cellular disassembly) that are ultimately released from the cell and influence the biological activity of other cells. Postcellular signaling factors can include proteins, peptides, carbohydrates, lipids, nucleic acids, small molecules, and cell fragments (e.g. vesicles and cell membrane fragments).

A “solid tumor” is a tumor that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. The tumor does not need to have measurable dimensions.

A “subject” to be treated by the methods of the invention can mean either a human or non-human animal, preferably a mammal, more preferably a human. In certain embodiments, a subject has a detectable or diagnosed cancer prior to initiation of treatments using the methods of the invention. In certain embodiments, a subject has a detectable or diagnosed infection, e.g., chronic infection, prior to initiation of treatments using the methods of the invention.

“Therapeutically effective amount” means the amount of a compound that, when administered to a patient for treating a disease, is sufficient to effect such treatment for the disease. When administered for preventing a disease, the amount is sufficient to avoid or delay onset of the disease. The “therapeutically effective amount” will vary depending on the compound, the disease and its severity and the age, weight, etc., of the patient to be treated. A therapeutically effective amount need not be curative. A therapeutically effective amount need not prevent a disease or condition from ever occurring. Instead a therapeutically effective amount is an amount that will at least delay or reduce the onset, severity, or progression of a disease or condition.

The “therapeutic index” is the dose ratio between toxic and therapeutic effects, and it can be expressed as the ratio LD₅₀/ED₅₀. LD₅₀ is the dose lethal to 50% of the population. ED₅₀ is the dose therapeutically effective in 50% of the population. Drugs with a higher therapeutic index are desirable.

As used herein, “treatment”, “treating” and cognates thereof refer to the medical management of a subject with the intent to improve, ameliorate, stabilize, prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy).

“Alkyl” refers to a straight or branched chain hydrocarbon group of 1 to 20 carbon atoms (C₁-C₂₀ or C₁₋₂₀), e.g., 1 to 12 carbon atoms (C₁-C₁₂ or C₁₋₁₂), or 1 to 8 carbon atoms (C₁-C₈ or C₁₋₈). Exemplary “alkyl” includes, but are not limited to, methyl, ethyl, n-propyl, i-propyl, n-butyl, s-butyl, t-butyl, n-pentyl, and s-pentyl, and the like.

“Alkenyl” refers to a straight or branched chain hydrocarbon group of 2 to 20 carbon atoms (C₂-C₂₀ or C₂₋₂₀), e.g., 2 to 12 carbon atoms (C₂-C₁₂ or C₂₋₁₂), or 2 to 8 carbon atoms (C₂-C₈ or C₂-8), having at least one double bond. Exemplary “alkenyl” includes, but are not limited to, vinyl ethenyl, allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl and 5-hexenyl, and the like.

“Alkynyl” refers to a straight or branched chain hydrocarbon group of 2 to 12 carbon atoms (C₂-C₁₂ or C₂₋₁₂), e.g., 2 to 8 carbon atoms (C₂-C₈ or C₂₋₈), containing at least one triple bond. Exemplary “alkynyl” includes ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl, and the like.

“Alkylene,” “alkenylene” and “alkynylene” refers to a straight or branched chain divalent hydrocarbon radical of the corresponding alkyl, alkenyl, and alkynyl, respectively. The “alkylene,” “alkenylene” and “alkynylene” may be optionally substituted, for example with alkyl, alkyloxy, hydroxyl, carbonyl, carboxyl, halo, nitro, and the like. In certain embodiments, “alkyl,” “alkenyl,” and “alkynyl” can represent the corresponding “alkylene,” “alkenylene” and “alkynylene,” such as, by way of example and not limitation, cycloalkylalkyl-, heterocycloalkylalkyl-, arylalkyl-, heteroarylalkyl-, cycloalkylalkenyl-, heterocycloalkylalkenyl-, arylalkenyl-, heteroarylalkenyl-, cycloalkylalkynyl-, heterocycloalkylalkynyl-, arylalkynyl-, heteroarylalkynyl-, and the like, wherein the cycloalkyl, heterocycloalkyl, aryl, and heteroaryl group is connected, as a substituent via the corresponding alkylene, alkenylene, or alkynylene group.

“Aliphatic” refers to an organic compound characterized by substituted or unsubstituted, straight or branched, and/or cyclic chain arrangements of constituent carbon atoms. Aliphatic compounds do not contain aromatic rings as part of the molecular structure of the compounds. Aliphatic compound can have 1-20 (C₁-C₂₀ or C₁₋₂₀) carbon atoms, 1-12 (C₁-C₁₂ or C₁₋₁₂) carbon atoms, or 1-8 (C₁-C₅ or C₁-C₁₋₈) carbon atoms.

“Lower” in reference to substituents refers to a group having between one and six carbon atoms.

“Alkylhalo” or “haloalkyl” refers to a straight or branched chain hydrocarbon group of 1 to 20 carbon atoms (C₁-C₂₀ or C₁₋₂₀), e.g., 1 to 12 carbon atoms (C₁-C₁₂ or C₁₋₁₂), or 1 to 8 carbon atoms (C₁-C₈ or C₁₋₈) wherein one or more (e.g., one to three, or one) hydrogen atom is replaced by a halogen (e.g., Cl, F, etc.). In certain embodiments, the term “alkylhalo” refers to an alkyl group as defined herein, wherein one hydrogen atom is replaced by a halogen (e.g., Cl, F, etc.). In certain embodiments, the term “alkylhalo” refers to an alkylchloride.

“Alkenylhalo” or “haloalkenyl” refers to a straight or branched chain hydrocarbon group of 2 to 20 carbon atoms (C₂-C₂₀ or C₂₋₂₀), e.g., 2 to 12 carbon atoms (C₂-C₁₂ or C₂₋₁₂), or 2 to 8 carbon atoms (C₂-C₈ or C₂₋₈), having at least one double bond, wherein one or more (e.g., one to three, or one) hydrogen atom is replaced by a halogen (e.g., Cl, F, etc.). In certain embodiments, the term “alkenylhalo” refers to an alkenyl group as defined herein, wherein one hydrogen atom is replaced by a halogen (e.g., Cl, F, etc.). In certain embodiments, the term “alkenylhalo” refers to an alkenylchloride.

“Cycloalkyl” refers to any stable monocyclic or polycyclic system which consists of carbon atoms, any ring of which being saturated. “Cycloalkenyl” refers to any stable monocyclic or polycyclic system which consists of carbon atoms, with at least one ring thereof being partially unsaturated. Examples of cycloalkyls include, but are not limited to, cyclopropyl, cyclobutyl, cyclopentyl, cyclohexyl, cycloheptyl, cyclooctyl, bicycloalkyls and tricycloalkyls (e.g., adamantyl).

“Heterocycloalkyl” or “heterocyclyl” refers to a substituted or unsubstituted 4 to 14 membered, mono- or polycyclic (e.g., bicyclic), non-aromatic hydrocarbon ring, wherein 1 to 3 carbon atoms are replaced by a heteroatom. Heteroatoms and/or heteroatomic groups which can replace the carbon atoms include, but are not limited to, —O—, —S—, S—O—, —NR⁴⁰—, —PH—, —C(O)—, —S(O)—, —S(O)₂—, —S(O)NR⁴⁰—, S(O)₂NR⁴⁰—, and the like, including combinations thereof, where each R⁴⁰ is independently hydrogen or lower alkyl. Examples include thiazolidinyl, thiadiazolyl, triazinyl, morpholinyl, pyrrolidinonyl, pyrrolidinyl, piperidinyl, piperazinyl, 2,3-dihydrofuranyl, dihydropyranyl, hydantoinyl, valerolactamyl, oxiranyl, oxetanyl, tetrahydrofuranyl, tetrahydropyranyl, dihydropyridinyl, tetrahydropyridinyl, tetrahydropyrimidinyl, tetrahydrothiophenyl, tetrahydrothiopyranyl, and the like. In certain embodiments, the “heterocycloalkyl” or “heterocyclyl” is a substituted or unsubstituted 4 to 7 membered monocyclic ring, wherein 1 to 3 carbon atoms are replaced by a heteroatom as described above.

In certain embodiments, the “heterocycloalkyl” or “heterocyclyl” is a substituted or unsubstituted 4 to 10, or 4 to 9, or 5 to 9, or 5 to 7, or 5 to 6 membered mono- or polycyclic (e.g., bicyclic) ring, wherein 1 to 3 carbon atoms are replaced by a heteroatom as described above. In certain embodiments, when the “heterocycloalkyl” or “heterocyclyl” is a substituted or unsubstituted bicyclic ring, one ring may be aromatic, provided at least one ring is non-aromatic, regardless of the point of attachment to the remainder of the molecule (e.g., indolinyl, isoindolinyl, and the like).

“Carbocycle,” “carbocyclyl,” and “carbocyclic,” as used herein, refer to a non-aromatic saturated or unsaturated ring in which each atom of the ring is carbon. The ring may be monocyclic, bicyclic, tricyclic, or even of higher order. Thus, the terms “carbocycle,” “carbocyclyl,” and “carbocyclic,” encompass fused, bridged and spirocyclic systems. Preferably a carbocycle ring contains from 3 to 14 atoms, including 3 to 8 or 5 to 7 atoms, such as for example, 5 or 6 atoms.

“Aryl” refers to a 6 to 14-membered, mono- or bi-carbocyclic ring, wherein the monocyclic ring is aromatic and at least one of the rings in the bicyclic ring is aromatic. Unless stated otherwise, the valency of the group may be located on any atom of any ring within the radical, valency rules permitting. Examples of “aryl” groups include phenyl, naphthyl, indenyl, biphenyl, phenanthrenyl, naphthacenyl, and the like.

“Heteroaryl” means an aromatic heterocyclic ring, including monocyclic and polycyclic (e.g., bicyclic) ring systems, where at least one carbon atom of one or both of the rings is replaced with a heteroatom independently selected from nitrogen, oxygen, and sulfur, or at least two carbon atoms of one or both of the rings are replaced with a heteroatom independently selected from nitrogen, oxygen, and sulfur. In certain embodiments, the heteroaryl can be a 5 to 6 membered monocyclic, or 7 to 11 membered bicyclic ring systems. Examples of “heteroaryl” groups include pyrrolyl, pyrazolyl, imidazolyl, pyrazinyl, oxazolyl, isoxazolyl, thiazolyl, furyl, thienyl, pyridyl, pyrimidyl, benzothiazolyl, purinyl, benzimidazolyl, indolyl, isoquinolyl, quinoxalinyl, quinolyl, and the like.

“Bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In certain embodiments, a bridged bicyclic group has 5-12 ring members and 0-4 heteroatoms independently selected from nitrogen, oxygen, and sulfur. Such bridged bicyclic groups include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include, but are not limited to:

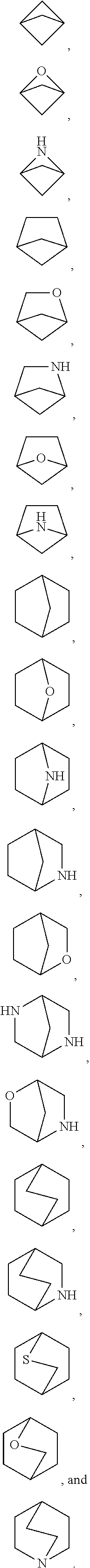

“Fused ring” refers a ring system with two or more rings having at least one bond and two atoms in common. A “fused aryl” and a “fused heteroaryl” refer to ring systems having at least one aryl and heteroaryl, respectively, that share at least one bond and two atoms in common with another ring.

“Carbonyl” refers to —C(O)—. The carbonyl group may be further substituted with a variety of substituents to form different carbonyl groups including acids, acid halides, aldehydes, amides, esters, and ketones. For example, an —C(O)R⁴¹, wherein R⁴¹ is an alkyl is referred to as an alkylcarbonyl. In certain embodiments, R⁴¹ is selected from an optionally substituted alkyl, cycloalkyl, cycloalkylalkyl, heterocycloalkyl, heterocycloalkylalkyl, aryl, arylalkyl, heteroaryl, and heteroarylalkyl.

“Halogen” or “halo” refers to fluorine, chlorine, bromine and iodine.

“Hydroxy” refers to —OH.

“Oxy” refer to group —O—, which may have various substituents to form different oxy groups, including ethers and esters. In certain embodiments, the oxy group is an OR⁴², wherein R⁴² is selected from an optionally substituted alkyl, cycloalkyl, cycloalkylalkyl, heterocycloalkyl, heterocycloalkylalkyl, aryl, arylalkyl, heteroaryl, and heteroarylalkyl.

“Acyl” refers to —C(O)R⁴³, where R⁴³ is hydrogen, or an optionally substituted alkyl, heteroalkyl, cycloalkyl, cycloalkylalkyl, heterocycloalkyl, heterocycloalkylalkyl, aryl, arylalkyl, heteroaryl, or heteroarylalkyl as defined herein. Exemplary acyl groups include, but are not limited to, formyl, acetyl, cyclohexylcarbonyl, cyclohexylmethylcarbonyl, benzoyl, benzylcarbonyl, and the like.

“Alkyloxy” or “alkoxy” refers to —OR⁴⁴, wherein R⁴⁴ is an optionally substituted alkyl.

“Aryloxy” refers to —OR⁴⁵, wherein R⁴⁵ is an optionally substituted aryl.

“Carboxy” refers to —COO— or COOM, wherein M is H or a counterion (e.g., a cation, such as Nat, Ca²⁺, Mg²⁺, etc.).

“Carbamoyl” refers to —C(O)NR⁴⁶R⁴⁶, wherein each R⁴⁶ is independently selected from H or an optionally substituted alkyl, cycloalkyl, cycloalkylalkyl, heterocycloalkyl, heterocylcoalkylalkyl, aryl, arylalkyl, heteroaryl, or heteroarylalkyl.

“Cyano” refers to —CN.

“Ester” refers to a group such as —C(═O)OR⁴⁷, alternatively illustrated as —C(O)OR⁴⁷, wherein R⁴⁷ is selected from an optionally substituted alkyl, cycloalkyl, cycloalkylalkyl, heterocycloalkyl, heterocyclolalkylalkyl, aryl, arylalkyl, heteroaryl, and heteroarylalkyl.

“Thiol” refers to —SH.

“Sulfanyl” refers to —SR⁴⁸, wherein R⁴⁸ is selected from an optionally substituted alkyl, cycloalkyl, cycloalkylalkyl, heterocycloalkyl, heterocycloalkylalkyl, aryl, arylalkyl, heteroaryl, and heteroarylalkyl. For example, —SR⁴⁸, wherein R⁴⁸ is an alkyl is an alkylsulfanyl.

“Sulfonyl” refers to —S(O)₂—, which may have various substituents to form different sulfonyl groups including sulfonic acids, sulfonamides, sulfonate esters, and sulfones. For example, —S(O)₂R⁴⁹ wherein R⁴⁹ is an alkyl refers to an alkylsulfonyl. In certain embodiments of —S(O)₂R⁴⁹, R⁴⁹ is selected from an optionally substituted alkyl, cycloalkyl, cycloalkylalkyl, heterocycloalkyl, heterocycloalkylalkyl, aryl, arylalkyl, heteroaryl, and heteroarylalkyl.

“Sulfinyl” refers to —S(O), which may have various substituents to form different sulfinyl groups including sulfinic acids, sulfinamides, and sulfinyl esters. For example, —S(O)R⁵⁰, wherein R⁵⁰ is an alkyl refers to an alkylsulfinyl. In certain embodiments of —S(O)R⁵⁰, R⁵⁰ is selected from an optionally substituted alkyl, cycloalkyl, cycloalkylalkyl, heterocycloalkyl, heterocycloalkylalkyl, aryl, arylalkyl, heteroaryl, and heteroarylalkyl.

“Silyl” refers to Si, which may have various substituents, for example —SiR⁵¹R⁵¹R⁵¹, where each R⁵¹ is independently selected from alkyl, cycloalkyl, cycloalkylalkyl, heterocycloalkyl, heterocycloalkylalkyl, aryl, arylalkyl, heteroaryl, and heteroarylalkyl. As defined herein, any heterocycloalkyl or heteroaryl group present in a silyl group has from 1 to 3 heteroatoms selected independently from O, N, and S.

“Amino” or “amine” refers to the group —NR⁵²R⁵² or —N+R⁵²R⁵²R⁵², wherein each R⁵² is independently selected from hydrogen and an optionally substituted alkyl, cycloalkyl, heterocycloalkyl, alkyloxy, aryl, heteroaryl, heteroarylalkyl, acyl, alkyloxycarbonyl, sulfanyl, sulfinyl, sulfonyl, and the like. Exemplary amino groups include, but are not limited to, dimethylamino, diethylamino, trimethylammonium, triethylammonium, methylysulfonylamino, furanyl-oxy-sulfamino, and the like.

“Amide” refers to a group such as, —C(═O)NR⁵³R⁵³, wherein each R⁵³ is independently selected from H and an optionally substituted alkyl, cycloalkyl, cycloalkylalkyl, heterocycloalkyl, heterocycloalkylalkyl, aryl, arylalkyl, heteroaryl, and heteroarylalkyl.

“Sulfonamide” refers to —S(O)₂NR⁵⁴R⁵⁴, wherein each R⁵⁴ is independently selected from H and an optionally substituted alkyl, heteroalkyl, heteroaryl, heterocycle, alkenyl, alkynyl, arylalkyl, heteroarylalkyl, heterocyclylalkyl, -alkylenecarbonyl-, or alkylene-O—C(O)—OR⁵⁵, where R⁵⁵ is selected from H, alkyl, heteroalkyl, cycloalkyl, heterocyclyl, aryl, heteroaryl, alkenyl, alkynyl, arylalkyl, heterocycloalkyl, heteroarylalkyl, amino, and sulfinyl.

“Adamantyl” refers to a compound of structural formula:

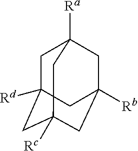

where optional substitutions can be present on one or more of R^{a}, Rb, R^{c}, and R^{d}. Adamantyl includes substituted adamantyl, e.g., 1- or 2-adamantyl, substituted by one or more substituents, including alkyl, halo, OH, NH₂, and alkoxy. Exemplary derivatives include methyladamatane, haloadamantane, hydroxyadamantane, and aminoadamantane (e.g., amantadine).

“N-protecting group” as used herein refers to those groups intended to protect a nitrogen atom against undesirable reactions during synthetic procedures. Exemplary N-protecting groups include, but is not limited to, acyl groups such acetyl and t-butylacetyl, pivaloyl, alkoxycarbonyl groups such as methyloxycarbonyl and t-butyloxycarbonyl (Boc), aryloxycarbonyl groups such as benzyloxycarbonyl (Cbz) and fluorenylmethoxycarbonyl (Fmoc and aroyl groups such as benzoyl. N-protecting groups are described in Greene's Protective Groups in Organic Synthesis, 5th Edition, P. G. M. Wuts, ed., Wiley (2014).

“Optional” or “optionally” refers to a described event or circumstance may or may not occur, and that the description includes instances where the event or circumstance occurs and instances where the event or circumstance does not. For example, “optionally substituted alkyl” refers to an alkyl group that may or may not be substituted and that the description encompasses both substituted alkyl group and unsubstituted alkyl group.

“Substituted” as used herein means one or more hydrogen atoms of the group is replaced with a substituent atom or group commonly used in pharmaceutical chemistry. Each substituent can be the same or different. Examples of suitable substituents include, but are not limited to, alkyl, alkenyl, alkynyl, cycloalkyl, aryl, arylalkyl, heterocycloalkyl, heteroaryl, —OR⁵⁶ (e.g., hydroxyl, alkyloxy (e.g., methoxy, ethoxy, and propoxy), aryloxy, heteroaryloxy, arylalkyloxy, ether, ester, carbamate, etc.), hydroxyalkyl, alkyloxycarbonyl, alkyloxyalkyloxy, perhaloalkyl, alkyloxyalkyl, SR⁵⁶ (e.g., thiol, alkylthio, arylthio, heteroarylthio, arylalkylthio, etc.), S⁺R⁵⁶₂, S(O)R⁵⁶, SO₂R⁵⁶, NR⁵⁶R⁵⁷ (e.g., primary amine (i.e., NH₂), secondary amine, tertiary amine, amide, carbamate, urea, etc.), hydrazide, halo, nitrile, nitro, sulfide, sulfoxide, sulfone, sulfonamide, thiol, carboxy, aldehyde, keto, carboxylic acid, ester, amide, imine, and imide, including seleno and thio derivatives thereof, wherein each R⁵⁶ and R⁵⁷ are independently alkyl, alkenyl, alkynyl, heteroalkyl, cycloalkyl, cycloalkylalkyl, heterocycloalkyl, heterocycloalkylalkyl, aryl, arylalkyl, heteroaryl, or heteroarylalkyl, and wherein each of the substituents can be optionally further substituted. In embodiments in which a functional group with an aromatic carbon ring is substituted, such substitutions will typically number less than about 10 substitutions, more preferably about 1 to 5, with about 1 or 2 substitutions being preferred.

“Pharmaceutically acceptable salt” is meant to include salts of the active compounds which are prepared with relatively nontoxic acids or bases, depending on the particular substituents found on the compounds described herein. When compounds as disclosed herein contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds as disclosed herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, phosphoric, partially neutralized phosphoric acids, sulfuric, partially neutralized sulfuric, hydroiodic, or phosphorous acids and the like, as well as the salts derived from relatively nontoxic organic acids like acetic, propionic, isobutyric, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure may contain both basic and acidic functionalities that allow the compounds to be converted into either base or acid addition salts. Lists of suitable salts are found in Remington's Pharmaceutical Sciences, 17th Ed., Mack Publishing Company, Easton, Pa., (1985) and Journal of Pharmaceutical Science, 66:2 (1977), each of which is incorporated herein by reference in its entirety.

“Pharmaceutically acceptable carrier” or “pharmaceutically acceptable excipient” refers to an excipient, carrier or adjuvant that can be administered to a subject, together with at least one therapeutic agent, and which does not destroy the pharmacological activity thereof and is generally safe, nontoxic and neither biologically nor otherwise undesirable when administered in doses sufficient to deliver a therapeutic amount of the agent.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

The compounds as disclosed herein, or their pharmaceutically acceptable salts include an asymmetric center and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are non-superimposable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

“Prodrugs” means any compound which releases an active parent drug according to a structure described herein in vivo when such prodrug is administered to a mammalian subject. Prodrugs of a compound described herein are prepared by modifying functional groups present in the compound described herein in such a way that the modifications may be cleaved in vivo to release the parent compound. Prodrugs include compounds described herein wherein a hydroxy, amino, carboxyl, or sulfhydryl group in a compound described herein is bonded to any group that may be cleaved in vivo to regenerate the free hydroxy, amino, or sulfhydryl group, respectively. Examples of prodrugs include, but are not limited to esters (e.g., acetate, formate and benzoate derivatives), amides, guanidines, carbamates (e.g., N,N-dimethylaminocarbonyl) of hydroxy functional groups in compounds described herein and the like. Specific prodrugs may include, but are not limited to, compounds provided herein where a solubility-enhancing moiety has been appended thereto. For example, a compound may be modified to include a polyethylene glycol group (e.g., —(OCH₂CH₂)ᵤ—OH, where u is from about 2 to about 6, or more) at or off a suitable functional group (e.g., an ester, amide, sulfonyl, or sulfonamide moiety) on R¹, R³ or R⁴, such as in Example 189 (below):

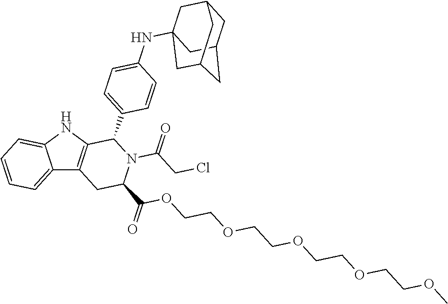 [SMILES: COCCOCCOCCOCCOC(=O)[C@H]1Cc2c([nH]c3ccccc23)[C@H](c2ccc(NC34CC5CC(CC(C5)C3)C4)cc2)N1C(=O)CCl]

Preparation, selection and use of prodrugs is discussed in T. Higuchi and V. Stella, “Pro-drugs as Novel Delivery Systems,” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

### II. Agents that Induce Iron-Dependent Cellular Disassembly

Cellular disassembly is a dynamic process that re-orders and disseminates the material within a cell, and which results in the production and release of postcellular signaling factors, or “effectors”, that can have a profound effect on the biological activity of other cells. Cellular disassembly occurs during the process of regulated cell death and is controlled by multiple molecular mechanisms. Different types of cellular disassembly result in the production of different postcellular signaling factors and thereby mediate different biological effects. For example, Applicants have surprisingly shown that induction of an iron-dependent cellular disassembly can increase immune response as evidenced by increases in NFKB and IRF activity in immune cells.

In some embodiments, the iron-dependent cellular disassembly is ferroptosis. Ferroptosis is a process of regulated cell death involving the production of iron-dependent reactive oxygen species (ROS). In some embodiments, ferroptosis involves the iron-dependent accumulation of lipid hydroperoxides to lethal levels. The sensitivity to ferroptosis is tightly linked to numerous biological processes, including amino acid, iron, and polyunsaturated fatty acid metabolism, and the biosynthesis of glutathione, phospholipids, NADPH, and Coenzyme Q10. Ferroptosis involves metabolic dysfunction that results in the production of both cytosolic and lipid ROS, independent of mitochondria but dependent on NADPH oxidases in some cell contexts (Dixon et al., 2012, Cell 149(5):1060-72).

Agents that Induce Iron-Dependent Cellular Disassembly

Provided herein are agents that induce iron-dependent cellular disassembly. Such agents are capable of inducing the process of iron-dependent cellular disassembly when present in sufficient amount and for a sufficient period of time. In certain embodiments, the agent that induces iron-dependent cellular disassembly induces the process of iron-dependent cellular disassembly in a cell such that post-cellular signaling factors, such as immunostimulatory post-cellular signaling factors, are produced by the cell, but does not result in cell death. In other embodiments, the agent that induces iron-dependent cellular disassembly induces the process of iron-dependent cellular disassembly in a portion of a cell population, e.g., 10%, 20%, 30%, 40%, 50%, 60%, 70%, 80%, 90%, 95% or more cells of the population, such that post-cellular signaling factors, e.g., immunostimulatory post-cellular signaling factors, are produced by the portion of cells in the cell population. Cell death may occur in all or only a fraction of the portion of cells in the cell population.

A broad range of agents that induce iron-dependent cellular disassembly, e.g., ferroptosis, are known in the art, and are useful in the various methods provided by the present invention. For example, two oncogenic RAS Selective Lethal (RSL) small molecules named eradicator of Ras and ST (erastin) and Ras Selective Lethal 3 (RSL3) were initially identified as small molecules that are selectively lethal to cells expressing oncogenic mutant RAS proteins, a family of small GTPases that are commonly mutated in cancer. (See Cao et al., 2016, Cell Mol Life Sci 73: 2195-2209, incorporated in its entirety herein.) Specifically, in engineered human fibroblast cell lines, the small molecule erastin was found to induce preferential lethality in cells overexpressing oncogenic HRAS (see Dolma et al., 2003, Cancer Cell. 3:285-296, incorporated in its entirety herein). Erastin functionally inhibits the cystine-glutamate antiporter system Xc−. System Xc− is a heterodimeric cell surface amino acid antiporter composed of the twelve-pass transmembrane transporter protein SLC7A11 (xCT) linked by a disulfide bridge to the single-pass transmembrane regulatory protein SLC3A2 (4F2hc, CD98hc). Antiporter system Xc-imports extracellular cystine, the oxidized form of cysteine, in exchange for intracellular glutamate. (See Cao et al., 2016, Cell Mol Life Sci 73: 2195-2209, incorporated in its entirety herein.) Cells treated with erastin are deprived of cysteine and are unable to synthesize the antioxidant glutathione. Depletion of glutathione eventually leads to excessive lipid peroxidation and increased ROS which triggers iron-dependent cellular disassembly. Erastin-induced ferroptotic cell death is distinct from apoptosis, necrosis, and autophagy, based on morphological, biochemical, and genetic criteria. (See Yang et al., 2014, Cell 156: 317-331, incorporated in its entirety herein.)

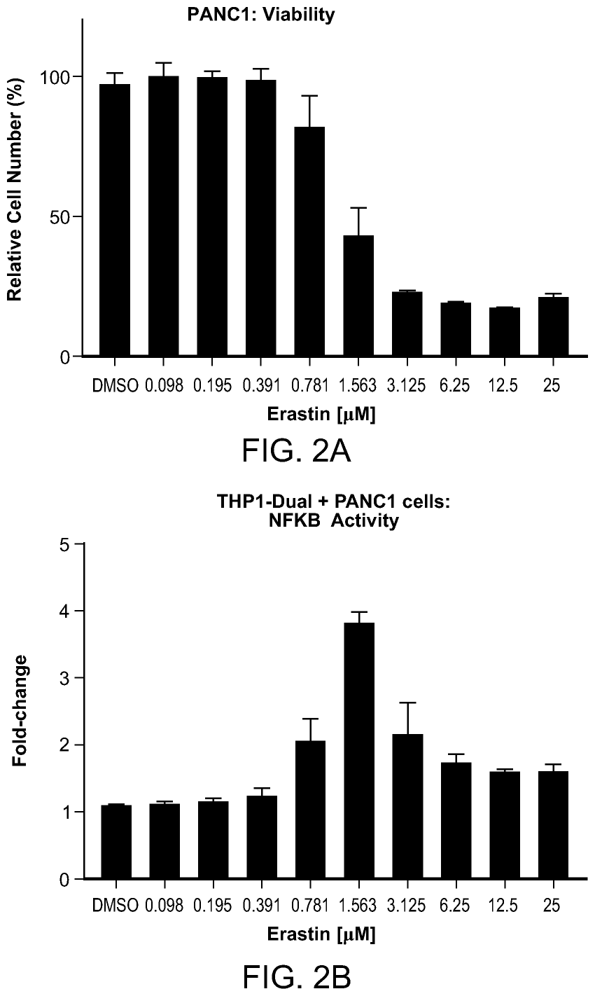

In some embodiments, an agent that induces iron-dependent cellular disassembly, e.g., ferroptosis, and is useful in the methods provided herein is an inhibitor of antiporter system Xc−. Inhibitors of antiporter system Xc−include antiporter system Xc-binding proteins (e.g., antibodies or antibody fragments), nucleic acid inhibitors (e.g., antisense oligonucleotides, or siRNAs), and small molecules that specifically inhibit antiporter system Xc−. For example, in some embodiments, the inhibitor of antiporter system Xc− is a binding protein, e.g., antibody or antibody fragment, that specifically inhibits SLC7A11 or SLC3A2. In some embodiments, the inhibitor of antiporter system Xc− is a nucleic acid inhibitor that specifically inhibits SLC7A11 or SLC3A2. In some embodiments, the inhibitor of antiporter system Xc− is small molecule that specifically inhibits SLC7A11 or SLC3A2. Antibody and nucleic acid inhibitors are well known in the art and are described in detail herein. Small molecule inhibitors of antiporter system Xc− include, but are not limited to, erastin, sulfasalazine, sorafenib, and analogs or derivatives thereof. (See Cao et al., 2016, Cell Mol Life Sci 73: 2195-2209, e.g., FIG. 2, incorporated in its entirety herein).

In a particular embodiment, an agent that induces iron-dependent cellular disassembly, e.g., ferroptosis, is erastin or an analog or derivative thereof. Analogs of erastin include, but are not limited to, the compounds listed in Table 1 below. Each of the references listed in Table 1 is incorporated by reference herein in its entirety.

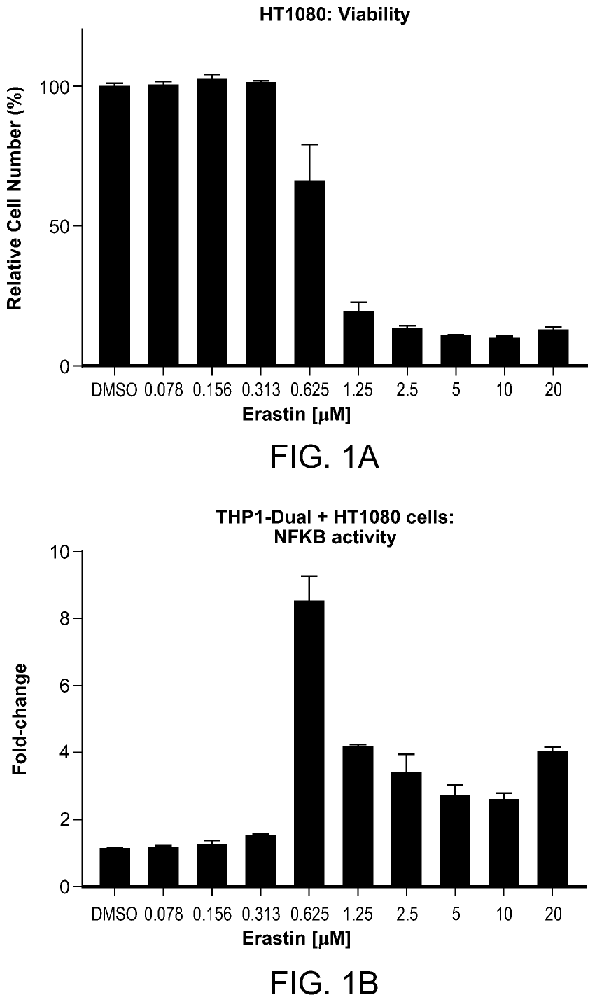

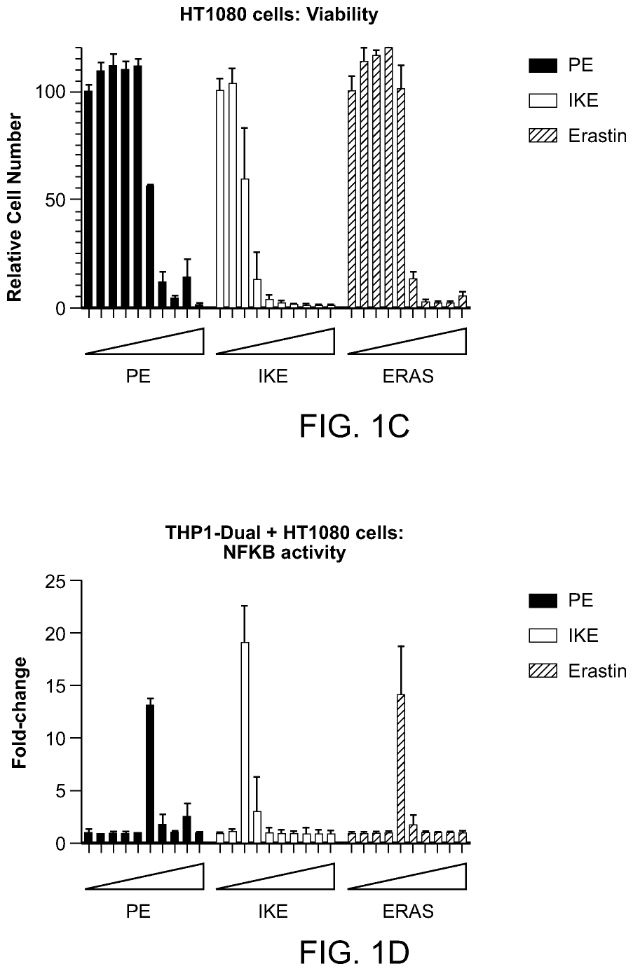

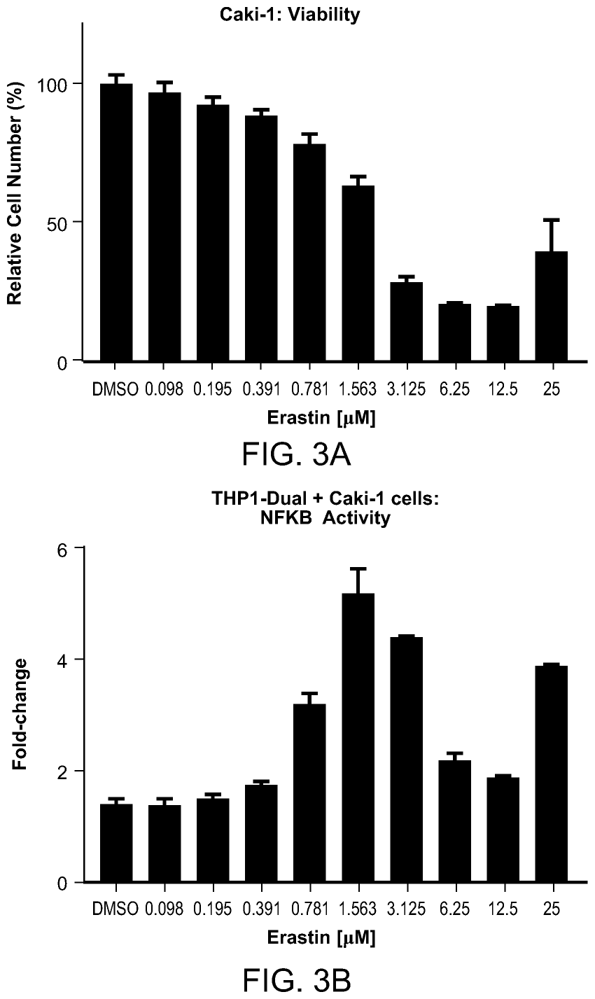

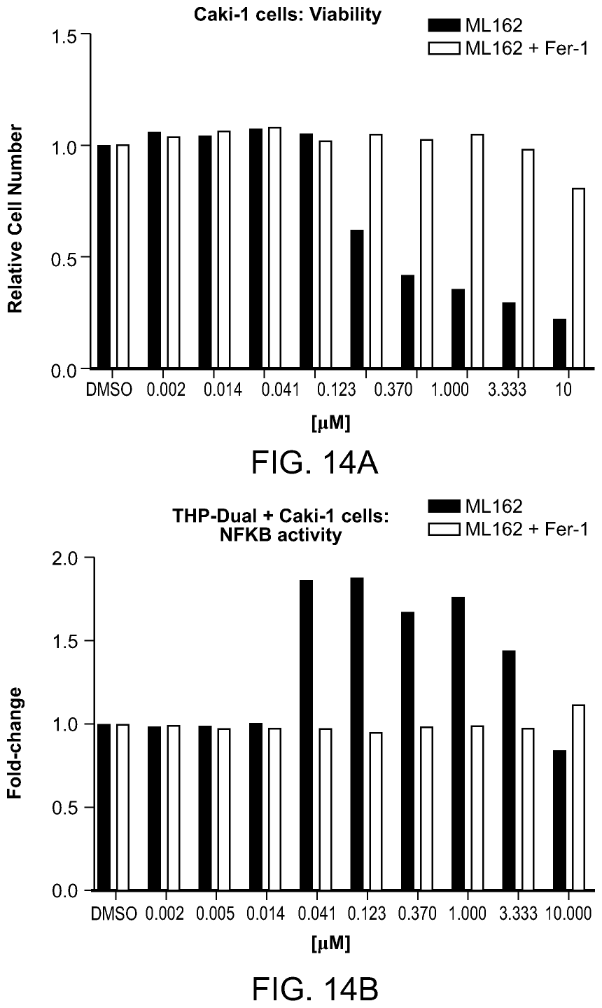

TABLE 1
Erastin Analogs

| Compound | Reference |
|---|---|
| PE | Yang et al., 2014, Cell 156: 317-331; FIG. 1C |
| AE | Yang et al., 2014, Cell 156: 317-331; FIG. 1C |
| IKE | Larraufie, et al., 2015, Bioorg MedChemLetters 25(21): |
| | 4787-4792; FIG. 3 |
| PKE | Larraufie, et al., 2015, Bioorg MedChemLetters 25(21): |
| | 4787-4792; FIG. 3 |
| 35MEW28, | Dixon et al. 2014, eLife Feb 25; 4. doi: |
| 21 | 10.7554/eLife.05608; FIG. 3B/C |
| KE | US 2016/0332974, pages 10-12, paragraph 18 |
| FKE | US 2016/0332974, pages 10-12, paragraph 18 |
| TFKE | US 2016/0332974, pages 10-12, paragraph 18 |
| APKE | US 2016/0332974, pages 10-12, paragraph 18 |
| MKE | US 2016/0332974, pages 10-12, paragraph 18 |
| PMB-PKE | US 2016/0332974, pages 10-12, paragraph 18 |
| MPKE | US 2016/0332974, pages 10-12, paragraph 18 |
| erastin B1 | U.S. Pat. No. 8,535,897, FIG. 14 |
| erastin B2 | U.S. Pat. No. 8,535,897, FIG. 14 |
| erastin A2 | U.S. Pat. No. 8,535,897, FIG. 14 |
| erastin A3 | U.S. Pat. No. 8,535,897, FIG. 14 |

As used herein, unless indicated otherwise, the term “erastin”, includes any pharmaceutically acceptable form of erastin, including, but not limited to, N-oxides, crystalline form, hydrates, salts, esters, and prodrugs thereof. As used herein, the term “erastin derivatives or erastin analogs” refers to compounds having similar structure and function to erastin. In some embodiments, erastin derivatives/erastin analogs include those of the following formula:

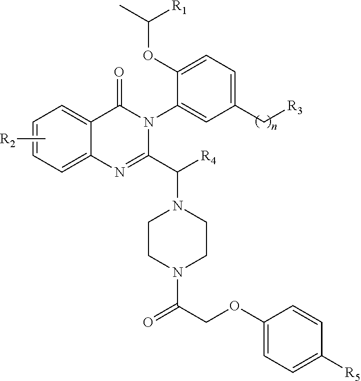

or pharmaceutically acceptable salts or esters thereof, wherein

R₁ is selected from the group consisting of H, C₁₋₄ alkyl, C₁₋₄ alkoxy, hydroxy, and halogen;

R₂ is selected from the group consisting of H, halo, and C₁₋₄ alkyl;

R₃ is selected from the group consisting of H, C₁₋₄ alkyl, C₁₋₄ alkoxy, 5-7 membered heterocycloalkyl, and 5-6 membered heteroaryl;

R₄ is selected from the group consisting of H and C₁₋₄ alkyl;

R₅ is halo;

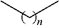

is optionally substituted with ═O; and

n is an integer from 0-4.

In one embodiment, the erastin derivative or analog is a compound represented by Structural Formula (I):

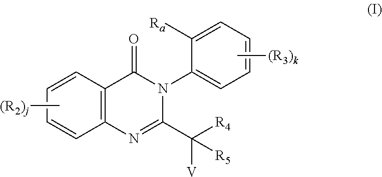

or a pharmaceutically acceptable salt thereof, wherein:

Ra is a halogen, substituted or unsubstituted alkyl, substituted or unsubstituted alkenyl, substituted or unsubstituted alkynyl, substituted or unsubstituted aryl-O—, substituted or unsubstituted alkyl-O—, substituted or unsubstituted alkenyl-O— or substituted or unsubstituted alkynyl-O—, where alkyl, alkenyl and alkynyl are optionally interrupted by NR, O or S(O)ₙ;

each R₂ is independently selected from the group consisting of halogen, substituted or unsubstituted alkyl, substituted or unsubstituted aryl, substituted or unsubstituted non-aromatic heterocyclic, —CN, —COOR′, —CON(R)₂, —NRC (O)R, —SO₂N(R)₂, —N(R)₂, —NO₂, —OH and —OR′;

each R₃ is independently selected from the group consisting of halogen, substituted or unsubstituted alkyl, substituted or unsubstituted aryl, substituted or unsubstituted non-aromatic heterocyclic, —(CO)R, —CN, —COOR′, —CON(R)₂, —NRC (O)R, —SO₂N(R)₂, —N(R)₂, —NO₂, —OH and —OR′;

R₄ and R₅ are independently selected from the group consisting of —H, substituted or unsubstituted alkyl, substituted or unsubstituted alkenyl, substituted or unsubstituted alkynyl, substituted or unsubstituted non-aromatic heterocyclic and substituted or unsubstituted aryl, where alkyl, alkenyl and alkynyl are optionally interrupted by NR, O or S(O)ₙ; or R₄ and R₅ taken together form a carbocyclic or heterocyclic group;

V is —NH-L-A-Q or

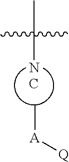

wherein Ring C is a substituted or unsubstituted heterocyclic aromatic or non-aromatic ring;

A is NR or O; or A is a covalent bond;

L is a substituted or unsubstituted hydrocarbyl group optionally interrupted by one or more heteroatoms selected from N, O and S;

Q is selected from the group consisting of —R, —C(O)R′, —C(O)N(R)₂, —C(O)OR′, and —S(O)₂R′;

each R is independently —H, alkyl, alkenyl, alkynyl, aryl, or non-aromatic heterocyclic, wherein said alkyl, alkenyl, alkynyl, aryl, or non-aromatic heterocyclic groups are substituted or unsubstituted;

each R′ is independently an alkyl, alkenyl, alkynyl group, non-aromatic heterocyclic or aryl group, wherein said alkyl, alkenyl, alkynyl, non-aromatic heterocyclic or aryl groups are substituted or unsubstituted;

j is an integer from 0 to 4;

k is an integer from 0 to 4, provided that at least one of j and k is an integer from 1 to 4;

and each n is independently 0, 1 or 2.

In another embodiment, the erastin derivative is a compound represented by Structural Formula (I) as disclosed in the above embodiment; wherein V is

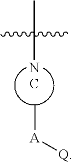

Suitable examples of V encompassed by the above structure include:

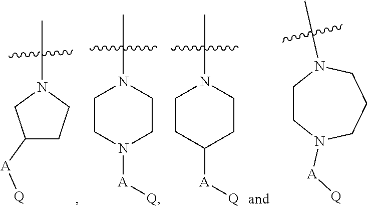

and wherein all other variables are as disclosed in the above mentioned embodiment.

In one embodiment, the erastin derivative or analog is a compound represented by Structural Formula (II):

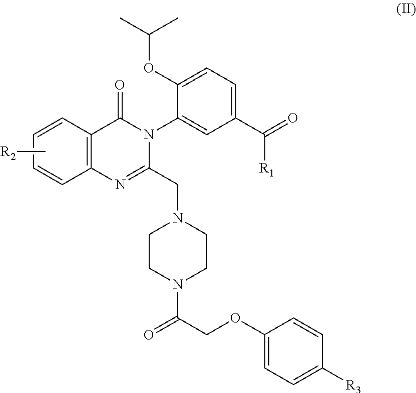

wherein R₁ is selected from the group consisting of H, C₁₋₆ alkyl, and CF₃, wherein each C₁₋₆ alkyl may be optionally substituted with an atom or a group selected from the group consisting of a halogen atom, a saturated or unsaturated C₃₋₆-heterocycle and an amine, each heterocycle optionally substituted with an atom or group selected from the group consisting of C₁₋₄ aliphatic, which C₁₋₄ aliphatic may be optionally substituted with an C₁₋₄ alkyl-aryl-O—C₁₋₄alkyl;

R₂ is selected from the group consisting of H, halo, and C₁₋₆ aliphatic; and

R₃ is a halo atom;

or an N-oxide, crystalline form, hydrate, or pharmaceutically acceptable salt thereof.

In another embodiment, the erastin derivative or analog is a compound represented by Structural Formula (III):

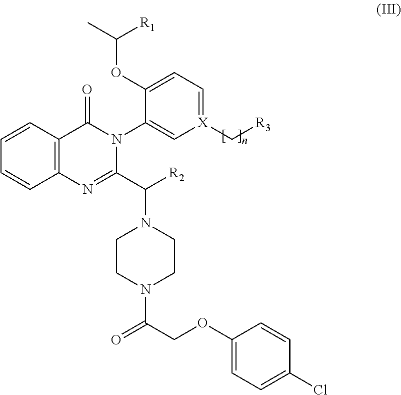

wherein

R₁ is selected from the group consisting of H, C₁₋₄ alkyl, C₁₋₄ alkoxy, hydroxy, and halogen;

R₂ is selected from the group consisting of H, C₁₋₄ alkyl, C₁₋₄ alkoxy, C₃₋₈ cycloalkyl, C₃₋₈ heterocycloalkyl, aryl, heteroaryl, and C₁₋₄ aralkyl;

R₃ is absent, or is selected from the group consisting of C₁₋₄ alkyl, C₁₋₄ alkoxy, carbonyl, C₃₋₈ cycloalkyl, and C₃₋₈ heterocycloalkyl;

X is selected from the group consisting of C, N, and O; and

n is an integer from 0-6,

with the proviso that when X is C, n=0, and R₃ is absent, R₁ cannot be H when R₂ is CH₃;

or an N-oxide, crystalline form, hydrate, or pharmaceutically acceptable salt thereof.

In a particular embodiment, the erastin derivative is a compound represented by Structural Formula (IV):

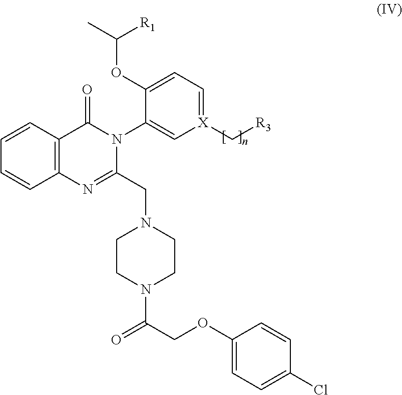

or an N-oxide, crystalline form, hydrate, or pharmaceutically acceptable salt thereof,

wherein the definitions for all the variables are as defined in the above embodiment disclosing compound of formula (III).

In another embodiment, the erastin derivative or analog is a compound represented by Structural Formula (V):

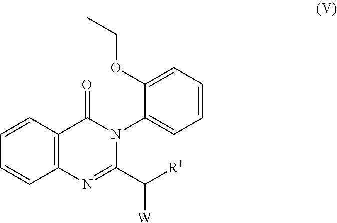

or a pharmaceutically acceptable salt thereof,

wherein R¹ is selected from H, —Z-Q-Z, —C₁₋₈ alkyl-N(R²)(R⁴), —C₁₋₈ alkyl-OR³, 3- to 8-membered carbocyclic or heterocyclic, aryl, heteroaryl, and C₁₋₄aralkyl;

R² and R⁴ are each independently for each occurrence selected from H, C₁₋₄ alkyl, C₁₋₄ aralkyl, aryl, heteroaryl, acyl, alkylsulfonyl, and arylsulfonyl, provided that when both R² and R⁴ are on the same N atom and not both H, they are different, and that when both R² and R⁴ are on the same N and either R² or R⁴ is acyl, alkylsulfonyl, or arylsulfonyl, then the other is selected from H, C₁₋₈ alkyl, C₁₋₄ aralkyl, aryl, and heteroaryl;

R³ is selected from H, C₁₋₄ alkyl, C₁₋₄ aralkyl, aryl, and heteroaryl;

W is selected from

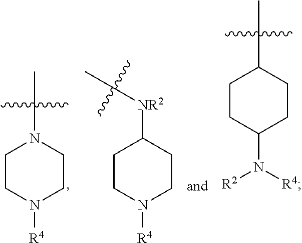

Q is selected from O and NR²; and

Z is independently for each occurrence selected from C₁₋₆ alkyl, C₂₋₆ alkenyl, and C₂₋₆ alkynyl. When Z is an alkenyl or alkynyl group, the double or triple bond or bonds are preferably not at the terminus of the group (thereby excluding, for example, enol ethers, alkynol ethers, enamines and/or ynamines).

In a particular embodiment, the compound is represented by Structural Formula (V) of the above disclosed embodiment; wherein

R² and R⁴ are each independently for each occurrence selected from H, C₁₋₄ alkyl, C₁₋₄ aralkyl, aryl, heteroaryl, acyl, alkylsulfonyl, and arylsulfonyl, provided that when both R² and R⁴ are on the same N atom and not both H, they are different;

R³ is selected from H, C₁₋₄ alkyl, aryl, and heteroaryl;

Z is independently for each occurrence selected from C₁₋₆ alkyl, C₂₋₆ alkenyl, and C₂₋₆ alkynyl;

wherein each heterocyclic group is a 3 to 10 membered non-aromatic ring including one to four heteroatoms selected from nitrogen, oxygen, and sulfur;

wherein each aryl is phenyl;

wherein each heteroaryl is a 5 to 7 membered aromatic ring including one to four heteroatoms selected from nitrogen, oxygen, and sulfur; and

wherein each heterocyclic, aryl, and heteroaryl group is optionally substituted by one or more moieties selected from the group consisting of halogen, hydroxyl, carboxyl, alkoxycarbonyl, formyl, acyl, thioester, thioacetate, thioformate, alkoxyl, phosphoryl, phosphate, phosphonate, phosphinate, amino, amido, amidino, imino, cyano, nitro, azido, sulfhydryl, alkylthio, sulfate, sulfonate, sulfamoyl, and sulfonamido.

The term “substituted” refers to moieties having substituents replacing a hydrogen on one or more carbons of the backbone. Substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

In a particular embodiment, the inhibitor of antiporter system Xc⁻ is

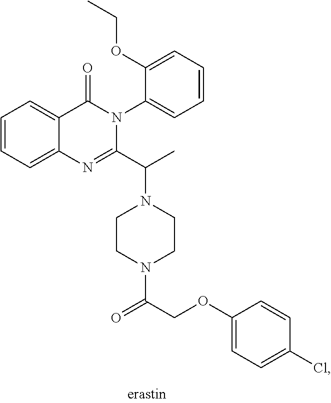 [SMILES: CCOc1ccccc1-n1c(C(C)N2CCN(C(=O)COc3ccc(Cl)cc3)CC2)nc2ccccc2c1=O]

or a pharmaceutically acceptable salt thereof.

In a particular embodiment, the inhibitor of antiporter system Xc⁻ is

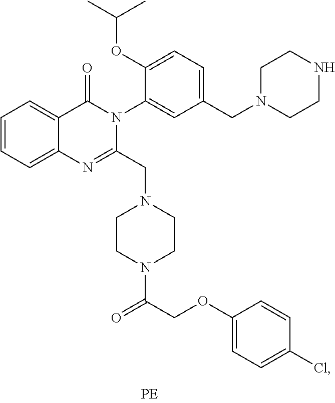 [SMILES: CC(C)Oc1ccc(CN2CCNCC2)cc1-n1c(CN2CCN(C(=O)COc3ccc(Cl)cc3)CC2)nc2ccccc2c1=O]

or a pharmaceutically acceptable salt thereof.

In a particular embodiment, the inhibitor of antiporter system Xc⁻ is

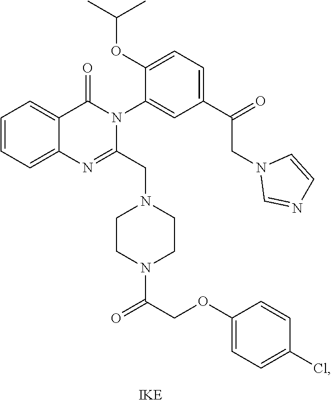 [SMILES: CC(C)Oc1ccc(C(=O)Cn2ccnc2)cc1-n1c(CN2CCN(C(=O)COc3ccc(Cl)cc3)CC2)nc2ccccc2c1=O]

or a pharmaceutically acceptable salt thereof.

Additional erastin derivatives or analogs are described, for example in WO 2015/109009, U.S. Pat. Nos. 9,695,133, 8,535,897, WO 2015/051149, US 2008/0299076, US2007/0161644, WO 2008/013987, U.S. Pat. Nos. 8,575,143, 8,518,959, WO 2007/076085, Bioorganic & Medicinal Chemistry Letters (2015), 25(21), 4787-4792, eLife (2014), 3, Letters in Organic Chemistry (2015), 12(6), 385-393, Pharmacia Lettre (2012), 4(5), 1344-1351, PLoS Pathogens (2014), 10(6), Bioorganic & Medicinal Chemistry Letters (2011), 21(18), 5239-5243, Indian Journal of Chemistry, Section B: Organic Chemistry Including Medicinal Chemistry (2010), 49B(7), 923-928, Synthetic Communications (2009), 39(18), 3217-3231, Indian Journal of Chemistry, Section B: Organic Chemistry Including Medicinal Chemistry (1994), 33B(3), 260-5, Journal of Heterocyclic Chemistry (1983), 20(5), 1339-49, Chemical & Pharmaceutical Bulletin (1979), 27(11), 2675-87, Journal of Medicinal Chemistry (1977), 20(3), 379-86, Indian Journal of Chemistry (1971), 9(3), 201-6, and Journal of Medicinal Chemistry (1968), 11(2), 392-5, each of which is incorporated by reference herein in its entirety.

In some embodiments, an agent that induces iron-dependent cellular disassembly (e.g., ferroptosis) and is useful in the methods provided herein is an inhibitor of glutathione peroxidase 4 (GPX4). GPX4 is a phospholipid hydroperoxidase that catalyzes the reduction of hydrogen peroxide and organic peroxides, thereby protecting cells against membrane lipid peroxidation, or oxidative stress. Thus, GPX4 contributes to a cell's ability to survive in oxidative environments. Inhibition of GPX4 can induce cell death by ferroptosis (see, Yang, W. S., et al. Regulation of ferroptotic cancer cell death by GPX4. Cell 156, 317-331 (2014)). Inhibitors of GPX4 include GPX4-binding proteins (e.g., antibodies or antibody fragments), nucleic acid inhibitors (e.g., antisense oligonucleotides or siRNAs), and small molecules that specifically inhibit GPX4. Small molecule inhibitors of GPX4 include, but are not limited to, the compounds listed in Table 2 below. Each of the references listed in Table 2 is incorporated by reference herein in its entirety.

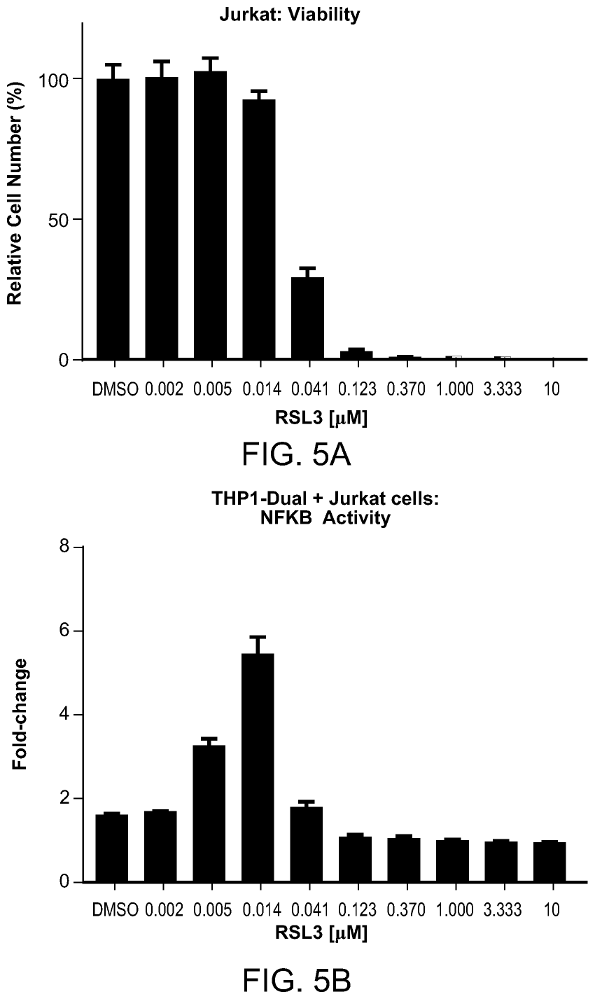

TABLE 2
GPX4 inhibitors

| Compound | Reference |
|---|---|
| DPI7 (ML162), | Yang et al., 2014, Cell 156: 317-331; FIGS. 5 and S5 |
| DPI19, DPI17, | |
| DPI13, DPI12 | |
| DPI10 (ML210) | Cao et al., 2016, Cell Mol Life Sci 73: 2195-2209; |
| | FIG. 2 |
| RSL3, or a | Cao et al., 2016, Cell Mol Life Sci 73: 2195-2209; |
| derivative or | FIG. 2 |
| analog thereof | |
| altretamine | Cao et al., 2016, Cell Mol Life Sci 73: 2195-2209; |
| | FIG. 2 |
| FIN56 | Shimada et al., 2016, Nat. Chem Biol. 12(7): 497-503 |
| FINO2 | Gaschler et al., 2018, Nature Chemical Biology 14: |
| | 507-515 |

In a particular embodiment, the GPX4 inhibitor is

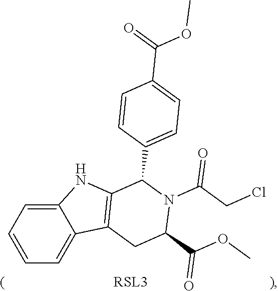 [SMILES: COC(=O)c1ccc([C@H]2c3[nH]c4ccccc4c3C[C@H](C(=O)OC)N2C(=O)CCl)cc1]

or a pharmaceutically acceptable salt thereof.

RSL3 is a known inhibitor of GPX4. In knockdown studies, RSL3 selectively mediated the death of RAS-expressing cells and was identified as increasing lipid ROS accumulation. See U.S. Pat. No. 8,546,421.

In some embodiments, the inhibitor of GPX4 is a diastereoisomer of RSL3.

In a particular embodiment, the diastereoisomer of RSL3 is

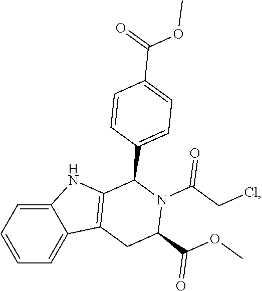 [SMILES: COC(=O)c1ccc([C@@H]2c3[nH]c4ccccc4c3C[C@H](C(=O)OC)N2C(=O)CCl)cc1]

or a pharmaceutically acceptable salt thereof.

In a particular embodiment, the diastereoisomer of RSL3 is

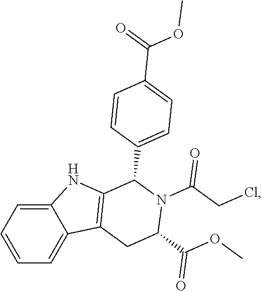 [SMILES: COC(=O)c1ccc([C@H]2c3[nH]c4ccccc4c3C[C@@H](C(=O)OC)N2C(=O)CCl)cc1]

or a pharmaceutically acceptable salt thereof.

In a particular embodiment, the diastereoisomer of RSL3 is

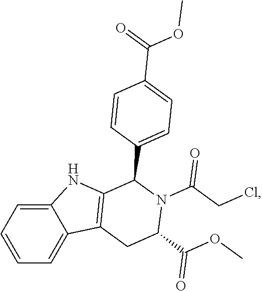 [SMILES: COC(=O)c1ccc([C@@H]2c3[nH]c4ccccc4c3C[C@@H](C(=O)OC)N2C(=O)CCl)cc1]

or a pharmaceutically acceptable salt thereof.

In some embodiments, the inhibitor of GPX4 is a pharmaceutically acceptable form of RSL3, including, but not limited to, N-oxides, crystalline form, hydrates, salts, esters, and prodrugs thereof.

In some embodiments, the inhibitor of GPX4 is RSL3 or a derivative or analog thereof. Derivatives and analogs of RSL3 are known in the art and are described, for example, in WO2008/103470, WO2017/120445, WO2018118711, U.S. Pat. No. 8,546,421, and CN108409737, each of which is incorporated by reference herein in its entirety.

In some embodiments, the RSL3 derivative or analog is a compound represented by Structural Formula (I):

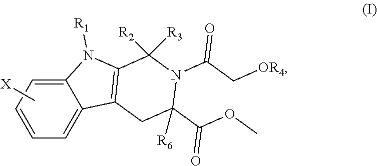

or an enantiomer, optical isomer, diastereomer, N-oxide, crystalline form, hydrate, or pharmaceutically acceptable salt thereof, wherein

R₁, R₂, R₃, and R₆ are independently selected from H, C₁₋₈alkyl, C₁₋₈alkoxy, C₁-8aralkyl, 3- to 8-membered carbocyclic, 3- to 8-membered heterocyclic, 3- to 8-membered aryl, or 3- to 8-membered heteroaryl, acyl, alkylsulfonyl, and arylsulfonyl, wherein each alkyl, alkoxy, aralkyl, carbocyclic, heterocyclic, aryl, heteroaryl, acyl, alkylsulfonyl, and arylsulfonyl is optionally substituted with at least one substituent;

R₄ and R₅ are independently selected from H₁ C₁₋₈alkyl, C₁₋₈alkoxy, 3- to 8-membered carbocyclic, 3- to 8-membered heterocyclic, 3- to 8-membered aryl, or 3- to 8-membered heteroaryl, carboxylate, ester, amide, carbohydrate, amino acid, acyl, alkoxy-substituted acyl, alditol, NR⁷R⁸, OC(R⁷)₂COOH, SC(R⁷)₂COOH, NHCHR⁷COOH, COR⁸, CO₂R⁸, sulfate, sulfonamide, sulfoxide, sulfonate, sulfone, thioalkyl, thioester, and thioether, wherein each alkyl, alkoxy, carbocyclic, heterocyclic, aryl, heteroaryl, carboxylate, ester, amide, carbohydrate, amino acid, acyl, alkoxy-substituted acyl, alditol, NR⁷R⁸, OC(R⁷)₂COOH, SC(R⁷)₂COOH, NHCHR⁷COOH, COR⁸, CO₂R⁸, sulfate, sulfonamide, sulfoxide, sulfonate, sulfone, thioalkyl, thioester, and thioether is optionally substituted with at least one substituent;

R⁷ is selected from H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;

R⁸ is selected from H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent; and

X is 0-4 substituents on the ring to which it is attached.

In one embodiment, the RSL3 derivative or analog is a compound represented by Structural Formula (II):

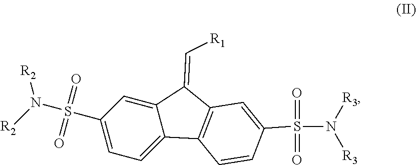

or an N-oxide, crystalline form, hydrate, or pharmaceutically acceptable salt thereof; wherein:

R₁ is selected from the group consisting of H, OH, and —(OCH₂CH₂)ₓOH;

X is an integer from 1 to 6; and

R₂, R₂′, R₃, and R₃′ independently are selected from the group consisting of H, C₃₋₈cycloalkyl, and combinations thereof, or R₂ and R₂′ may be joined together to form a pyridinyl or pyranyl and R₃ and R₃′ may be joined together to form a pyridinyl or pyranyl.

In one embodiment, the RSL3 derivative or analog is a compound represented by Structural Formula (III):

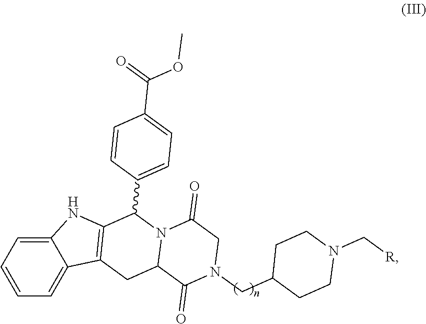

or a stereoisomer thereof, or a pharmaceutically acceptable salt thereof; wherein: n is 2, 3 or 4; and R is a substituted or unsubstituted C₁-C₆ alkyl group, a substituted or unsubstituted C₃-C₁₀ cycloalkyl group, a substituted or unsubstituted C₂-C₈ heterocycloalkyl group, a substituted or unsubstituted C₆-C₁₀ aromatic ring group, or a substituted or unsubstituted C₃-C₈ heteroaryl ring group; wherein the substitution means that one or more hydrogen atoms in each group are substituted by the following groups selected from the group consisting of: halogen, cyano, nitro, hydroxy, C₁-C₆ alkyl, halogenated C₁-C₆ alkyl, C₁-C₆ alkoxy, halogenated C₁-C₆ alkoxy, COOH (carboxy), COOC₁-C₆ alkyl, OCOC₁-C₆ alkyl.

Additional RSL3 derivatives or analogs are described, for example, in US2019/0263802, which is incorporated by reference herein in its entirety. For example, in some embodiments, the RSL3 derivative or analog is a compound represented by Structural Formula (VI):

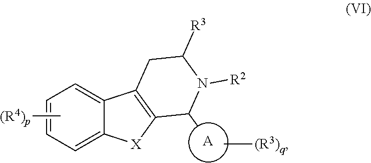

or an enantiomer, optical isomer, diastereomer, N-oxide, crystalline form, hydrate, or pharmaceutically acceptable salt thereof, wherein

ring A is C₄-C₁₀cycloalkyl, heterocyclyl, aryl, or heteroaryl;

X is NR⁵, O or S;

p is 0, 1, 2 or 3;

q is 0, 1, 2 or 3;

R¹ is C₁-C₆alkyl, C₂-C₆alkenyl, C₂-C₆alkynyl, C₁-C₆haloalkyl, C₃-C₁₀cycloalkyl, —CN, —OH, —C(O)OR⁶, —C(O)N(R⁷)₂, —OC(O)R⁶, —S(O)₂R⁸, —S(O)₂N(R⁷)₂, —S(O)N(R⁷)₂, —S(O)R⁸, —NH₂, —NHR⁸, —N(R⁸)₂, —NO₂, —OR⁸, —C₁-C₆alkyl-OH, —C₁-C₆alkyl-OR, or —Si(R¹⁵)₃;

R² is C(O)R⁹;

each R³ is independently halo, —CN, —OH, —OR, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R, —NR¹²C(O)OR⁸, —OC(O)N(R⁷)₂, —OC(O)R⁸, —C(O)R⁶, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R³ is independently optionally substituted with one to three R¹⁰;

each R⁴ is independently halo, —CN, —OH, —OR, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R⁴ is optionally independently optionally substituted with one to three R¹⁰;

R⁵ is hydrogen or C₁-C₆alkyl;

each R⁶ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each R⁶ is independently further substituted with one to three R¹¹;

each R⁷ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₆cycloalkyl, —C₂-C₆alkenylC₃-C₆cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, —C₂-C₆alkenylheteroaryl, or two R⁷ together with the nitrogen atom to which they are attached, form a 4 to 7 membered heterocyclyl; wherein each R⁷ or ring formed thereby is independently further substituted with one to three R¹¹;

each R⁸ is independently C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁸ is independently further substituted with one to three R¹¹;

R⁹ is —C₁-C₂haloalkyl, —C₂-C₃alkenyl, C₂-C₃ haloalkenyl, C₂alkynyl, or —CH₂OS(O)₂-phenyl, wherein the C₁-C₂alkylhalo and —C₂-C₃alkenylhalo are optionally substituted with one or two —CH₃, and the C₂alkynyl and phenyl are optionally substituted with one —CH₃;

each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl, wherein each C₁-C₆alkyl, C₁-C₆haloalkyl, C₂-C₆alkenyl, C₂-C₆alkynyl, C₃-C₁₀cycloalkyl, heterocyclyl, aryl, or heteroaryl of R¹⁰ is optionally independently substituted with one to three R¹¹;

each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl;

each R¹² is independently hydrogen, C₁-C₆alkyl or C₃-C₁₀cycloalkyl;

each R¹³ is independently C₁-C₆alkyl or C₃-C₁₀cycloalkyl; and

each R¹⁵ is independently C₁-C₆alkyl, C₂-C₆alkenyl, aryl, heteroaryl, —C₁-C₆alkylaryl, —C₂-C₆alkenylaryl, —C₁-C₆alkylheteroaryl, and —C₂-C₆alkenylheteroaryl.

In certain embodiments, when X is NR⁵, then R⁹ is C₂alkynyl.

In certain embodiments, when X is NR⁵, and R⁹ is —C₁-C₂haloalkyl, —C₂-C₃alkenyl, —C₂-C₃haloalkenyl, or —CH₂OS(O)₂-phenyl, wherein the C₁-C₂alkylhalo and —C₂-C₃alkenylhalo are optionally substituted with one or two —CH₃, and the phenyl is optionally substituted with —CH₃, then R¹ is other than —C(O)OR⁶ and —C(O)N(R⁷)₂.

In certain embodiments, when X is NR⁵, then (i) R⁹ is C₂alkynyl; or (ii) R⁹ is —C₁-C₂haloalkyl, —C₂-C₃alkenyl, —C₂-C₃haloalkenyl, or —CH₂OS(O)₂-phenyl, wherein the C₁-C₂alkylhalo and —C₂-C₃alkenylhalo are optionally substituted with one or two —CH₃, and the phenyl is optionally substituted with —CH₃, and R¹ is other than —C(O)OR⁶ and —C(O)N(R⁷)₂.

In certain embodiments, when X is NH, R¹ is —C(O)OR⁶, R² is —C(O)CH₂Cl or C(O)CH₂F, q is 1, p is 0, and ring A with the R³ is

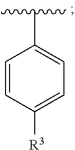

then (i) R³ and R⁶ are not simultaneously —NO₂ and —CH₃, respectively, and (ii) when R⁶ is —CH₃, then R³ is other than H, halo, and —NO₂.

In certain embodiments, when X is NH, R¹ is —C(O)OR⁶, R² is —C(O)CH₂Cl or —C(O)CH₂F, q is 1, p is 0, ring A with the R³ is

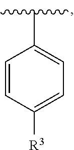

R³, and R³ is —C(O)OR⁶; then both R⁶ are not simultaneously

(i) —CH₃;

(ii) —CH₃ and C₂-C₆alkynyl, respectively; or

(iii) —CH₂CH₃ and —CH₃, respectively.

In certain embodiments, when X is NH, R¹ is —C(O)OCH₃, R² is —C(O)CH₂Cl or —C(O)CH₂F, q is 1, p is 0, and R³ is H; then ring A is other than phenyl.

In certain embodiments, and when X is NH, R¹ is —C(O)N(R⁷)₂, wherein R⁷ are H, R² is —C(O)CH₂Cl or —C(O)CH₂F, q is 0, or 1, p is 0, and ring A is phenyl; then q is not 0, or when q is 1, R³ is other than halo.

In certain embodiments, the compound is not:

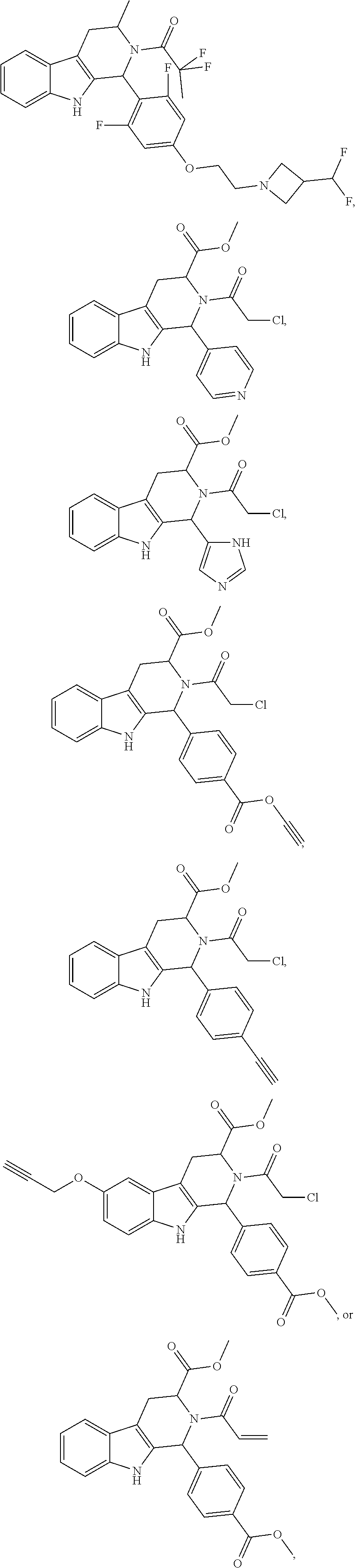

or an enantiomer, optical isomer, diastereomer, N-oxide, crystalline form, hydrate, or pharmaceutically acceptable salt thereof.

In one embodiment, the RSL3 derivative or analog is a compound represented by Structural Formula (VI-1):

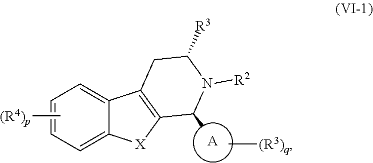

or an enantiomer, optical isomer, diastereomer, N-oxide, crystalline form, hydrate, or pharmaceutically acceptable salt thereof; wherein each of ring A, p, q, R¹, R³, R⁴ and R⁹ are as defined herein.

In one embodiment, the RSL3 derivative or analog is a compound represented by Structural Formula (VI-2):

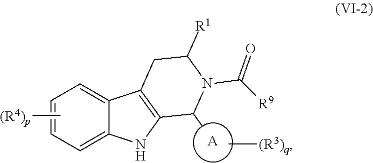

or an enantiomer, optical isomer, diastereomer, N-oxide, crystalline form, hydrate, or a pharmaceutically acceptable salt thereof; wherein each of ring A, p, q, R¹, R³, R⁴ and R⁹ are as defined herein.

In one embodiment, the RSL3 derivative or analog is a compound represented by Structural Formula (VI-2a):

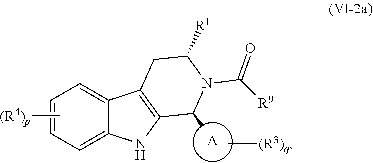

or an enantiomer, optical isomer, diastereomer, N-oxide, crystalline form, hydrate, or a pharmaceutically acceptable salt thereof; wherein each of ring A, p, q, R¹, R³, R⁴ and R⁹ are as defined herein.

In certain embodiments, R⁹ is C₂alkynyl.

In certain embodiments, X is NR⁵, and R⁹ is C₂alkynyl.

In one embodiment, the RSL3 derivative or analog is a compound represented by Structural Formula (VI-3):

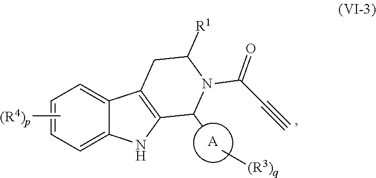

or an enantiomer, optical isomer, diastereomer, N-oxide, crystalline form, hydrate, or a pharmaceutically acceptable salt thereof; wherein each of ring A, p, q, R¹, R³, and R⁴ are as defined herein.

In one embodiment, the RSL3 derivative or analog is a compound represented by Structural Formula (VI-3a):

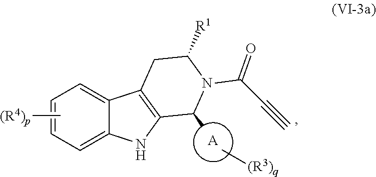

or an enantiomer, optical isomer, diastereomer, N-oxide, crystalline form, hydrate, or a pharmaceutically acceptable salt thereof; wherein each of ring A, p, q, R¹, R³, and R⁴ are as defined herein.

In one embodiment, the RSL3 derivative or analog is a compound represented by Structural Formula (VI-4):

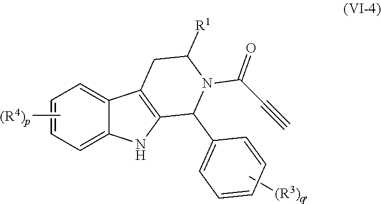

or an enantiomer, optical isomer, diastereomer, N-oxide, crystalline form, hydrate, or a pharmaceutically acceptable salt thereof; wherein each of p, q, R¹, R³, and R⁴ are as defined herein.

In one embodiment, the RSL3 derivative or analog is a compound represented by Structural Formula (VI-4a):

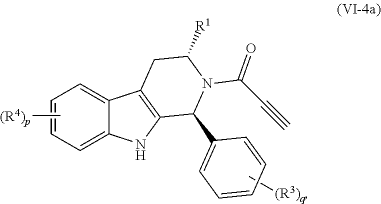

or an enantiomer, optical isomer, diastereomer, N-oxide, crystalline form, hydrate, or a pharmaceutically acceptable salt thereof; wherein each of p, q, R¹, R³, and R⁴ are as defined herein.

In one embodiment, the RSL3 derivative or analog is a compound represented by Structural Formula (VI-5):

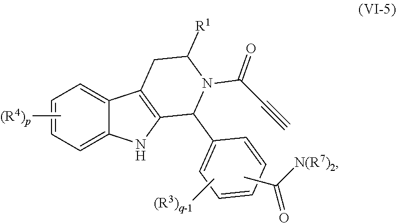

or an enantiomer, optical isomer, diastereomer, N-oxide, crystalline form, hydrate, or a pharmaceutically acceptable salt thereof; wherein each of p, q, R¹, R³, and R⁴ are as defined herein.

In one embodiment, the RSL3 derivative or analog is a compound represented by Structural Formula (VI-5a):

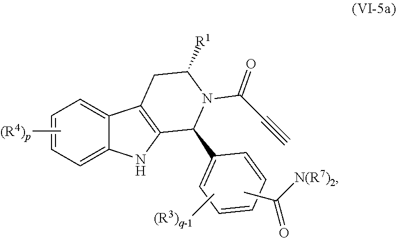

or an enantiomer, optical isomer, diastereomer, N-oxide, crystalline form, hydrate, or a pharmaceutically acceptable salt thereof; wherein each of p, q, R¹, R³, R⁴, and R⁷ are as defined herein.

In one embodiment, the RSL3 derivative or analog is a compound represented by Structural Formula (VI-6):

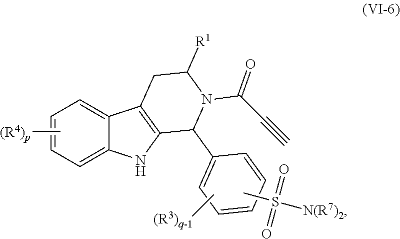

or an enantiomer, optical isomer, diastereomer, N-oxide, crystalline form, hydrate, or a pharmaceutically acceptable salt thereof; wherein each of p, q, R¹, R³, R⁴, and R⁷ are as defined herein.

In one embodiment, the RSL3 derivative or analog is a compound represented by Structural Formula (VI-6a):

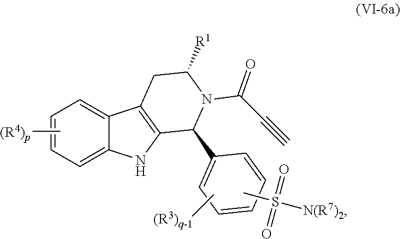

or an enantiomer, optical isomer, diastereomer, N-oxide, crystalline form, hydrate, or a pharmaceutically acceptable salt thereof; wherein each of p, q, R¹, R³, R⁴, and R⁷ are as defined herein.

In certain embodiments of formula (VI-5), (VI-5a), (VI-6) or (VI-6a), the two R¹¹ groups together with the nitrogen atom to which they are attached form a 4 to 7 membered heterocyclyl, wherein the heterocyclyl formed by the two R¹¹ groups is optionally substituted with a 4- to 6-membered heterocyclyl or Structural Formula (VI-5): N(C₁-C₆alkyl)₂, wherein the 4- to 6-membered heterocyclyl when containing 2 or more N atoms is optionally substituted with an N-protecting group. In certain embodiments, the 4 to 7 membered heterocyclyl is selected from azetidinyl, pyrrolidinyl, piperidinyl, pyrazolidinyl, isoxazolidinyl, oxazolidinyl, thiazolidinyl, imidazolidinyl, piperazinyl, morpholinyl, thiomorpholinyl, 1,3-oxazinanyl, 1,3-thiazinanyl, dihydropyridinyl, tetrahydropyranyl, 1,3-tetrahydropyrimidinyl, dihydropyrimidinyl, azepanyl and 1,4-diazepanyl. In certain embodiments, the 4- to 6-membered heterocyclyl, when present as a substituent, is selected from azetidinyl, oxetanyl, thietanyl, piperidinyl, 1,2,3,6-tetrahydropyridinyl, pyranyl, dioxanyl, 1,3-dioxolanyl, dihydropyranyl, dihydrothienyl, dihydrofuranyl, imidazolinyl, pyrrolidinyl, piperidinyl, pyrazolidinyl, isoxazolidinyl, oxazolidinyl, thiazolidinyl, piperazinyl, morpholinyl, thiomorpholinyl, 1,3-oxazinanyl, 1,3-thiazinanyl, dihydropyridinyl, 1,3-tetrahydropyrimidinyl, and dihydropyrimidinyl. In certain embodiments, the N-protecting group when present is t-Boc.

In one embodiment, the RSL3 derivative or analog is a compound represented by Structural Formula (VI-7):

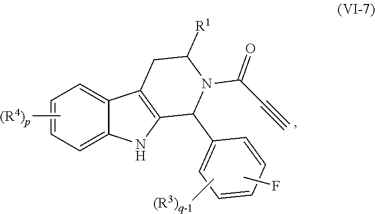

or an enantiomer, optical isomer, diastereomer, N-oxide, crystalline form, hydrate, or a pharmaceutically acceptable salt thereof; wherein each of p, q, R¹, R³, and R⁴ are as defined herein.

In one embodiment, the RSL3 derivative or analog is a compound represented by Structural Formula (VI-7a):

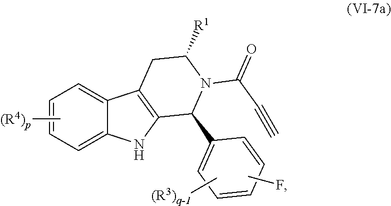

or an enantiomer, optical isomer, diastereomer, N-oxide, crystalline form, hydrate, or a pharmaceutically acceptable salt thereof; wherein each of p, q, R¹, R³, and R⁴ are as defined herein.

In certain embodiments, R¹ is C₁-C₆alkyl, C₂-C₆alkenyl, C₂-C₆alkynyl, C₁-C₆haloalkyl, C₃-C₁₀cycloalkyl, —CN, —C(O)OR⁶, —C(O)N(R⁷)₂, —NH₂, —NHR⁸, —N(R⁸)₂, —OH, —OR⁸, —C₁-C₆alkyl-OH or —C₁-C₆alkyl-OR⁸. In certain embodiments, R¹ is C₁-C₆alkyl, C₂-C₆alkenyl, C₂-C₆alkynyl, C₁-C₆haloalkyl, —C(O)OR⁶, —C(O)N(R⁷)₂, —NH₂, —NHR⁸, —N(R⁸)₂, —OH, —OR⁸, —C₁-C₆alkyl-OH or —C₁-C₆alkyl-OR⁸.

In certain embodiments, R¹ is C₁-C₆alkyl, C₂-C₆alkenyl, C₂-C₆alkynyl, C₁-C₆haloalkyl, —CN, C₃-C₁₀cycloalkyl, —NH₂, —NHR⁸, —N(R⁸)₂, —OH, —OR⁸, —C₁-C₆alkyl-OH or —C₁-C₆alkyl-OR⁸. In certain embodiments, R¹ is C₁-C₆alkyl, C₂-C₆alkenyl, C₂-C₆alkynyl, C₁-C₆haloalkyl, —NH₂, —NHR⁸, —N(R⁸)₂, —OH, —OR⁸, —C₁-C₆alkyl-OH or —C₁-C₆alkyl-OR⁸.

In certain embodiments, R¹ is —C(O)OR⁶ or —C(O)N(R⁷)₂. In certain embodiments, R¹ is C₁-C₆alkyl. In certain embodiments, R¹ is C₃-C₁₀cycloalkyl.

In one embodiment, the RSL3 derivative or analog is a compound represented by Structural Formula (VI-8):

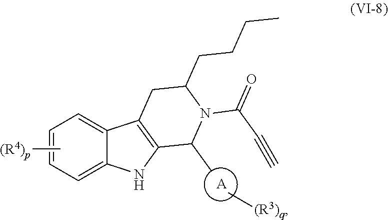

or an enantiomer, optical isomer, diastereomer, N-oxide, crystalline form, hydrate, or a pharmaceutically acceptable salt thereof; wherein each of ring A, p, q, R³, and R⁴ are as defined herein.

In one embodiment, the RSL3 derivative or analog is a compound represented by Structural Formula (VI-8a):

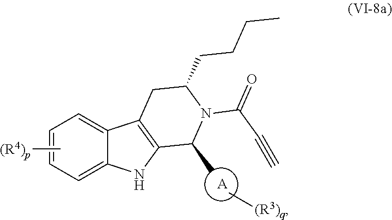

or an enantiomer, optical isomer, diastereomer, N-oxide, crystalline form, hydrate, or a pharmaceutically acceptable salt thereof; wherein each of ring A, p, q, R³, and R⁴ are as defined herein.

In one embodiment, the RSL3 derivative or analog is a compound represented by Structural Formula (VI-9):

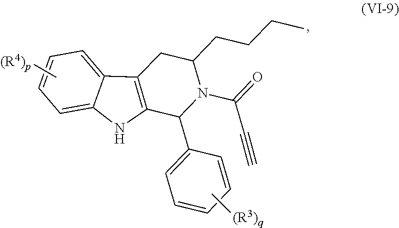

or an enantiomer, optical isomer, diastereomer, N-oxide, crystalline form, hydrate, or a pharmaceutically acceptable salt thereof; wherein each of p, q, R³, and R⁴ are as defined herein.

In one embodiment, the RSL3 derivative or analog is a compound represented by Structural Formula (VI-9a):

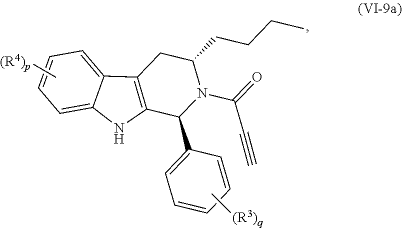

or an enantiomer, optical isomer, diastereomer, N-oxide, crystalline form, hydrate, or a pharmaceutically acceptable salt thereof; wherein each of p, q, R³, and R⁴ are as defined herein.

In certain embodiments, p is 1, 2 or 3. In certain embodiments, p is 1. In certain embodiments, p is 2. In certain embodiments, p is 3.

In certain embodiments, p is 0. In certain embodiments, p is 0 or 1. In certain embodiments, p is 1 or 2.

In certain embodiments, q is 1, 2 or 3. In certain embodiments, q is 1. In certain embodiments, q is 2. In certain embodiments, q is 3. In certain embodiments, q is 0.

In certain embodiments, X is NR⁵ or S.

In certain embodiments, R¹ is C₁-C₆alkyl, C₁-C₆haloalkyl, C₃-C₁₀cycloalkyl, —CN, —C(O)OR⁶, —C(O)N(R⁷)₂, —C₁-C₆alkyl-OH or —C₁-C₆alkyl-OR⁸.

In certain embodiments, at least one R³ is halo, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —C(O)R⁶, or —OC(O)CHR⁸N(R¹²)₂.

In certain embodiments, at least one R³ is halo.

In certain embodiments, at least one R³ is —NHR⁸. In certain embodiments, at least one R³ is —N(R⁸)₂. In certain embodiments, q is 2, and one R³ is halo and the other R³ is —N(R⁸)₂. In certain embodiments, q is 3, and two R³ are independently halo and one R³ is —N(R⁸)₂.

In certain embodiments, at least one R³ is —C(O)OR⁶ or —C(O)R⁶.

In certain embodiments, at least one R³ is —S(O)₂N(R⁷)₂, —S(O)N(R⁷)₂, or —C(O)N(R⁷)₂.

In certain embodiments, at least one R³ is —S(O)₂R⁸, —S(O)R⁸, —NR¹²C(O)R⁸, —NR¹²C(O)OR⁸, —OC(O)R⁸, or —OC(O)CHR⁸N(R¹²)₂.

In certain embodiments, each R³ is independently halo, —CN, —OR, —NHR⁸, —S(O)₂R⁸, —S(O)₂N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —OC(O)CHR⁸N(R¹²)₂, C₁-C₆alkyl, C₃-C₁₀cycloalkyl, heterocyclyl, heteroaryl, or —C₁-C₆alkylheterocyclyl; wherein each C₁-C₆alkyl, C₃-C₁₀cycloalkyl, heterocyclyl, heteroaryl, or C₁-C₆alkylheterocyclyl of R³ is independently optionally substituted with one to three R¹⁰.

In certain embodiments, each R³ is independently halo, —CN, —OR⁸, —NHR⁸, —S(O)₂R⁸, —S(O)₂N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —OC(O)CHR⁸N(R¹²)₂, C₁-C₆alkyl, C₃-C₁₀cycloalkyl, heterocyclyl, heteroaryl, or —C₁-C₆alkylheterocyclyl; wherein each C₁-C₆alkyl, C₃-C₁₀cycloalkyl, heterocyclyl, heteroaryl, or C₁-C₆alkylheterocyclyl is independently optionally substituted with one to three substituents independently selected from —OR¹², —N(R¹²)₂, —S(O)₂R¹³, —OC(O)CHR¹²N(R¹²)₂, and C₁-C₆alkyl optionally substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein

each R¹² is independently hydrogen, C₁-C₆alkyl or C₃-C₁₀cycloalkyl; and

each R¹³ is independently C₁-C₆alkyl or C₃-C₁₀cycloalkyl.

In certain embodiments, each R⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R, —OC(O)R⁸, —C(O)R⁶, C₁-C₆alkyl, C₂-C₆alkenyl, C₂-C₆alkynyl, or C₃-C₁₀cycloalkyl; wherein each C₁-C₆alkyl, C₂-C₆alkenyl, C₂-C₆alkynyl, or C₃-C₁₀cycloalkyl of R⁴ is independently optionally substituted with one to three R¹⁰. In certain embodiments, each R⁴ is independently halo, —CN, —OH, —OR⁸, C₁-C₆alkyl, C₂-C₆alkynyl, or C₃-C₁₀cycloalkyl; wherein each C₁-C₆alkyl, C₂-C₆alkynyl, or C₃-C₁₀cycloalkyl of R⁴ is independently optionally substituted with one to three R¹⁰.

In certain embodiments, each R⁴ is independently halo, —CN, —OH, —OR⁸, C₁-C₆alkyl, or C₂-C₆alkynyl; wherein the C₁-C₆alkyl of R⁴ is optionally substituted with one to three R¹⁰.

In certain embodiments, each R⁴ is independently halo, —CN, —OH, —OR⁸, C₁-C₆alkyl, C₂-C₆alkynyl; wherein the C₁-C₆alkyl of R⁴ is optionally substituted with one to three substituents independently selected from —OR¹², —N(R¹²)₂, —S(O)₂R¹³, —OC(O)CHR¹²N(R¹²)₂, and C₁-C₆alkyl optionally substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein

each R¹² is independently hydrogen, C₁-C₆alkyl or C₃-C₁₀cycloalkyl; and

each R¹³ is independently C₁-C₆alkyl or C₃-C₁₀cycloalkyl.

In certain embodiments, each R⁶ is independently hydrogen, C₁-C₆alkyl, C₂-C₆alkenyl, or C₁-C₆alkylC₃-C₁₀cycloalkyl; wherein each R⁶ is independently further substituted with one to three R¹¹.

In certain embodiments, each R⁶ is independently hydrogen, C₁-C₆alkyl, C₂-C₆alkenyl, or —C₁-C₆alkylC₃-C₁₀cycloalkyl; wherein each R⁶ is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein

each R¹² is independently hydrogen, C₁-C₆alkyl or C₃-C₁₀cycloalkyl.

In certain embodiments, each R⁷ is independently hydrogen, C₁-C₆alkyl, C₃-C₁₀cycloalkyl, heterocyclyl, heteroaryl, —C₁-C₆alkylC₃-C₆cycloalkyl, —C₁-C₆alkylheterocyclyl, or two R⁷ together with the nitrogen atom to which they are attached, form a 4 to 7 membered heterocyclyl; wherein each R⁷ or ring formed thereby is independently further substituted with one to three R¹¹.

In certain embodiments, each R⁷ is independently hydrogen, C₁-C₆alkyl, C₃-C₁₀cycloalkyl, heterocyclyl, heteroaryl, —C₁-C₆alkylC₃-C₆cycloalkyl, —C₁-C₆alkylheterocyclyl, or two R⁷ together with the nitrogen atom to which they are attached, form a 4 to 7 membered heterocyclyl; wherein each R⁷ or ring formed thereby is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein

each R¹² is independently hydrogen, C₁-C₆alkyl or C₃-C₁₀cycloalkyl.

In certain embodiments, each R⁸ is independently C₁-C₆alkyl, C₂-C₆alkynyl, C₃-C₁₀cycloalkyl, —C₁-C₆alkylC₃-C₁₀cycloalkyl, or —C₁-C₆alkylaryl; wherein each R⁸ is independently further substituted with one to three R¹¹.

In certain embodiments, each R⁸ is independently C₁-C₆alkyl, C₂-C₆alkynyl, C₃-C₁₀cycloalkyl, —C₁-C₆alkylC₃-C₁₀cycloalkyl, or —C₁-C₆alkylaryl; wherein each R⁸ is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein

each R¹² is independently hydrogen, C₁-C₆alkyl or C₃-C₁₀cycloalkyl.

In certain embodiments, each R¹⁰ is independently —OR¹², —N(R¹²)₂, —S(O)₂R¹³, —OC(O)CHR¹²N(R¹²)₂, or C₁-C₆alkyl, wherein the C₁-C₆alkyl, of R¹⁰ is optionally independently substituted with one to three R¹¹;

each R¹¹ is independently halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl;

each R¹² is independently hydrogen, C₁-C₆alkyl or C₃-C₁₀cycloalkyl; and

each R¹³ is independently C₁-C₆alkyl or C₃-C₁₀cycloalkyl.

In certain embodiments, ring A is C₄-C₁₀cycloalkyl, heterocyclyl, aryl, or heteroaryl;

X is NR⁵ or S;

p is 0, 1, 2 or 3;

q is 0, 1, 2 or 3;

R¹ is C₁-C₆alkyl, C₁-C₆haloalkyl, C₃-C₁₀cycloalkyl, —CN, —C(O)OR⁶, —C(O)N(R⁷)₂, —C₁-C₆alkyl-OH or —C₁-C₆alkyl-OR⁸;

R² is —C(O)R⁹;

each R³ is independently halo, —CN, —OR, —NHR⁸, —S(O)₂R⁸, —S(O)₂N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —OC(O)CHR⁸N(R¹²)₂, C₁-C₆alkyl, C₃-C₁₀cycloalkyl, heterocyclyl, heteroaryl, or —C₁-C₆alkylheterocyclyl; wherein each C₁-C₆alkyl, C₃-C₁₀cycloalkyl, heterocyclyl, heteroaryl, or C₁-C₆alkylheterocyclyl of R³ is independently optionally substituted with one to three R¹⁰;

each R⁴ is independently halo, —CN, —OH, —OR, C₁-C₆alkyl, or C₂-C₆alkynyl; wherein the C₁-C₆alkyl of R⁴ is optionally independently optionally substituted with one to three R¹⁰;

R⁵ is hydrogen or C₁-C₆alkyl;

each R⁶ is independently hydrogen, C₁-C₆alkyl, C₂-C₆alkenyl, or C₁-C₆alkylC₃-C₁₀cycloalkyl; wherein each R⁶ is independently further substituted with one to three R¹¹;

each R⁷ is independently hydrogen, C₁-C₆alkyl, C₃-C₁₀cycloalkyl, heterocyclyl, heteroaryl, —C₁-C₆alkylC₃-C₆cycloalkyl, —C₁-C₆alkylheterocyclyl, or two R⁷ together with the nitrogen atom to which they are attached, form a 4 to 7 membered heterocyclyl; wherein each R⁷ or ring formed thereby is independently further substituted with one to three R¹¹;

each R⁸ is independently C₁-C₆alkyl, C₂-C₆alkynyl, C₃-C₁₀cycloalkyl, —C₁-C₆alkylC₃-C₁₀cycloalkyl, or —C₁-C₆alkylaryl; wherein each R⁸ is independently further substituted with one to three R¹¹;

R⁹ is —C₁-C₂haloalkyl, —C₂-C₃alkenyl, —C₂-C₃haloalkenyl, C₂alkynyl, or —CH₂OS(O)₂-phenyl, wherein the C₁-C₂alkylhalo and —C₂-C₃alkenylhalo are optionally substituted with one or two —CH₃, and the C₂alkynyl and phenyl are optionally substituted with one —CH₃;

each R¹⁰ is independently —OR¹², —N(R¹²)₂, —S(O)₂R¹³, —OC(O)CHR¹²N(R¹²)₂, or C₁-C₆alkyl, wherein the C₁-C₆alkyl, of R¹⁰ is optionally independently substituted with one to three R¹¹;

each R¹¹ is independently halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl;

each R¹² is independently hydrogen, C₁-C₆alkyl or C₃-C₁₀cycloalkyl; and

each R¹³ is independently C₁-C₆alkyl or C₃-C₁₀cycloalkyl.

In certain embodiments, each R¹⁵ is independently C₁-C₆alkyl.

In some embodiments, the RSL3 derivative or analog is a compound represented by Structural Formula (VII):

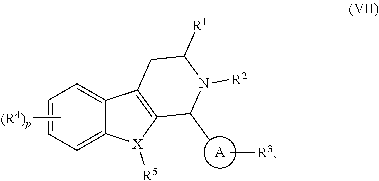

or an enantiomer or pharmaceutically acceptable salt thereof, wherein:

X is N, O or S;

A is a 4 to 7 membered cycloalkyl, 4 to 7 membered heterocyclyl, aryl, heteroaryl, or bridged bicyclic ring having 0-2 heteroatoms independently selected from nitrogen, oxygen, and sulfur;

R¹ is H, —C₁-C₆alkyl, —C₁-C₆alkylhalo, —C(O)OR⁶, —C(O)N(R⁷)₂, —OC(O)R⁶, —SO₂R⁸, —SOR⁸, —NO₂, —OR, —C₁-C₆alkyl-OR¹², or —Si(R¹⁵)—;

R² is —C(O)R⁹;

R³ is H, halo, —C(O)OR¹⁰, —C(O)N(R¹¹)₂, O—C(O)R¹⁰, —C₀-C₆alkylC₃-C₈cycloalkyl, —C₀-C₆alkylheterocyclyl, —N(R¹¹)₂, —SO₂R⁸, —SOR⁸, —NO₂ or —Si(R¹⁵)₃;

R⁴ is independently halo, CN, —NH₂, —SO₂, C₁-C₈alkyl, —OR¹², —C₁-C₆alkyl-OR¹², —C₁-C₆alkyl-NR¹² or —OC(O)R¹²;

R⁵ is H, C₁-C₆alkyl, or is absent when X is S or O;

p is 0, 1, 2 or 3;

R⁶ is C₁-C₆alkyl, C₃-C₆alkyl, C₂-C₆alkenyl, C₂-C₆alkynyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, (R¹¹)₂NC₁-C₆alkyl-, or (R¹¹)₂NC₂-C₆alkenyl;

each R⁷ is independently H, C₁-C₆alkyl, C₂-C₆alkenyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, (R¹¹)₂NC₁-C₆alkyl-, (R¹¹)₂NC₂-C₆alkenyl-, R¹²O—C₁-C₆alkyl-, or R¹²O(O)C—C₁-C₆alkyl-, or two R⁷ together with the nitrogen atom to which they are attached form a 4 to 7 membered heterocyclyl, wherein the heterocyclyl formed by the two R⁷ groups is optionally substituted with OH, halo, C₁-C₆alkyl, a 4- to 6-membered heterocyclyl, or (R¹¹)₂N—, wherein the 4- to 6-membered heterocyclyl when containing 2 or more N atoms is optionally substituted with an N-protecting group;

each R⁸ is independently C₁-C₆alkyl, C₃-C₆alkyl, C₂-C₆alkenyl, C₂-C₆alkynyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, (R¹¹)₂NC₁-C₆alkyl-, (R¹¹)₂N—, or R¹⁴C₀-C₆alkyl-;

R⁹ is —C₁-C₂alkylhalo, —C₂-C₃alkenylhalo, or C₂alkynyl, wherein the C₁-C₂alkyl is optionally substituted with one or two halo, one or two —CH₃;

R¹⁰ is C₁-C₆alkyl, C₂-C₆alkyl, C₂-C₆alkenyl, C₂-C₆alkynyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, (R¹¹)₂NC₁-C₆alkyl-, (R¹¹)₂NC₂-C₆alkenyl-, R¹³(NH₂)CH—, R¹⁴C₀-C₆alkyl- or (R¹⁵)₃SiC₀-C₆alkyl;

each R¹¹ is independently H, C₁-C₆alkyl, C₂-C₆alkenyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, R¹²O—C₁-C₆alkyl-, (R¹⁶)₂NC₁-C₆alkyl-, (R¹⁶)₂NC₂-C₆alkenyl-, R¹²O(O)C—C₁-C₆alkyl-, R¹³(NH₂)CH—, R¹⁴C₀-C₆alkyl-, (R¹⁵)₃SiC₀-C₆alkyl-, or an N-protecting group; or two R¹¹ together with the nitrogen atom to which they are attached form a 4 to 7 membered heterocyclyl, wherein the heterocyclyl formed by the two R¹¹ groups has optionally 0, 1 or 2 additional heteroatoms selected from nitrogen, oxygen, and sulfur, and the heterocyclyl is optionally substituted with OH, halo, C₁-C₆alkyl, C₁-C₆alkyl-O(O)C—, (R¹⁶)₂N—, or a 4- to 6-membered heterocyclyl, wherein the 4- to 6-membered heterocyclyl is optionally substituted with OH, halo, —NH₂, or C₁-C₆alkyl, or when containing 2 or more N atoms is optionally substituted with an N-protecting group;

each R¹² is independently H or C₁-C₆alkyl;

each R¹³ is independently H, C₁-C₆alkyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, or an N protecting group;

R¹⁴ is a bridged bicyclic ring having 0-2 heteroatoms independently selected from nitrogen, oxygen, and sulfur; and

each R¹⁵ is independently C₁-C₆alkyl, C₂-C₆alkenyl, aryl, heteroaryl, arylC₁-C₆alkyl-, arylC₂-C₆alkenyl-, heteroarylC₁-C₆alkyl-, and heteroarylC₂-C₆alkenyl-;

wherein the C₁-C₆alkyl, C₃-C₆cycloalkyl, heterocyclyl, aryl, heteroaryl, or bridged bicyclic ring, by itself or attached to another moiety, are independently optionally substituted with 1-3 substituents selected from the group consisting of OH, halo, —NH₂, C₁-C₆alkyl, C₁-C₆alkyl-O—, R¹²O—C₁-C₆alkyl(O)C—, and R¹²O(O)C—;

each R¹⁶ is independently H, C₁-C₆alkyl, C₂-C₆alkenyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, R¹²O—C₁-C₆alkyl-, (R¹³)₂NC₁-C₆alkyl-, (R¹³)₂NC₂-C₆alkenyl-, R¹²O(O)C—C₁-C₆alkyl-, R¹³(NH₂)CH—, R¹⁴C₀-C₆alkyl-, (R¹⁵)₃SiC₀-C₆alkyl-, or an N-protecting group; or two R¹⁶ together with the nitrogen atom to which they are attached form a 4 to 7 membered heterocyclyl, wherein the heterocyclyl formed by the two R¹⁶ groups has optionally 0, 1 or 2 additional heteroatoms selected from nitrogen, oxygen, and sulfur, and the heterocyclyl is optionally substituted with OH, halo, C₁-C₆alkyl, C₁-C₆alkyl-O(O)C—, (R¹³)₂N—, or a 4- to 6-membered heterocyclyl, wherein the 4- to 6-membered heterocyclyl is optionally substituted with OH, halo, —NH₂, or C₁-C₆alkyl, or when containing 2 or more N atoms is optionally substituted with an N-protecting group;

with the proviso that:

(a) when

X is N;

R¹ is —C(O)OR⁶

R² is —C(O)CH₂Cl or C(O)CH₂F;

A with the R³ is

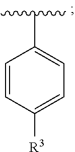

p is 0; and

R⁵ is H;

then (i) R³ and R⁶ are not simultaneously-NO₂ and —CH₃, respectively, and (ii) when R⁶ is —CH₃, then R³ is other than H, halo, and —NO₂; and

(b) when

X is N;

R¹ is —C(O)OR⁶

R² is —C(O)CH₂Cl or C(O)CH₂F;

A with the R³ is

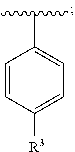

R³ is C(O)OR¹⁰;

p is 0; and

R⁵ is H;

then R⁶ and R¹⁰ are not simultaneously

(i) —CH₃;

(ii) —CH₃ and C₂-C₆alkynyl, respectively; and

(iii) —CH₂CH₃ and —CH₃, respectively; and

(c) when

X is N;

R¹ is —C(O)OR⁶, wherein R⁶ is —CH₃;

R² is —C(O)CH₂Cl or —C(O)CH₂F;

p is 0;

R³ is H; and

R⁵ is H;

then ring A is other than phenyl; and

(d) when

X is N;

R¹ is —C(O)N(R⁷)₂, wherein R⁷ are H;

R² is —C(O)CH₂Cl or —C(O)CH₂F;

p is 0;

R⁵ is H; and

ring A is phenyl;

then R³ is other than H or halo.

In certain embodiments, ring A is aryl or heteroaryl. In certain embodiments, ring A is a monocyclic aryl or monocyclic heteroaryl. In certain embodiments, ring A is heterocyclyl. In certain embodiments, ring A is a 4 to 7 membered heterocyclyl. In certain embodiments, ring A is aryl. In certain embodiments, ring A is phenyl. In certain embodiments, ring A is heteroaryl. In certain embodiments, ring A is pyridyl. In certain embodiments, ring A is phenyl, pyridyl, piperidynyl, piperazinyl, or morpholinyl.

In certain embodiments, ring A is aryl or heteroaryl, each of which is substituted by one to three R³. In certain embodiments, ring A is aryl or heteroaryl, each of which is substituted by one to three R³, where at least one R³ is C₃-C₁₀cycloalkyl, heterocyclyl, aryl, or heteroaryl; wherein each C₃-C₁₀cycloalkyl, heterocyclyl, aryl, and heteroaryl of R³ is optionally substituted with one to three R¹⁰.

In certain embodiments, ring A is aryl or heteroaryl, each of which is substituted by two or three R³. In certain embodiments, ring A is aryl or heteroaryl, each of which is substituted by two or three R³; wherein at least one R³ is halo.

In certain embodiments, ring A is:

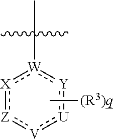

wherein 0 to 3 of U, V, W, X, Y, and Z is independently N, S, or O, and each

[symbol]

independently represents a single or double bond, which comply with valency requirements based on U, V, W, X, Y and Z.

In certain embodiments, ring A is:

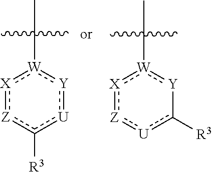

wherein 1 to 3 of U, W, X, Y, and Z is N, S, or O, and

[symbol]

represents a single or double bond, which comply with valency requirements based on U, W, X, Y and Z.

In certain embodiments, ring A is cyclohexyl. In certain embodiments, ring A is C₄-C₁₀cycloalkyl, substituted with one to three R³. In certain embodiments, ring A is a C₄-C₇cycloalkyl, substituted with one to three R³. In certain embodiments, ring A is bicyclo[1.1.1]pentanyl, substituted with one to three R³. In certain embodiments, ring A is selected from cyclobutyl, cyclopentyl, cyclohexyl, and cycloheptyl, wherein each is substituted with one to three R³.

In certain embodiments, ring A is cyclohexyl. In certain embodiments, ring A is C₄-C₁₀cycloalkyl. In certain embodiments, ring A is a C₄-C₇cycloalkyl. In certain embodiments, ring A is bicyclo[1.1.1]pentanyl. In certain embodiments, ring A is selected from cyclobutyl, cyclopentyl, cyclohexyl, and cycloheptyl.

In certain embodiments, ring A is:

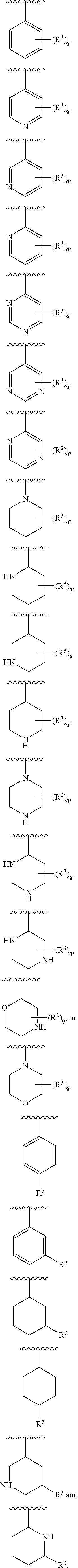

where q and each R³ is independently as defined herein.

In certain embodiments, ring A is selected from:

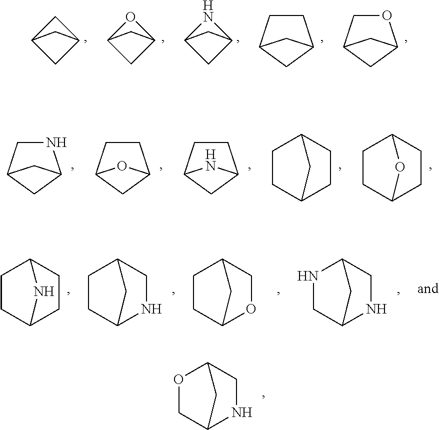

In certain embodiments, ring A is a bridged bicyclic ring selected from:

wherein each is substituted with one to three R³. In certain embodiments, ring A is a bridged bicyclic ring selected from:

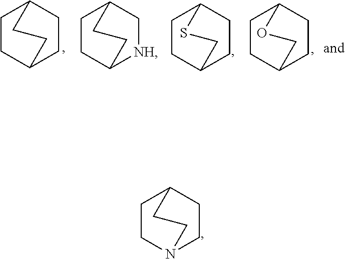

wherein each R³ is attached to a carbon atom on the bridged bicyclic ring.

In certain embodiments, ring A is a bridged bicyclic ring selected from:

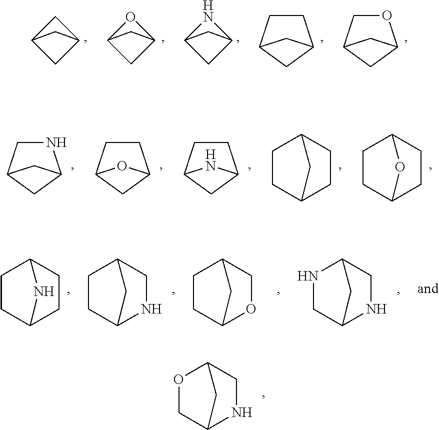

and wherein R³ is attached to a carbon atom on the bridged bicyclic ring. In certain embodiments, ring A is a bridged bicyclic ring selected from:

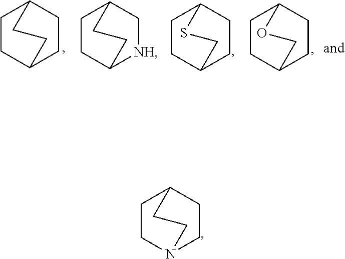

In certain embodiments, ring A is:

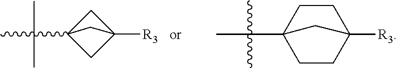

In certain embodiments, at least one R³ is —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R, —C(O)R⁶, or —OC(O)CHR⁸N(R¹²)₂.

In certain embodiments, at least one R³ is —NHR⁸ or —N(R)₂.

In certain embodiments, at least one R³ is —C(O)OR⁶ or —C(O)R⁶.

In certain embodiments, at least one R³ is —S(O)₂N(R⁷)₂, —S(O)N(R⁷)₂, or —C(O)N(R⁷)₂.

In certain embodiments, at least one R³ is —S(O)₂R⁸, —S(O)R⁸, —NR¹²C(O)R⁸, —NR¹²C(O)OR⁸, —OC(O)R, or —OC(O)CHR⁸N(R¹²)₂.

In one embodiment, the RSL3 derivative or analog is a compound represented by Structural Formula (VII-1):

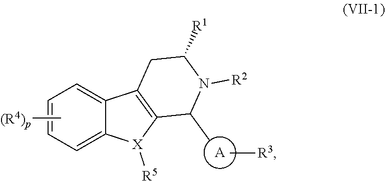

or an enantiomer or pharmaceutically acceptable salt thereof; wherein each of ring A, R¹, R², R³, R⁴, and R⁵ are as defined herein.

In certain embodiments, each of the compounds described herein can have the stereochemical structure depicted for formula (VII-1).

In one embodiment, the RSL3 derivative or analog is a compound represented by Structural Formula (VII-2):

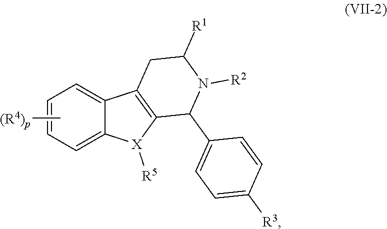

or an enantiomer or pharmaceutically acceptable salt thereof; wherein:

X is N, O or S;

R¹ is H, C₁-C₆alkyl, —C₁-C₆alkylhalo, —C(O)OR⁶, —C(O)N(R⁷)₂, —OC(O)R⁶, —SO₂R⁸, —SOR⁸, —NO₂, —OR⁸, —C₁-C₆alkyl-OR¹², or —Si(R¹⁵)₃;

R² is —C(O)R⁹;

R³ is —C(O)OR¹⁰, —C(O)N(R¹¹)₂, —OC(O)R¹⁰, —C₀-C₆alkylC₃-C₈cycloalkyl, —C₀-C₆alkylheterocyclyl, —N(R¹¹)₂, —SO₂R⁸, —SOR⁸, —NO₂ or —Si(R¹⁵)₃;

R⁴ is independently halo, CN, —NH₂, —SO₂, C₁-C₈alkyl, —OR¹², —C₁-C₆alkyl-OR¹², —C₁-C₆alkyl-NR¹² or —OC(O)R¹²;

R⁵ is H, C₁-C₆alkyl, or is absent when X is S or O;

p is 0, 1, 2 or 3;

R⁶ is C₁-C₆alkyl, C₃-C₆alkyl, C₂-C₆alkenyl, C₂-C₆alkynyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, (R¹¹)₂NC₁-C₆alkyl-, or (R¹¹)₂NC₂-C₆alkenyl;

each R⁷ is independently H, C₁-C₆alkyl, C₂-C₆alkenyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, (R¹¹)₂NC₁-C₆alkyl-, (R¹¹)₂NC₂-C₆alkenyl-, R¹²—O—C₁-C₆alkyl-, or R¹²O(O)C—C₁-C₆alkyl-, or two R⁷ together with the nitrogen atom to which they are attached form a 4 to 7 membered heterocyclyl, wherein the heterocyclyl formed by the two R⁷ groups is optionally substituted with OH, halo, C₁-C₆alkyl, a 4- to 6-membered heterocyclyl, or (R¹¹)₂N—, wherein the 4- to 6-membered heterocyclyl when containing 2 or more N atoms is optionally substituted with an N-protecting group;

each R⁸ is independently C₁-C₆alkyl, C₃-C₆alkyl, C₂-C₆alkenyl, C₂-C₆alkynyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, (R¹¹)₂NC₁-C₆alkyl-, (R¹¹)₂N—, or R¹⁴C₀-C₆alkyl-;

R⁹ is —C₁-C₂alkylhalo, —C₂-C₃alkenylhalo, or C₂alkynyl, wherein the C₁-C₂alkyl is optionally substituted with one or two halo, one or two —CH₃;

R¹⁰ is C₁-C₆alkyl, C₂-C₆alkyl, C₂-C₆alkenyl, C₂-C₆alkynyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, (R¹¹)₂NC₁-C₆alkyl-, (R¹¹)₂NC₂-C₆alkenyl-, R¹³(NH₂)CH—, R¹⁴C₀-C₆alkyl-, or (R¹⁵)₃SiC₀-C₆alkyl-;

each R¹¹ is independently H, C₁-C₆alkyl, C₂-C₆alkenyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, R¹²O—C₁-C₆alkyl-, (R¹¹)₂NC₁-C₆alkyl-, (R¹¹)₂NC₂-C₆alkenyl-, R¹²O(O)C—C₁-C₆alkyl-, R¹³(NH₂)CH—, R¹⁴C₀-C₆alkyl-, (R¹⁵)₃SiC₀-C₆alkyl-, or an N-protecting group; or two R¹¹ together with the nitrogen atom to which they are attached form a 4 to 7 membered heterocyclyl, wherein the heterocyclyl formed by the two R¹¹ groups has optionally 0, 1 or 2 additional heteroatoms selected from nitrogen, oxygen, and sulfur, and the heterocyclyl is optionally substituted with OH, halo, C₁-C₆alkyl, C₁-C₆alkyl-O(O)C—, (R¹¹)₂N—, or a 4- to 6-membered heterocyclyl, wherein the 4- to 6-membered heterocyclyl is optionally substituted with OH, halo, —NH₂, or C₁-C₆alkyl, or when containing 2 or more N atoms is optionally substituted with an N-protecting group;
each R¹² is independently H or C₁-C₆alkyl;
each R¹³ is independently H, C₁-C₆alkyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, or an N protecting group;

R¹⁴ is a bridged bicyclic ring having 0-2 heteroatoms independently selected from nitrogen, oxygen, and sulfur; and

each R¹⁵ is independently C₁-C₆alkyl, C₂-C₆alkenyl, aryl, heteroaryl, arylC₁-C₆alkyl-, arylC₂-C₆alkenyl-, heteroarylC₁-C₆alkyl-, and heteroarylC₂-C₆alkenyl-;

wherein the C₁-C₆alkyl, C₃-C₆cycloalkyl, heterocyclyl, aryl, heteroaryl, or bridged bicyclic ring, by itself or attached to another moiety, are independently optionally substituted with 1-3 substituents selected from the group consisting of OH, halo, —NH₂, C₁-C₆alkyl, C₁-C₆alkyl-O—, R¹²O—C₁-C₆alkyl(O)C—, and R¹²O(O)C—;

with the proviso that:

(a) when

X is N;

R¹ is —C(O)OR⁶

R² is —C(O)CH₂Cl or —C(O)CH₂F;

p is 0; and

R⁵ is H;

then (i) R³ and R⁶ are not simultaneously-NO₂ and —CH₃, respectively;

(b) when

X is N;

R¹ is —C(O)OR⁶

R² is —C(O)CH₂Cl or —C(O)CH₂F;

R³ is —C(O)OR¹⁰;

p is 0; and

R⁵ is H;

then R⁶ and R¹⁰ are not simultaneously

(i) —CH₃;

(ii) —CH₃ and C₂-C₆alkynyl, respectively; and

(iii) —CH₂CH₃ and —CH₃, respectively.

In one embodiment, the RSL3 derivative or analog is a compound represented by Structural Formula (VII-2a):

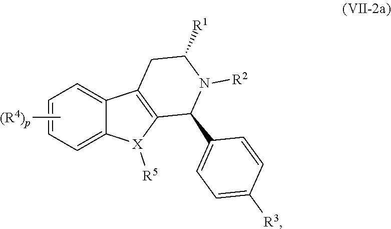

or an enantiomer or pharmaceutically acceptable salt thereof.

In certain embodiments, the aryl, when used alone or as part of a larger moiety, e.g., arylC₁-C₆alkyl, is selected from phenyl, naphthyl, and biphenyl.

In certain embodiments, the heteroaryl, when used alone or as part of a larger moiety, e.g., heteroarylC₁-C₆alkyl, is selected from furanyl, imidazolyl, benzimidazolyl, isoxazolyl, oxazolyl, pyrrolyl, pyridyl, pyrimidinyl, pyridazinyl, thiazolyl, thienyl, 3-thienyl, benzofuryl, indolyl, pyrazolyl, isothiazolyl, oxadiazolylpurinyl, pyrazinyl, and quinolinyl.

In certain embodiments, the 4 to 7-membered heterocyclyl is selected from azetidinyl, pyrrolidinyl, piperidinyl, pyrazolidinyl, isoxazolidinyl, oxazolidinyl, thiazolidinyl, imidazolidinyl, piperazinyl, morpholinyl, thiomorpholinyl, 1,3-oxazinanyl, 1,3-thiazinanyl, dihydropyridinyl, 1,3-tetrahydropyrimidinyl, dihydropyrimidinyl, azepanyl and 1,4-diazepanyl.

In certain embodiments, the 4 to 6-membered heterocyclyl when present is selected from azetidinyl, oxetanyl, thietanyl, piperidinyl, 1,2,3,6-tetrahydropyridinyl, pyranyl, tetrahydropyranyl, dioxanyl, 1,3-dioxolanyl, dihydropyranyl, dihydrothienyl, dihydrofuranyl, imidazolinyl, pyrrolidinyl, piperidinyl, pyrazolidinyl, isoxazolidinyl, oxazolidinyl, thiazolidinyl, piperazinyl, morpholinyl, thiomorpholinyl, 1,3-oxazinanyl, 1,3-thiazinanyl, dihydropyridinyl, 1,3-tetrahydropyrimidinyl, and dihydropyrimidinyl.

In certain embodiments, R⁴ is halo. In certain embodiments, R⁴ is Br, Cl, or F, and p is 1 or 2.

In certain embodiments, R⁹ is —C₁-C₂alkylhalo. In certain embodiments, R⁹ is —C₁-C₂alkylC₁ or —C₁-C₂alkylF. In certain embodiments, R⁹ is —CH₂CH₂C₁. In certain embodiments, R⁹ is —CD₂CD₂Cl. In certain embodiments, R⁹ is —CH₂Cl or —CH₂F. In certain embodiments, R⁹ is —CH₂Cl. In certain embodiments, R⁹ is —CD₂Cl or —CD₂F. In certain embodiments, R⁹ is CD₂C₁.

In certain embodiments, R¹ is —C(O)OR⁶, wherein R⁶ is a C₁-C₆alkyl, C₁-C₄alkyl, or C₃-C₆alkyl. In certain embodiments, the R⁶ of —C(O)OR⁶ is methyl, ethyl, n-propyl, n-butyl, isopropyl, t-butyl, pentyl, or hexyl.

In certain embodiments, R³ is —C(O)OR¹⁰, wherein R¹⁰ is a C₁-C₆alkyl, C₁-C₄alkyl, or C₃-C₆alkyl.

In certain embodiments, the R¹⁰ of —C(O)OR¹⁰ is methyl, ethyl, n-propyl, n-butyl, isopropyl, t-butyl, pentyl, or hexyl.

As noted for the compounds of formula (VII-2) and (VII-2a), when R¹ is —C(O)OR⁶, R² is —C(O)CH₂Cl; and R³ is —C(O)OR¹⁰, then R⁶ and R¹⁰ are not simultaneously: (i) —CH₃, (ii) —CH₃ and C₂-C₆alkynyl respectively; and (iii) CH₂CH₃ and —CH₃, respectively.

In one embodiment, the RSL3 derivative or analog is a compound represented by Structural Formula (VII-3):

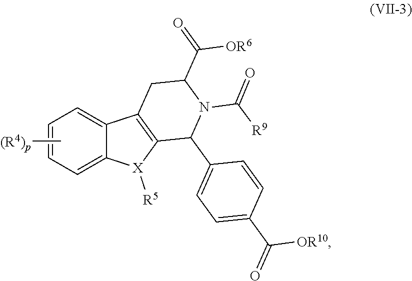

or an enantiomer or pharmaceutically acceptable salt thereof; wherein:

X is N, O or S;

R⁴ is independently halo, CN, —NH₂, —SO₂, C₁-C₈alkyl, —OR¹², —C₁-C₆alkyl-OR¹², —C₁-C₆alkyl-NR¹² or —OC(O)R¹²;

R⁵ is H, C₁-C₆alkyl, or is absent when X is S or O;

p is 0, 1, 2 or 3;

R⁶ is C₁-C₆alkyl, C₃-C₆alkyl, C₂-C₆alkenyl, C₂-C₆alkynyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, (R¹¹)₂NC₁-C₆alkyl-, or (R¹¹)₂NC₂-C₆alkenyl;

R⁹ is —C₁-C₂alkylhalo, —C₂-C₃alkenylhalo, or C₂alkynyl, wherein the C₁-C₂alkyl is optionally substituted with one or two halo, one or two —CH₃;

R¹⁰ is C₁-C₆alkyl, C₂-C₆alkyl, C₂-C₆alkenyl, C₂-C₆alkynyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, (R¹¹)₂NC₁-C₆alkyl-, (R¹¹)₂NC₂-C₆alkenyl-, R¹³(NH₂)CH—, R¹⁴C₀-C₆alkyl-, or (R¹⁵)₃SiC₀-C₆alkyl-;

each R¹¹ is independently H, C₁-C₆alkyl, C₂-C₆alkenyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, R¹²O—C₁-C₆alkyl-, (R¹¹)₂NC₁-C₆alkyl-, (R¹¹)₂NC₂-C₆alkenyl-, R²O(O)C—C₁-C₆alkyl-, R¹³(NH₂)CH—, R¹⁴C₀-C₆alkyl-, (R¹⁵)₃SiC₀-C₆alkyl-, or an N-protecting group; or two R¹¹ together with the nitrogen atom to which they are attached form a 4 to 7 membered heterocyclyl, wherein the heterocyclyl formed by the two R¹¹ groups has optionally 0, 1 or 2 additional heteroatoms selected from nitrogen, oxygen, and sulfur, and the heterocyclyl is optionally substituted with OH, halo, C₁-C₆alkyl, C₁-C₆alkyl-O(O)C—, (R¹¹)₂N—, or a 4- to 6-membered heterocyclyl, wherein the 4- to 6-membered heterocyclyl is optionally substituted with OH, halo, —NH₂, or C₁-C₆alkyl, or when containing 2 or more N atoms is optionally substituted with an N-protecting group;

each R¹² is independently H or C₁-C₆alkyl;

each R¹³ is independently H, C₁-C₆alkyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, or an N protecting group;

R¹⁴ is a bridged bicyclic ring having 0-2 heteroatoms independently selected from nitrogen, oxygen, and sulfur; and

each R¹⁵ is independently C₁-C₆alkyl, C₂-C₆alkenyl, aryl, heteroaryl, arylC₁-C₆alkyl-, arylC₂-C₆alkenyl-, heteroarylC₁-C₆alkyl-, and heteroarylC₂-C₆alkenyl-;

wherein the C₁-C₆alkyl, C₃-C₆cycloalkyl, heterocyclyl, aryl, heteroaryl, or bridged bicyclic ring, by itself or attached to another moiety, are independently optionally substituted with 1-3 substituents selected from the group consisting of OH, halo, —NH₂, C₁-C₆alkyl, C₁-C₆alkyl-O—, R¹²O—C₁-C₆alkyl(O)C—, and R¹²O(O)C—;

with the proviso that when

X is N;

R⁹ is —CH₂Cl, —CH₂F, —CD₂Cl, or —CD₂F;

p is 0; and

R⁵ is H, then R⁶ and R¹⁰ are not simultaneously

(i) —CH₃;

(ii) —CH₃ and C₂-C₆alkynyl, respectively; and

(iii) —CH₂CH₃ and —CH₃, respectively.

In one embodiment, the RSL3 derivative or analog is a compound represented by Structural Formula (VII-3a):

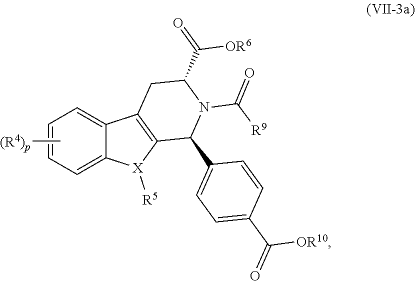

or an enantiomer or pharmaceutically acceptable salt thereof.

In certain embodiments of the compound of formula (VII-3) or (VII-3a), wherein:

X is N, O or S;

R⁴ is independently halo, CN, —NH₂, —SO₂, C₁-C₈alkyl, —OR¹², —C₁-C₆alkyl-OR¹², —C₁-C₆alkyl-NR¹² or —OC(O)R¹²;

R⁵ is H, C₁-C₆alkyl, or is absent when X is S or O;

R⁶ is C₁-C₆alkyl;

R⁹ is —C₁-C₂alkylCl;

R¹⁰ is C₂-C₆alkyl, C₃-C₆cycloalkyl, or C₃-C₆cycloalkylC₁-C₆alkyl-;

R¹² is H or C₁-C₆alkyl; and

p is 0, 1, 2 or 3.

In certain embodiments of the compound of formula (VII-3) or (VII-3a), X is N. In certain embodiments of formula (VII-3) or (VII-3a), R⁴ is halo or absent. In certain embodiments, R⁴ is Br, Cl, or F, and p is 1 or 2. In certain embodiments of formula (VII-3) or (VII-3a), R⁶ is C₃-C₆alkyl. In certain embodiments of formula (VII-3) or (VII-3a), R¹⁰ is C₃-C₆alkyl. In certain embodiments of formula (VII-3) or (VII-3a), X is N, R⁴ is halo or absent, and R⁶ is C₃-C₆alkyl. In certain embodiments of formula (VII-3) or (VII-3a), X is N, R⁴ is halo or absent, and R¹⁰ is C₃-C₆alkyl.

In certain embodiments of the compound of formula (VII-3) or (VII-3a), or an enantiomer or pharmaceutically acceptable salt thereof, wherein:

X is N, O or S;

R⁴ is independently halo, CN, —NH₂, —SO₂, C₁-C₈alkyl, —OR¹², —C₁-C₆alkyl-OR¹², —C₁-C₆alkyl-NR¹² or —OC(O)R¹²;

R⁵ is H, C₁-C₆alkyl, or is absent when X is S or O;

R⁶ is C₃-C₆alkyl;

R⁹ is —C₁-C₂alkylCl;

R¹⁰ is C₁-C₆alkyl;

R¹² is H or C₁-C₆alkyl; and

p is 0, 1, 2 or 3.

In certain embodiments of the compound of formula (VII-3) or (VII-3a), R⁶ is t-butyl and R¹⁰ is C₁-C₆alkyl.

In certain embodiments of the compound of formula (VII-3) or (VII-3a), R⁶ is C₃-C₆alkyl; and R¹⁰ is CH₃. In certain embodiments, R⁶ is t-butyl.

In certain embodiments, R¹⁰ is t-butyl.

In certain embodiments of the compound of formula (VII-3) or (VII-3a), or an enantiomer or pharmaceutically acceptable salt thereof, wherein:

X is N, O or S;

R⁴ is independently halo, CN, —NH₂, —SO₂, C₁-C₈alkyl, —OR¹², —C₁-C₆alkyl-OR¹², —C₁-C₆alkyl-NR¹² or —OC(O)R¹²;

R⁵ is H, C₁-C₆alkyl, or is absent when X is S or O;

R⁶ is C₁-C₆alkyl;

R⁹ is —C₁-C₂alkylCl;

R¹⁰ is C₃-C₆alkyl;

R¹² is H or C₁-C₆alkyl; and

p is 0, 1, 2 or 3.

In certain embodiments of the compound of formula (VII-3) and (VII-3a), R⁶ is C₁-C₆alkyl and R¹⁰ is t-butyl.

In certain embodiments of the compound of formula (VII-3) and (VII-3a), R⁶ is —CH₃; and R¹⁰ is C₃-C₆alkyl. In certain embodiments, R⁶ is —CH₃ and R¹⁰ is t-butyl.

In certain embodiments of the compound of formula (VII-3) and (VII-3a), R⁶ is ethyl; and R¹⁰ is t-butyl.

In certain embodiments of the compound of structural formula (VII-3) and (VII-3a), or an enantiomer or pharmaceutically acceptable salt thereof, wherein:

X is N, O or S;

R⁴ is independently halo, CN, —NH₂, —SO₂, C₁-C₈alkyl, —OR¹², —C₁-C₆alkyl-OR¹², —C₁-C₆alkyl-NR¹² or —OC(O)R¹²;

R⁵ is H, C₁-C₆alkyl, or is absent when X is S or O;

R⁶ is C₁-C₆alkyl;

R⁹ is —C₁-C₂alkylCl; and

R¹⁰ is adamantyl or adamantylC₁-C₆aliphatic-;

R¹² is H or C₁-C₆alkyl; and

p is 0, 1, 2 or 3.

In certain embodiments, the adamantyl is selected from the following:

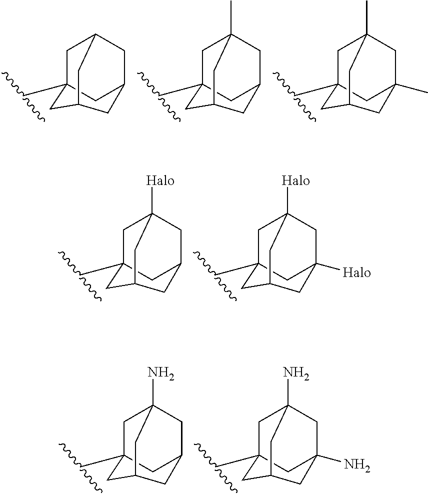

In certain embodiments of the compound of formula (VII), (VII-1), (VII-2), (VII-2a), (VII-3), and (VII-3a), R⁶ is methyl, ethyl, n-propyl, n-butyl, isopropyl, t-butyl, pentyl, or hexyl; and R¹⁰ is adamantylC₁-C₆aliphatic-.

In certain embodiments of the compound of formula (VII-3) or (VII-3a), X is N. In certain embodiments of the compound of formula (VII-3) or (VII-3a), X is N; and R⁵ is H.

In certain embodiments of the compound of formula (VII-3) or (VII-3a), R⁴ is halo. In certain embodiments of formula (VII-3) or (VII-3a), p is 0.

In certain embodiments of the compound of formula (VII), (VII-1), (VII-2), or (VII-2a), R¹ is —C(O)N(R⁷)₂ or —OC(O)R⁶; and R³ is —C(O)OR¹⁰. In certain embodiments, R¹ is —C(O)N(R⁷)₂.

In certain embodiments of the compound of formula (VII), (VII-1), (VII-2), or (VII-2a), R¹ is —C(O)OR⁶; and R³ is —C(O)N(R¹¹)₂, or —OC(O)R¹⁰. In certain embodiments, R³ is —C(O)N(R¹¹)₂.

In one embodiment, the RSL3 derivative or analog is a compound represented by Structural Formula (VII-4):

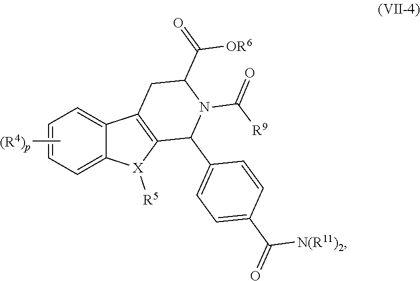

or an enantiomer or pharmaceutically acceptable salt thereof; wherein:

X is N, O or S;

R⁴ is independently halo, CN, —NH₂, —SO₂, —C₁-C₈alkyl, —OR¹², —C₁-C₆alkyl-OR¹², —C₁-C₆alkyl-NR¹² or —OC(O)R¹².

R⁵ is H, C₁-C₆alkyl, or is absent when X is S or O;

p is 0, 1, 2 or 3;

each R⁶ is independently C₁-C₆alkyl, C₃-C₆alkyl, C₂-C₆alkenyl, C₂-C₆alkynyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, (R¹¹)₂NC₁-C₆alkyl-, or (R¹¹)₂NC₂-C₆alkenyl-;

R⁹ is —C₁-C₂alkylhalo, —C₂-C₃alkenylhalo, or C₂alkynyl, wherein the C₁-C₂alkyl is optionally substituted with one or two halo, one or two —CH₃;

each R¹¹ is independently H, C₁-C₆alkyl, C₂-C₆alkenyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, R¹²O—C₁-C₆alkyl-, (R¹¹)₂NC₁-C₆alkyl-, (R¹¹)₂NC₂-C₆alkenyl-, R¹²O(O)C—C₁-C₆alkyl-, R¹³(NH₂)CH—, R¹⁴C₀-C₆alkyl-, (R¹⁵)₃SiC₀-C₆alkyl-, or an N-protecting group; or two R¹¹ together with the nitrogen atom to which they are attached form a 4 to 7 membered heterocyclyl, wherein the heterocyclyl formed by the two R¹¹ groups has optionally 0, 1 or 2 additional heteroatoms selected from nitrogen, oxygen, and sulfur, and the heterocyclyl is optionally substituted with OH, halo, C₁-C₆alkyl, C₁-C₆alkyl-O(O)C—, (R¹¹)₂N—, or a 4- to 6-membered heterocyclyl, wherein the 4- to 6-membered heterocyclyl is optionally substituted with OH, halo, —NH₂, or C₁-C₆alkyl, or when containing 2 or more N atoms is optionally substituted with an N-protecting group;

each R¹² is independently H or C₁-C₆alkyl;

each R¹³ is independently H, C₁-C₆alkyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, or an N protecting group;

R¹⁴ is a bridged bicyclic ring having 0-2 heteroatoms independently selected from nitrogen, oxygen, and sulfur; and

each R¹⁵ is independently C₁-C₆alkyl, C₂-C₆alkenyl, aryl, heteroaryl, arylC₁-C₆alkyl-, arylC₂-C₆alkenyl-, heteroarylC₁-C₆alkyl-, and heteroarylC₂-C₆alkenyl-;

wherein the C₁-C₆alkyl, C₃-C₆cycloalkyl, heterocyclyl, aryl, heteroaryl, or bridged bicyclic ring, by itself or attached to another moiety, are independently optionally substituted with 1-3 substituents selected from the group consisting of OH, halo, —NH₂, C₁-C₆alkyl, C₁-C₆alkyl-O—, R¹²O—C₁-C₆alkyl(O)C—, and R¹²O(O)C—.

In one embodiment, the RSL3 derivative or analog is a compound represented by Structural Formula (VII-5):

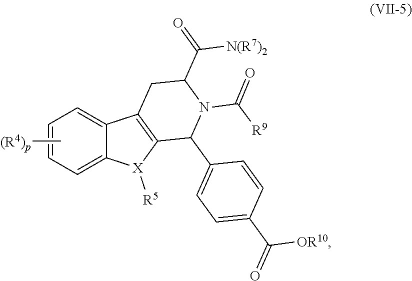

or an enantiomer or pharmaceutically acceptable salt thereof; wherein

X is N, O or S;

R⁴ is independently halo, CN, —NH₂, —SO₂, C₁-C₈alkyl, —OR¹², —C₁-C₆alkyl-OR¹², —C₁-C₆alkyl-NR¹² or —OC(O)R¹²;

R⁵ is H, C₁-C₆alkyl, or is absent when X is S or O;

p is 0, 1, 2 or 3;

each R⁷ is independently H, C₁-C₆alkyl, C₂-C₆alkenyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, (R¹¹)₂NC₁-C₆alkyl-, (R¹¹)₂NC₂-C₆alkenyl-, R¹²O—C₁-C₆alkyl-, or R¹²O(O)C—C₁-C₆alkyl-, or two R⁷ together with the nitrogen atom to which they are attached form a 4 to 7 membered heterocyclyl, wherein the heterocyclyl formed by the two R⁷ groups is optionally substituted with OH, halo,

C₁-C₆alkyl, a 4- to 6-membered heterocyclyl, or (R¹¹)₂N—, wherein the 4- to 6-membered heterocyclyl when containing 2 or more N atoms is optionally substituted with an N-protecting group;

R⁹ is —C₁-C₂alkylhalo, —C₂-C₃alkenylhalo, or C₂alkynyl, wherein the C₁-C₂alkyl is optionally substituted with one or two halo, one or two —CH₃;

R¹⁰ is C₁-C₆alkyl, C₂-C₆alkyl, C₂-C₆alkenyl, C₂-C₆alkynyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, (R¹¹)₂NC₁-C₆alkyl-, (R¹¹)₂NC₂-C₆alkenyl-, R¹³(NH₂)CH—, R¹⁴C₀-C₆alkyl-, (R¹⁵)₃SiC₀-C₆alkyl-;

each R¹¹ is independently H, C₁-C₆alkyl, C₂-C₆alkenyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, R¹²O—C₁-C₆alkyl-, (R¹¹)₂NC₁-C₆alkyl-, (R¹¹)₂NC₂-C₆alkenyl-, R¹²O(O)C—C₁-C₆alkyl-, R¹³(NH₂)CH—, R¹⁴C₀-C₆alkyl-, (R¹⁵)₃SiC₀-C₆alkyl-, or an N-protecting group; or two R¹¹ together with the nitrogen atom to which they are attached form a 4 to 7 membered heterocyclyl, wherein the heterocyclyl formed by the two R¹¹ groups has optionally 0, 1 or 2 additional heteroatoms selected from nitrogen, oxygen, and sulfur, and the heterocyclyl is optionally substituted with OH, halo, C₁-C₆alkyl, C₁-C₆alkyl-O(O)C—, (R¹¹)₂N—, or a 4- to 6-membered heterocyclyl, wherein the 4- to 6-membered heterocyclyl is optionally substituted with OH, halo, —NH₂, or C₁-C₆alkyl, or when containing 2 or more N atoms is optionally substituted with an N-protecting group;

each R¹² is independently H or C₁-C₆alkyl;

each R¹³ is independently H, C₁-C₆alkyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, or an N protecting group;

R¹⁴ is a bridged bicyclic ring having 0-2 heteroatoms independently selected from nitrogen, oxygen, and sulfur; and

each R¹⁵ is independently C₁-C₆alkyl, C₂-C₆alkenyl, aryl, heteroaryl, arylC₁-C₆alkyl-, arylC₂-C₆alkenyl-, heteroarylC₁-C₆alkyl-, and heteroarylC₂-C₆alkenyl-;

wherein the C₁-C₆alkyl, —C₃-C₆cycloalkyl, heterocyclyl, aryl, heteroaryl, or bridged bicyclic ring, by itself or attached to another moiety, are independently optionally substituted with 1-3 substituents selected from the group consisting of OH, halo, —NH₂, C₁-C₆alkyl, C₁-C₆alkyl-O—, R¹²O—C₁-C₆alkyl(O)C—, and R¹²O(O)C—.

In one embodiment, the RSL3 derivative or analog is a compound represented by Structural Formula (VII-4a) or (VII-5a):

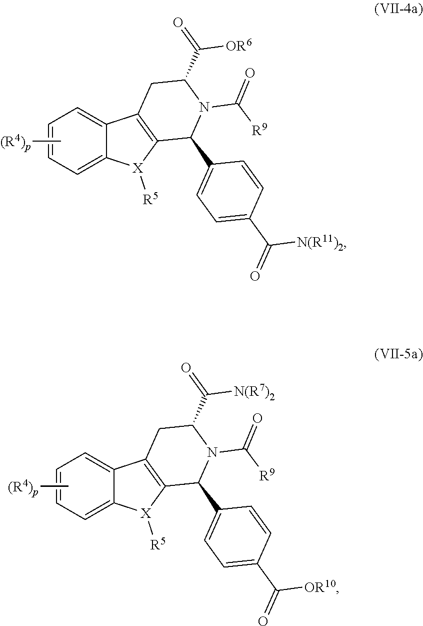

or an enantiomer or pharmaceutically acceptable salt thereof.

In certain embodiments of the compound of formula (VII-4) or (VII-4a), or an enantiomer or pharmaceutically acceptable salt thereof,

X is N, O or S;

R⁴ is independently halo, CN, —NH₂, —SO₂, C₁-C₈alkyl, —OR¹², —C₁-C₆alkyl-OR¹², —C₁-C₆alkyl-NR¹² or —OC(O)R¹²;

R⁵ is H, C₁-C₆alkyl, or is absent when X is S or O;

R⁶ is C₁-C₆ alkyl;

R⁹ is —C₁-C₂alkylC;

R¹¹ are as defined for formula (VII) above;

R¹² is H or C₁-C₆alkyl; and

p is 0, 1, 2 or 3.

In certain embodiments of the compound of formula (VII-4) and (VII-4a), each R¹¹ is a C₁-C₆alkyl. In certain embodiments of the compound of formula (VII-4) and (VII-4a), each R¹¹ is —CH₃. In certain embodiments of the compound of formula (VII-4) and (VII-4a), one of R¹¹ is R¹²O(O)C—C₁-C₆alkyl- or R¹²O—C₁-C₆alkyl-, wherein the C₁-C₆ alkyl is optionally substituted with C₁-C₆alkyl or —NH₂, and R¹² is H or C₁-C₆alkyl.

In certain embodiments of formula (VII-4) and (VII-4a), the two R¹¹ group together with the nitrogen atom to which they are attached form a 4 to 7 membered heterocyclyl, wherein the heterocyclyl formed by the two R¹¹ groups is optionally substituted with a 4- to 6-membered heterocyclyl or N(C₁-C₆alkyl)₂, wherein the 4- to 6-membered heterocyclyl when containing 2 or more N atoms is optionally substituted with an N-protecting group. In certain embodiments, the 4 to 7 membered heterocyclyl is selected from azetidinyl, pyrrolidinyl, piperidinyl, pyrazolidinyl, isoxazolidinyl, oxazolidinyl, thiazolidinyl, imidazolidinyl, piperazinyl, morpholinyl, thiomorpholinyl, 1,3-oxazinanyl, 1,3-thiazinanyl, dihydropyridinyl, tetrahydropyranyl, 1,3-tetrahydropyrimidinyl, dihydropyrimidinyl, azepanyl and 1,4-diazepanyl. In certain embodiments, the 4- to 6-membered heterocyclyl, when present as a substituent, is selected from azetidinyl, oxetanyl, thietanyl, piperidinyl, 1,2,3,6-tetrahydropyridinyl, pyranyl, dioxanyl, 1,3-dioxolanyl, dihydropyranyl, dihydrothienyl, dihydrofuranyl, imidazolinyl, pyrrolidinyl, piperidinyl, pyrazolidinyl, isoxazolidinyl, oxazolidinyl, thiazolidinyl, piperazinyl, morpholinyl, thiomorpholinyl, 1,3-oxazinanyl, 1,3-thiazinanyl, dihydropyridinyl, 1,3-tetrahydropyrimidinyl, and dihydropyrimidinyl. In certain embodiments, the N-protecting group when present is t-Boc.

In certain embodiments of the compound of formula (VII-5) or (VII-5a), or an enantiomer or pharmaceutically acceptable salt thereof, wherein:

X is N, O or S;

R⁴ is independently halo, CN, —NH₂, —SO₂, C₁-C₈alkyl, —OR¹², —C₁-C₆alkyl-OR¹², —C₁-C₆alkyl-NR¹² or —OC(O)R¹²;

R⁵ is H, C₁-C₆alkyl, or is absent when X is S or O;

R⁷ is as defined for formula (VII) above;

R⁹ is —C₁-C₂alkylCl;

R¹⁰ is C₁-C₆alkyl;

R¹² is H or C₁-C₆alkyl; and

p is 0, 1, 2 or 3.

In certain embodiments of the compound of formula (VII-5) and (VII-5a), each R⁷ is a H or C₁-C₆alkyl. In certain embodiments of the compound of formula (VII-5) and (VII-5a), R⁷ is C₁-C₆alkyl. In certain embodiments of the compound of formula (VII-5) and (VII-5a), R⁷ is C₁-C₆alkyl and R¹⁰ is C₁-C₆alkyl. In certain embodiments of the compound of formula (VII-5), R⁷ is R¹²O(O)C—C₁-C₆alkyl-, wherein C₁-C₆ alkyl is optionally substituted with C₁-C₆alkyl or NH₂, and each R¹² is independently H or C₁-C₆alkyl.

In certain embodiments of the compound of formula (VII-4) or (VII-4a), or an enantiomer or pharmaceutically acceptable salt thereof, wherein

X is N, O or S;

R⁴ is independently halo, CN, —NH₂, —SO₂, C₁-C₈alkyl, —OR¹², —C₁-C₆alkyl-OR¹², —C₁-C₆alkyl-NR² or —OC(O)R¹²;

R⁵ is H, C₁-C₆alkyl, or is absent when X is S or O;

R⁷ is C₁-C₆alkyl;

R⁹ is —C₁-C₂alkylCl;

R¹⁰ is C₁-C₆alkyl;

R¹² is H or C₁-C₆alkyl; and

p is 0, 1, 2 or 3.

In certain embodiments of the compound of formula (VII-4) or (VII-4a), or an enantiomer or pharmaceutically acceptable salt thereof, R¹⁰ is t-butyl.

In certain embodiments of the compound of formula (VII-4), (VII-4a), (VII-5), and (VII-5a), X is N. In certain embodiments of the compound of formula (VII-4), (VII-4a), (VII-5), and (VII-5a), X is N; and R⁵ is H.

In certain embodiments of the compound of formula (VII-4), (VII-4a), (VII-5), and (VII-5a), R⁴ is halo. In certain embodiments of formula (VII-4), (VII-4a), (VII-5), and (VII-5a), p is 0.

In one embodiment, the RSL3 derivative or analog is a compound represented by Structural Formula (VII-6) or (VII-7):

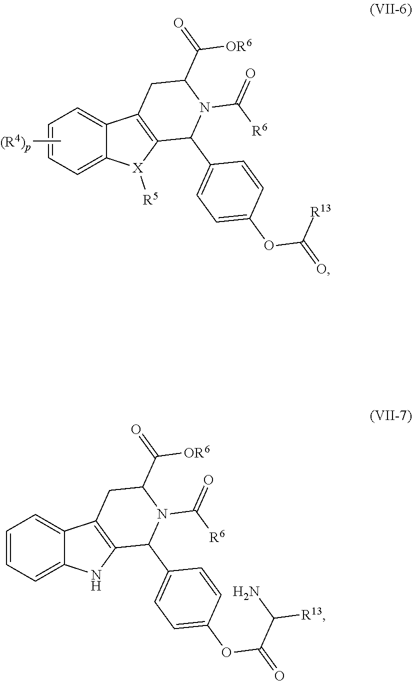

or an enantiomer or pharmaceutically acceptable salt thereof; wherein:

X is N, O or S;

R⁴ is independently halo, CN, —NH₂, —SO₂, C₁-C₈alkyl, —OR¹², —C₁-C₆alkyl-OR¹², —C₁-C₆alkyl-NR¹² or —OC(O)R¹²;

R⁵ is H, C₁-C₆alkyl, or is absent when X is S or O;

p is 0, 1, 2 or 3;

R⁶ is C₁-C₆alkyl, C₃-C₆alkyl, C₂-C₆alkenyl, C₂-C₆alkynyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, (R¹¹)₂NC₁-C₆alkyl-, or (R¹¹)₂NC₂-C₆alkenyl;

R⁹ is —C₁-C₂alkylhalo, —C₂-C₃alkenylhalo, or C₂alkynyl, wherein the C₁-C₂alkyl is optionally substituted with one or two halo, one or two —CH₃;

R¹⁰ is C₁-C₆alkyl, C₂-C₆alkyl, C₂-C₆alkenyl, C₂-C₆alkynyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, (R¹¹)₂NC₁-C₆alkyl-, (R¹¹)₂NC₂-C₆alkenyl-, R¹³(NH₂)CH—, R¹⁴C₀-C₆alkyl- or (R¹⁵)₃SiC₀-C₆alkyl-;

each R¹¹ is independently H, C₁-C₆alkyl, C₂-C₆alkenyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, R¹²O—C₁-C₆alkyl-, (R¹¹)₂NC₁-C₆alkyl-, (R¹¹)₂NC₂-C₆alkenyl-, R²O(O)C—C₁-C₆alkyl-, R¹³(NH₂)CH—, R¹⁴C₀-C₆alkyl-, (R¹⁵)₃SiC₀-C₆alkyl-, or an N-protecting group; or two R¹¹ together with the nitrogen atom to which they are attached form a 4 to 7 membered heterocyclyl, wherein the heterocyclyl formed by the two R¹¹ groups has optionally 0, 1 or 2 additional heteroatoms selected from nitrogen, oxygen, and sulfur, and the heterocyclyl is optionally substituted with OH, halo, C₁-C₆alkyl, C₁-C₆alkyl-O(O)C—, (R¹¹)₂N—, or a 4- to 6-membered heterocyclyl, wherein the 4- to 6-membered heterocyclyl is optionally substituted with OH, halo, —NH₂, or C₁-C₆alkyl, or when containing 2 or more N atoms is optionally substituted with an N-protecting group;

each R¹² is independently H or C₁-C₆alkyl;

each R¹³ is independently H, C₁-C₆alkyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, or an N protecting group;

R¹⁴ is a bridged bicyclic ring having 0-2 heteroatoms independently selected from nitrogen, oxygen, and sulfur; and

each R¹⁵ is independently C₁-C₆alkyl, C₂-C₆alkenyl, aryl, heteroaryl, arylC₁-C₆alkyl-, arylC₂-C₆alkenyl-, heteroarylC₁-C₆alkyl-, and heteroarylC₂-C₆alkenyl-;

wherein the C₁-C₆alkyl, C₃-C₆cycloalkyl, heterocyclyl, aryl, heteroaryl, or bridged bicyclic ring, by itself or attached to another moiety, are independently optionally substituted with 1-3 substituents selected from the group consisting of OH, halo, —NH₂, C₁-C₆alkyl, C₁-C₆alkyl-O—, R¹²O—C₁-C₆alkyl(O)C—, and R¹²O(O)C—.

In one embodiment, the RSL3 derivative or analog is a compound represented by Structural Formula (VII-6a) or (VII-7a):

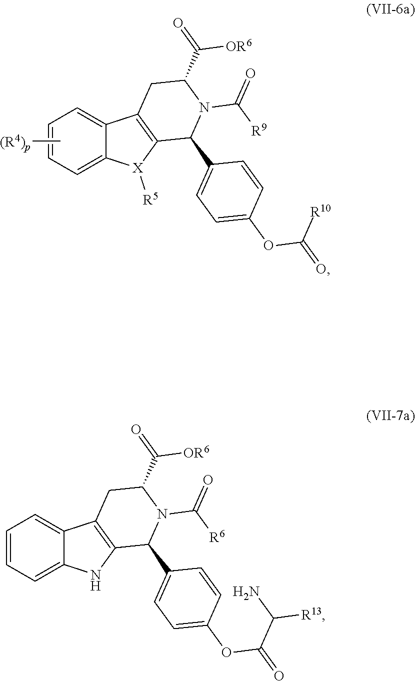

or an enantiomer or pharmaceutically acceptable salt thereof.

In certain embodiments, the compound of formula (VII-6), (VII-6a), (VII-7) and (VII-7a), or an enantiomer or pharmaceutically acceptable salt thereof, wherein:

X is N, O or S;

R⁴ is independently halo, CN, —NH₂, —SO₂, C₁-C₈alkyl, —OR¹², —C₁-C₆alkyl-OR¹², —C₁-C₆alkyl-NR¹² or —OC(O)R¹²;

R⁵ is H, C₁-C₆alkyl, or is absent when X is S or O;

R⁶ is C₁-C₆alkyl;

R⁹ is —C₁-C₂alkylCl; and

R¹⁰, R¹² and R¹³ are as defined for the compound of formula (VII-6) or (VII-7), above.

In certain embodiments of the compound of formula (VII-6) or (VII-6a), R⁶ is methyl, ethyl, n-propyl, n-butyl, isopropyl, t-butyl, pentyl, or hexyl. In certain embodiments of the compound of formula (VII-6) or (VII-6a), R⁶ is t-butyl. In certain embodiments of the compound of formula (VII-6) or (VII-6a), R¹⁰ is a C₁-C₆alkyl.

In certain embodiments of the compound of formula (VII-7) or (VII-7a), R¹³ is a C₁-C₆alkyl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, or heteroarylC₂-C₆alkenyl-, wherein the C₃-C₆cycloalkyl, heterocyclyl, aryl, and heteroaryl are optionally substituted with 1-3 substituents selected from the group consisting of OH, halo, C₁-C₆alkyl, and C₁-C₆alkyl-O—, HOCH₂(O)C—, R¹²O(O)C—, wherein R¹² is as defined for formula (VII).

In certain embodiments of the compound of formula (VII-6), (VII-6a), (VII-7), or (VII-7a), R⁶ is t-butyl.

In certain embodiments of the compound of formula (VII-6), (VII-6a), (VII-7), or (VII-7a) above, the aryl when present is selected from phenyl and naphthyl. In certain embodiments of the compound of (VII-6), (VII-6a), (VII-7), or (VII-7a) above, the heteroaryl when present is selected from furanyl, imidazolyl, benzimidazolyl, isoxazolyl, oxazolyl, pyrrolyl, pyridyl, pyrimidinyl, pyridazinyl, thiazolyl, thienyl, 3-thienyl, benzofuryl, indolyl, pyrazolyl, isothiazolyl, oxadiazolylpurinyl, pyrazinyl, and quinolinyl. In certain embodiments of the compound of formula (VII-6), (VII-6a), (VII-7), or (VII-7a) above, the heterocyclyl when present is selected from azetidinyl, pyrrolidinyl, piperidinyl, pyrazolidinyl, isoxazolidinyl, oxazolidinyl, thiazolidinyl, imidazolidinyl, piperazinyl, morpholinyl, thiomorpholinyl, 1,3-oxazinanyl, 1,3-thiazinanyl, dihydropyridinyl, 1,3-tetrahydropyrimidinyl, dihydropyrimidinyl, azepanyl and 1,4-diazepanyl.

In certain embodiments of the compound of formula (VII-6), (VII-6a), (VII-7), and (VII-7a), X is N. In certain embodiments of the compound of formula (VII-6), (VII-6a), (VII-7), and (VII-7a), X is N; and R⁵ is H.

In certain embodiments of the compound of formula (VII-6), (VII-6a), (VII-7), and (VII-7a), R⁴ is halo. In certain embodiments of formula (VII-6), (VII-6a), (VII-7), and (VII-7a), p is 0.

In one embodiment, the RSL3 derivative or analog is a compound represented by Structural Formula (VII-8):

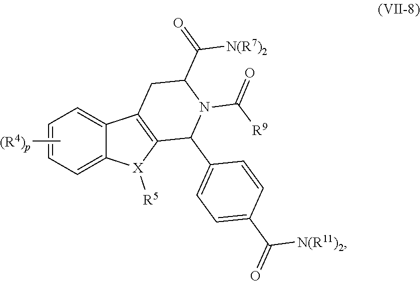

or an enantiomer or pharmaceutically acceptable salt thereof; wherein:

X is N, O or S;

R⁴ is independently halo, CN, —NH₂, —SO₂, C₁-C₈alkyl, —OR¹², —C₁-C₆alkyl-OR¹², —C₁-C₆alkyl-NR¹² or —OC(O)R¹²;

R⁵ is H, C₁-C₆alkyl, or is absent when X is S or O;

p is 0, 1, 2 or 3;

each R⁷ is independently H, C₁-C₆alkyl, C₂-C₆alkenyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, (R¹¹)₂NC₁-C₆alkyl-, (R¹¹)₂NC₂-C₆alkenyl-, R¹²O—C₁-C₆alkyl-, or R¹²O(O)C—C₁-C₆alkyl-, or two R⁷ together with the nitrogen atom to which they are attached form a 4 to 7 membered heterocyclyl, wherein the heterocyclyl formed by the two R⁷ groups is optionally substituted with OH, halo, C₁-C₆alkyl, a 4- to 6-membered heterocyclyl, or (R¹¹)₂N—, wherein the 4- to 6-membered heterocyclyl when containing 2 or more N atoms is optionally substituted with an N-protecting group;

R⁹ is —C₁-C₂alkylhalo, —C₂-C₃alkenylhalo, or C₂alkynyl, wherein the C₁-C₂alkyl is optionally substituted with one or two halo, one or two —CH₃;

each R¹¹ is independently H, C₁-C₆alkyl, C₂-C₆alkenyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, R¹²O—C₁-C₆alkyl-, (R¹¹)₂NC₁-C₆alkyl-, (R¹¹)₂NC₂-C₆alkenyl-, R¹²O(O)C—C₁-C₆alkyl-, R¹³(NH₂)CH—, R¹⁴C₀-C₆alkyl-, (R¹⁵)₃SiC₀-C₆alkyl-, or an N-protecting group; or two R¹¹ together with the nitrogen atom to which they are attached form a 4 to 7 membered heterocyclyl, wherein the heterocyclyl formed by the two R¹¹ groups has optionally 0, 1 or 2 additional heteroatoms selected from nitrogen, oxygen, and sulfur, and the heterocyclyl is optionally substituted with OH, halo, C₁-C₆alkyl, C₁-C₆alkyl-O(O)C—, (R¹¹)₂N—, or a 4- to 6-membered heterocyclyl, wherein the 4- to 6-membered heterocyclyl is optionally substituted with OH, halo, NH₂, or C₁-C₆alkyl, or when containing 2 or more N atoms is optionally substituted with an N-protecting group;

each R¹² is independently H or C₁-C₆alkyl;

each R¹³ is independently H, C₁-C₆alkyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, or an N protecting group;

R¹⁴ is a bridged bicyclic ring having 0-2 heteroatoms independently selected from nitrogen, oxygen, and sulfur; and

each R¹⁵ is independently C₁-C₆alkyl, C₂-C₆alkenyl, aryl, heteroaryl, arylC₁-C₆alkyl-, arylC₂-C₆alkenyl-, heteroarylC₁-C₆alkyl-, and heteroarylC₂-C₆alkenyl-;

wherein the C₁-C₆alkyl, C₃-C₆cycloalkyl, heterocyclyl, aryl, heteroaryl, or bridged bicyclic ring, by itself or attached to another moiety, are independently optionally substituted with 1-3 substituents selected from the group consisting of OH, halo, —NH₂, C₁-C₆alkyl, C₁-C₆alkyl-O—, R¹²O—C₁-C₆alkyl(O)C—, and R¹²O(O)C—.

In one embodiment, the RSL3 derivative or analog is a compound represented by Structural Formula (VII-8a):

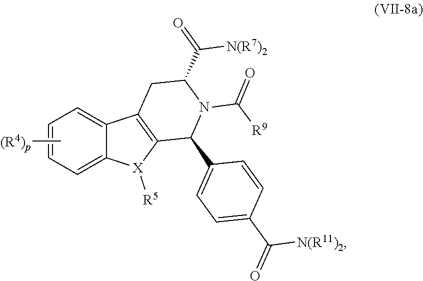

or an enantiomer or pharmaceutically acceptable salt thereof.

In certain embodiments of the compound of formula (VII-8) and (VII-8a), X is N. In certain embodiments of the compound of formula (VII-8) and (VII-8a), X is N; and R⁵ is H.

In certain embodiments of the compound of formula (VII-8) and (VII-8a), R⁴ is halo. In certain embodiments of formula (VII-8) and (VII-8a), p is 0.

In one embodiment, the RSL3 derivative or analog is a compound represented by Structural Formula (VII-9):

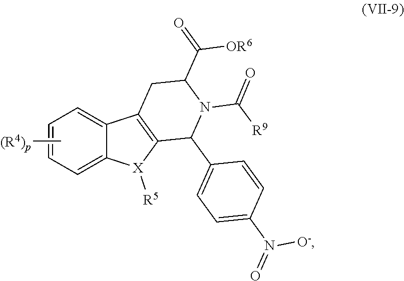

or an enantiomer or pharmaceutically acceptable salt thereof; wherein:

X is N, O or S;

R⁴ is independently halo, CN, —NH₂, —SO₂, C₁-C₈alkyl, —OR¹², —C₁-C₆alkyl-OR¹², —C₁-C₆alkyl-NR¹² or —OC(O)R¹²;

R⁵ is H, C₁-C₆alkyl, or is absent when X is S or O;

p is 0, 1, 2 or 3;

R⁶ is C₃-C₆alkyl, C₂-C₆alkenyl, C₂-C₆alkynyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, (R¹¹)₂NC₁-C₆alkyl-, or (R¹¹)₂NC₂-C₆alkenyl-;

R⁹ is —C₁-C₂alkylhalo, —C₂-C₃alkenylhalo, or C₂alkynyl, wherein the C₁-C₂alkyl is optionally substituted with one or two halo, one or two —CH₃;

each R¹¹ is independently H, C₁-C₆alkyl, C₂-C₆alkenyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, R¹²O—C₁-C₆alkyl-, (R¹¹)₂NC₁-C₆alkyl-, (R¹¹)₂NC₂-C₆alkenyl-, R¹²O(O)C—C₁-C₆alkyl-, R¹³(NH₂)CH—, R¹⁴C₀-C₆alkyl-, (R¹⁵)₃SiC₀-C₆alkyl-, or an N-protecting group; or two R¹¹ together with the nitrogen atom to which they are attached form a 4 to 7 membered heterocyclyl, wherein the heterocyclyl formed by the two R¹¹ groups has optionally 0, 1 or 2 additional heteroatoms selected from nitrogen, oxygen, and sulfur, and the heterocyclyl is optionally substituted with OH, halo, C₁-C₆alkyl, C₁-C₆alkyl-O(O)C—, (R¹¹)₂N—, or a 4- to 6-membered heterocyclyl, wherein the 4- to 6-membered heterocyclyl is optionally substituted with OH, halo, NH₂, or C₁-C₆alkyl, or when containing 2 or more N atoms is optionally substituted with an N-protecting group;

each R¹² is independently H or C₁-C₆alkyl;

each R¹³ is independently H, C₁-C₆alkyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, or an N protecting group;

R¹⁴ is a bridged bicyclic ring having 0-2 heteroatoms independently selected from nitrogen, oxygen, and sulfur; and

each R¹⁵ is independently C₁-C₆alkyl, C₂-C₆alkenyl, aryl, heteroaryl, arylC₁-C₆alkyl-, arylC₂-C₆alkenyl-, heteroarylC₁-C₆alkyl-, and heteroarylC₂-C₆alkenyl-;

wherein the C₁-C₆alkyl, C₃-C₆cycloalkyl, heterocyclyl, aryl, heteroaryl, or bridged bicyclic ring, by itself or attached to another moiety, are independently optionally substituted with 1-3 substituents selected from the group consisting of OH, halo, —NH₂, C₁-C₆alkyl, C₁-C₆alkyl-O—, R¹²O—C₁-C₆alkyl(O)C—, and R¹²O(O)C—.

In one embodiment, the RSL3 derivative or analog is a compound represented by Structural Formula (VII-9a):

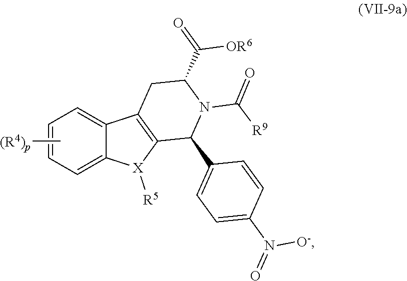

or an enantiomer or pharmaceutically acceptable salt thereof.

In certain embodiments of the compound of structural formula (VII-9) or (VII-9a), or an enantiomer or pharmaceutically acceptable salt thereof, wherein:

X is N, O or S;

R⁴ is independently halo, CN, —NH₂, —SO₂, C₁-C₈alkyl, —OR¹², —C₁-C₆alkyl-OR¹², —C₁-C₆alkyl-NR¹² or —OC(O)R¹²;

R⁵ is H, C₁-C₆alkyl, or is absent when X is S or O;

R⁶ is C₃-C₆alkyl;

R⁹ is —C₁-C₂alkylhalo;

R¹² is H or C₁-C₆alkyl; and

p is 0, 1, 2 or 3.

In certain embodiments, R⁶ is t-butyl.

In certain embodiments of the compound of formula (VII-9) and (VII-9a), X is N. In certain embodiments of the compound of formula (VII-9) and (VII-9a), X is N; and R⁵ is H.

In certain embodiments of the compound of formula (VII-9) and (VII-9a), R⁴ is halo. In certain embodiments of formula (VII-9) and (VII-9a), p is 0.

In one embodiment, the RSL3 derivative or analog is a compound represented by Structural Formula (VII-10):

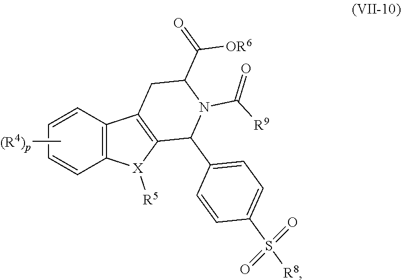

or an enantiomer or pharmaceutically acceptable salt thereof; wherein:

X is N, O or S;

R⁴ is independently halo, CN, —NH₂, —SO₂, C₁-C₈alkyl, —OR¹², —C₁-C₆alkyl-OR¹², —C₁-C₆alkyl-NR¹² or —OC(O)R¹²;

R⁵ is H, C₁-C₆alkyl, or is absent when X is S or O;

p is 0, 1, 2 or 3;

R⁶ is C₁-C₆alkyl, C₃-C₆alkyl, C₂-C₆alkenyl, C₂-C₆alkynyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, (R¹¹)₂NC₁-C₆alkyl-, or (R¹¹)₂NC₂-C₆alkenyl;

R⁸ is C₁-C₆alkyl, C₃-C₆alkyl, C₂-C₆alkenyl, C₂-C₆alkynyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, (R¹¹)₂NC₁-C₆alkyl-, or (R¹¹)₂N—;

R⁹ is —C₁-C₂alkylhalo, —C₂-C₃alkenylhalo, or C₂alkynyl, wherein the C₁-C₂alkyl is optionally substituted with one or two halo, one or two —CH₃;

each R¹¹ is independently H, C₁-C₆alkyl, C₂-C₆alkenyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, R¹²O—C₁-C₆alkyl-, (R¹¹)₂NC₁-C₆alkyl-, (R¹¹)₂NC₂-C₆alkenyl-, R¹²O(O)C—C₁-C₆alkyl-, R¹³(NH₂)CH—, R¹⁴C₀-C₆alkyl-, (R¹⁵)₃SiC₀-C₆alkyl-, or an N-protecting group; or two R¹¹ together with the nitrogen atom to which they are attached form a 4 to 7 membered heterocyclyl, wherein the heterocyclyl formed by the two R¹¹ groups has optionally 0, 1 or 2 additional heteroatoms selected from nitrogen, oxygen, and sulfur, and the heterocyclyl is optionally substituted with OH, halo, C₁-C₆alkyl, C₁-C₆alkyl-O(O)C—, (R¹¹)₂N—, or a 4- to 6-membered heterocyclyl, wherein the 4- to 6-membered heterocyclyl is optionally substituted with OH, halo, —NH₂, or C₁-C₆alkyl, or when containing 2 or more N atoms is optionally substituted with an N-protecting group;

each R¹² is independently H or C₁-C₆alkyl;

each R¹³ is independently H, C₁-C₆alkyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, or an N protecting group;

R¹⁴ is a bridged bicyclic ring having 0-2 heteroatoms independently selected from nitrogen, oxygen, and sulfur; and

each R¹⁵ is independently C₁-C₆alkyl, C₂-C₆alkenyl, aryl, heteroaryl, arylC₁-C₆alkyl-, arylC₂-C₆alkenyl-, heteroarylC₁-C₆alkyl-, and heteroarylC₂-C₆alkenyl-;

wherein the C₁-C₆alkyl, C₃-C₆cycloalkyl, heterocyclyl, aryl, heteroaryl, bridged bicyclic ring, by itself or attached to another moiety, are independently optionally substituted with 1-3 substituents selected from the group consisting of OH, halo, —NH₂, C₁-C₆alkyl, C₁-C₆alkyl-O—, R¹²O—C₁-C₆alkyl(O)C—, and R¹²O(O)C—.

In one embodiment, the RSL3 derivative or analog is a compound represented by Structural Formula (VII-10a):

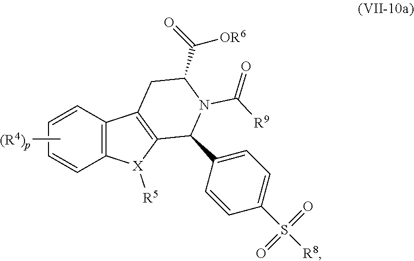

or an enantiomer or pharmaceutically acceptable salt thereof.

In certain embodiments of the compound, or an enantiomer or pharmaceutically acceptable salt thereof, of structural formula (VII-10) or (VII-10a):

R⁶ is C₁-C₆alkyl, C₃-C₆alkyl, C₃-C₆cycloalkyl, heterocyclyl, aryl, heteroaryl, C₃-C₆cycloalkylC₁-C₆alkyl-, heterocyclylC₁-C₆alkyl-, arylC₁-C₆alkyl-, or heteroarylC₁-C₆alkyl-;

R⁸ is C₁-C₆alkyl, C₃-C₆alkyl, C₂-C₆alkenyl, C₂-C₆alkynyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, (R¹¹)₂NC₁-C₆alkyl-, or (R¹¹)₂N—;

R⁹ is —C₁-C₂alkylhalo, —C₂-C₃alkenylhalo, or C₂alkynyl, wherein the C₁-C₂alkyl is optionally substituted with one or two halo, one or two —CH₃;

each R¹¹ is independently H, C₁-C₆alkyl, C₂-C₆alkenyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, R¹²O—C₁-C₆alkyl-, or (R¹¹)₂NC₁-C₆alkyl-; or two R¹¹ together with the nitrogen atom to which they are attached form a 4 to 7 membered heterocyclyl, wherein the heterocyclyl formed by the two R¹¹ groups is optionally substituted with OH, halo, C₁-C₆alkyl, a 4- to 6-membered heterocyclyl, or (R¹¹)₂N—, wherein the 4- to 6-membered heterocyclyl is optionally substituted with OH, halo, or C₁-C₆alkyl, or when containing 2 or more N atoms is optionally substituted with an N-protecting group;

each R¹² is independently H or C₁-C₆alkyl; and

wherein the C₁-C₆alkyl, C₃-C₆cycloalkyl, heterocyclyl, aryl, and heteroaryl, by itself or as part of larger moiety are independently optionally substituted with 1-3 substituents selected from the group consisting of OH, halo, C₁-C₆alkyl, C₁-C₆alkyl-O—, R¹²O—C₁-C₆alkyl(O)C—, and R¹²O(O)C—.

In certain embodiments of the compound of formula (VII-10) and (VII-10a), wherein:

X is N, O or S;

R⁴ is independently halo, CN, —NH₂, —SO₂, C₁-C₈alkyl, —OR¹², —C₁-C₆alkyl-OR¹², —C₁-C₆alkyl-NR¹² or —OC(O)R¹²;

R⁵ is H, C₁-C₆alkyl, or is absent when X is S or O;

p is 0, 1, 2 or 3;

R⁶ is C₁-C₆alkyl;

R⁸ is (R¹¹)₂N—;

R⁹ is —C₁-C₂alkylhalo;

each R¹¹ is independently H, C₁-C₆alkyl, adamantyl, or adamantylC₁-C₆aliphatic-; and

R¹² is H or C₁-C₆alkyl.

In certain embodiments of the compound of formula (VII-10) and (VII-10a), wherein:

X is N, O or S;

R⁴ is independently halo or C₁-C₈alkyl;

R⁵ is H, C₁-C₆alkyl, or is absent when X is S or O;

p is 0, 1, 2 or 3;

R⁶ is C₁-C₆alkyl;

R⁸ is (R¹¹)₂N—;

R⁹ is —C₁-C₂alkylhalo; and

R¹¹ is H, and adamantyl or adamantylC₁-C₆aliphatic-.

In certain embodiments of the compound of formula (VII-10) and (VII-10a), wherein:

X is N, O or S;

R⁴ is independently halo or C₁-C₈alkyl;

R⁵ is H, C₁-C₆alkyl, or is absent when X is S or O;

p is 0, 1, 2 or 3;

R⁶ is C₁-C₆alkyl;

R⁸ is (R¹¹)₂N—;

R⁹ is —C₁-C₂alkylhalo; and

two R¹¹ together with the nitrogen atom to which they are attached form a 4 to 7 membered heterocyclyl, wherein the heterocyclyl formed by the two R¹¹ groups is optionally substituted with OH, halo, C₁-C₆alkyl, a 4- to 6-membered heterocyclyl, or (R¹¹)₂N—, wherein the 4- to 6-membered heterocyclyl is optionally substituted with OH, halo, or C₁-C₆alkyl, or when containing 2 or more N atoms is optionally substituted with an N-protecting group.

In certain embodiments of the compound of formula (VII-10) and (VII-10a), X is N. In certain embodiments of the compound of formula (VII-10) and (VII-10a), X is N; and R⁵ is H.

In certain embodiments of the compound of formula (VII-10) and (VII-10a), R⁴ is halo. In certain embodiments of formula (VII-10) and (VII-10a), p is 0.

In one embodiment, the RSL3 derivative or analog is a compound represented by Structural Formula (VII-11):

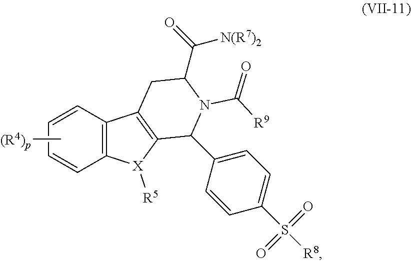

or an enantiomer or pharmaceutically acceptable salt thereof; wherein:

X is N, O or S;

R⁴ is independently halo, CN, —NH₂, —SO₂, —C₁-C₈alkyl, —OR¹², —C₁-C₆alkyl-OR¹², —C₁-C₆alkyl-NR¹² or —OC(O)R¹²;

R⁵ is H, C₁-C₆alkyl, or is absent when X is S or O;

p is 0, 1, 2 or 3;

each R⁷ is independently H, C₁-C₆alkyl, C₂-C₆alkenyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, (R¹¹)₂NC₁-C₆alkyl-, (R¹¹)₂NC₂-C₆alkenyl-, R¹²O—C₁-C₆alkyl-, or R¹²O(O)C—C₁-C₆alkyl-, or two R⁷ together with the nitrogen atom to which they are attached form a 4 to 7 membered heterocyclyl, wherein the heterocyclyl formed by the two R⁷ groups is optionally substituted with OH, halo, C₁-C₆alkyl, a 4- to 6-membered heterocyclyl, or (R¹¹)₂N—, wherein the 4- to 6-membered heterocyclyl when containing 2 or more N atoms is optionally substituted with an N-protecting group;

R⁸ is independently C₁-C₆alkyl, C₃-C₆alkyl, C₂-C₆alkenyl, C₂-C₆alkynyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, (R¹¹)₂NC₁-C₆alkyl-, or (R¹¹)₂N—;

R⁹ is —C₁-C₂alkylhalo, —C₂-C₃alkenylhalo, or C₂alkynyl, wherein the C₁-C₂alkyl is optionally substituted with one or two halo, one or two —CH₃;

each R¹¹ is independently H, C₁-C₆alkyl, C₂-C₆alkenyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, R¹²O—C₁-C₆alkyl-, (R¹¹)₂NC₁-C₆alkyl-, (R¹¹)₂NC₂-C₆alkenyl-, R²O(O)C—C₁-C₆alkyl-, R¹³(NH₂)CH—, R¹⁴C₀-C₆alkyl-, (R¹⁵)₃SiC₀-C₆alkyl-, or an N-protecting group; or two R¹¹ together with the nitrogen atom to which they are attached form a 4 to 7 membered heterocyclyl, wherein the heterocyclyl formed by the two R¹¹ groups has optionally 0, 1 or 2 additional heteroatoms selected from nitrogen, oxygen, and sulfur, and the heterocyclyl is optionally substituted with OH, halo, C₁-C₆alkyl, C₁-C₆alkyl-O(O)C—, (R¹¹)₂N—, or a 4- to 6-membered heterocyclyl, wherein the 4- to 6-membered heterocyclyl is optionally substituted with OH, halo, —NH₂, or C₁-C₆alkyl, or when containing 2 or more N atoms is optionally substituted with an N-protecting group;

each R¹² is independently H or C₁-C₆alkyl;

each R¹³ is independently H, C₁-C₆alkyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, or an N protecting group;

R¹⁴ is a bridged bicyclic ring having 0-2 heteroatoms independently selected from nitrogen, oxygen, and sulfur; and

each R¹⁵ is independently C₁-C₆alkyl, C₂-C₆alkenyl, aryl, heteroaryl, arylC₁-C₆alkyl-, arylC₂-C₆alkenyl-, heteroarylC₁-C₆alkyl-, and heteroarylC₂-C₆alkenyl-;

wherein the C₁-C₆alkyl, C₃-C₆cycloalkyl, heterocyclyl, aryl, heteroaryl, or bridged bicyclic ring, by itself or attached to another moiety, are independently optionally substituted with 1-3 substituents selected from the group consisting of OH, halo, —NH₂, C₁-C₆alkyl, C₁-C₆alkyl-O—, R¹²O—C₁-C₆alkyl(O)C—, and R¹²O(O)C—.

In one embodiment, the RSL3 derivative or analog is a compound represented by Structural Formula (VII-11a):

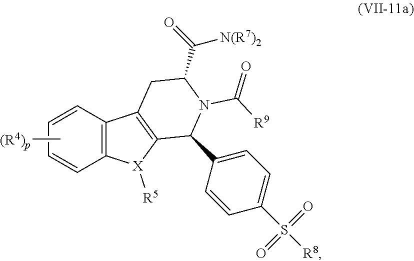

or an enantiomer or pharmaceutically acceptable salt thereof.

In certain embodiments of the compound of formula (VII-11) and (VII-11a), X is N.

In certain embodiments of the compound of formula (VII-11) and (VII-11a), X is N; and R⁵ is H.

In certain embodiments of the compound of formula (VII-11) and (VII-11a), R⁴ is halo. In certain embodiments of formula (VII-11) and (VII-11a), p is 0.

In one embodiment, the RSL3 derivative or analog is a compound represented by Structural Formula (VII-12):

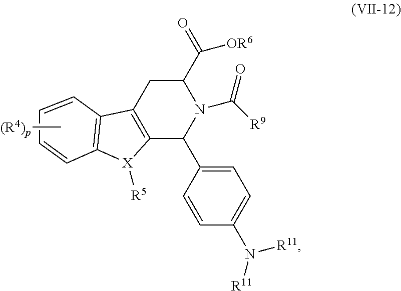

or an enantiomer or pharmaceutically acceptable salt thereof; wherein:

X is N, O or S;

R⁴ is independently halo, CN, —NH₂, —SO₂, C₁-C₈alkyl, —OR¹², —C₁-C₆alkyl-OR¹², —C₁-C₆alkyl-NR¹² or —OC(O)R¹²;

R⁵ is H, C₁-C₆alkyl, or is absent when X is S or O;

p is 0, 1, 2 or 3;

R⁶ is C₁-C₆alkyl, C₃-C₆alkyl, C₂-C₆alkenyl, C₂-C₆alkynyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, (R¹¹)₂NC₁-C₆alkyl-, or (R¹¹)₂NC₂-C₆alkenyl;

R⁹ is —C₁-C₂alkylhalo, —C₂-C₃alkenylhalo, or C₂alkynyl, wherein the C₁-C₂alkyl is optionally substituted with one or two halo, one or two —CH₃;

each R¹¹ is independently H, C₁-C₆alkyl, C₂-C₆alkenyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, R¹²O—C₁-C₆alkyl-, (R¹¹)₂NC₁-C₆alkyl-, (R¹¹)₂NC₂-C₆alkenyl-, R¹²O(O)C—C₁-C₆alkyl-, R¹³(NH₂)CH—, R¹⁴C₀-C₆alkyl-, (R¹⁵)₃SiC₀-C₆alkyl-, or an N-protecting group; or two R¹¹ together with the nitrogen atom to which they are attached form a 4 to 7 membered heterocyclyl, wherein the heterocyclyl formed by the two R¹¹ groups has optionally 0, 1 or 2 additional heteroatoms selected from nitrogen, oxygen, and sulfur, and the heterocyclyl is optionally substituted with OH, halo, C₁-C₆alkyl, C₁-C₆alkyl-O(O)C—, (R¹¹)₂N—, or a 4- to 6-membered heterocyclyl, wherein the 4- to 6-membered heterocyclyl is optionally substituted with OH, halo, —NH₂, or C₁-C₆alkyl, or when containing 2 or more N atoms is optionally substituted with an N-protecting group;

each R¹² is independently H or C₁-C₆alkyl;

each R¹³ is independently H, C₁-C₆alkyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, or an N protecting group;

R¹⁴ is a bridged bicyclic ring having 0-2 heteroatoms independently selected from nitrogen, oxygen, and sulfur; and

each R¹⁵ is independently C₁-C₆alkyl, C₂-C₆alkenyl, aryl, heteroaryl, arylC₁-C₆alkyl-, arylC₂-C₆alkenyl-, heteroarylC₁-C₆alkyl-, and heteroarylC₂-C₆alkenyl-;

wherein the C₁-C₆alkyl, C₃-C₆cycloalkyl, heterocyclyl, aryl, heteroaryl, or bridged bicyclic ring, by itself or attached to another moiety, are independently optionally substituted with 1-3 substituents selected from the group consisting of OH, halo, —NH₂, C₁-C₆alkyl, C₁-C₆alkyl-O—, R¹²O—C₁-C₆alkyl(O)C—, and R¹²O(O)C—.

In certain embodiments of the compound of formula (VII-12), R⁶ is C₁-C₆alkyl, C₃-C₆alkyl, C₂-C₆alkenyl, or C₂-C₆alkynyl. In certain embodiments of the compound of formula (VII-12), R⁶ is C₁-C₆alkyl.

In certain embodiments, R⁶ is methyl, ethyl, n-propyl, n-butyl, isopropyl, t-butyl, pentyl, or hexyl. In certain embodiments of the compound of formula (VII-12), R⁶ is C₃-C₆alkyl, such as t-butyl.

In certain embodiments of the compound of formula (VII-12), one of R¹¹ is H and the other of R¹¹ is C₁-C₆alkyl, C₂-C₆alkenyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, R¹²O—C₁-C₆alkyl-, (R¹¹)₂NC₁-C₆alkyl-, (R¹¹)₂NC₂-C₆alkenyl-, R¹²O(O)C—C₁-C₆alkyl-, R¹³(NH₂)CH—, R¹⁴C₀-C₆alkyl-, (R¹⁵)₃SiC₀-C₆alkyl-, or an N-protecting group.

In certain embodiments of the compound of formula (VII-12), one of R¹¹ is H and the other of R¹¹ is C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic- or an N-protecting group.

In certain embodiments of the compound of formula (VII-12), R⁴ is halo or absent. In certain embodiments, R⁴ is Br, Cl, or F.

In one embodiment, the RSL3 derivative or analog is a compound represented by Structural Formula (VII-12a):

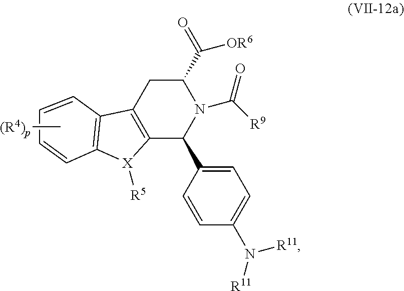

or an enantiomer or pharmaceutically acceptable salt thereof.

In one embodiment, the RSL3 derivative or analog is a compound represented by Structural Formula (VII-13):

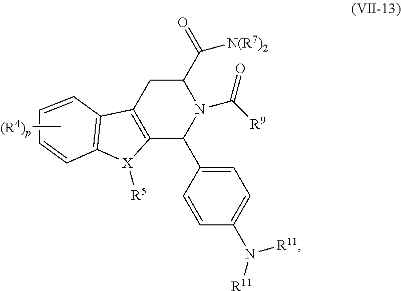

or an enantiomer or pharmaceutically acceptable salt thereof; wherein:

X is N, O or S;

R⁴ is independently halo, CN, —NH₂, —SO₂, —C₁-C₈alkyl, —OR¹², —C₁-C₆alkyl-OR¹², —C₁-C₆alkyl-NR¹² or —OC(O)R¹²;

R⁵ is H, C₁-C₆alkyl, or is absent when X is S or O;

p is 0, 1, 2 or 3;

each R⁷ is independently H, C₁-C₆alkyl, C₂-C₆alkenyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, (R¹¹)₂NC₁-C₆alkyl-, (R¹¹)₂NC₂-C₆alkenyl-, R¹²—O—C₁-C₆alkyl-, or R¹²O(O)C—C₁-C₆alkyl-, or two R⁷ together with the nitrogen atom to which they are attached form a 4 to 7 membered heterocyclyl, wherein the heterocyclyl formed by the two R⁷ groups is optionally substituted with OH, halo, C₁-C₆alkyl, a 4- to 6-membered heterocyclyl, or (R¹¹)₂N—, wherein the 4- to 6-membered heterocyclyl when containing 2 or more N atoms is optionally substituted with an N-protecting group;

R⁹ is —C₁-C₂alkylhalo, —C₂-C₃alkenylhalo, or C₂alkynyl, wherein the C₁-C₂alkyl is optionally substituted with one or two halo, one or two —CH₃;

each R¹¹ is independently H, C₁-C₆alkyl, C₂-C₆alkenyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, R¹²O—C₁-C₆alkyl-, (R¹¹)₂NC₁-C₆alkyl-, (R¹¹)₂NC₂-C₆alkenyl-, R¹²(O)C—C₁-C₆alkyl-, R¹³(NH₂)CH—, R¹⁴C₀-C₆alkyl-, (R¹⁵)₃SiC₀-C₆alkyl-, or an N-protecting group; or two R¹¹ together with the nitrogen atom to which they are attached form a 4 to 7 membered heterocyclyl, wherein the heterocyclyl formed by the two R¹¹ groups has optionally 0, 1 or 2 additional heteroatoms selected from nitrogen, oxygen, and sulfur, and the heterocyclyl is optionally substituted with OH, halo, C₁-C₆alkyl, C₁-C₆alkyl-O(O)C—, (R¹¹)₂N—, or a 4- to 6-membered heterocyclyl, wherein the 4- to 6-membered heterocyclyl is optionally substituted with OH, halo, NH₂, or C₁-C₆alkyl, or when containing 2 or more N atoms is optionally substituted with an N-protecting group;

each R¹² is independently H or C₁-C₆alkyl;

each R¹³ is independently H, C₁-C₆alkyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, or an N protecting group;

R¹⁴ is a bridged bicyclic ring having 0-2 heteroatoms independently selected from nitrogen, oxygen, and sulfur; and

each R¹⁵ is independently C₁-C₆alkyl, C₂-C₆alkenyl, aryl, heteroaryl, arylC₁-C₆alkyl-, arylC₂-C₆alkenyl-, heteroarylC₁-C₆alkyl-, and heteroarylC₂-C₆alkenyl-;

wherein the C₁-C₆alkyl, C₃-C₆cycloalkyl, heterocyclyl, aryl, heteroaryl, or bridged bicyclic ring, by itself or attached to another moiety, are independently optionally substituted with 1-3 substituents selected from the group consisting of OH, halo, NH₂, C₁-C₆alkyl, C₁-C₆alkyl-O—, R¹²O—C₁-C₆alkyl(O)C—, and R²O(O)C—.

In certain embodiments of the compound of formula (VII-13), each R⁷ is independently H, C₁-C₆alkyl. In certain embodiments, each R⁷ is C₁-C₆alkyl. In certain embodiments, wherein R⁷ is an alkyl, R⁷ is methyl, ethyl, n-propyl, n-butyl, isopropyl, t-butyl, pentyl, or hexyl.

In certain embodiments of the compound of formula (VII-13), one of R¹¹ is H and the other of R¹¹ is C₁-C₆alkyl, C₂-C₆alkenyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, R¹²O—C₁-C₆alkyl-, (R¹¹)₂NC₁-C₆alkyl-, (R¹¹)₂NC₂-C₆alkenyl-, R¹²O(O)C—C₁-C₆alkyl-, R¹³(NH₂)CH—, R¹⁴C₀-C₆alkyl-, (R¹⁵)₃SiC₀-C₆alkyl-, or an N-protecting group.

In certain embodiments of the compound of formula (VII-13), one of R¹¹ is H and the other of R¹¹ is C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic- or an N-protecting group.

In certain embodiments of the compound of formula (VII-13), R⁴ is halo or absent. In certain embodiments, R⁴ is Br, Cl, or F.

In one embodiment, the RSL3 derivative or analog is a compound represented by Structural Formula (VII-13a):

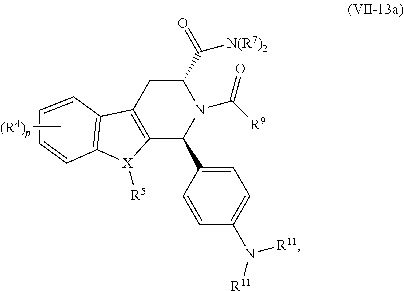

or an enantiomer or pharmaceutically acceptable salt thereof.

In one embodiment, the RSL3 derivative or analog is a compound represented by Structural Formula (VII-14):

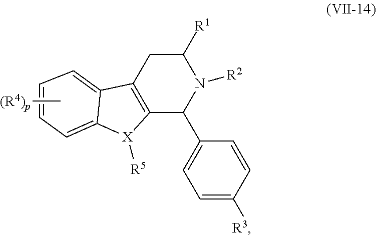

or an enantiomer or pharmaceutically acceptable salt thereof; wherein:

X is N, O or S;

R¹ is C₁-C₆alkyl, —C₁-C₆alkylhalo or —C₁-C₆alkyl-OR¹²;

R² is —C(O)R⁹;

R³ is —C(O)OR¹⁰, —C(O)N(R¹¹)₂, —OC(O)R¹⁰, —C₀-C₆alkylC₃-C₈cycloalkyl, —C₀-C₆alkylheterocyclyl, —N(R¹¹)₂, —SO₂R⁸, —SOR⁸, —NO₂ or —Si(R¹⁵)₃;

R⁴ is independently halo, CN, —NH₂, —SO₂, C₁-C₈alkyl, —OR¹², —C₁-C₆alkyl-OR¹², —C₁-C₆alkyl-NR¹² or —OC(O)R¹²;

R⁵ is H, C₁-C₆alkyl, or is absent when X is S or O;

p is 0, 1, 2 or 3;

R⁸ is independently C₁-C₆alkyl, C₃-C₆alkyl, C₂-C₆alkenyl, C₂-C₆alkynyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, (R¹¹)₂NC₁-C₆alkyl-, (R¹¹)₂N—, or R¹⁴C₀-C₆alkyl-;

R⁹ is —C₁-C₂alkylhalo, —C₂-C₃alkenylhalo, or C₂alkynyl, wherein the C₁-C₂alkyl is optionally substituted with one or two halo, one or two —CH₃;

R¹⁰ is C₁-C₆alkyl, C₂-C₆alkyl, C₂-C₆alkenyl, C₂-C₆alkynyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, (R¹¹)₂NC₁-C₆alkyl-, (R¹¹)₂NC₂-C₆alkenyl-, R¹³(NH₂)CH—, R¹⁴C₀-C₆alkyl-, or (R¹⁵)₃SiC₀-C₆alkyl-;

each R¹¹ is independently H, C₁-C₆alkyl, C₂-C₆alkenyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, R¹²O—C₁-C₆alkyl-, (R¹¹)₂NC₁-C₆alkyl-, (R¹¹)₂NC₂-C₆alkenyl-, R²O(O)C—C₁-C₆alkyl-, R¹³(NH₂)CH—, R¹⁴C₀-C₆alkyl-, (R¹⁵)₃SiC₀-C₆alkyl-, or an N-protecting group; or two R¹¹ together with the nitrogen atom to which they are attached form a 4 to 7 membered heterocyclyl, wherein the heterocyclyl formed by the two R¹¹ groups has optionally 0, 1 or 2 additional heteroatoms selected from nitrogen, oxygen, and sulfur, and the heterocyclyl is optionally substituted with OH, halo, C₁-C₆alkyl, C₁-C₆alkyl-O(O)C—, (R¹¹)₂N—, or a 4- to 6-membered heterocyclyl, wherein the 4- to 6-membered heterocyclyl is optionally substituted with OH, halo, NH₂, or C₁-C₆alkyl, or when containing 2 or more N atoms is optionally substituted with an N-protecting group;

each R¹² is independently H or C₁-C₆alkyl;

each R¹³ is independently H, C₁-C₆alkyl, C₃-C₆cycloalkyl, heterocyclyl, aryl, heteroaryl, C₃-C₆cycloalkylC₁-C₆alkyl-, C₃-C₆cycloalkylC₂-C₆alkenyl-, heterocyclylC₁-C₆alkyl-, heterocyclylC₂-C₆alkenyl-, arylC₁-C₆alkyl-, arylC₂-C₆alkenyl-, heteroarylC₁-C₆alkyl-, heteroarylC₂-C₆alkenyl-, adamantyl, adamantylC₁-C₆aliphatic-, or an N protecting group;

R¹⁴ is a bridged bicyclic ring having 0-2 heteroatoms independently selected from nitrogen, oxygen, and sulfur; and

each R¹⁵ is independently C₁-C₆alkyl, C₂-C₆alkenyl, aryl, heteroaryl, arylC₁-C₆alkyl-, arylC₂-C₆alkenyl-, heteroarylC₁-C₆alkyl-, and heteroarylC₂-C₆alkenyl-;

wherein the C₁-C₆alkyl, C₃-C₆cycloalkyl, heterocyclyl, aryl, heteroaryl, or bridged bicyclic ring, by itself or attached to another moiety, are independently optionally substituted with 1-3 substituents selected from the group consisting of OH, halo, —NH₂, C₁-C₆alkyl, C₁-C₆alkyl-O—, R¹²O—C₁-C₆alkyl(O)C—, and R¹²O(O)C—.

In one embodiment, the RSL3 derivative or analog is a compound represented by Structural Formula (VII-15):

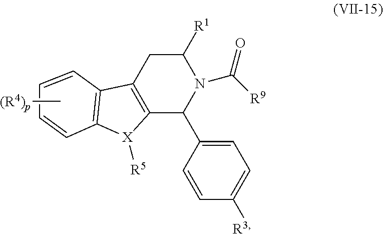

or an enantiomer or pharmaceutically acceptable salt thereof; wherein:

X is N, O or S;

R¹ is C₁-C₆alkyl, —C₁-C₆alkylhalo or —C₁-C₆alkyl-OR¹²;

R³ is —C₀-C₆alkylC₃-C₈cycloalkyl or —C₀-C₆alkylheterocyclyl;

R⁴ is independently halo, CN, —NH₂, —SO₂, C₁-C₈alkyl, —OR¹², —C₁-C₆alkyl-OR¹², —C₁-C₆alkyl-NR¹² or —OC(O)R¹²;

R⁵ is H, C₁-C₆alkyl, or is absent when X is S or O;

p is 0, 1, 2 or 3;

R⁹ is —C₁-C₂alkylhalo, —C₂-C₃alkenylhalo, or C₂alkynyl, wherein the C₁-C₂alkyl is optionally substituted with one or two halo, one or two —CH₃;

R¹² is independently H or C₁-C₆alkyl;

wherein the C₀-C₆alkyl or —C₃-C₈cycloalkyl are independently optionally substituted with 1-3 substituents selected from the group consisting of OH, halo, —NH₂, C₁-C₆alkyl, C₁-C₆alkyl-O—, R¹²O—C₁-C₆alkyl(O)C—, and R¹²O(O)C—.

In one embodiment, the RSL3 derivative or analog is a compound represented by Structural Formula (VII-14a):

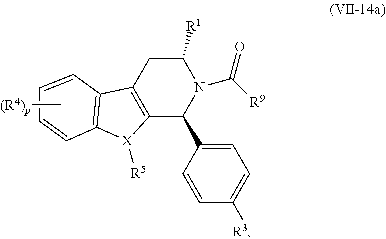

or an enantiomer or pharmaceutically acceptable salt thereof.

In certain embodiments of the compounds of formula (VII-14), (VII-15), and (VII-14a), R⁹ is —C₁-C₂alkylhalo. In certain embodiments, R⁹ is —C₁-C₂alkylCl or —C₁-C₂alkylF. In certain embodiments, R⁹ is —CH₂CH₂Cl. In certain embodiments, R⁹ is —CD₂CD₂Cl. In certain embodiments, R⁹ is —CH₂Cl or —CH₂F.

In certain embodiments, R⁹ is —CH₂Cl. In certain embodiments, R⁹ is —CD₂Cl or —CD₂F. In certain embodiments, R⁹ is-CD₂Cl.

In certain embodiments of the compounds of formula (VII-2), (VII-14), (VII-15), and (VII-14a), the C₃-C₈cycloalkyl group of the —C₀-C₆alkylC₃-C₈cycloalkyl is cyclopropyl, cyclobutyl, cyclopentyl, cyclohexyl, cycloheptyl, or cyclooctyl. In certain embodiments, the C₃-C₈cycloalkyl is cyclopropyl, cyclobutyl, cyclopentyl, or cyclohexyl.

In certain embodiments of the compounds of formula (VII-2), (VII-14), (VII-15), and (VII-14a), the heterocyclyl group of the —C₀-C₆alkylheterocyclyl is a 4-7 membered heterocyclic ring containing 1, 2 or 3 heteroatoms selected from S, N, and O, wherein the heterocyclic ring is optionally substituted with 1, 2 or 3 substituents selected from OH, halo, —NH₂, and C₁-C₆alkyl, or when containing 2 or more N atoms is optionally substituted with an N-protecting group. In certain embodiments the heterocyclic ring is selected from azetidinyl, pyrrolidinyl, piperidinyl, pyrazolidinyl, isoxazolidinyl, oxazolidinyl, thiazolidinyl, imidazolidinyl, tetrahydropyranyl, piperazinyl, morpholinyl, thiomorpholinyl, 1,3-oxazinanyl, 1,3-thiazinanyl, dihydropyridinyl, 1,3-tetrahydropyrimidinyl, dihydropyrimidinyl, azepanyl and 1,4-diazepanyl. In certain embodiments, the heterocycloalkyl is tetrahydropyranyl, piperidinyl, piperazinyl, or morpholinyl.

In certain embodiments, the compound, or a pharmaceutical acceptable salt thereof, is selected from the group consisting of the compounds of Table 3A.

TABLE 3A

| No. | Structure |
|---|---|
| 1 | 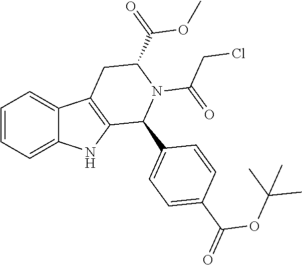 [SMILES: COC(=O)[C@H]1Cc2c([nH]c3ccccc23)[C@H](c2ccc(C(=O)OC(C)(C)C)cc2)N1C(=O)CCl] |
| 2 | 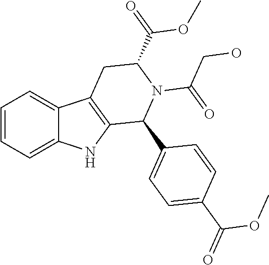 [SMILES: COC(=O)c1ccc([C@H]2c3[nH]c4ccccc4c3C[C@H](C(=O)OC)N2C(=O)CO)cc1] |
| 3 | 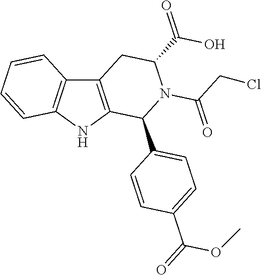 [SMILES: COC(=O)c1ccc([C@H]2c3[nH]c4ccccc4c3C[C@H](C(=O)O)N2C(=O)CCl)cc1] |
| 4 | 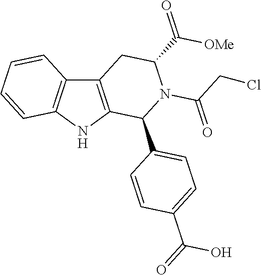 [SMILES: COC(=O)[C@H]1Cc2c([nH]c3ccccc23)[C@H](c2ccc(C(=O)O)cc2)N1C(=O)CCl] |
| 5 | 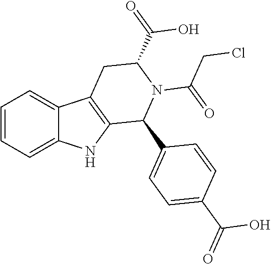 [SMILES: O=C(O)c1ccc([C@H]2c3[nH]c4ccccc4c3C[C@H](C(=O)O)N2C(=O)CCl)cc1] |
| 6 | 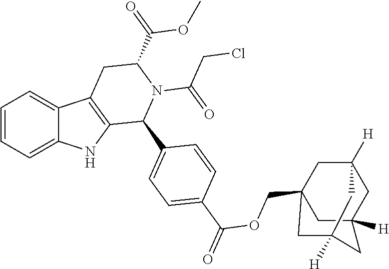 [SMILES: COC(=O)[C@H]1Cc2c([nH]c3ccccc23)[C@H](c2ccc(C(=O)OC[C@]34C[C@H]5C[C@H](C[C@H](C5)C3)C4)cc2)N1C(=O)CCl] |
| 7 | 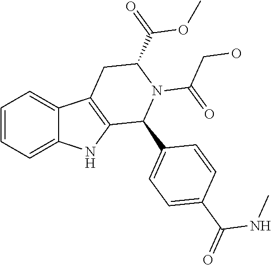 [SMILES: CNC(=O)c1ccc([C@H]2c3[nH]c4ccccc4c3C[C@H](C(=O)OC)N2C(=O)CO)cc1] |
| 8 | 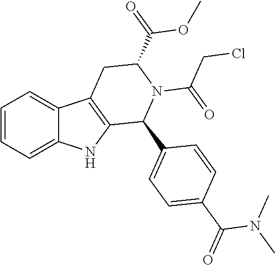 [SMILES: COC(=O)[C@H]1Cc2c([nH]c3ccccc23)[C@H](c2ccc(C(=O)N(C)C)cc2)N1C(=O)CCl] |
| 9 | 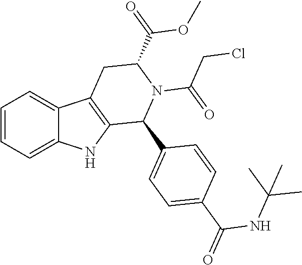 [SMILES: COC(=O)[C@H]1Cc2c([nH]c3ccccc23)[C@H](c2ccc(C(=O)NC(C)(C)C)cc2)N1C(=O)CCl] |
| 10 | 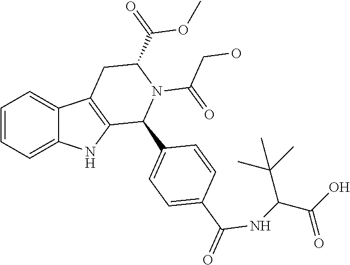 [SMILES: COC(=O)[C@H]1Cc2c([nH]c3ccccc23)[C@H](c2ccc(C(=O)NC(C(=O)O)C(C)(C)C)cc2)N1C(=O)CO] |
| 11 | 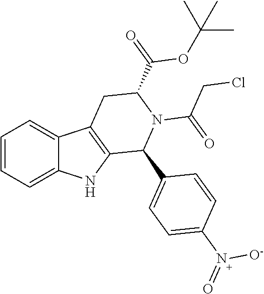 |
| 12 | 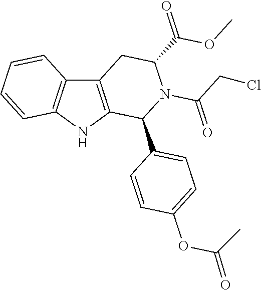 [SMILES: COC(=O)[C@H]1Cc2c([nH]c3ccccc23)[C@H](c2ccc(OC(C)=O)cc2)N1C(=O)CCl] |
| 13 | 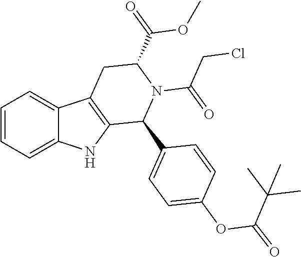 [SMILES: COC(=O)[C@H]1Cc2c([nH]c3ccccc23)[C@H](c2ccc(OC(=O)C(C)(C)C)cc2)N1C(=O)CCl] |
| 14 | 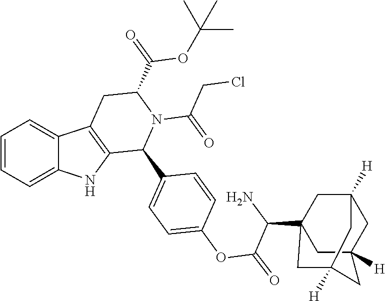 [SMILES: CC(C)(C)OC(=O)[C@H]1Cc2c([nH]c3ccccc23)[C@H](c2ccc(OC(=O)[C@@H](N)C34C[C@H]5C[C@@H](C3)C[C@@H](C4)C5)cc2)N1C(=O)CCl] |
| 15 | 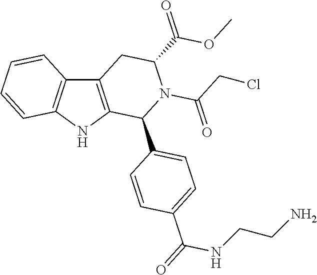 [SMILES: COC(=O)[C@H]1Cc2c([nH]c3ccccc23)[C@H](c2ccc(C(=O)NCCN)cc2)N1C(=O)CCl] |
| 16 | 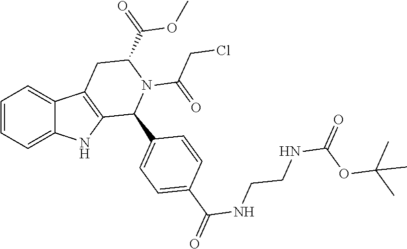 [SMILES: COC(=O)[C@H]1Cc2c([nH]c3ccccc23)[C@H](c2ccc(C(=O)NCCNC(=O)OC(C)(C)C)cc2)N1C(=O)CCl] |
| 17 | 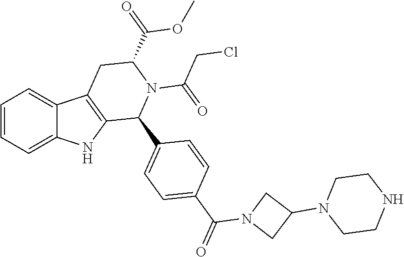 [SMILES: COC(=O)[C@H]1Cc2c([nH]c3ccccc23)[C@H](c2ccc(C(=O)N3CC(N4CCNCC4)C3)cc2)N1C(=O)CCl] |
| 18 | 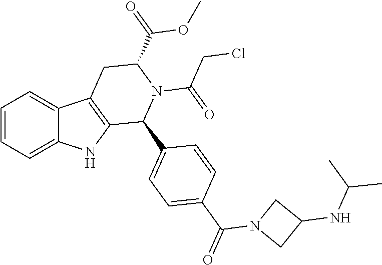 [SMILES: COC(=O)[C@H]1Cc2c([nH]c3ccccc23)[C@H](c2ccc(C(=O)N3CC(NC(C)C)C3)cc2)N1C(=O)CCl] |
| 19 | 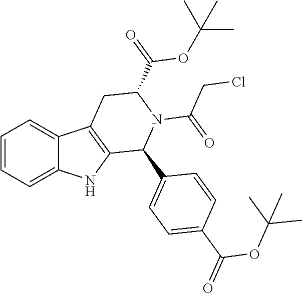 [SMILES: CC(C)(C)OC(=O)c1ccc([C@H]2c3[nH]c4ccccc4c3C[C@H](C(=O)OC(C)(C)C)N2C(=O)CCl)cc1] |
| 20 | 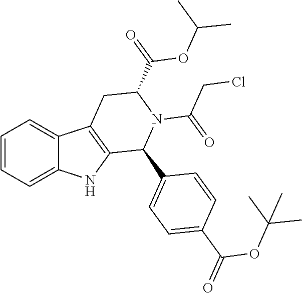 [SMILES: CC(C)OC(=O)[C@H]1Cc2c([nH]c3ccccc23)[C@H](c2ccc(C(=O)OC(C)(C)C)cc2)N1C(=O)CCl] |
| 21 | 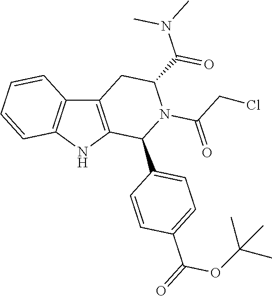 [SMILES: CN(C)C(=O)[C@H]1Cc2c([nH]c3ccccc23)[C@H](c2ccc(C(=O)OC(C)(C)C)cc2)N1C(=O)CCl] |
| 22 | 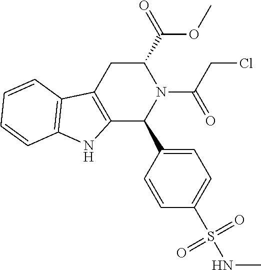 [SMILES: CNS(=O)(=O)c1ccc([C@H]2c3[nH]c4ccccc4c3C[C@H](C(=O)OC)N2C(=O)CCl)cc1] |
| 23 | 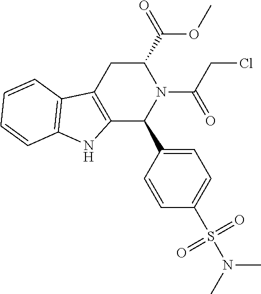 [SMILES: COC(=O)[C@H]1Cc2c([nH]c3ccccc23)[C@H](c2ccc(S(=O)(=O)N(C)C)cc2)N1C(=O)CCl] |
| 24 | 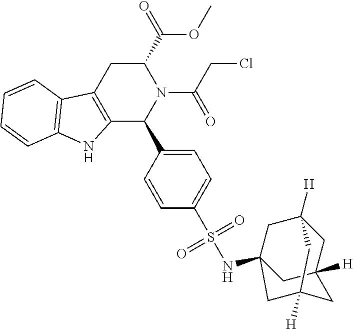 [SMILES: COC(=O)[C@H]1Cc2c([nH]c3ccccc23)[C@H](c2ccc(S(=O)(=O)N[C@]34C[C@H]5C[C@H](C[C@H](C5)C3)C4)cc2)N1C(=O)CCl] |
| 25 | 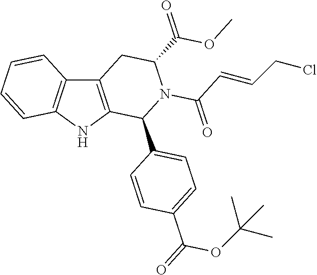 [SMILES: COC(=O)[C@H]1Cc2c([nH]c3ccccc23)[C@H](c2ccc(C(=O)OC(C)(C)C)cc2)N1C(=O)/C=C/CCl] |
| 26 | 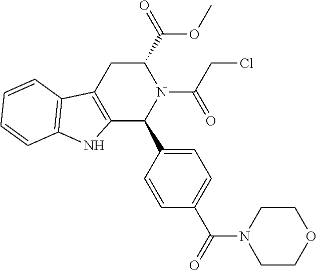 [SMILES: COC(=O)[C@H]1Cc2c([nH]c3ccccc23)[C@H](c2ccc(C(=O)N3CCOCC3)cc2)N1C(=O)CCl] |
| 27 | 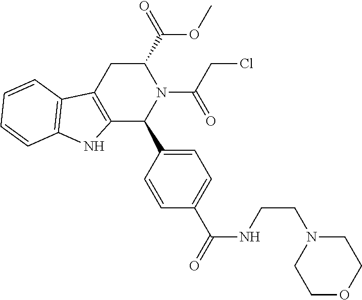 [SMILES: COC(=O)[C@H]1Cc2c([nH]c3ccccc23)[C@H](c2ccc(C(=O)NCCN3CCOCC3)cc2)N1C(=O)CCl] |
| 28 | 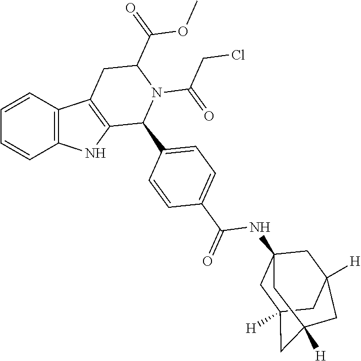 [SMILES: COC(=O)C1Cc2c([nH]c3ccccc23)[C@H](c2ccc(C(=O)N[C@]34CC5C[C@H](C[C@H](C5)C3)C4)cc2)N1C(=O)CCl] |
| 29 | 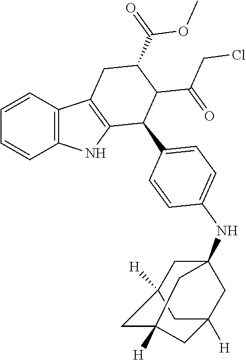 [SMILES: COC(=O)[C@H]1Cc2c([nH]c3ccccc23)[C@H](c2ccc(N[C@]34CC5C[C@H](C[C@H](C5)C3)C4)cc2)C1C(=O)CCl] |
| 30 | 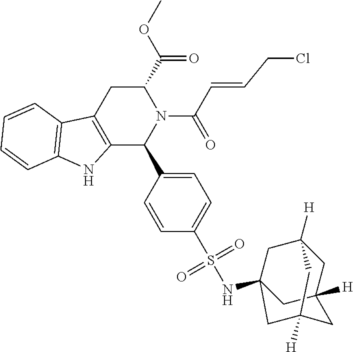 [SMILES: COC(=O)[C@H]1Cc2c([nH]c3ccccc23)[C@H](c2ccc(S(=O)(=O)N[C@]34C[C@H]5C[C@H](C[C@H](C5)C3)C4)cc2)N1C(=O)/C=C/CCl] |
| 31 | 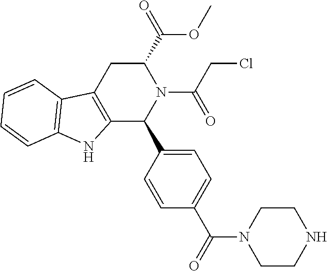 [SMILES: COC(=O)[C@H]1Cc2c([nH]c3ccccc23)[C@H](c2ccc(C(=O)N3CCNCC3)cc2)N1C(=O)CCl] |
| 32 | 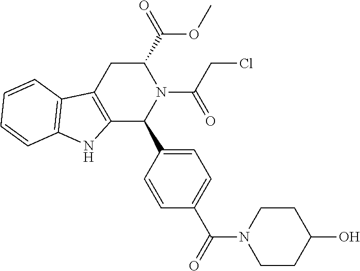 [SMILES: COC(=O)[C@H]1Cc2c([nH]c3ccccc23)[C@H](c2ccc(C(=O)N3CCC(O)CC3)cc2)N1C(=O)CCl] |
| 33 | 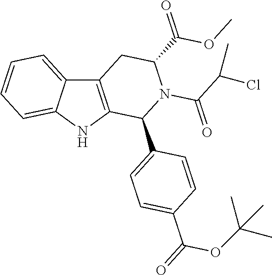 [SMILES: COC(=O)[C@H]1Cc2c([nH]c3ccccc23)[C@H](c2ccc(C(=O)OC(C)(C)C)cc2)N1C(=O)C(C)Cl] |
| 34 | 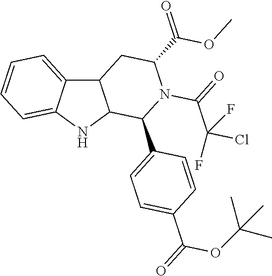 [SMILES: COC(=O)[C@H]1CC2c3ccccc3NC2[C@H](c2ccc(C(=O)OC(C)(C)C)cc2)N1C(=O)C(F)(F)Cl] |
| 35 | 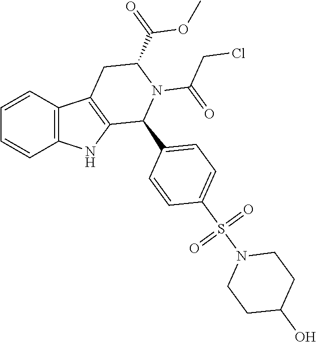 [SMILES: COC(=O)[C@H]1Cc2c([nH]c3ccccc23)[C@H](c2ccc(S(=O)(=O)N3CCC(O)CC3)cc2)N1C(=O)CCl] |
| 36 | 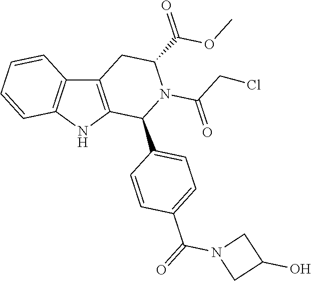 [SMILES: COC(=O)[C@H]1Cc2c([nH]c3ccccc23)[C@H](c2ccc(C(=O)N3CC(O)C3)cc2)N1C(=O)CCl] |
| 37 | 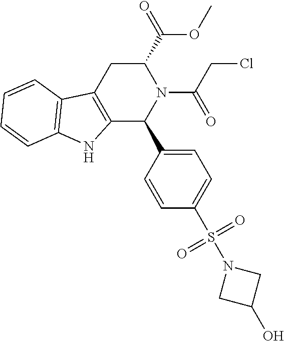 [SMILES: COC(=O)[C@H]1Cc2c([nH]c3ccccc23)[C@H](c2ccc(S(=O)(=O)N3CC(O)C3)cc2)N1C(=O)CCl] |
| 38 | 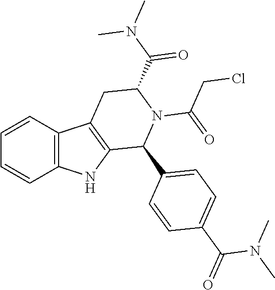 [SMILES: CN(C)C(=O)c1ccc([C@H]2c3[nH]c4ccccc4c3C[C@H](C(=O)N(C)C)N2C(=O)CCl)cc1] |
| 39 | 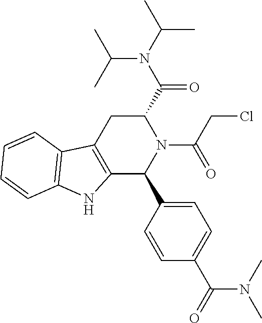 [SMILES: CC(C)N(C(=O)[C@H]1Cc2c([nH]c3ccccc23)[C@H](c2ccc(C(=O)N(C)C)cc2)N1C(=O)CCl)C(C)C] |
| 40 | 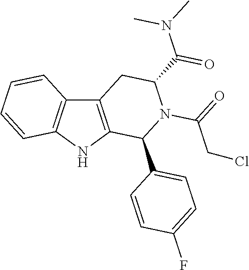 [SMILES: CN(C)C(=O)[C@H]1Cc2c([nH]c3ccccc23)[C@H](c2ccc(F)cc2)N1C(=O)CCl] |
| 41 | 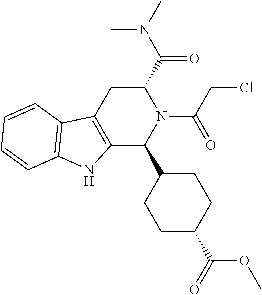 |
| 42 | 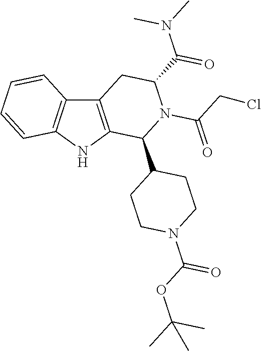 [SMILES: CN(C)C(=O)[C@H]1Cc2c([nH]c3ccccc23)[C@H](C2CCN(C(=O)OC(C)(C)C)CC2)N1C(=O)CCl] |
| 43 | 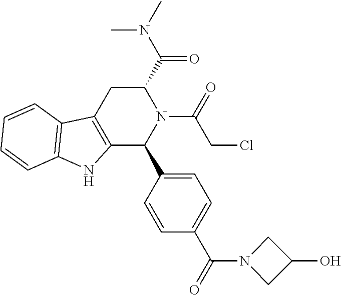 [SMILES: CN(C)C(=O)[C@H]1Cc2c([nH]c3ccccc23)[C@H](c2ccc(C(=O)N3CC(O)C3)cc2)N1C(=O)CCl] |
| 44 | 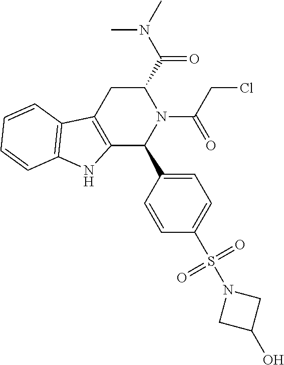 [SMILES: CN(C)C(=O)[C@H]1Cc2c([nH]c3ccccc23)[C@H](c2ccc(S(=O)(=O)N3CC(O)C3)cc2)N1C(=O)CCl] |
| 45 | 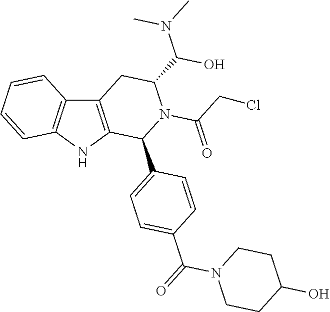 [SMILES: CN(C)C(O)[C@H]1Cc2c([nH]c3ccccc23)[C@H](c2ccc(C(=O)N3CCC(O)CC3)cc2)N1C(=O)CCl] |
| 46 | 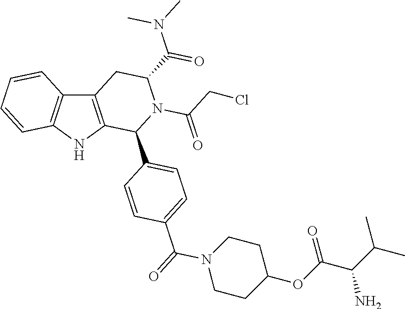 [SMILES: CC(C)[C@H](N)C(=O)OC1CCN(C(=O)c2ccc([C@H]3c4[nH]c5ccccc5c4C[C@H](C(=O)N(C)C)N3C(=O)CCl)cc2)CC1] |
| 47 | 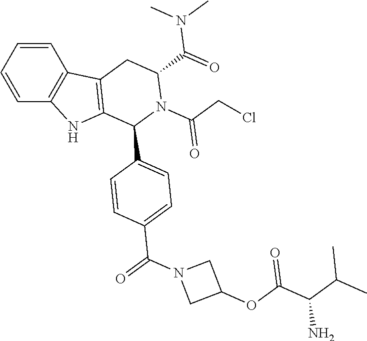 [SMILES: CC(C)[C@H](N)C(=O)OC1CN(C(=O)c2ccc([C@H]3c4[nH]c5ccccc5c4C[C@H](C(=O)N(C)C)N3C(=O)CCl)cc2)C1] |
| 48 | 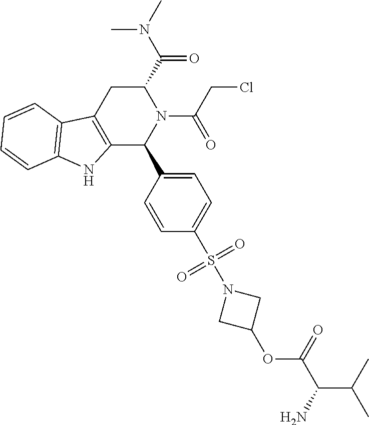 [SMILES: CC(C)[C@H](N)C(=O)OC1CN(S(=O)(=O)c2ccc([C@H]3c4[nH]c5ccccc5c4C[C@H](C(=O)N(C)C)N3C(=O)CCl)cc2)C1] |
| 49 | 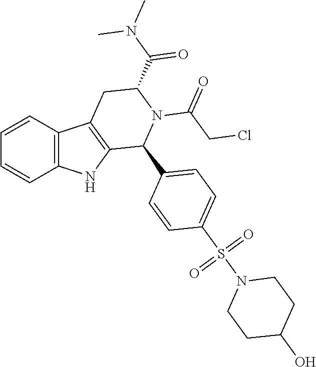 [SMILES: CN(C)C(=O)[C@H]1Cc2c([nH]c3ccccc23)[C@H](c2ccc(S(=O)(=O)N3CCC(O)CC3)cc2)N1C(=O)CCl] |
| 50 | 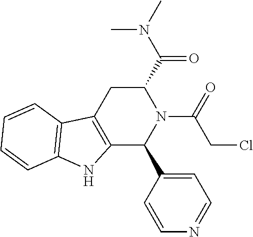 [SMILES: CN(C)C(=O)[C@H]1Cc2c([nH]c3ccccc23)[C@H](c2ccncc2)N1C(=O)CCl] |
| 51 | 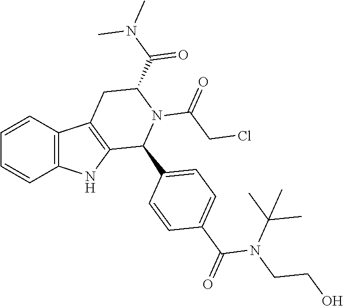 [SMILES: CN(C)C(=O)[C@H]1Cc2c([nH]c3ccccc23)[C@H](c2ccc(C(=O)N(CCO)C(C)(C)C)cc2)N1C(=O)CCl] |
| 52 | 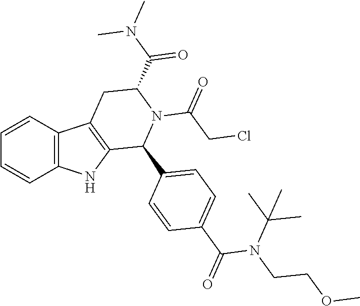 [SMILES: COCCN(C(=O)c1ccc([C@H]2c3[nH]c4ccccc4c3C[C@H](C(=O)N(C)C)N2C(=O)CCl)cc1)C(C)(C)C] |
| 53 | 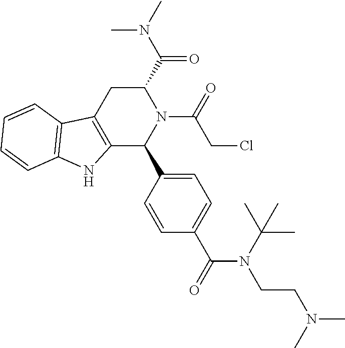 [SMILES: CN(C)CCN(C(=O)c1ccc([C@H]2c3[nH]c4ccccc4c3C[C@H](C(=O)N(C)C)N2C(=O)CCl)cc1)C(C)(C)C] |
| 54 | 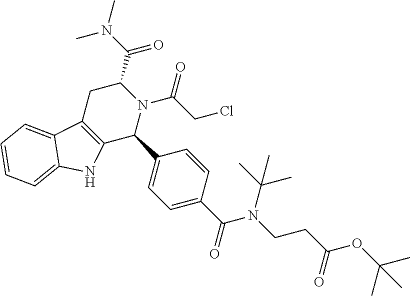 [SMILES: CN(C)C(=O)[C@H]1Cc2c([nH]c3ccccc23)[C@H](c2ccc(C(=O)N(CCC(=O)OC(C)(C)C)C(C)(C)C)cc2)N1C(=O)CCl] |
| 55 | 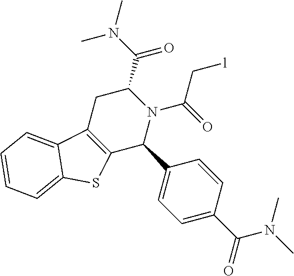 |
| 56 | 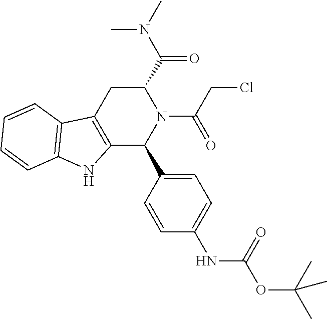 [SMILES: CN(C)C(=O)[C@H]1Cc2c([nH]c3ccccc23)[C@H](c2ccc(NC(=O)OC(C)(C)C)cc2)N1C(=O)CCl] |
| 57 | 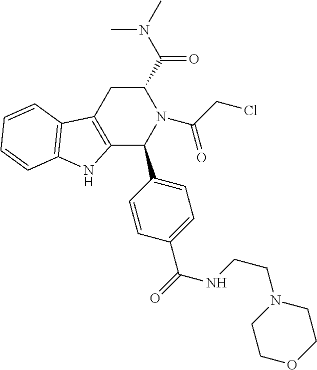 [SMILES: CN(C)C(=O)[C@H]1Cc2c([nH]c3ccccc23)[C@H](c2ccc(C(=O)NCCN3CCOCC3)cc2)N1C(=O)CCl] |
| 58 | 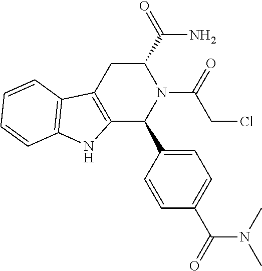 [SMILES: CN(C)C(=O)c1ccc([C@H]2c3[nH]c4ccccc4c3C[C@H](C(N)=O)N2C(=O)CCl)cc1] |
| 59 | 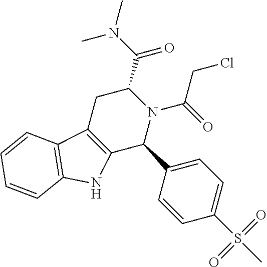 [SMILES: CN(C)C(=O)[C@H]1Cc2c([nH]c3ccccc23)[C@H](c2ccc(S(C)(=O)=O)cc2)N1C(=O)CCl] |
| 60 | 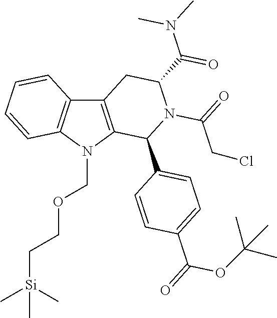 [SMILES: CN(C)C(=O)[C@H]1Cc2c(n(COCC[Si](C)(C)C)c3ccccc23)[C@H](c2ccc(C(=O)OC(C)(C)C)cc2)N1C(=O)CCl] |
| 61 | 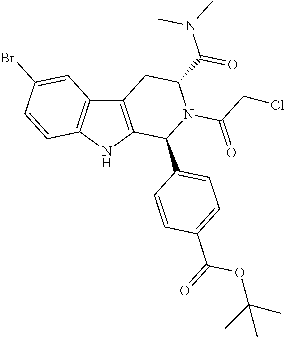 [SMILES: CN(C)C(=O)[C@H]1Cc2c([nH]c3ccc(Br)cc23)[C@H](c2ccc(C(=O)OC(C)(C)C)cc2)N1C(=O)CCl] |
| 62 | 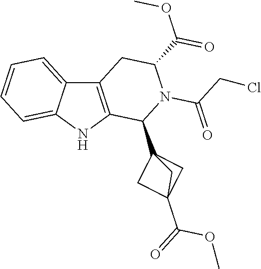 [SMILES: COC(=O)[C@H]1Cc2c([nH]c3ccccc23)[C@H](C23CC(C(=O)OC)(C2)C3)N1C(=O)CCl] |
| 63 | 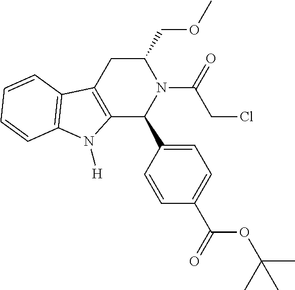 [SMILES: COC[C@H]1Cc2c([nH]c3ccccc23)[C@H](c2ccc(C(=O)OC(C)(C)C)cc2)N1C(=O)CCl] |
| 64 | 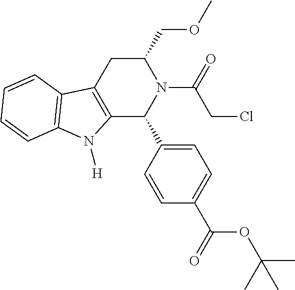 [SMILES: COC[C@H]1Cc2c([nH]c3ccccc23)[C@@H](c2ccc(C(=O)OC(C)(C)C)cc2)N1C(=O)CCl] |
| 65 | 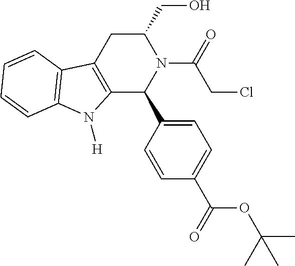 [SMILES: CC(C)(C)OC(=O)c1ccc([C@H]2c3[nH]c4ccccc4c3C[C@H](CO)N2C(=O)CCl)cc1] |
| 66 | 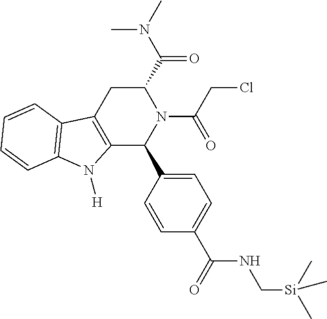 [SMILES: CN(C)C(=O)[C@H]1Cc2c([nH]c3ccccc23)[C@H](c2ccc(C(=O)NC[Si](C)(C)C)cc2)N1C(=O)CCl] |
| 67 | 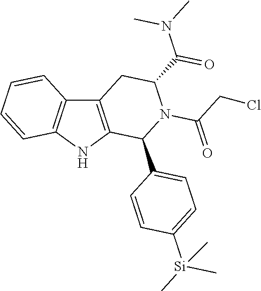 [SMILES: CN(C)C(=O)[C@H]1Cc2c([nH]c3ccccc23)[C@H](c2ccc([Si](C)(C)C)cc2)N1C(=O)CCl] |
| 68 | 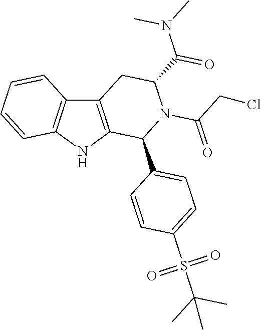 [SMILES: CN(C)C(=O)[C@H]1Cc2c([nH]c3ccccc23)[C@H](c2ccc(S(=O)(=O)C(C)(C)C)cc2)N1C(=O)CCl] |
| 69 | 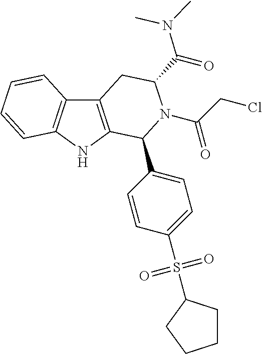 [SMILES: CN(C)C(=O)[C@H]1Cc2c([nH]c3ccccc23)[C@H](c2ccc(S(=O)(=O)C3CCCC3)cc2)N1C(=O)CCl] |
| 70 | 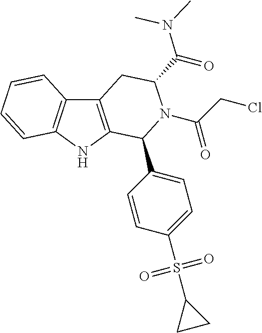 [SMILES: CN(C)C(=O)[C@H]1Cc2c([nH]c3ccccc23)[C@H](c2ccc(S(=O)(=O)C3CC3)cc2)N1C(=O)CCl] |
| 71 | 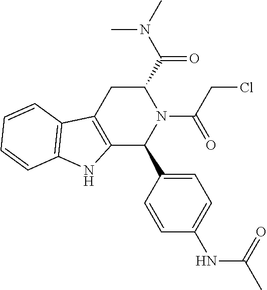 [SMILES: CC(=O)Nc1ccc([C@H]2c3[nH]c4ccccc4c3C[C@H](C(=O)N(C)C)N2C(=O)CCl)cc1] |
| 72 | 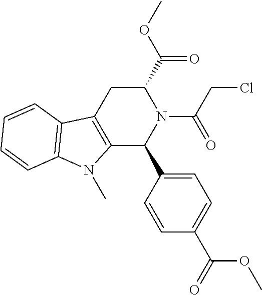 [SMILES: COC(=O)c1ccc([C@H]2c3c(c4ccccc4n3C)C[C@H](C(=O)OC)N2C(=O)CCl)cc1] |
| 73 | 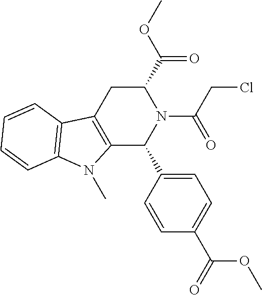 [SMILES: COC(=O)c1ccc([C@@H]2c3c(c4ccccc4n3C)C[C@H](C(=O)OC)N2C(=O)CCl)cc1] |
| 74 | 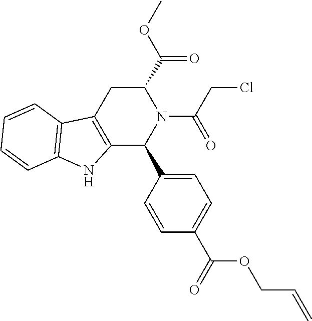 [SMILES: C=CCOC(=O)c1ccc([C@H]2c3[nH]c4ccccc4c3C[C@H](C(=O)OC)N2C(=O)CCl)cc1] |
| 75 | 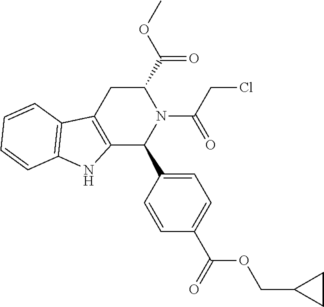 [SMILES: COC(=O)[C@H]1Cc2c([nH]c3ccccc23)[C@H](c2ccc(C(=O)OCC3CC3)cc2)N1C(=O)CCl] |
| 76 | 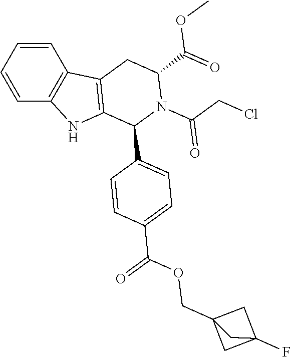 [SMILES: COC(=O)[C@H]1Cc2c([nH]c3ccccc23)[C@H](c2ccc(C(=O)OCC34CC(F)(C3)C4)cc2)N1C(=O)CCl] |
| 77 | 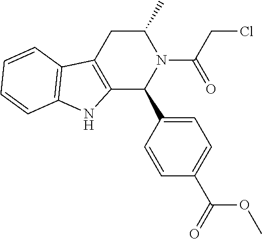 [SMILES: COC(=O)c1ccc([C@H]2c3[nH]c4ccccc4c3C[C@H](C)N2C(=O)CCl)cc1] |
| 78 | 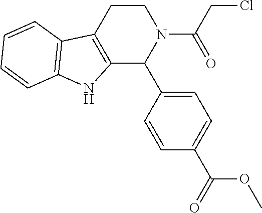 [SMILES: COC(=O)c1ccc(C2c3[nH]c4ccccc4c3CCN2C(=O)CCl)cc1] |
| 79 | 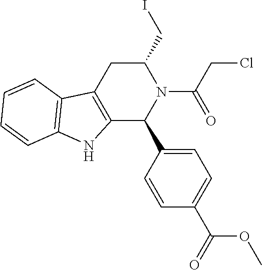 [SMILES: COC(=O)c1ccc([C@H]2c3[nH]c4ccccc4c3C[C@H](CI)N2C(=O)CCl)cc1] |
| 80 | 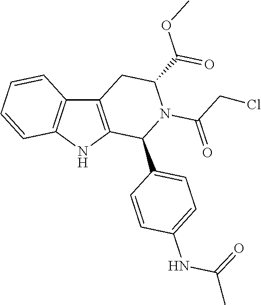 [SMILES: COC(=O)[C@H]1Cc2c([nH]c3ccccc23)[C@H](c2ccc(NC(C)=O)cc2)N1C(=O)CCl] |
| 81 | 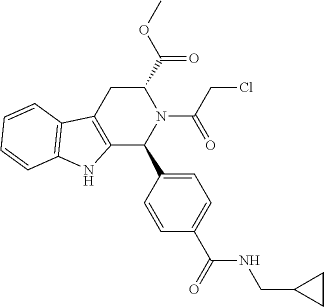 [SMILES: COC(=O)[C@H]1Cc2c([nH]c3ccccc23)[C@H](c2ccc(C(=O)NCC3CC3)cc2)N1C(=O)CCl] |
| 82 | 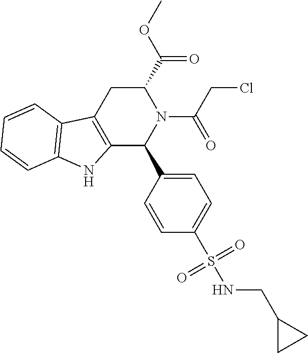 [SMILES: COC(=O)[C@H]1Cc2c([nH]c3ccccc23)[C@H](c2ccc(S(=O)(=O)NCC3CC3)cc2)N1C(=O)CCl] |
| 83 | 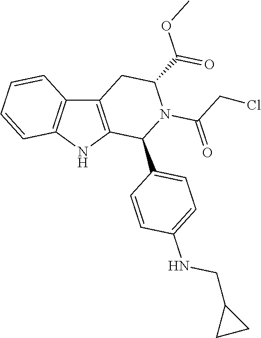 [SMILES: COC(=O)[C@H]1Cc2c([nH]c3ccccc23)[C@H](c2ccc(NCC3CC3)cc2)N1C(=O)CCl] |
| 84 | 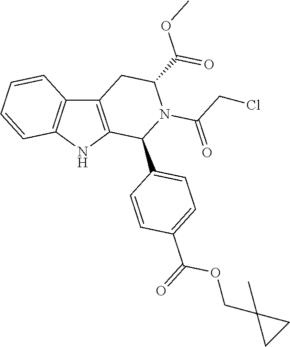 [SMILES: COC(=O)[C@H]1Cc2c([nH]c3ccccc23)[C@H](c2ccc(C(=O)OCC3(C)CC3)cc2)N1C(=O)CCl] |
| 85 | 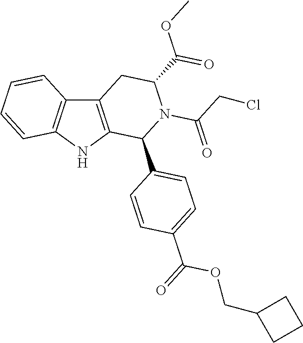 [SMILES: COC(=O)[C@H]1Cc2c([nH]c3ccccc23)[C@H](c2ccc(C(=O)OCC3CCC3)cc2)N1C(=O)CCl] |
| 86 | 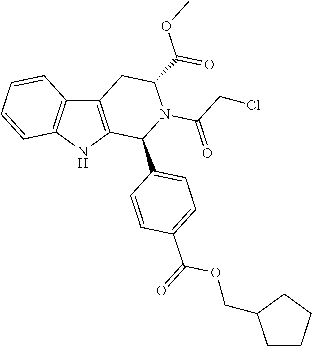 [SMILES: COC(=O)[C@H]1Cc2c([nH]c3ccccc23)[C@H](c2ccc(C(=O)OCC3CCCC3)cc2)N1C(=O)CCl] |
| 87 | 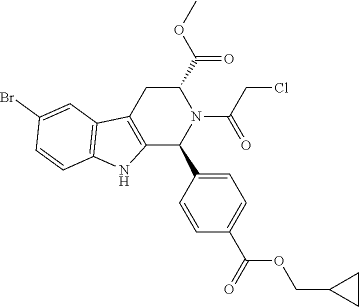 [SMILES: COC(=O)[C@H]1Cc2c([nH]c3ccc(Br)cc23)[C@H](c2ccc(C(=O)OCC3CC3)cc2)N1C(=O)CCl] |
| 88 | 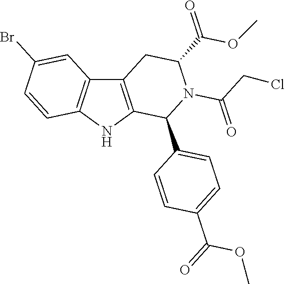 [SMILES: COC(=O)c1ccc([C@H]2c3[nH]c4ccc(Br)cc4c3C[C@H](C(=O)OC)N2C(=O)CCl)cc1] |
| 89 | 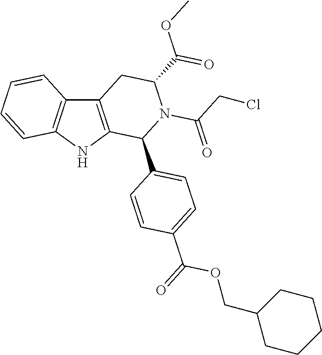 [SMILES: COC(=O)[C@H]1Cc2c([nH]c3ccccc23)[C@H](c2ccc(C(=O)OCC3CCCCC3)cc2)N1C(=O)CCl] |
| 90 | 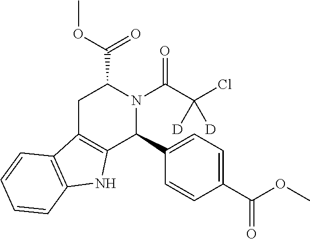 [SMILES: [2H]C([2H])(Cl)C(=O)N1[C@@H](c2ccc(C(=O)OC)cc2)c2[nH]c3ccccc3c2C[C@@H]1C(=O)OC] |
| 91 | 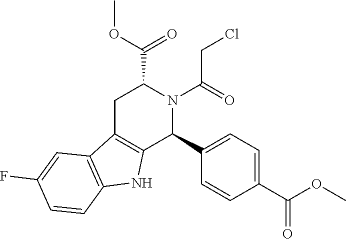 [SMILES: COC(=O)c1ccc([C@H]2c3[nH]c4ccc(F)cc4c3C[C@H](C(=O)OC)N2C(=O)CCl)cc1] |
| 92 | 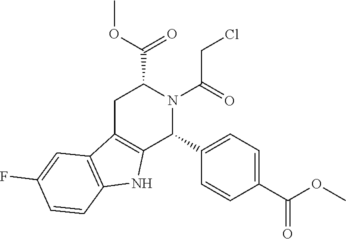 [SMILES: COC(=O)c1ccc([C@@H]2c3[nH]c4ccc(F)cc4c3C[C@H](C(=O)OC)N2C(=O)CCl)cc1] |
| 93 | 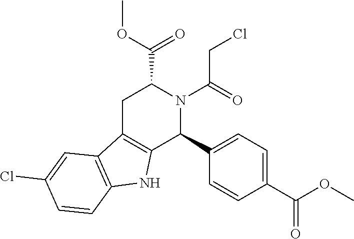 [SMILES: COC(=O)c1ccc([C@H]2c3[nH]c4ccc(Cl)cc4c3C[C@H](C(=O)OC)N2C(=O)CCl)cc1] |
| 94 | 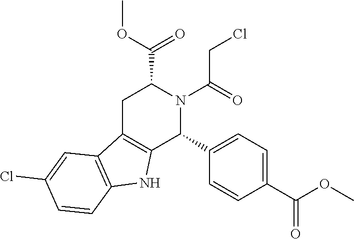 [SMILES: COC(=O)c1ccc([C@@H]2c3[nH]c4ccc(Cl)cc4c3C[C@H](C(=O)OC)N2C(=O)CCl)cc1] |
| 95 | 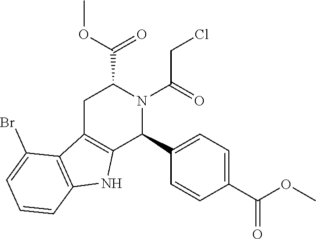 [SMILES: COC(=O)c1ccc([C@H]2c3[nH]c4cccc(Br)c4c3C[C@H](C(=O)OC)N2C(=O)CCl)cc1] |
| 96 | 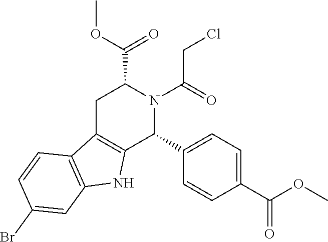 [SMILES: COC(=O)c1ccc([C@@H]2c3[nH]c4cc(Br)ccc4c3C[C@H](C(=O)OC)N2C(=O)CCl)cc1] |
| 97 | 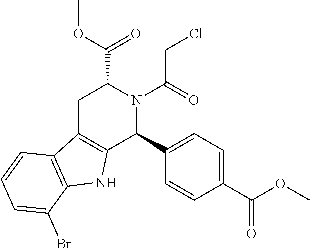 [SMILES: COC(=O)c1ccc([C@H]2c3[nH]c4c(Br)cccc4c3C[C@H](C(=O)OC)N2C(=O)CCl)cc1] |
| 98 | 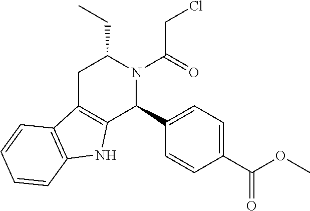 [SMILES: CC[C@H]1Cc2c([nH]c3ccccc23)[C@H](c2ccc(C(=O)OC)cc2)N1C(=O)CCl] |
| 99 | 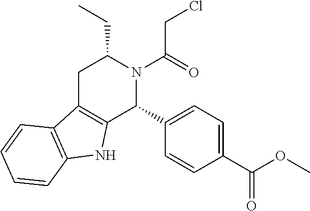 [SMILES: CC[C@H]1Cc2c([nH]c3ccccc23)[C@@H](c2ccc(C(=O)OC)cc2)N1C(=O)CCl] |
| 100 | 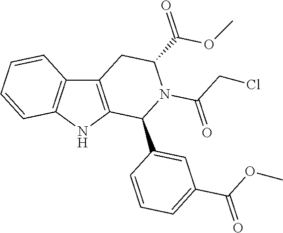 [SMILES: COC(=O)c1cccc([C@H]2c3[nH]c4ccccc4c3C[C@H](C(=O)OC)N2C(=O)CCl)c1] |
| 101 | 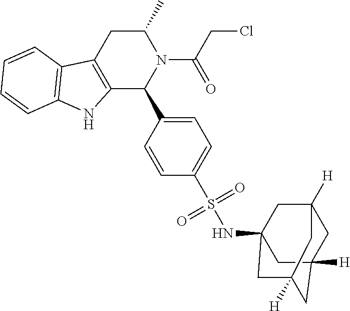 [SMILES: C[C@H]1Cc2c([nH]c3ccccc23)[C@H](c2ccc(S(=O)(=O)N[C@]34CC5C[C@H](C[C@H](C5)C3)C4)cc2)N1C(=O)CCl] |
| 102 | 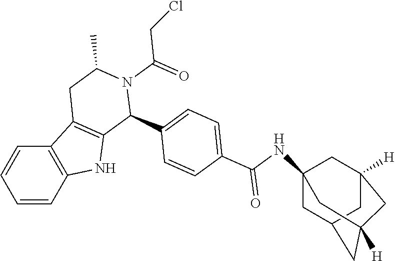 [SMILES: C[C@H]1Cc2c([nH]c3ccccc23)[C@H](c2ccc(C(=O)N[C@]34CC5C[C@H](C[C@H](C5)C3)C4)cc2)N1C(=O)CCl] |
| 103 | 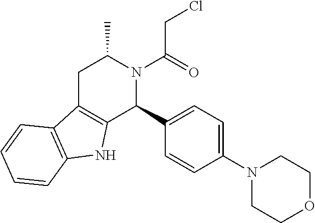 [SMILES: C[C@H]1Cc2c([nH]c3ccccc23)[C@H](c2ccc(N3CCOCC3)cc2)N1C(=O)CCl] |
| 104 | 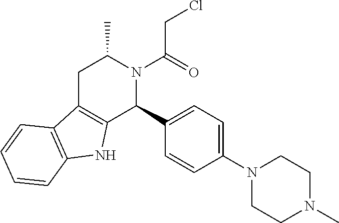 [SMILES: C[C@H]1Cc2c([nH]c3ccccc23)[C@H](c2ccc(N3CCN(C)CC3)cc2)N1C(=O)CCl] |
| 105 | 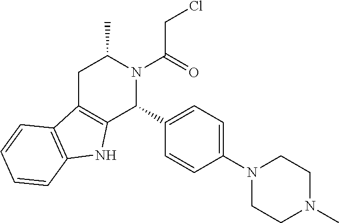 [SMILES: C[C@H]1Cc2c([nH]c3ccccc23)[C@@H](c2ccc(N3CCN(C)CC3)cc2)N1C(=O)CCl] |
| 106 | 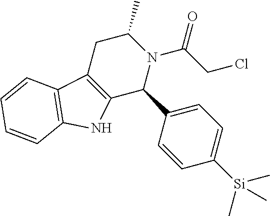 [SMILES: C[C@H]1Cc2c([nH]c3ccccc23)[C@H](c2ccc([Si](C)(C)C)cc2)N1C(=O)CCl] |
| 107 | 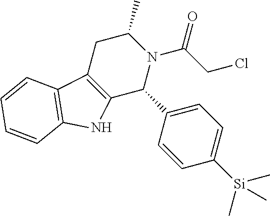 [SMILES: C[C@H]1Cc2c([nH]c3ccccc23)[C@@H](c2ccc([Si](C)(C)C)cc2)N1C(=O)CCl] |
| 108 | 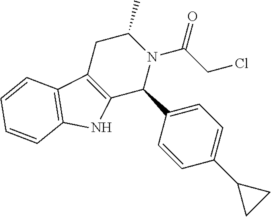 [SMILES: C[C@H]1Cc2c([nH]c3ccccc23)[C@H](c2ccc(C3CC3)cc2)N1C(=O)CCl] |
| 109 | 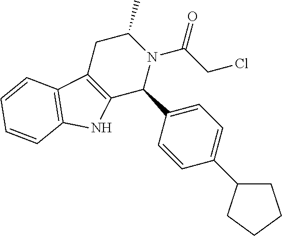 [SMILES: C[C@H]1Cc2c([nH]c3ccccc23)[C@H](c2ccc(C3CCCC3)cc2)N1C(=O)CCl] |
| 110 | 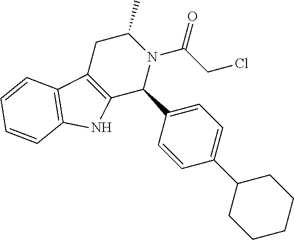 [SMILES: C[C@H]1Cc2c([nH]c3ccccc23)[C@H](c2ccc(C3CCCCC3)cc2)N1C(=O)CCl] |
| 111 | 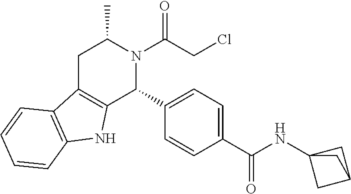 |
| 112 | 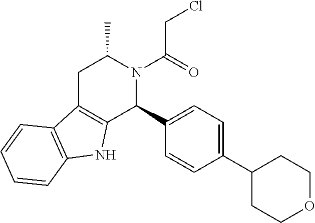 [SMILES: C[C@H]1Cc2c([nH]c3ccccc23)[C@H](c2ccc(C3CCOCC3)cc2)N1C(=O)CCl] |
| 113 | 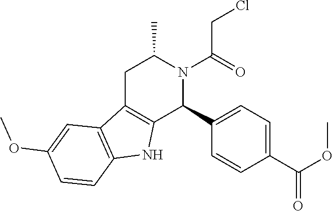 |
| 114 | 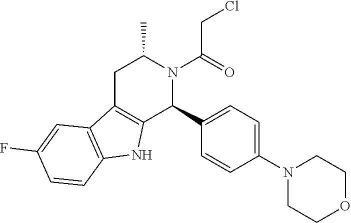 [SMILES: C[C@H]1Cc2c([nH]c3ccc(F)cc23)[C@H](c2ccc(N3CCOCC3)cc2)N1C(=O)CCl] |
| 115 | 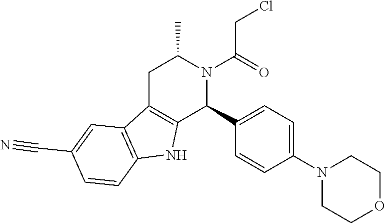 [SMILES: C[C@H]1Cc2c([nH]c3ccc(C#N)cc23)[C@H](c2ccc(N3CCOCC3)cc2)N1C(=O)CCl] |
| 116 | 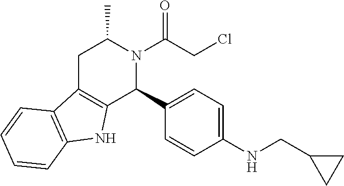 [SMILES: C[C@H]1Cc2c([nH]c3ccccc23)[C@H](c2ccc(NCC3CC3)cc2)N1C(=O)CCl] |
| 117 | 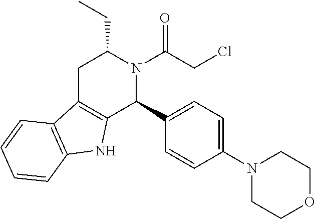 [SMILES: CC[C@H]1Cc2c([nH]c3ccccc23)[C@H](c2ccc(N3CCOCC3)cc2)N1C(=O)CCl] |
| 118 | 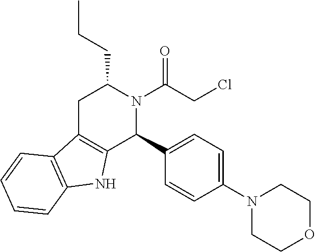 [SMILES: CCC[C@H]1Cc2c([nH]c3ccccc23)[C@H](c2ccc(N3CCOCC3)cc2)N1C(=O)CCl] |
| 119 | 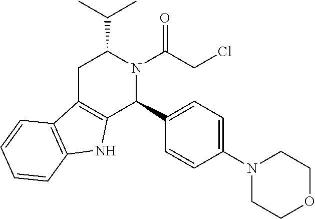 [SMILES: CC(C)[C@H]1Cc2c([nH]c3ccccc23)[C@H](c2ccc(N3CCOCC3)cc2)N1C(=O)CCl] |
| 120 | 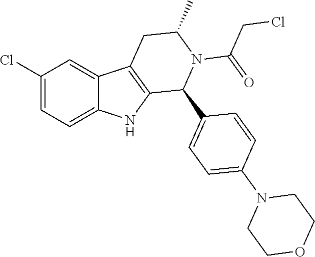 [SMILES: C[C@H]1Cc2c([nH]c3ccc(Cl)cc23)[C@H](c2ccc(N3CCOCC3)cc2)N1C(=O)CCl] |
| 121 | 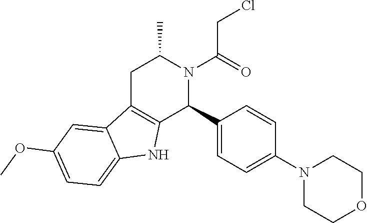 [SMILES: COc1ccc2[nH]c3c(c2c1)C[C@H](C)N(C(=O)CCl)[C@H]3c1ccc(N2CCOCC2)cc1] |
| 122 | 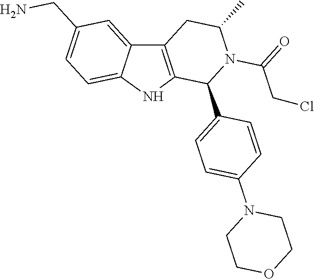 [SMILES: C[C@H]1Cc2c([nH]c3ccc(CN)cc23)[C@H](c2ccc(N3CCOCC3)cc2)N1C(=O)CCl] |
| 123 | 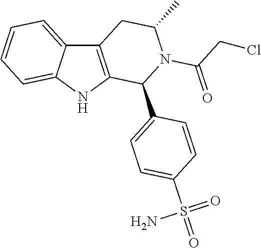 [SMILES: C[C@H]1Cc2c([nH]c3ccccc23)[C@H](c2ccc(S(N)(=O)=O)cc2)N1C(=O)CCl] |
| 124 | 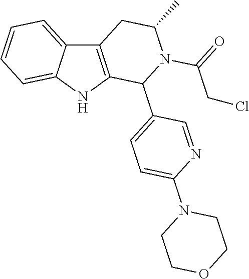 [SMILES: C[C@H]1Cc2c([nH]c3ccccc23)C(c2ccc(N3CCOCC3)nc2)N1C(=O)CCl] |
| 125 | 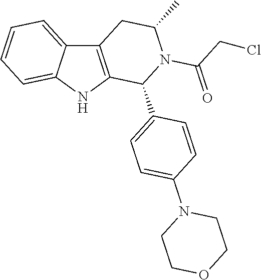 [SMILES: C[C@H]1Cc2c([nH]c3ccccc23)[C@@H](c2ccc(N3CCOCC3)cc2)N1C(=O)CCl] |
| 127 | 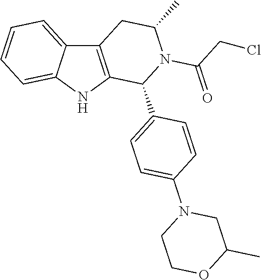 [SMILES: CC1CN(c2ccc([C@@H]3c4[nH]c5ccccc5c4C[C@H](C)N3C(=O)CCl)cc2)CCO1] |
| 128 | 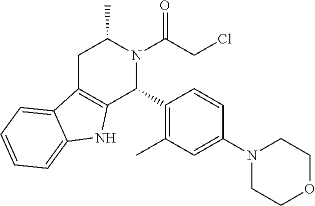 [SMILES: Cc1cc(N2CCOCC2)ccc1[C@@H]1c2[nH]c3ccccc3c2C[C@H](C)N1C(=O)CCl] |
| 129 | 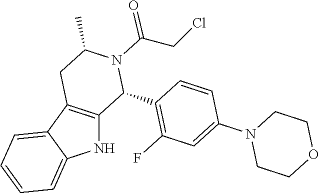 [SMILES: C[C@H]1Cc2c([nH]c3ccccc23)[C@@H](c2ccc(N3CCOCC3)cc2F)N1C(=O)CCl] |
| 130 | 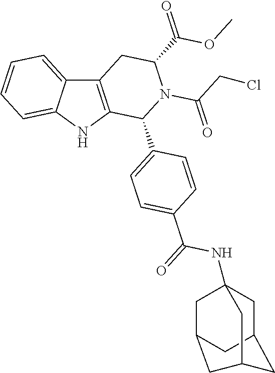 [SMILES: COC(=O)[C@H]1Cc2c([nH]c3ccccc23)[C@@H](c2ccc(C(=O)NC34CC5CC(CC(C5)C3)C4)cc2)N1C(=O)CCl] |
| 132 | 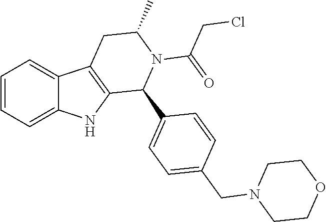 [SMILES: C[C@H]1Cc2c([nH]c3ccccc23)[C@H](c2ccc(CN3CCOCC3)cc2)N1C(=O)CCl] |
| 133 | 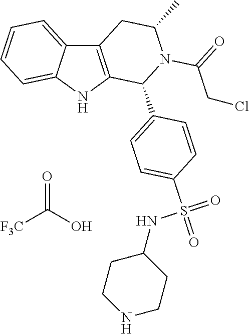 [SMILES: C[C@H]1Cc2c([nH]c3ccccc23)[C@@H](c2ccc(S(=O)(=O)NC3CCNCC3)cc2)N1C(=O)CCl ; O=C(O)C(F)(F)F] |
| 134 | 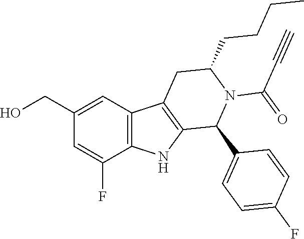 [SMILES: C#CC(=O)N1[C@@H](CCCC)Cc2c([nH]c3c(F)cc(CO)cc23)[C@@H]1c1ccc(F)cc1] |
| 135 | 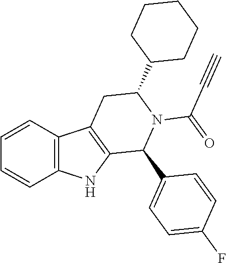 [SMILES: C#CC(=O)N1[C@@H](c2ccc(F)cc2)c2[nH]c3ccccc3c2C[C@@H]1C1CCCCC1] |
| 136 | 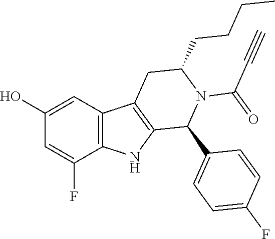 [SMILES: C#CC(=O)N1[C@@H](CCCC)Cc2c([nH]c3c(F)cc(O)cc23)[C@@H]1c1ccc(F)cc1] |
| 137 | 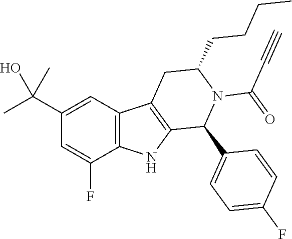 [SMILES: C#CC(=O)N1[C@@H](CCCC)Cc2c([nH]c3c(F)cc(C(C)(C)O)cc23)[C@@H]1c1ccc(F)cc1] |
| 138 | 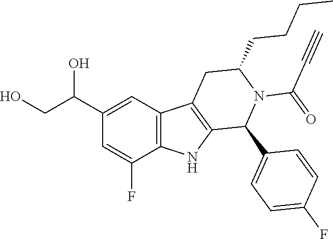 [SMILES: C#CC(=O)N1[C@@H](CCCC)Cc2c([nH]c3c(F)cc(C(O)CO)cc23)[C@@H]1c1ccc(F)cc1] |
| 139 | 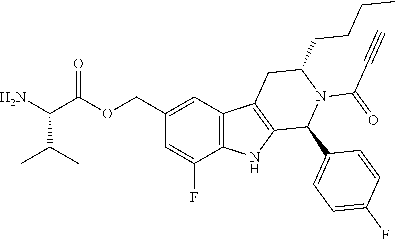 [SMILES: C#CC(=O)N1[C@@H](CCCC)Cc2c([nH]c3c(F)cc(COC(=O)[C@@H](N)C(C)C)cc23)[C@@H]1c1ccc(F)cc1] |
| 140 | 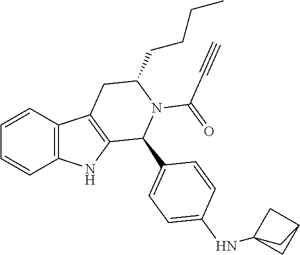 [SMILES: C#CC(=O)N1[C@@H](CCCC)Cc2c([nH]c3ccccc23)[C@@H]1c1ccc(NC23CC(C2)C3)cc1] |
| 141 | 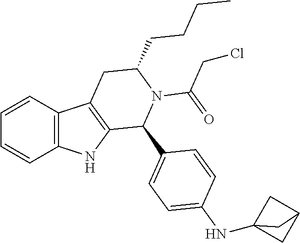 [SMILES: CCCC[C@H]1Cc2c([nH]c3ccccc23)[C@H](c2ccc(NC34CC(C3)C4)cc2)N1C(=O)CCl] |
| 142 | 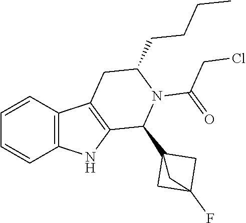 [SMILES: CCCC[C@H]1Cc2c([nH]c3ccccc23)[C@H](C23CC(F)(C2)C3)N1C(=O)CCl] |
| 143 | 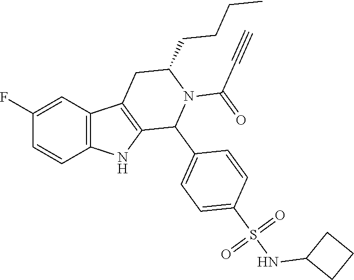 [SMILES: C#CC(=O)N1C(c2ccc(S(=O)(=O)NC3CCC3)cc2)c2[nH]c3ccc(F)cc3c2C[C@@H]1CCCC] |
| 144 | 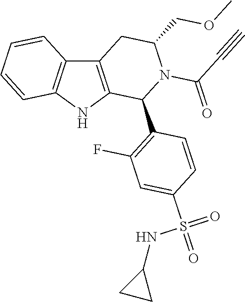 [SMILES: C#CC(=O)N1[C@@H](COC)Cc2c([nH]c3ccccc23)[C@@H]1c1ccc(S(=O)(=O)NC2CC2)cc1F] |
| 145 | 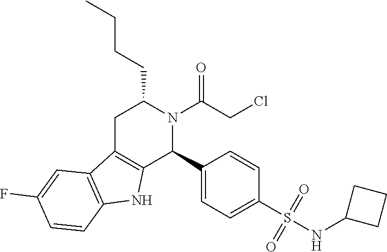 [SMILES: CCCC[C@H]1Cc2c([nH]c3ccc(F)cc23)[C@H](c2ccc(S(=O)(=O)NC3CCC3)cc2)N1C(=O)CCl] |
| 146 | 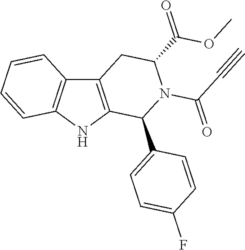 [SMILES: C#CC(=O)N1[C@@H](c2ccc(F)cc2)c2[nH]c3ccccc3c2C[C@@H]1C(=O)OC] |
| 147 | 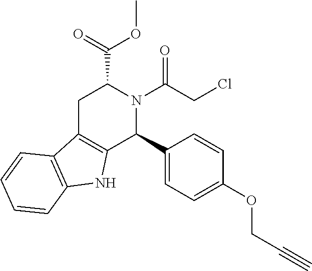 [SMILES: C#CCOc1ccc([C@H]2c3[nH]c4ccccc4c3C[C@H](C(=O)OC)N2C(=O)CCl)cc1] |
| 148 | 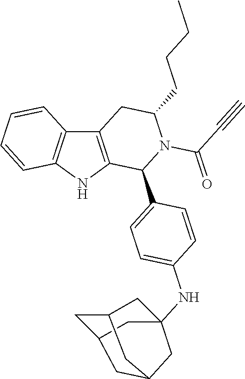 [SMILES: C#CC(=O)N1[C@@H](CCCC)Cc2c([nH]c3ccccc23)[C@@H]1c1ccc(NC23CC4CC(CC(C4)C2)C3)cc1] |
| 149 | 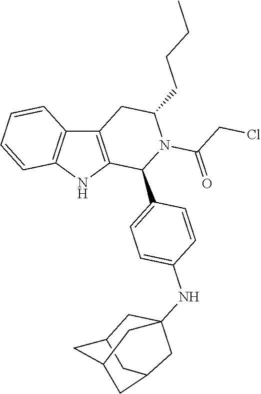 [SMILES: CCCC[C@H]1Cc2c([nH]c3ccccc23)[C@H](c2ccc(NC34CC5CC(CC(C5)C3)C4)cc2)N1C(=O)CCl] |
| 150 | 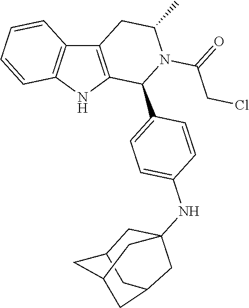 [SMILES: C[C@H]1Cc2c([nH]c3ccccc23)[C@H](c2ccc(NC34CC5CC(CC(C5)C3)C4)cc2)N1C(=O)CCl] |
| 151 | 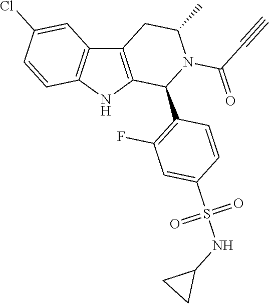 [SMILES: C#CC(=O)N1[C@@H](c2ccc(S(=O)(=O)NC3CC3)cc2F)c2[nH]c3ccc(Cl)cc3c2C[C@@H]1C] |
| 152 | 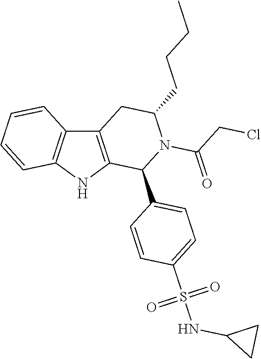 [SMILES: CCCC[C@H]1Cc2c([nH]c3ccccc23)[C@H](c2ccc(S(=O)(=O)NC3CC3)cc2)N1C(=O)CCl] |
| 153 | 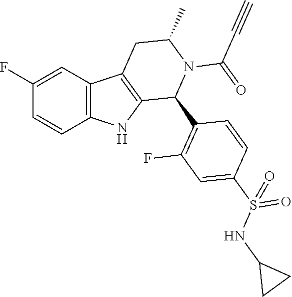 [SMILES: C#CC(=O)N1[C@@H](c2ccc(S(=O)(=O)NC3CC3)cc2F)c2[nH]c3ccc(F)cc3c2C[C@@H]1C] |
| 154 | 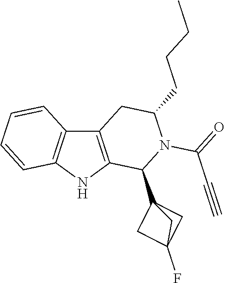 [SMILES: C#CC(=O)N1[C@@H](CCCC)Cc2c([nH]c3ccccc23)[C@@H]1C12CC(F)(C1)C2] |
| 155 | 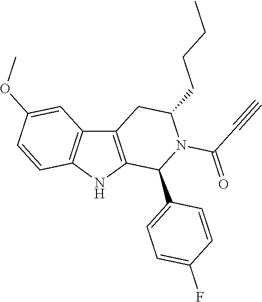 [SMILES: C#CC(=O)N1[C@@H](CCCC)Cc2c([nH]c3ccc(OC)cc23)[C@@H]1c1ccc(F)cc1] |
| 156 | 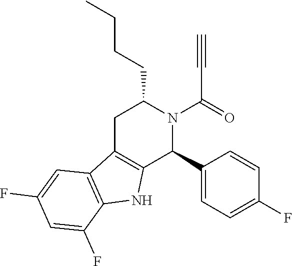 [SMILES: C#CC(=O)N1[C@@H](CCCC)Cc2c([nH]c3c(F)cc(F)cc23)[C@@H]1c1ccc(F)cc1] |
| 157 | 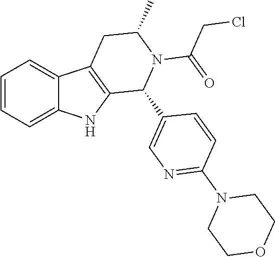 [SMILES: C[C@H]1Cc2c([nH]c3ccccc23)[C@@H](c2ccc(N3CCOCC3)nc2)N1C(=O)CCl] |
| 158 | 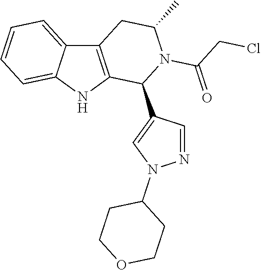 [SMILES: C[C@H]1Cc2c([nH]c3ccccc23)[C@H](c2cnn(C3CCOCC3)c2)N1C(=O)CCl] |
| 159 | 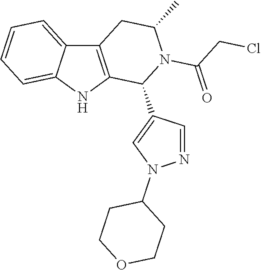 [SMILES: C[C@H]1Cc2c([nH]c3ccccc23)[C@@H](c2cnn(C3CCOCC3)c2)N1C(=O)CCl] |
| 160 | 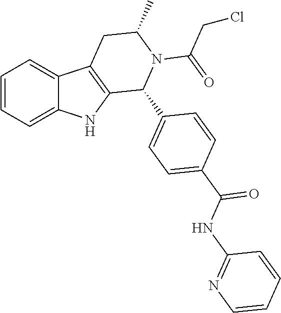 [SMILES: C[C@H]1Cc2c([nH]c3ccccc23)[C@@H](c2ccc(C(=O)Nc3ccccn3)cc2)N1C(=O)CCl] |
| 161 | 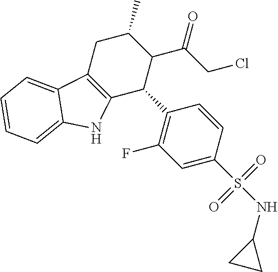 [SMILES: C[C@H]1Cc2c([nH]c3ccccc23)[C@@H](c2ccc(S(=O)(=O)NC3CC3)cc2F)C1C(=O)CCl] |
| 162 | 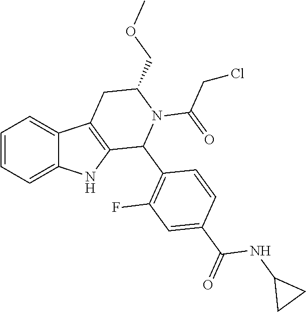 [SMILES: COC[C@H]1Cc2c([nH]c3ccccc23)C(c2ccc(C(=O)NC3CC3)cc2F)N1C(=O)CCl] |
| 163 | 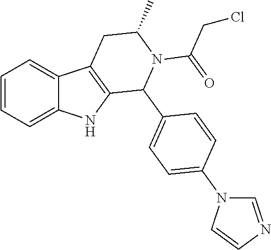 [SMILES: C[C@H]1Cc2c([nH]c3ccccc23)C(c2ccc(-n3ccnc3)cc2)N1C(=O)CCl] |
| 164 | 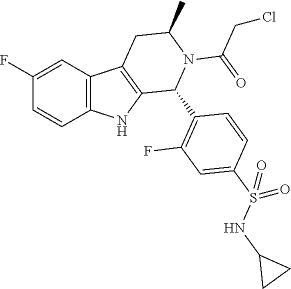 [SMILES: C[C@@H]1Cc2c([nH]c3ccc(F)cc23)[C@@H](c2ccc(S(=O)(=O)NC3CC3)cc2F)N1C(=O)CCl] |
| 165 | 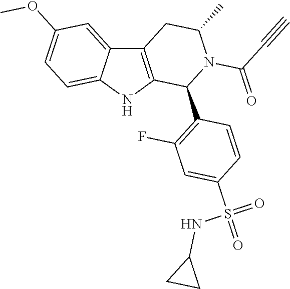 [SMILES: C#CC(=O)N1[C@@H](c2ccc(S(=O)(=O)NC3CC3)cc2F)c2[nH]c3ccc(OC)cc3c2C[C@@H]1C] |
| 166 | 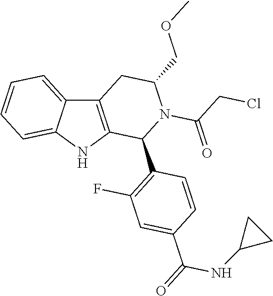 [SMILES: COC[C@H]1Cc2c([nH]c3ccccc23)[C@H](c2ccc(C(=O)NC3CC3)cc2F)N1C(=O)CCl] |
| 167 | 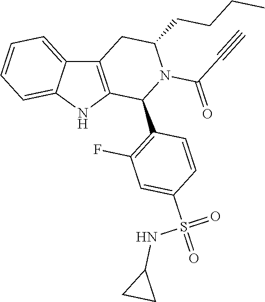 [SMILES: C#CC(=O)N1[C@@H](CCCC)Cc2c([nH]c3ccccc23)[C@@H]1c1ccc(S(=O)(=O)NC2CC2)cc1F] |
| 168 | 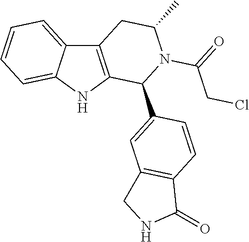 [SMILES: C[C@H]1Cc2c([nH]c3ccccc23)[C@H](c2ccc3c(c2)CNC3=O)N1C(=O)CCl] |
| 169 | 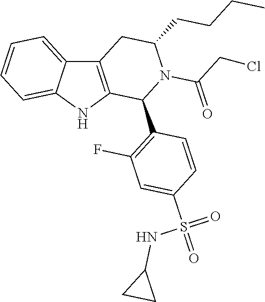 [SMILES: CCCC[C@H]1Cc2c([nH]c3ccccc23)[C@H](c2ccc(S(=O)(=O)NC3CC3)cc2F)N1C(=O)CCl] |
| 170 | 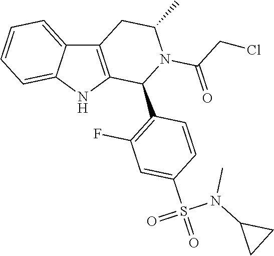 [SMILES: C[C@H]1Cc2c([nH]c3ccccc23)[C@H](c2ccc(S(=O)(=O)N(C)C3CC3)cc2F)N1C(=O)CCl] |
| 171 | 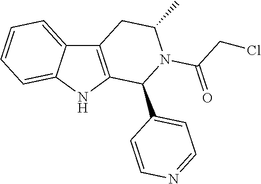 [SMILES: C[C@H]1Cc2c([nH]c3ccccc23)[C@H](c2ccncc2)N1C(=O)CCl] |
| 172 | 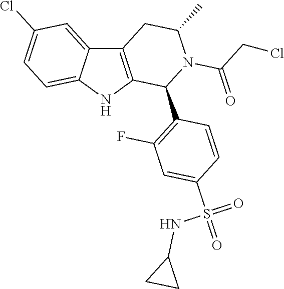 [SMILES: C[C@H]1Cc2c([nH]c3ccc(Cl)cc23)[C@H](c2ccc(S(=O)(=O)NC3CC3)cc2F)N1C(=O)CCl] |
| 173 | 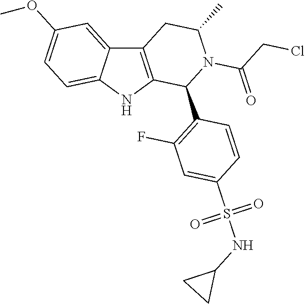 [SMILES: COc1ccc2[nH]c3c(c2c1)C[C@H](C)N(C(=O)CCl)[C@H]3c1ccc(S(=O)(=O)NC2CC2)cc1F] |
| 174 | 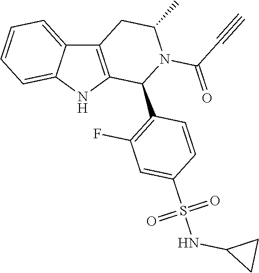 [SMILES: C#CC(=O)N1[C@@H](c2ccc(S(=O)(=O)NC3CC3)cc2F)c2[nH]c3ccccc3c2C[C@@H]1C] |
| 175 | 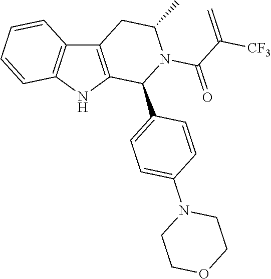 [SMILES: C=C(C(=O)N1[C@@H](c2ccc(N3CCOCC3)cc2)c2[nH]c3ccccc3c2C[C@@H]1C)C(F)(F)F] |
| 176 | 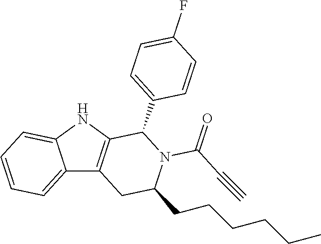 [SMILES: C#CC(=O)N1[C@@H](CCCCCC)Cc2c([nH]c3ccccc23)[C@@H]1c1ccc(F)cc1] |
| 177 | 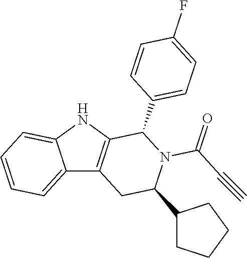 [SMILES: C#CC(=O)N1[C@@H](c2ccc(F)cc2)c2[nH]c3ccccc3c2C[C@@H]1C1CCCC1] |
| 178 | 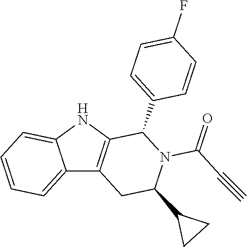 [SMILES: C#CC(=O)N1[C@@H](c2ccc(F)cc2)c2[nH]c3ccccc3c2C[C@@H]1C1CC1] |
| 179 | 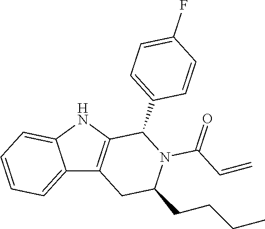 [SMILES: C=CC(=O)N1[C@@H](CCCC)Cc2c([nH]c3ccccc23)[C@@H]1c1ccc(F)cc1] |
| 180 | 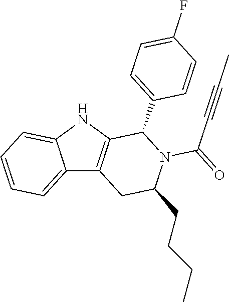 [SMILES: CC#CC(=O)N1[C@@H](CCCC)Cc2c([nH]c3ccccc23)[C@@H]1c1ccc(F)cc1] |
| 181 | 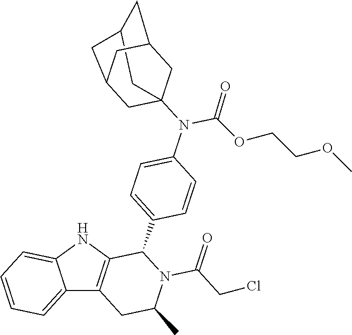 [SMILES: COCCOC(=O)N(c1ccc([C@H]2c3[nH]c4ccccc4c3C[C@H](C)N2C(=O)CCl)cc1)C12CC3CC(CC(C3)C1)C2] |
| 182 | 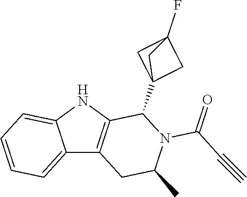 [SMILES: C#CC(=O)N1[C@@H](C)Cc2c([nH]c3ccccc23)[C@@H]1C12CC(F)(C1)C2] |
| 183 | 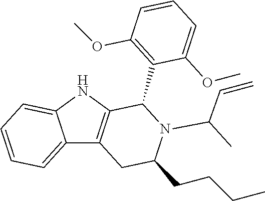 [SMILES: C=CC(C)N1[C@@H](CCCC)Cc2c([nH]c3ccccc23)[C@@H]1c1c(OC)cccc1OC] |
| 184 | 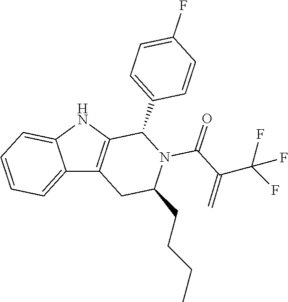 [SMILES: C=C(C(=O)N1[C@@H](CCCC)Cc2c([nH]c3ccccc23)[C@@H]1c1ccc(F)cc1)C(F)(F)F] |
| 185 | 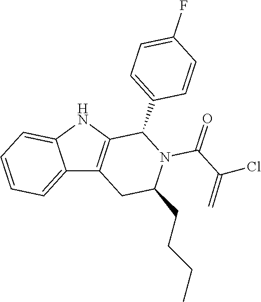 [SMILES: C=C(Cl)C(=O)N1[C@@H](CCCC)Cc2c([nH]c3ccccc23)[C@@H]1c1ccc(F)cc1] |
| 186 | 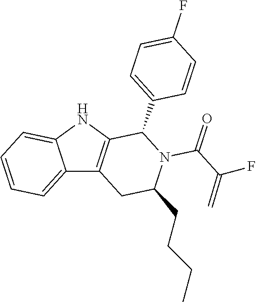 [SMILES: C=C(F)C(=O)N1[C@@H](CCCC)Cc2c([nH]c3ccccc23)[C@@H]1c1ccc(F)cc1] |
| 187 | 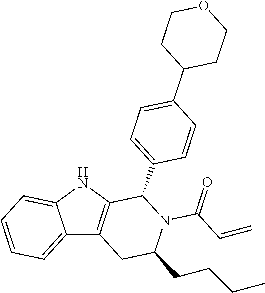 [SMILES: C=CC(=O)N1[C@@H](CCCC)Cc2c([nH]c3ccccc23)[C@@H]1c1ccc(C2CCOCC2)cc1] |
| 188 | 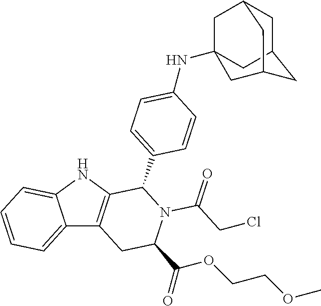 [SMILES: COCCOC(=O)[C@H]1Cc2c([nH]c3ccccc23)[C@H](c2ccc(NC34CC5CC(CC(C5)C3)C4)cc2)N1C(=O)CCl] |
| 189 | 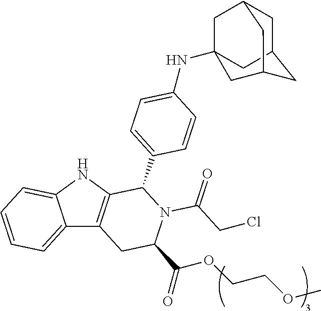 |
| 190 | 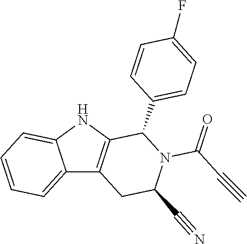 [SMILES: C#CC(=O)N1[C@@H](c2ccc(F)cc2)c2[nH]c3ccccc3c2C[C@@H]1C#N] |
| 191 | 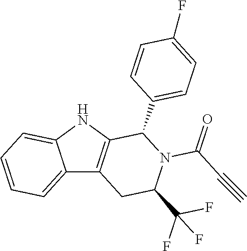 [SMILES: C#CC(=O)N1[C@@H](c2ccc(F)cc2)c2[nH]c3ccccc3c2C[C@@H]1C(F)(F)F] |
| 192 | 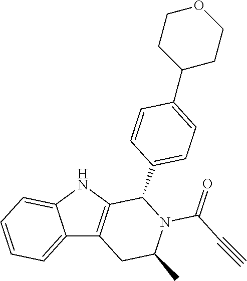 [SMILES: C#CC(=O)N1[C@@H](c2ccc(C3CCOCC3)cc2)c2[nH]c3ccccc3c2C[C@@H]1C] |
| 193 | 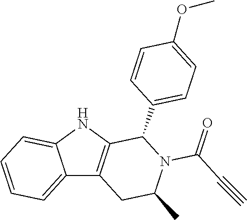 [SMILES: C#CC(=O)N1[C@@H](c2ccc(OC)cc2)c2[nH]c3ccccc3c2C[C@@H]1C] |
| 194 | 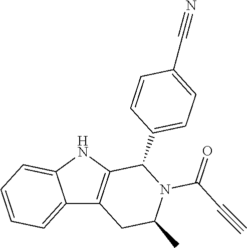 [SMILES: C#CC(=O)N1[C@@H](c2ccc(C#N)cc2)c2[nH]c3ccccc3c2C[C@@H]1C] |
| 195 | 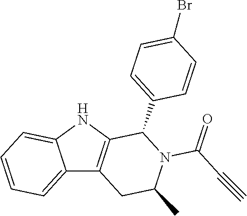 [SMILES: C#CC(=O)N1[C@@H](c2ccc(Br)cc2)c2[nH]c3ccccc3c2C[C@@H]1C] |
| 196 | 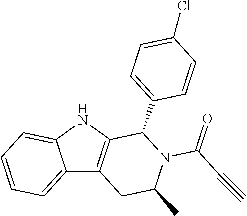 [SMILES: C#CC(=O)N1[C@@H](c2ccc(Cl)cc2)c2[nH]c3ccccc3c2C[C@@H]1C] |
| 197 | 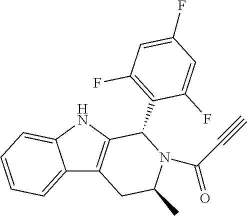 [SMILES: C#CC(=O)N1[C@@H](c2c(F)cc(F)cc2F)c2[nH]c3ccccc3c2C[C@@H]1C] |
| 198 | 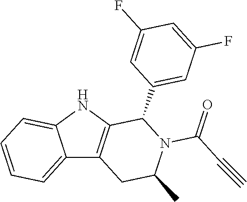 [SMILES: C#CC(=O)N1[C@@H](c2cc(F)cc(F)c2)c2[nH]c3ccccc3c2C[C@@H]1C] |
| 199 | 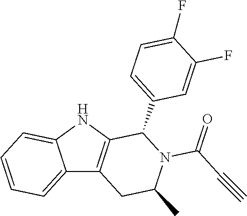 [SMILES: C#CC(=O)N1[C@@H](c2ccc(F)c(F)c2)c2[nH]c3ccccc3c2C[C@@H]1C] |
| 200 | 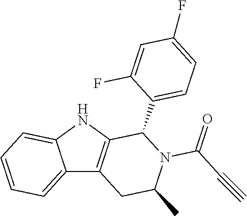 [SMILES: C#CC(=O)N1[C@@H](c2ccc(F)cc2F)c2[nH]c3ccccc3c2C[C@@H]1C] |
| 201 | 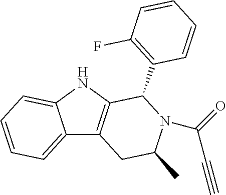 [SMILES: C#CC(=O)N1[C@@H](c2ccccc2F)c2[nH]c3ccccc3c2C[C@@H]1C] |
| 202 | 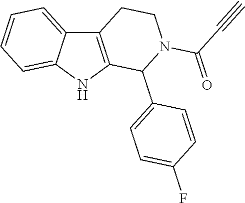 [SMILES: C#CC(=O)N1CCc2c([nH]c3ccccc23)C1c1ccc(F)cc1] |
| 204 | 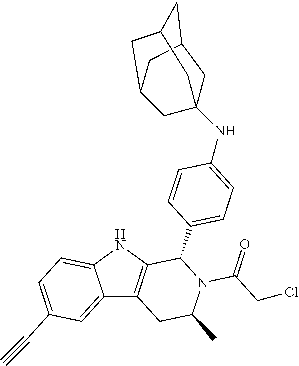 [SMILES: C#Cc1ccc2[nH]c3c(c2c1)C[C@H](C)N(C(=O)CCl)[C@H]3c1ccc(NC23CC4CC(CC(C4)C2)C3)cc1] |
| 205 | 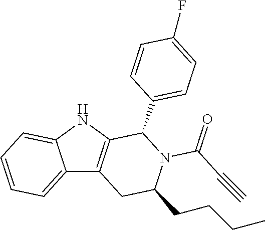 [SMILES: C#CC(=O)N1[C@@H](CCCC)Cc2c([nH]c3ccccc23)[C@@H]1c1ccc(F)cc1] |
| 206 | 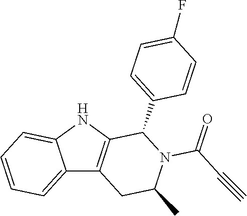 [SMILES: C#CC(=O)N1[C@@H](c2ccc(F)cc2)c2[nH]c3ccccc3c2C[C@@H]1C] |
| 207 | 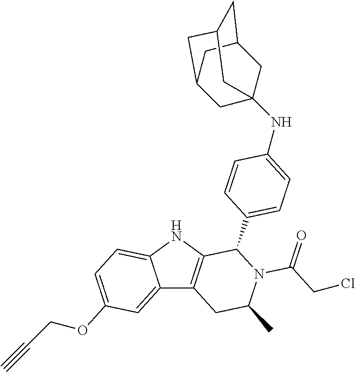 [SMILES: C#CCOc1ccc2[nH]c3c(c2c1)C[C@H](C)N(C(=O)CCl)[C@H]3c1ccc(NC23CC4CC(CC(C4)C2)C3)cc1] |
| 208 | 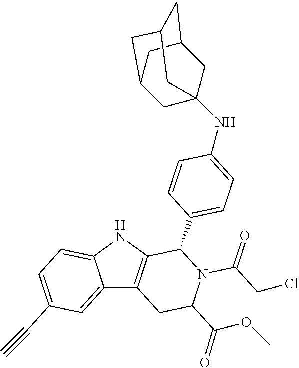 [SMILES: C#Cc1ccc2[nH]c3c(c2c1)CC(C(=O)OC)N(C(=O)CCl)[C@H]3c1ccc(NC23CC4CC(CC(C4)C2)C3)cc1] |
| 209 | 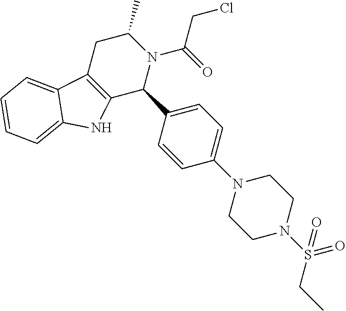 [SMILES: CCS(=O)(=O)N1CCN(c2ccc([C@H]3c4[nH]c5ccccc5c4C[C@H](C)N3C(=O)CCl)cc2)CC1] |
| 210 | 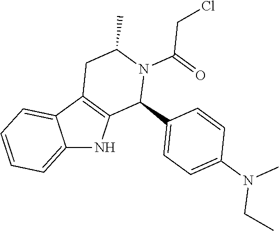 [SMILES: CCN(C)c1ccc([C@H]2c3[nH]c4ccccc4c3C[C@H](C)N2C(=O)CCl)cc1] |
| 211 | 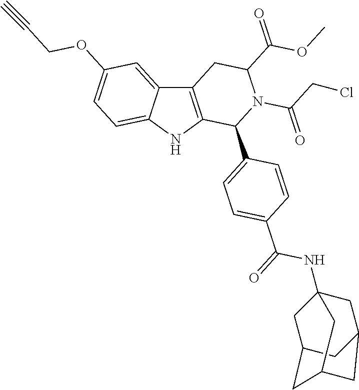 [SMILES: C#CCOc1ccc2[nH]c3c(c2c1)CC(C(=O)OC)N(C(=O)CCl)[C@H]3c1ccc(C(=O)NC23CC4CC(CC(C4)C2)C3)cc1] |
| 212 | 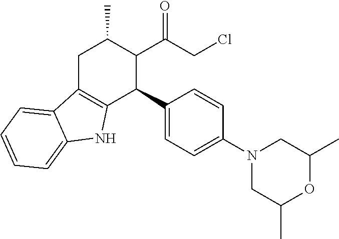 [SMILES: CC1CN(c2ccc([C@H]3c4[nH]c5ccccc5c4C[C@H](C)C3C(=O)CCl)cc2)CC(C)O1] |
| 213 | 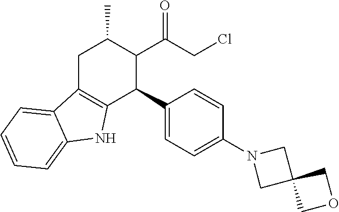 |
| 214 | 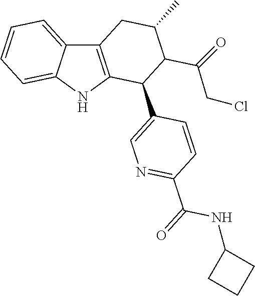 [SMILES: C[C@H]1Cc2c([nH]c3ccccc23)[C@H](c2ccc(C(=O)NC3CCC3)nc2)C1C(=O)CCl] |
| 215 | 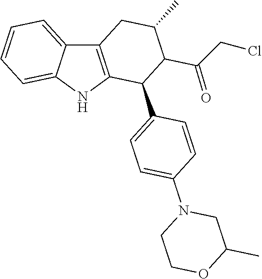 [SMILES: CC1CN(c2ccc([C@H]3c4[nH]c5ccccc5c4C[C@H](C)C3C(=O)CCl)cc2)CCO1] |
| 216 | 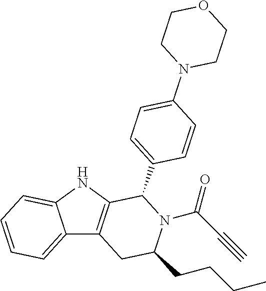 [SMILES: C#CC(=O)N1[C@@H](CCCC)Cc2c([nH]c3ccccc23)[C@@H]1c1ccc(N2CCOCC2)cc1] |
| 217 | 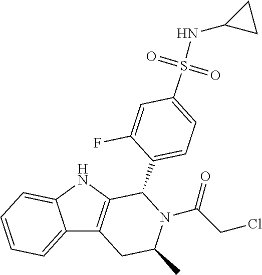 [SMILES: C[C@H]1Cc2c([nH]c3ccccc23)[C@H](c2ccc(S(=O)(=O)NC3CC3)cc2F)N1C(=O)CCl] |
| 218 | 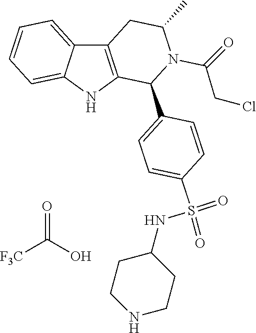 |
| 219 | 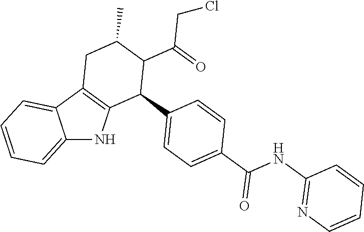 [SMILES: C[C@H]1Cc2c([nH]c3ccccc23)[C@H](c2ccc(C(=O)Nc3ccccn3)cc2)C1C(=O)CCl] |
| 220 | 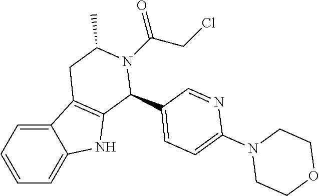 [SMILES: C[C@H]1Cc2c([nH]c3ccccc23)[C@H](c2ccc(N3CCOCC3)nc2)N1C(=O)CCl] |
| 221 | 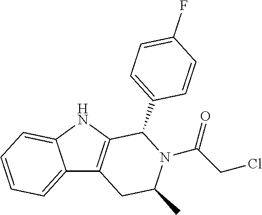 [SMILES: C[C@H]1Cc2c([nH]c3ccccc23)[C@H](c2ccc(F)cc2)N1C(=O)CCl] |
| 222 | 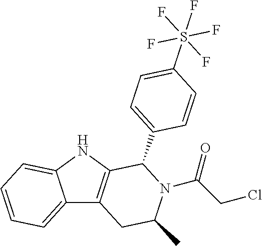 [SMILES: C[C@H]1Cc2c([nH]c3ccccc23)[C@H](c2ccc(S(F)(F)(F)(F)F)cc2)N1C(=O)CCl] |
| 223 | 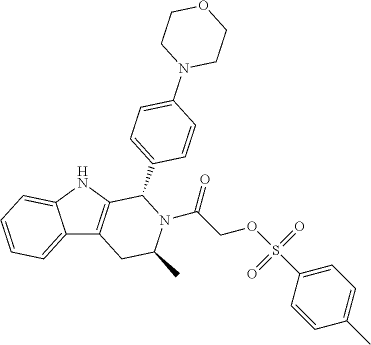 [SMILES: Cc1ccc(S(=O)(=O)OCC(=O)N2[C@@H](c3ccc(N4CCOCC4)cc3)c3[nH]c4ccccc4c3C[C@@H]2C)cc1] |
| 224 | 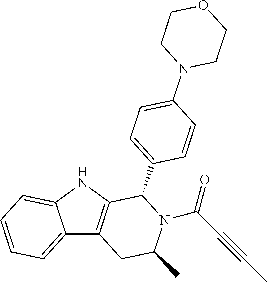 [SMILES: CC#CC(=O)N1[C@@H](c2ccc(N3CCOCC3)cc2)c2[nH]c3ccccc3c2C[C@@H]1C] |
| 225 | 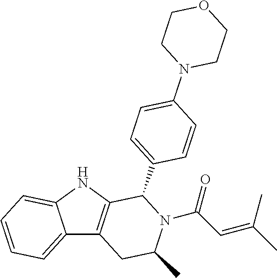 [SMILES: CC(C)=CC(=O)N1[C@@H](c2ccc(N3CCOCC3)cc2)c2[nH]c3ccccc3c2C[C@@H]1C] |
| 226 | 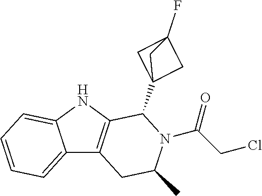 [SMILES: C[C@H]1Cc2c([nH]c3ccccc23)[C@H](C23CC(F)(C2)C3)N1C(=O)CCl] |
| 227 | 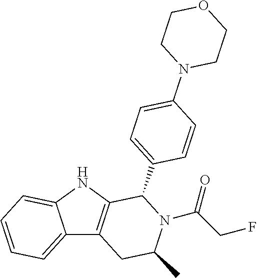 [SMILES: C[C@H]1Cc2c([nH]c3ccccc23)[C@H](c2ccc(N3CCOCC3)cc2)N1C(=O)CF] |
| 228 | 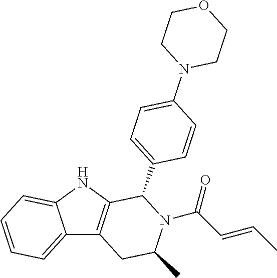 [SMILES: C/C=C/C(=O)N1[C@@H](c2ccc(N3CCOCC3)cc2)c2[nH]c3ccccc3c2C[C@@H]1C] |
| 229 | 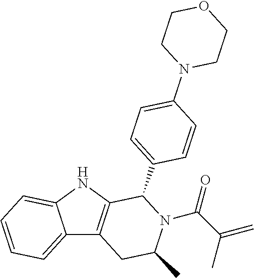 [SMILES: C=C(C)C(=O)N1[C@@H](c2ccc(N3CCOCC3)cc2)c2[nH]c3ccccc3c2C[C@@H]1C] |
| 230 | 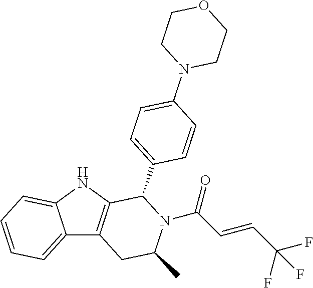 [SMILES: C[C@H]1Cc2c([nH]c3ccccc23)[C@H](c2ccc(N3CCOCC3)cc2)N1C(=O)/C=C/C(F)(F)F] |
| 231 | 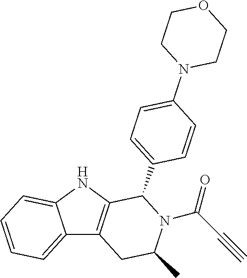 [SMILES: C#CC(=O)N1[C@@H](c2ccc(N3CCOCC3)cc2)c2[nH]c3ccccc3c2C[C@@H]1C] |
| 232 | 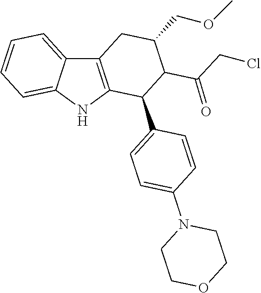 [SMILES: COC[C@H]1Cc2c([nH]c3ccccc23)[C@H](c2ccc(N3CCOCC3)cc2)C1C(=O)CCl] |
| 233 | 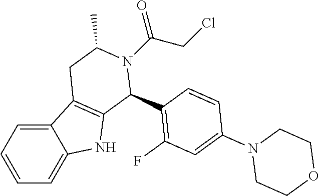 [SMILES: C[C@H]1Cc2c([nH]c3ccccc23)[C@H](c2ccc(N3CCOCC3)cc2F)N1C(=O)CCl] |
| 234 | 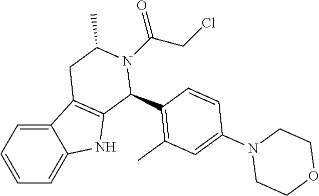 [SMILES: Cc1cc(N2CCOCC2)ccc1[C@H]1c2[nH]c3ccccc3c2C[C@H](C)N1C(=O)CCl] |
| 235 | 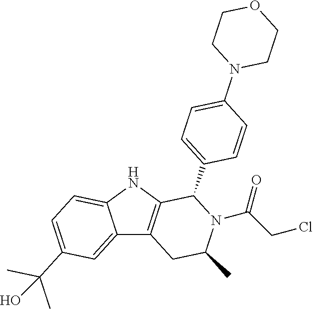 [SMILES: C[C@H]1Cc2c([nH]c3ccc(C(C)(C)O)cc23)[C@H](c2ccc(N3CCOCC3)cc2)N1C(=O)CCl] |
| 236 | 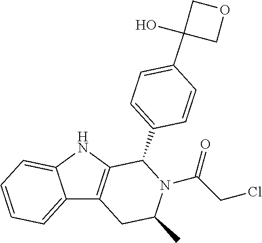 [SMILES: C[C@H]1Cc2c([nH]c3ccccc23)[C@H](c2ccc(C3(O)COC3)cc2)N1C(=O)CCl] |
| 237 | 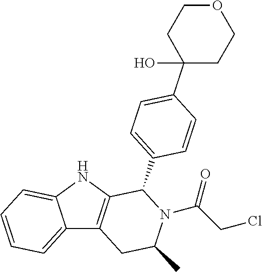 [SMILES: C[C@H]1Cc2c([nH]c3ccccc23)[C@H](c2ccc(C3(O)CCOCC3)cc2)N1C(=O)CCl] |
| 238 | 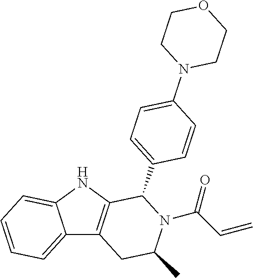 [SMILES: C=CC(=O)N1[C@@H](c2ccc(N3CCOCC3)cc2)c2[nH]c3ccccc3c2C[C@@H]1C] |
| 239 | 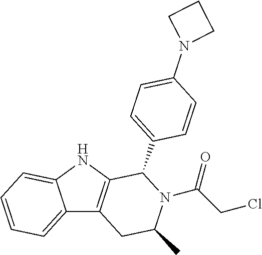 [SMILES: C[C@H]1Cc2c([nH]c3ccccc23)[C@H](c2ccc(N3CCC3)cc2)N1C(=O)CCl] |
| 240 | 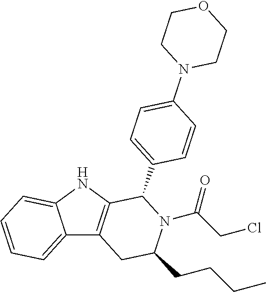 [SMILES: CCCC[C@H]1Cc2c([nH]c3ccccc23)[C@H](c2ccc(N3CCOCC3)cc2)N1C(=O)CCl] |
| 241 | 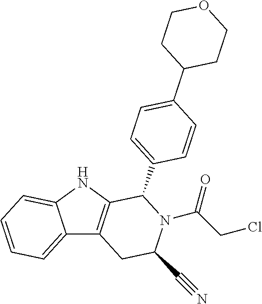 [SMILES: N#C[C@H]1Cc2c([nH]c3ccccc23)[C@H](c2ccc(C3CCOCC3)cc2)N1C(=O)CCl] |
| 242 | 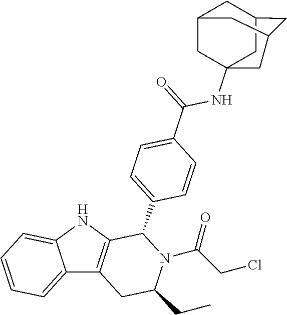 [SMILES: CC[C@H]1Cc2c([nH]c3ccccc23)[C@H](c2ccc(C(=O)NC34CC5CC(CC(C5)C3)C4)cc2)N1C(=O)CCl] |
| 243 | 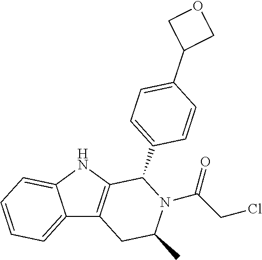 [SMILES: C[C@H]1Cc2c([nH]c3ccccc23)[C@H](c2ccc(C3COC3)cc2)N1C(=O)CCl] |
| 244 | 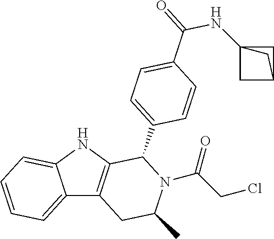 [SMILES: C[C@H]1Cc2c([nH]c3ccccc23)[C@H](c2ccc(C(=O)NC34CC(C3)C4)cc2)N1C(=O)CCl] |
| 245 | 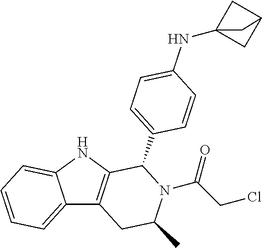 [SMILES: C[C@H]1Cc2c([nH]c3ccccc23)[C@H](c2ccc(NC34CC(C3)C4)cc2)N1C(=O)CCl] |
| 246 | 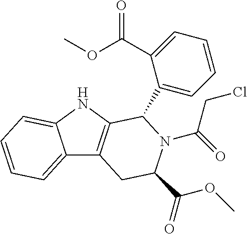 [SMILES: COC(=O)c1ccccc1[C@H]1c2[nH]c3ccccc3c2C[C@H](C(=O)OC)N1C(=O)CCl] |
| 247 | 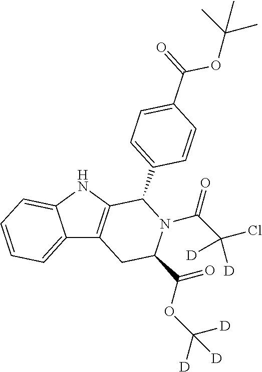 [SMILES: [2H]C([2H])([2H])OC(=O)[C@H]1Cc2c([nH]c3ccccc23)[C@H](c2ccc(C(=O)OC(C)(C)C)cc2)N1C(=O)C([2H])([2H])Cl] |
| 249 | 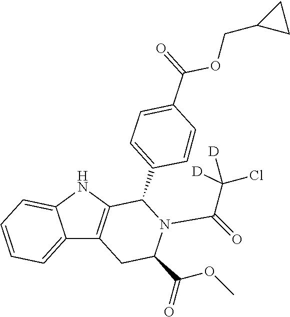 [SMILES: [2H]C([2H])(Cl)C(=O)N1[C@@H](c2ccc(C(=O)OCC3CC3)cc2)c2[nH]c3ccccc3c2C[C@@H]1C(=O)OC] |
| 250 | 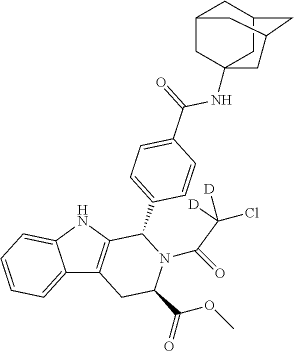 [SMILES: [2H]C([2H])(Cl)C(=O)N1[C@@H](c2ccc(C(=O)NC34CC5CC(CC(C5)C3)C4)cc2)c2[nH]c3ccccc3c2C[C@@H]1C(=O)OC] |
| 251 | 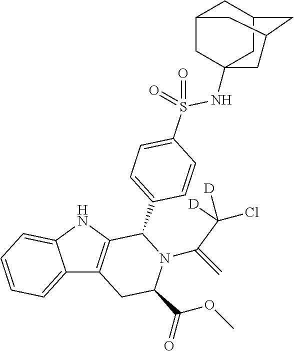 [SMILES: [2H]C([2H])(Cl)C(=C)N1[C@@H](c2ccc(S(=O)(=O)NC34CC5CC(CC(C5)C3)C4)cc2)c2[nH]c3ccccc3c2C[C@@H]1C(=O)OC] |
| 257 | 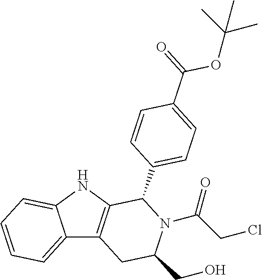 [SMILES: CC(C)(C)OC(=O)c1ccc([C@H]2c3[nH]c4ccccc4c3C[C@H](CO)N2C(=O)CCl)cc1] |
| 258 | 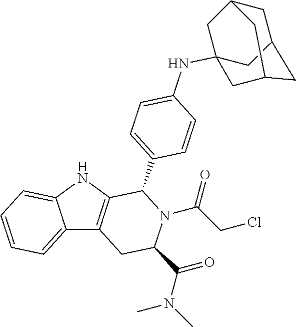 [SMILES: CN(C)C(=O)[C@H]1Cc2c([nH]c3ccccc23)[C@H](c2ccc(NC34CC5CC(CC(C5)C3)C4)cc2)N1C(=O)CCl] |
| 259 | 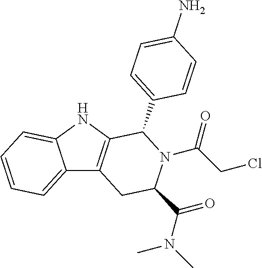 [SMILES: CN(C)C(=O)[C@H]1Cc2c([nH]c3ccccc23)[C@H](c2ccc(N)cc2)N1C(=O)CCl] |
| 260 | 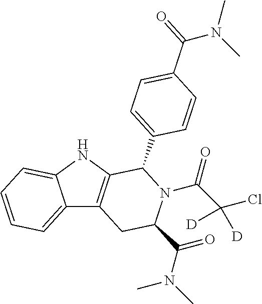 [SMILES: [2H]C([2H])(Cl)C(=O)N1[C@@H](c2ccc(C(=O)N(C)C)cc2)c2[nH]c3ccccc3c2C[C@@H]1C(=O)N(C)C] |
| 261 | 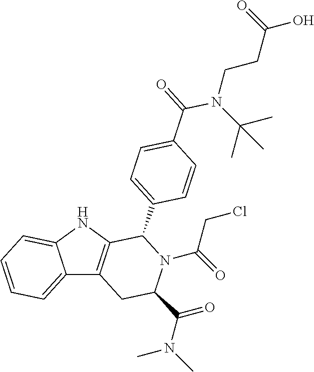 [SMILES: CN(C)C(=O)[C@H]1Cc2c([nH]c3ccccc23)[C@H](c2ccc(C(=O)N(CCC(=O)O)C(C)(C)C)cc2)N1C(=O)CCl] |
| 262 | 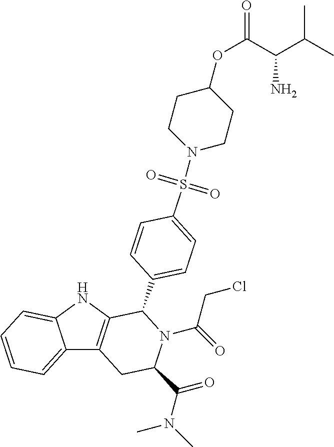 [SMILES: CC(C)[C@H](N)C(=O)OC1CCN(S(=O)(=O)c2ccc([C@H]3c4[nH]c5ccccc5c4C[C@H](C(=O)N(C)C)N3C(=O)CCl)cc2)CC1] |
| 263 | 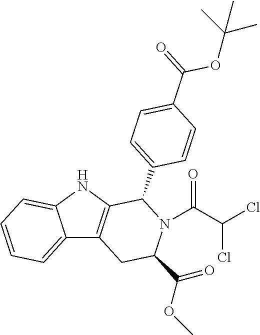 [SMILES: COC(=O)[C@H]1Cc2c([nH]c3ccccc23)[C@H](c2ccc(C(=O)OC(C)(C)C)cc2)N1C(=O)C(Cl)Cl] |
| 264 | 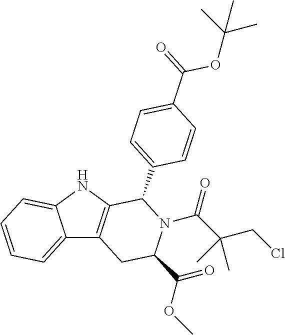 [SMILES: COC(=O)[C@H]1Cc2c([nH]c3ccccc23)[C@H](c2ccc(C(=O)OC(C)(C)C)cc2)N1C(=O)C(C)(C)CCl] |
| 265 | 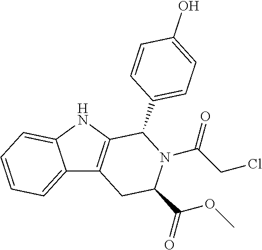 [SMILES: COC(=O)[C@H]1Cc2c([nH]c3ccccc23)[C@H](c2ccc(O)cc2)N1C(=O)CCl] |
| 266 | 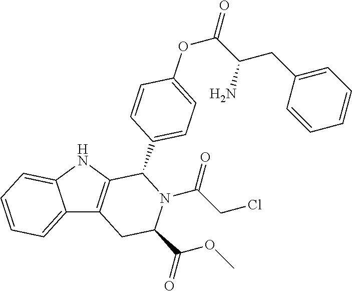 [SMILES: COC(=O)[C@H]1Cc2c([nH]c3ccccc23)[C@H](c2ccc(OC(=O)[C@@H](N)Cc3ccccc3)cc2)N1C(=O)CCl] |
| 268 | 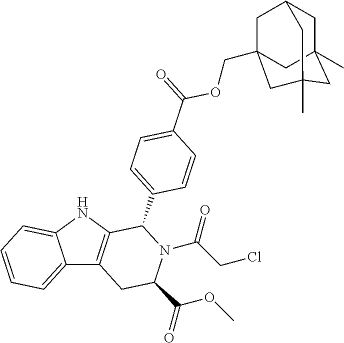 [SMILES: COC(=O)[C@H]1Cc2c([nH]c3ccccc23)[C@H](c2ccc(C(=O)OCC34CC5CC(C)(CC(C)(C5)C3)C4)cc2)N1C(=O)CCl] |
| 269 | 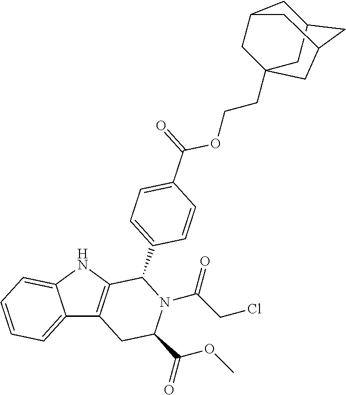 [SMILES: COC(=O)[C@H]1Cc2c([nH]c3ccccc23)[C@H](c2ccc(C(=O)OCCC34CC5CC(CC(C5)C3)C4)cc2)N1C(=O)CCl] |
| 270 | 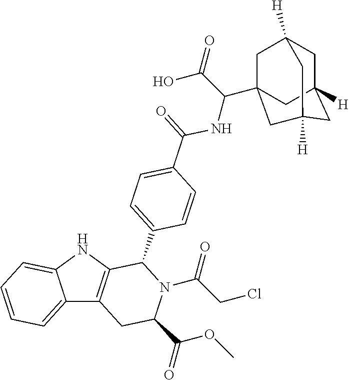 [SMILES: COC(=O)[C@H]1Cc2c([nH]c3ccccc23)[C@H](c2ccc(C(=O)NC(C(=O)O)C34C[C@H]5C[C@@H](C3)C[C@@H](C4)C5)cc2)N1C(=O)CCl] |
| 271 | 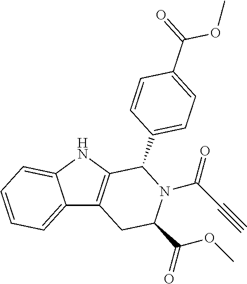 [SMILES: C#CC(=O)N1[C@@H](c2ccc(C(=O)OC)cc2)c2[nH]c3ccccc3c2C[C@@H]1C(=O)OC] |
| 272 | 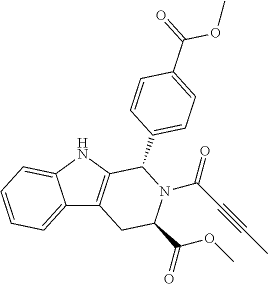 [SMILES: CC#CC(=O)N1[C@@H](c2ccc(C(=O)OC)cc2)c2[nH]c3ccccc3c2C[C@@H]1C(=O)OC] |
| 273 | 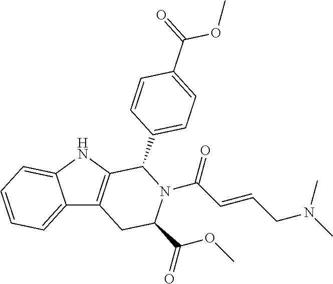 [SMILES: COC(=O)c1ccc([C@H]2c3[nH]c4ccccc4c3C[C@H](C(=O)OC)N2C(=O)/C=C/CN(C)C)cc1] |
| 274 | 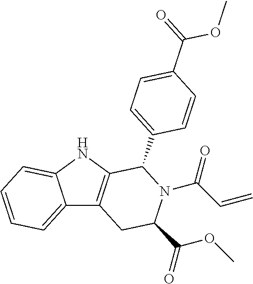 [SMILES: C=CC(=O)N1[C@@H](c2ccc(C(=O)OC)cc2)c2[nH]c3ccccc3c2C[C@@H]1C(=O)OC] |
| 275 | 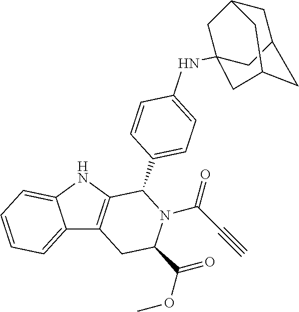 [SMILES: C#CC(=O)N1[C@@H](c2ccc(NC34CC5CC(CC(C5)C3)C4)cc2)c2[nH]c3ccccc3c2C[C@@H]1C(=O)OC] |
| 276 | 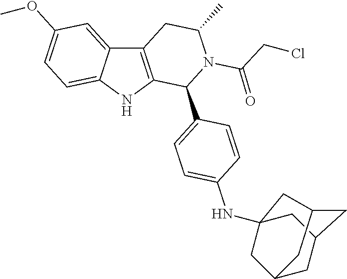 [SMILES: COc1ccc2[nH]c3c(c2c1)C[C@H](C)N(C(=O)CCl)[C@H]3c1ccc(NC23CC4CC(CC(C4)C2)C3)cc1] |
| 277 | 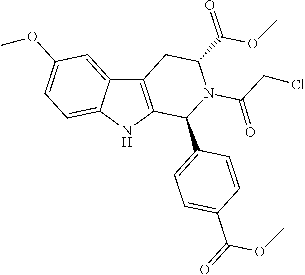 [SMILES: COC(=O)c1ccc([C@H]2c3[nH]c4ccc(OC)cc4c3C[C@H](C(=O)OC)N2C(=O)CCl)cc1] |
| 278 | 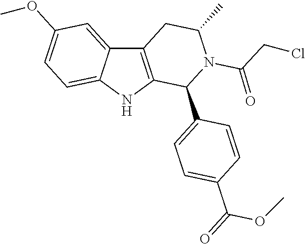 [SMILES: COC(=O)c1ccc([C@H]2c3[nH]c4ccc(OC)cc4c3C[C@H](C)N2C(=O)CCl)cc1] |
| 279 | 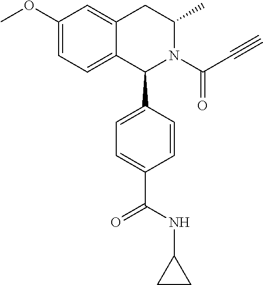 [SMILES: C#CC(=O)N1[C@@H](c2ccc(C(=O)NC3CC3)cc2)c2ccc(OC)cc2C[C@@H]1C] |
| 280 | 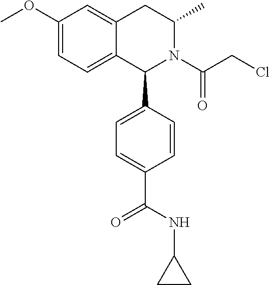 [SMILES: COc1ccc2c(c1)C[C@H](C)N(C(=O)CCl)[C@H]2c1ccc(C(=O)NC2CC2)cc1] |
| 281 | 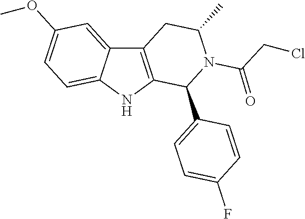 [SMILES: COc1ccc2[nH]c3c(c2c1)C[C@H](C)N(C(=O)CCl)[C@H]3c1ccc(F)cc1] |
| 282 | 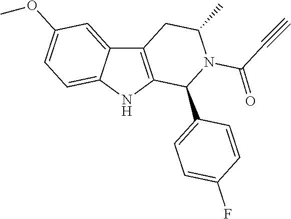 [SMILES: C#CC(=O)N1[C@@H](c2ccc(F)cc2)c2[nH]c3ccc(OC)cc3c2C[C@@H]1C] |
| 283 | 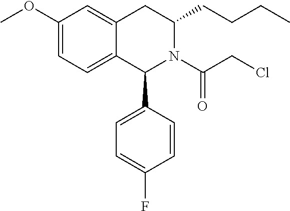 [SMILES: CCCC[C@H]1Cc2cc(OC)ccc2[C@H](c2ccc(F)cc2)N1C(=O)CCl] |
| 284 | 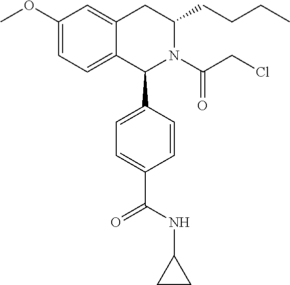 [SMILES: CCCC[C@H]1Cc2cc(OC)ccc2[C@H](c2ccc(C(=O)NC3CC3)cc2)N1C(=O)CCl] |
| 285 | 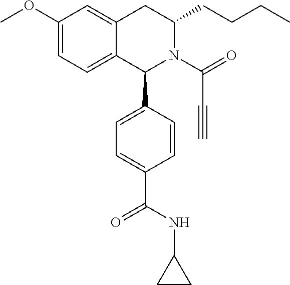 [SMILES: C#CC(=O)N1[C@@H](CCCC)Cc2cc(OC)ccc2[C@@H]1c1ccc(C(=O)NC2CC2)cc1] |
| 286 | 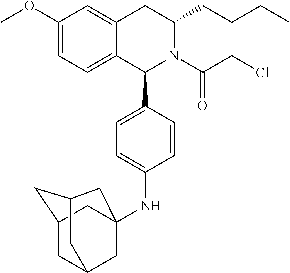 [SMILES: CCCC[C@H]1Cc2cc(OC)ccc2[C@H](c2ccc(NC34CC5CC(CC(C5)C3)C4)cc2)N1C(=O)CCl] |
| 287 | 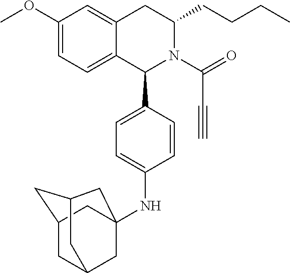 [SMILES: C#CC(=O)N1[C@@H](CCCC)Cc2cc(OC)ccc2[C@@H]1c1ccc(NC23CC4CC(CC(C4)C2)C3)cc1] |
| 288 | 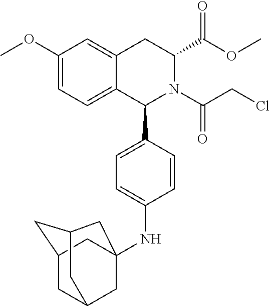 [SMILES: COC(=O)[C@H]1Cc2cc(OC)ccc2[C@H](c2ccc(NC34CC5CC(CC(C5)C3)C4)cc2)N1C(=O)CCl] |
| 290 | 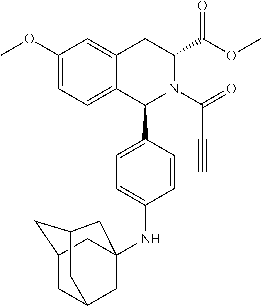 |
| 291 | 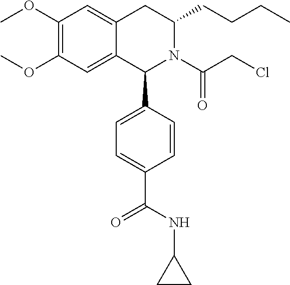 [SMILES: CCCC[C@H]1Cc2cc(OC)c(OC)cc2[C@H](c2ccc(C(=O)NC3CC3)cc2)N1C(=O)CCl] |
| 292 | 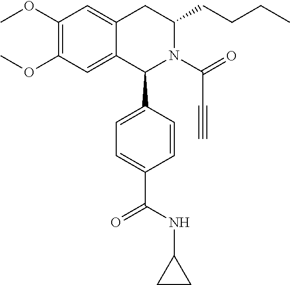 [SMILES: C#CC(=O)N1[C@@H](CCCC)Cc2cc(OC)c(OC)cc2[C@@H]1c1ccc(C(=O)NC2CC2)cc1] |
| 293 | 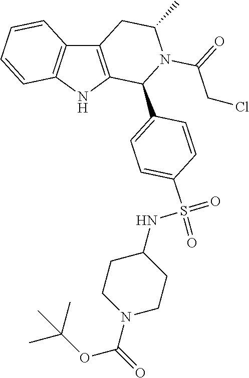 [SMILES: C[C@H]1Cc2c([nH]c3ccccc23)[C@H](c2ccc(S(=O)(=O)NC3CCN(C(=O)OC(C)(C)C)CC3)cc2)N1C(=O)CCl] |

Additional compounds that induce iron-dependent cellular disassembly are known in the art and are described, for example, in US2020/0299283, which is incorporated by reference herein in its entirety.

In certain embodiments, provided herein is a compound of Formula A1 or a tautomer, stereoisomer, mixture of stereoisomers, isotopically enriched analog, or pharmaceutically acceptable salt thereof:

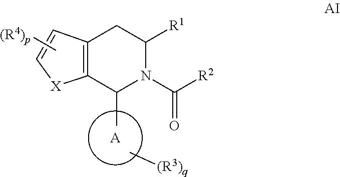

wherein:

ring A is C₄-C₁₀cycloalkyl, heterocyclyl, aryl, or heteroaryl;

X is —O—, —S—, —NR⁹—, —CR⁵═CR⁵, or CR⁵═N—;

p is 0, 1 or 2;

q is 0, 1, 2 or 3;

R¹ is C₁-C₆alkyl, C₂-C₆alkenyl, C₂-C₆alkynyl, C₂-C₆haloalkyl, C₃-C₁₀cycloalkyl, —CN, —OR⁷, —C(O) OR⁶, —C(O)N(R⁷)₂, —OC(O)R⁶, —S(O)₂R⁸, —S(O)₂N(R⁷)₂, —S(O)N(R⁷)₂, —S(O)R⁸, —N(R⁷)₂, —NO₂, —C₁-C₆alkyl-OR⁷, or —Si(R⁵)₃;

R² is —C₁-C₂haloalkyl, —C₂-C₃alkenyl, —C₂-C₃haloalkenyl, C₂alkynyl, or —CH₂OS(O)₂— phenyl, wherein the C₁-C₂haloalkyl and —C₂-C₃alkenylhalo are optionally substituted with one or two —CH₃, and the C₂alkynyl and phenyl are optionally substituted with one —CH₃; each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₂-C₆alkylaryl, —C₂-C₆alkenylaryl, C₂-C₆alkylheteroaryl, or —C₂-C₆alkenylheteroaryl of R³ is independently optionally substituted with one to three R¹⁰; each R⁴ is independently halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₃-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₂-C₆alkylheteroaryl, or —C₂-C₆alkenylheteroaryl of R⁴ is optionally independently optionally substituted with one to three R¹⁰;

each R⁵ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHRN(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl of R⁵ is optionally independently optionally substituted with one to three R¹⁰;

each R⁶ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁶ is independently further substituted with one to three R¹¹;

each R⁷ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₆cycloalkyl, —C₂-C₆alkenylC₃-C₆cycloalkyl, —C₁-C₆alkylheterocyclyl, —C₂-C₆alkenylheterocyclyl, —C₂-C₆alkylaryl, —C₂-C₆alkenylaryl, —C₁-C₆alkylheteroaryl, —C₂-C₆alkenylheteroaryl, or two R⁷ together with the nitrogen atom to which they are attached, form a 4 to 7 membered heterocyclyl; wherein each R⁷ or ring formed thereby is independently further substituted with one to three R¹¹;

each R⁸ is independently C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, C₂-C₆alkenylaryl, —C₁-C₆alkylheteroaryl, or —C₂-C₆alkenylheteroaryl; wherein each R⁸ is independently further substituted with one to three R¹¹;

R⁹ is hydrogen or C₁-C₆alkyl;

each R¹⁰ is independently halo, —CN, —OR¹², —NO₂, —N(R¹²)₂, —S(O)R¹³, —S(O)₂R₁, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl, wherein each C₁-C₆alkyl, C₁-C₆haloalkyl, C₂-C₆alkenyl, C₂-C₆alkynyl, C₃-C₁₀cycloalkyl, heterocyclyl, aryl, or heteroaryl of R¹⁰ is optionally independently substituted with one to three R¹¹;

each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₂-C₆haloalkyl, C₂-C₆alkenyl, C₂-C₆alkynyl, C₃-C₁₀cycloalkyl, heterocyclyl, aryl, or heteroaryl;

each R¹² is independently hydrogen, C₁-C₆alkyl or C₃-C₁₀cycloalkyl;

each R¹³ is independently C₁-C₆alkyl or C₃-C₁₀cycloalkyl; and

each R¹⁵ is independently C₂-C₆alkyl, C₂-C₆alkenyl, aryl, heteroaryl, —C₁-C₆alkylaryl, —C₂-C₆alkenylaryl, —C₁-C₆alkylheteroaryl, and —C₂-C₆alkenylheteroaryl.

In certain embodiments, provided is a compound of Formula A1 or a tautomer, stereoisomer, mixture of stereoisomers, isotopically enriched analog, or pharmaceutically acceptable salt thereof:

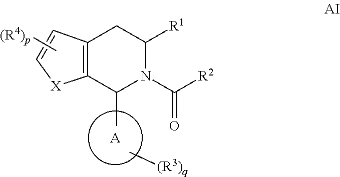

wherein:

ring A is C₄-C₁₀cycloalkyl, heterocyclyl, aryl, or heteroaryl;

X is —O—, —S—, —NR⁹—, —CR⁵═CR⁵—, or —CR⁵═N—;

p is 0, 1 or 2;

q is 0, 1, 2 or 3;

R¹ is C₁-C₆alkyl, C₂-C₆alkenyl, C₂-C₆alkynyl, C₁-C₆haloalkyl, C₃-C₁₀cycloalkyl, —CN, —OR⁷, —C(O)OR⁶, —C(O)N(R⁷)₂, —OC(O)R⁶, —S(O)₂R⁸, —S(O)₂N(R⁷)₂, —S(O)N(R⁷)₂, —S(O)R⁸, —N(R⁷)₂, —NO₂, —Cᵣ C₆alkyl-OR⁷, or —Si(R¹⁵)₃;

R² is —C₁-C₂haloalkyl, —C₂-C₃alkenyl, —C₂-C₃haloalkenyl, C₂alkynyl, or —CH₂OS(O)₂— phenyl, wherein the C₁-C₂alkylhalo and —C₂-C₃alkenylhalo are optionally substituted with one or two —CH₃, and the C₂alkynyl and phenyl are optionally substituted with one CH₃;

each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₃-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R³ is independently optionally substituted with one to three R¹⁰;

each R⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R⁵)₃, —C(O) OR⁶, —C(O)N(R⁷)₂, —NR¹²C(O)R⁸, —OC(O)R⁸, —C(O) R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₃-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R⁴ is optionally independently optionally substituted with one to three R¹⁰;

each R⁵ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHRN(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R⁵ is optionally independently optionally substituted with one to three R¹⁰;

each R⁶ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each R⁶ is independently further substituted with one to three R¹¹;

each R⁷ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₆cycloalkyl, —C₂-C₆alkenylC₃-C₆cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, —C₂-C₆alkenylheteroaryl, or two R⁷ together with the nitrogen atom to which they are attached, form a 4 to 7 membered heterocyclyl; wherein each R⁷ or ring formed thereby is independently further substituted with one to three R¹¹;

each R⁸ is independently C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁸ is independently further substituted with one to three R¹¹;

R⁹ is hydrogen or C₁-C₆alkyl;

each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl, wherein each C₁-C₆alkyl, C₁-C₆haloalkyl, C₂-C₆alkenyl, C₂-C₆alkynyl, C₃-C₁₀cycloalkyl, heterocyclyl, aryl, or heteroaryl of R¹⁰ is optionally independently substituted with one to three R¹¹;

each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl;

each R¹² is independently hydrogen, C₁-C₆alkyl or C₃-C₁₀cycloalkyl;

each R¹³ is independently C₁-C₆alkyl or C₃-C₁₀cycloalkyl; and

each R¹⁵ is independently C₁-C₆alkyl, C₂-C₆alkenyl, aryl, heteroaryl, —C₁-C₆alkylaryl, —C₂-C₆alkenylaryl, —C₁-C₆alkylheteroaryl, and —C₂-C₆alkenylheteroaryl; provided that at least one of the following is true:

1) R¹ is other than —C(O)OCH₃;

2) R² is —C₂alkynyl optionally substituted with one —CH₃; or

3) when R¹ is —C(O)OCH₃ and R² is —CH₂Cl, then the moiety

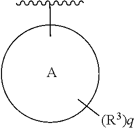

is other than 1,3-benzodioxol-5-yl, 4-nitrophenyl, 4-bro-mophenyl, cyclohexyl, furyl, or 4-methoxyphenyl.

In certain embodiments, R¹ is other than —C(O) OR⁶ or R² is —C₂alkynyl optionally substituted with one —CH₃. In certain embodiments, R¹ is other than —C(O)OCH₃ or R² is —C₂alkynyl optionally substituted with one —CH₃. In certain embodiments, R¹ is other than —C(O)OR⁶ and R² is —C₂alkynyl optionally substituted with one —CH₃. In certain embodiments, R¹ is other than —C(O)OCH₃ and R² is —C₂alkynyl optionally substituted with one —CH₃. In certain embodiments, R¹ is other than —C(O)OR⁶. In certain embodiments, R¹ is other than —C(O)OCH₃. In certain embodiments, R² is —C₂alkynyl optionally substituted with one —CH₃. In certain embodiments, R² is —C₂alkynyl.

Also provided is a compound of Formula AIA, or a tautomer, stereoisomer, mixture of stereoisomers, isotopically enriched analog, or pharmaceutically acceptable salt thereof:

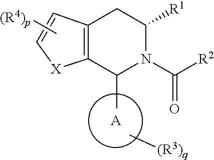

wherein each of ring A, X, R¹, R², R³, R⁴, p, and q are independently as defined herein.

Also provided is a compound of Formula AIB, or a tautomer, stereoisomer, mixture of stereoisomers, isotopically enriched analog, or pharmaceutically acceptable salt thereof:

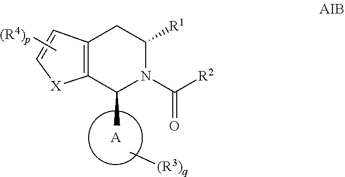

wherein each of ring A, X, R¹, R², R³, R⁴, p, and q are independently as defined herein.

Also provided is a compound of Formula AIB, or a tautomer, stereoisomer, mixture of stereoisomers, isotopically enriched analog, or pharmaceutically acceptable salt thereof:

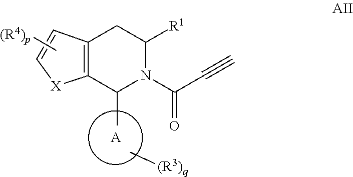

wherein each of ring A, X, R¹, R³, R⁴, p, and q are independently as defined herein.

Also provided is a compound of Formula AIIA, or a tautomer, stereoisomer, mixture of stereoisomers, isotopically enriched analog, or pharmaceutically acceptable salt thereof:

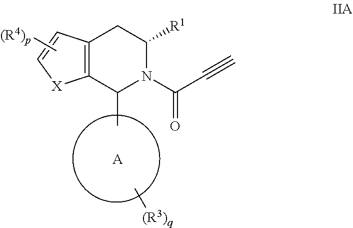

wherein each of ring A, X, R¹, R³, R⁴, p, and q are independently as defined herein.

Also provided is a compound of Formula AIIB, or a tautomer, stereoisomer, mixture of stereoisomers, isotopically enriched analog, or pharmaceutically acceptable salt thereof:

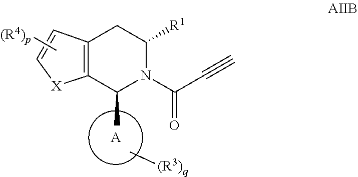

wherein each of ring A, X, R¹, R³, R⁴, p, and q are independently as defined herein.

Also provided is a compound of Formula AIII, or a tautomer, stereoisomer, mixture of stereoisomers, isotopically enriched analog, or pharmaceutically acceptable salt thereof:

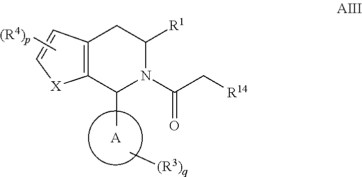

wherein each of ring A, X, R¹, R³, R⁴, p, and q are independently as defined herein, and R¹⁴ is halo.

Also provided is a compound of Formula AIIIA, or a tautomer, stereoisomer, mixture of stereoisomers, isotopically enriched analog, or pharmaceutically acceptable salt thereof:

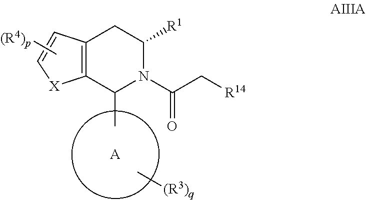

wherein each of ring A, X, R¹, R³, R⁴, p, and q are independently as defined herein, and R¹⁴ is halo.

Also provided is a compound of Formula AIIIB, or a tautomer, stereoisomer, mixture of stereoisomers, isotopically enriched analog, or pharmaceutically acceptable salt thereof:

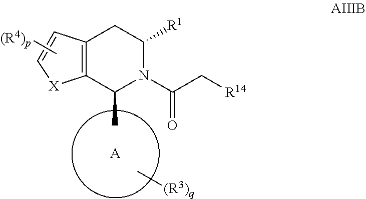

wherein each of ring A, X, R¹, R³, R⁴, p, and q are independently as defined herein, and R¹⁴ is halo.

In certain embodiments,

ring A is aryl or heteroaryl;

X is —O—, —S—, —NH—, —CH═CH—, or —CH═N—;

p is 0, 1 or 2;

q is 1;

R¹ is C₁-C₆alkyl, —C(O)O—C₁-C₆alkyl, or —C(O)N(C₁-C₆alkyl)₂;

R³ is halo, —NHR⁸, —S(O)₂N(R⁷)₂, —C(O)OR⁶, —C(O)N(R⁷)₂, or heterocyclyl;

each R⁴ is independently —OR⁸;

R⁶ is C₁-C₆alkyl;

each R⁷ is independently hydrogen, C₁-C₆alkyl, or C₃-C₁₀cycloalkyl, wherein each R⁷ is independently further substituted with one to three R¹¹;

each R⁸ is independently C₁-C₆alkyl or C₃-C₁₀cycloalkyl; wherein each R⁸ is independently further substituted with one to three R¹¹;

each R¹¹ is independently —O—C₁-C₆alkyl; and R¹⁴ is halo.

In certain embodiments,

ring A is aryl or heteroaryl;

X is —O—, —S—, —NH—, —CH═CH—, or —CH═N—;

p is 0, 1 or 2;

q is 1;

R¹ is C₁-C₆alkyl or —C(O)N(C₁-C₆alkyl)₂;

R³ is halo, —NHR⁸, —S(O)₂N(R⁷)₂, —C(O)OR⁶, —C(O)N(R⁷)₂, or heterocyclyl;

each R⁴ is independently —OR⁸;

R⁶ is C₁-C₆alkyl;

each R⁷ is independently hydrogen, C₁-C₆alkyl, or C₃-C₁₀cycloalkyl, wherein each R⁷ is independently further substituted with one to three R¹¹;

each R⁸ is independently C₁-C₆alkyl or C₃-C₁₀cycloalkyl; wherein each R⁸ is independently further substituted with one to three R¹¹;

each R¹¹ is independently —O—C₁-C₆alkyl; and R¹⁴ is halo.

In certain embodiments, X is —O—, —S—, or —NR⁹—. In certain embodiments, X is —O—, —S—, or —NH—. In certain embodiments, X is —O—. In certain embodiments, X is —S—. In certain embodiments, X is —NR⁹—. In certain embodiments, X is —NH—.

In certain embodiments, X is —CR⁵═CR⁵— or —CR⁵═N—. In certain embodiments, X is —CH═CH— or —CH═N—. In certain embodiments, X is —CR⁵═CR⁵—. In certain embodiments, X is —CR⁵═N—.

In certain embodiments, R⁵ is R⁴.

In certain embodiments, when X is —CH═CH—, p is 1 or 2, each R⁴ is methoxy, ring A is phenyl, and q is 1, then R³ is other than adamantylamine, fluoro, or —C(O) NH-cyclopropyl. In certain embodiments, when X is —CH═CH—, p is 1 or 2, each R⁴ is methoxy, R¹ is methyl, n-butyl or —C(O)OCH₃, ring A is phenyl, and q is 1, then R³ is other than adamantylamine, fluoro, and —C(O)NH-cyclopropyl. In certain embodiments, when X is —CH═CH—, p is 1 or 2, each R⁴ is methoxy, R¹ is methyl, n-butyl or —C(O)OCH₃, R² is —CH₂Cl or C₂alkynyl, ring A is phenyl, and q is 1, then R³ is other than adamantylamine, fluoro, or —C(O)NH-cyclopropyl.

In certain embodiments, when X is —CR⁵═CR⁵—, p is 1 or 2, ring A is phenyl, cyclohexyl, or furyl, and q is 0 or 1, then at least one R⁴ is other than methoxy. In certain embodiments, when R¹ is —C(O)OCH₃ and R² is —CH₂Cl, ring A is phenyl, cyclohexyl, or furyl, q is 0 or 1, R³ is —NO₂, Br, or —OCH₃, and p is 1 or 2, then at least one R⁴ is other than methoxy. In certain embodiments, when R² is —CH₂Cl, X is —CR⁵═CR⁵—, p is 1 or 2, ring A is phenyl, cyclohexyl, or furyl, and q is 0 or 1, then at least one R⁴ is other than methoxy. In certain embodiments, when R¹ is —C(O)OCH₃, R² is —CH₂Cl, X is —CR⁵═CR⁵—, p is 1 or 2, ring A is phenyl, cyclohexyl, or furyl, and q is 0 or 1, then at least one R⁴ is other than methoxy.

In certain embodiments, when R¹ is —C(O)OCH₃ and R² is CH₂Cl, then X is other than —CR⁵═CR⁵—. In certain embodiments, when X is —CH═CH—, p is 1 or 2, ring A is phenyl, and q is 1, then at least one R⁴ is other than methoxy.

In certain embodiments, the compound is not N-cyclopropyl-4-((1S,3S)-6-methoxy-3-methyl-2-propioloyl-1,2,3,4-tetrahydroisoquinolin-1-yl)benzamide, 4-((1S,3S)-2-(2-chloroacetyl)-6-methoxy-3-methyl-1,2,3,4-tetrahydroisoquinolin-1-yl)-N-cyclopropylbenzamide, 1-((1S,3S)-3-butyl-1-(4-fluorophenyl)-6-methoxy-3,4-dihy-droisoquinolin-2(1H)-yl)-2-chloroethan-1-one, 4-((1S,3S)-3-butyl-2-(2-chloroacetyl)-6-methoxy-1,2,3,4-tetrahy-droisoquinolin-1-yl)-N-cyclopropylbenzamide, 4-((1S,3S)-3-butyl-6-methoxy-2-propioloyl-1,2,3,4-tetrahydroisoquinolin-1-yl)-N-cyclopropylbenzamide, 1-((1S,3S)-1-(4-(adamantan-1-ylamino)phenyl)-3-butyl-6-methoxy-3,4-dihydroisoquinolin-2(1H)-yl)-2-chloroethan-1-one, 1-((1S,3S)-1-(4-(adamantan-1-ylamino)phenyl)-3-butyl-6-methoxy-3,4-dihydroisoquinolin-2(1H)-yl)prop-2-yn-1-one, methyl(1S,3R)-1-(4-(adamantan-1-ylamino)phenyl)-2-(2-chloroacetyl)-6-methoxy-1,2,3,4-tetrahydroisoquinoline-3-carboxylate, methyl(1S,3R)-1-(4-(adamantan-1-ylamino)phenyl)-6-methoxy-2-propioloyl-1,2,3,4-tetrahydroisoquinoline-3-carboxylate, 4-((1S,3S)-3-butyl-2-(2-chloroacetyl)-6,7-dimethoxy-1,2,3,4-tetrahydroisoquinolin-1-yl)-N-cyclopropylbenzamide, or 4-((1S,3S)-3-butyl-6,7-dimethoxy-2-propioloyl-1,2,3,4-tet-rahydroisoquinolin-1-yl)-N-cyclopropylbenzamide.

In certain embodiments, when R² is —C₃-C₂haloalkyl, then ring A is not benzo[d][1,3]dioxole.

In certain embodiments, R⁵ is R⁴.

Also provided is a compound of Formula AIV, or a tautomer, stereoisomer, mixture of stereoisomers, isotopically enriched analog, or pharmaceutically acceptable salt thereof:

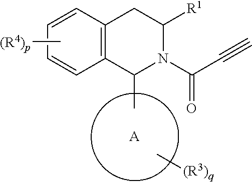

wherein each of ring A, R¹, R³, R⁴, p, and q are independently as defined herein.

Also provided is a compound of Formula AIV, or a tautomer, stereoisomer, mixture of stereoisomers, isotopically enriched analog, or pharmaceutically acceptable salt thereof:

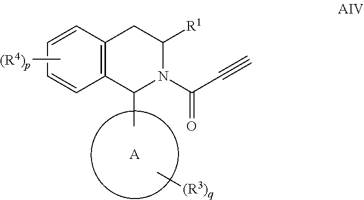

wherein:

ring A is aryl or heteroaryl;

p is 0, 1 or 2;

q is 1;

R¹ is C₁-C₆alkyl, —C(O)O—C₁-C₆alkyl, or —C(O)N(C₁-C₆alkyl)₂;

R³ is halo, —NHR⁸, —S(O)₂N(R⁷)₂, —C(O)OR⁶, —C(O)N(R⁷)₂, or heterocyclyl; each R⁴ is independently —OR⁸;

R⁶ is C₁-C₆alkyl;

each R⁷ is independently hydrogen, C₁-C₆alkyl, or C₃-C₁₀cycloalkyl, wherein each R⁷ is independently further substituted with one to three R¹¹;

each R⁸ is independently C₁-C₆alkyl or C₃-C₁₀cycloalkyl, wherein each R⁸ is independently further substituted with one to three R¹¹; and each R¹¹ is independently —O—C₁-C₆alkyl.

Also provided is a compound of Formula AIVA, or a tautomer, stereoisomer, mixture of stereoisomers, isotopically enriched analog, or pharmaceutically acceptable salt thereof:

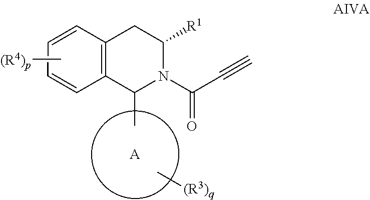

wherein each of ring A, R¹, R³, R⁴, p, and q are independently as defined herein.

Also provided is a compound of Formula AIVB, or a tautomer, stereoisomer, mixture of stereoisomers, isotopically enriched analog, or pharmaceutically acceptable salt thereof:

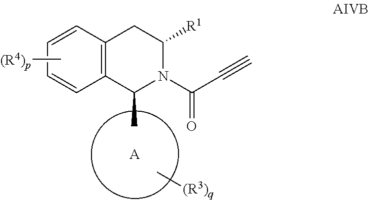

wherein each of ring A, R¹, R³, R⁴, p, and q are independently as defined herein.

Also provided is a compound of Formula AV, or a tautomer, stereoisomer, mixture of stereoisomers, isotopically enriched analog, or pharmaceutically acceptable salt thereof:

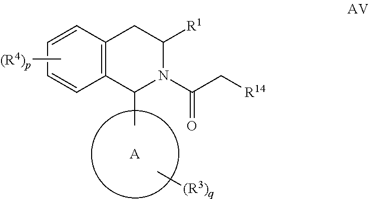

wherein each of ring A, R¹, R³, R⁴, p, and q are independently as defined herein, and R¹⁴ is halo.

Also provided is a compound of Formula AV, or a tautomer, stereoisomer, mixture of stereoisomers, isotopically enriched analog, or pharmaceutically acceptable salt thereof:

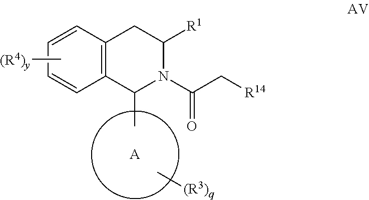

wherein:

ring A is aryl or heteroaryl;

p is 0, 1 or 2;

q is 1;

R¹ is C₁-C₆alkyl, —C(O)O—C₁-C₆alkyl, or —C(O)N(C₁-C₆alkyl)₂;

R³ is halo, —NHR⁸, —S(O)₂N(R⁷)₂, —C(O)OR⁶, —C(O)N(R⁷)₂, or heterocyclyl; each R⁴ is independently —OR⁸;

R⁶ is C₁-C₆alkyl;

each R⁷ is independently hydrogen, C₁-C₆alkyl, or C₃-C₁₀cycloalkyl, wherein each R⁷ is independently further substituted with one to three R¹¹;

each R⁸ is independently C₁-C₆alkyl or C₃-C₁₀cycloalkyl; wherein each R⁸ is independently further substituted with one to three R¹¹;

each R¹¹ is independently —O—C₁-C₆alkyl; and

R¹⁴ is halo.

In certain embodiments, R¹ is C₁-C₆alkyl, C₂-C₆alkenyl, C₂-C₆alkynyl, C₁-C₆haloalkyl, C₃-C₁₀cycloalkyl, —CN, —OR⁷, —C(O)OR⁶, —C(O)N(R⁷)₂, —OC(O)R⁶, —S(O)₂R⁸, —S(O)₂N(R⁷)₂, —S(O)N(R⁷)₂, —S(O)R⁸, —N(R⁷)₂, —NO₂, —C₁-C₆alkyl-OR⁷, or —Si(R⁵)₃.

In certain embodiments, R¹ is C₁-C₆alkyl.

Also provided is a compound of Formula AVA, or a tautomer, stereoisomer, mixture of stereoisomers, isotopically enriched analog, or pharmaceutically acceptable salt thereof:

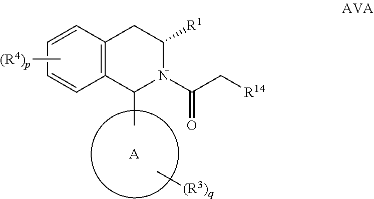

wherein each of ring A, RU, R³, R⁴, p, and q are independently as defined herein, and R¹⁴ is halo.

Also provided is a compound of Formula AVB, or a tautomer, stereoisomer, mixture of stereoisomers, isotopically enriched analog, or pharmaceutically acceptable salt thereof:

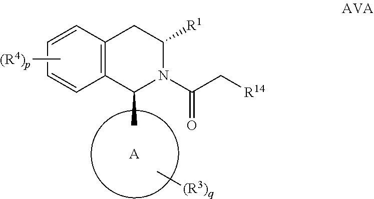

wherein each of ring A, R¹, R³, R⁴, p, and q are independently as defined herein, and R¹⁴ is halo.

In certain embodiments, R¹ is C₁-C₆alkyl, C₂-C₆alkenyl, C₂-C₆alkynyl, C₁-C₆haloalkyl, C₃-C₁₀cycloalkyl, —CN, —OR⁷, —C(O)OR⁶, —C(O)N(R⁷)₂, —OC(O)R⁶, —S(O)₂R⁸, —S(O)₂N(R⁷)₂, —S(O)N(R⁷)₂, —S(O)R⁸, —N(R⁷)₂, —NO₂, —C₁-C₆alkyl-OR⁷, or —Si(R⁵)₃.

In certain embodiments, R¹ is C₁-C₆alkyl.

Also provided is a compound of Formula AVI, or a tautomer, stereoisomer, mixture of stereoisomers, isotopically enriched analog, or pharmaceutically acceptable salt thereof:

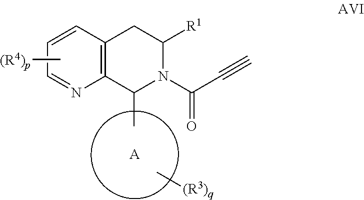

wherein each of ring A, R¹, R³, R⁴, p, and q are independently as defined herein.

Also provided is a compound of Formula AVI, or a tautomer, stereoisomer, mixture of stereoisomers, isotopically enriched analog, or pharmaceutically acceptable salt thereof:

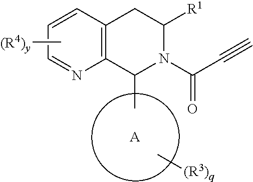

wherein:

ring A is aryl or heteroaryl;

p is 0, 1 or 2;

q is 1;

R¹ is C₁-C₆alkyl, —C(O)O—C₁-C₆alkyl, or —C(O)N(C₁-C₆alkyl)₂;

R³ is halo, —NHR⁸, —S(O)₂N(R⁷)₂, —C(O)OR⁶, —C(O)N(R⁷)₂, or heterocyclyl;

each R⁴ is independently —OR⁸;

R⁶ is C₁-C₆alkyl;

each R⁷ is independently hydrogen, C₁-C₆alkyl, or C₃-C₁₀cycloalkyl, wherein each R⁷ is independently further substituted with one to three R¹¹;

each R⁸ is independently C₁-C₆alkyl or C₃-C₁₀cycloalkyl; wherein each R⁸ is independently further substituted with one to three R¹¹; and

each R¹¹ is independently —O—C₁-C₆alkyl.

Also provided is a compound of Formula AVIA, or a tautomer, stereoisomer, mixture of stereoisomers, isotopically enriched analog, or pharmaceutically acceptable salt thereof:

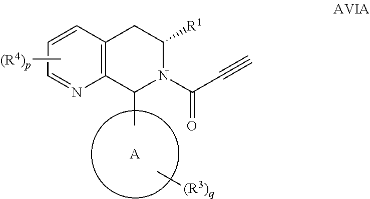

wherein each of ring A, R¹, R³, R⁴, p, and q are independently as defined herein.

Also provided is a compound of Formula AVIB, or a tautomer, stereoisomer, mixture of stereoisomers, isotopically enriched analog, or pharmaceutically acceptable salt thereof:

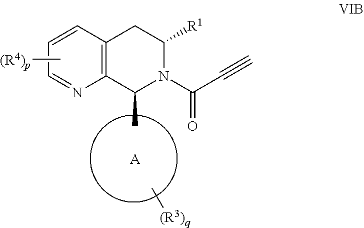

wherein each of ring A, R¹, R³, R⁴, p, and q are independently as defined herein.

Also provided is a compound of Formula AVII, or a tautomer, stereoisomer, mixture of stereoisomers, isotopically enriched analog, or pharmaceutically acceptable salt thereof:

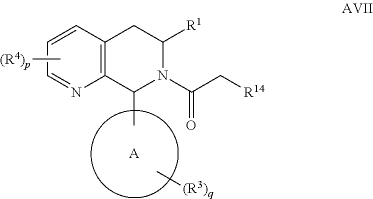

wherein each of ring A, R¹, R³, R⁴, p, and q are independently as defined herein, and R¹⁴ is halo.

Also provided is a compound of Formula AVII, or a tautomer, stereoisomer, mixture of stereoisomers, isotopically enriched analog, or pharmaceutically acceptable salt thereof:

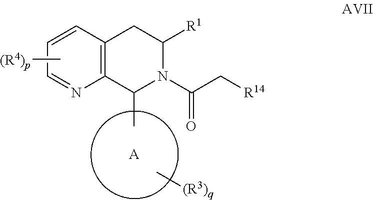

wherein:

ring A is aryl or heteroaryl;

p is 0, 1 or 2;

q is 1;

R¹ is C₁-C₆alkyl, —C(O)O—C₁-C₆alkyl, or —C(O)N(C₁-C₆alkyl)₂;

R³ is halo, —NHR⁸, —S(O)₂N(R⁷)₂, —C(O)OR⁶, —C(O)N(R⁷)₂, or heterocyclyl;

each R⁴ is independently —OR⁸;

R⁶ is C₁-C₆alkyl;

each R⁷ is independently hydrogen, C₁-C₆alkyl, or C₃-C₁₀cycloalkyl, wherein each R⁷ is independently further substituted with one to three R¹¹;

each R⁸ is independently C₁-C₆alkyl or C₃-C₁₀cycloalkyl; wherein each R⁸ is independently further substituted with one to three R¹¹; and

each R¹¹ is independently —O—C₁-C₆alkyl; and R¹⁴ is halo.

Also provided is a compound of Formula AVIIA, or a tautomer, stereoisomer, mixture of stereoisomers, isotopically enriched analog, or pharmaceutically acceptable salt thereof:

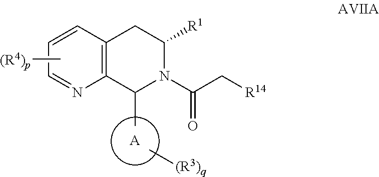

wherein each of ring A, R¹, R³, R⁴, p, and q are independently as defined herein, and R¹⁴ is halo.

Also provided is a compound of Formula AVIIB, or a tautomer, stereoisomer, mixture of stereoisomers, isotopically enriched analog, or pharmaceutically acceptable salt thereof:

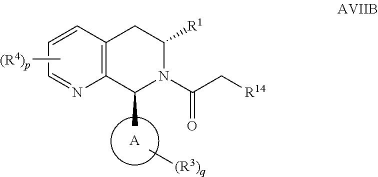

wherein each of ring A, R¹, R³, R⁴, p, and q are independently as defined herein, and R¹⁴ is halo.

Also provided is a compound of Formula AVIII, or a tautomer, stereoisomer, mixture of stereoisomers, isotopically enriched analog, or pharmaceutically acceptable salt thereof:

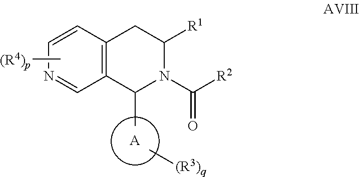

wherein each of ring A, R¹, R², R³, R⁴, p, and q are independently as defined herein.

In certain embodiments, ring A, or the moiety is:

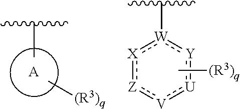

wherein 0 to 3 of U, V, W, X, Y, and Z is independently N, S, or O, and the remaining variables are CH or CR³ and each [symbol] independently represents a single or double bond, which comply with valency requirements based on U, V, W, X, Y and Z.

In certain embodiments, ring A, or the moiety

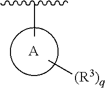

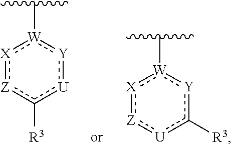

wherein 1 to 3 of U, W, X, Y, and Z is N, S, or O, and the remaining variables are CH or CR³ and [symbol] represents a single or double bond, which comply with valency requirements based on U, W, X, Y and Z.

In certain embodiments, ring A is aryl or heteroaryl. In certain embodiments, ring A is a monocyclic aryl or monocyclic heteroaryl. In certain embodiments, ring A is heterocyclyl. In certain embodiments, ring A is a 4 to 7 membered heterocyclyl. In certain embodiments, ring A is aryl. In certain embodiments, ring A is phenyl. In certain embodiments, ring A is heteroaryl. In certain embodiments, ring A is pyridyl. In certain embodiments, ring A is pyrazolyl. In certain embodiments, ring A is phenyl, pyridyl, piperidynyl, piperazinyl, or morpholinyl.

In certain embodiments, ring A is aryl or heteroaryl, each of which is substituted by one to three R³. In certain embodiments, ring A is aryl or heteroaryl, each of which is substituted by one to three R³, where at least one R³ is C₃-C₁₀cycloalkyl, heterocyclyl, aryl, or heteroaryl; wherein each C₃-C₁₀cycloalkyl, heterocyclyl, aryl, and heteroaryl of R³ is optionally substituted with one to three R¹⁰.

In certain embodiments, ring A is aryl or heteroaryl, each of which is substituted by one to three R³, where at least one R³ is C₃-C₁₀cycloalkyl, heterocyclyl, aryl, or heteroaryl; and wherein each C₃-C₁₀cycloalkyl, heterocyclyl, aryl, and heteroaryl of R³ is optionally substituted with one to three R¹⁰;

each R¹⁰ is independently —OR¹², —N(R¹²)₂, —S(O)₂R¹³, —OC(O)CHR¹²N(R¹²)₂, or C₁-C₆alkyl, wherein the C₁-C₆alkyl, of R¹⁰ is optionally independently substituted with one to three R¹¹;

each R¹¹ is independently halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl;

each R¹² is independently hydrogen, C₁-C₆alkyl or C₃-C₁₀cycloalkyl; and

each R¹³ is independently C₁-C₆alkyl or C₃-C₁₀cycloalkyl.

In certain embodiments, ring A is bicycle[1.1.1] pentan-1-yl, phenyl, piperidinyl, pyrazolyl, pyridyl, or qui-nolinyl, each of which is optionally substituted by one, two or three R³. In certain embodiments, ring A is bicyclo[1.1.1]pentan-1-yl, phenyl, piperidinyl, pyrazolyl, pyridyl, or quinolinyl, each of which is substituted by one, two or three R³. In certain embodiments, ring A is bicyclo[1.1.1]pentan-1-yl, phenyl, piperidinyl, pyrazolyl, pyridyl, or quinolinyl, each of which is substituted by two or three R³.

In certain embodiments, ring A is aryl or heteroaryl, each of which is substituted by two or three R³. In certain embodiments, ring A is aryl or heteroaryl, each of which is substituted by two or three R³; wherein at least one R³ is halo.

In certain embodiments, ring A is cyclohexyl. In certain embodiments, ring A is C₄-C₁₀cycloalkyl. In certain embodiments, ring A is a C₄-C₇cycloalkyl. In certain embodiments, ring A is bicyclo[1.1.1]pentanyl. In certain embodiments, ring A is selected from cyclobutyl, cyclopentyl, cyclohexyl, and cycloheptyl.

In certain embodiments, ring A, or the moiety

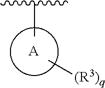

is:

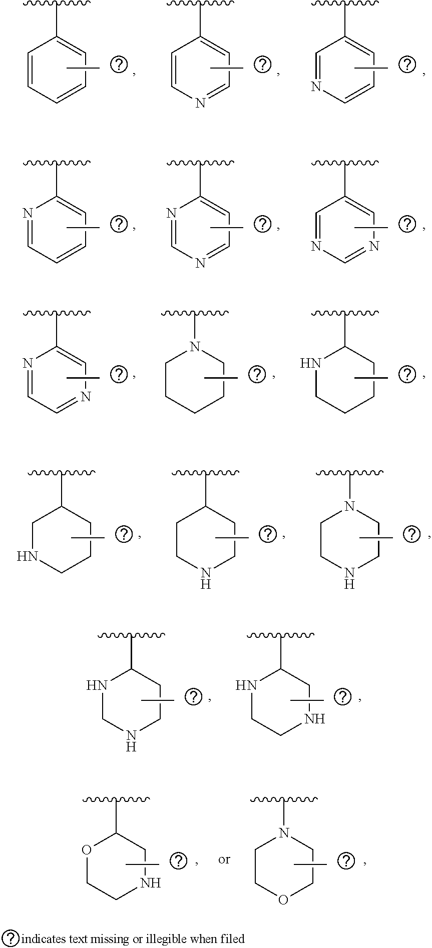

where q and each R³ is independently as defined herein.

In certain embodiments, ring A, or the moiety is:

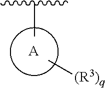

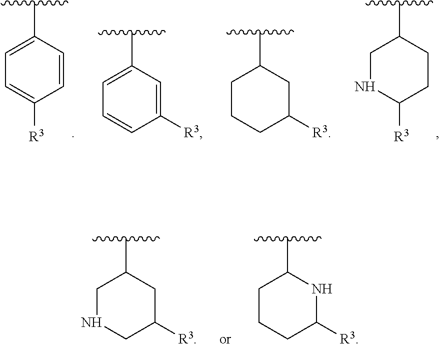

where R³ is independently as defined herein.

In certain embodiments, ring A is a bridged bicyclic ring selected from:

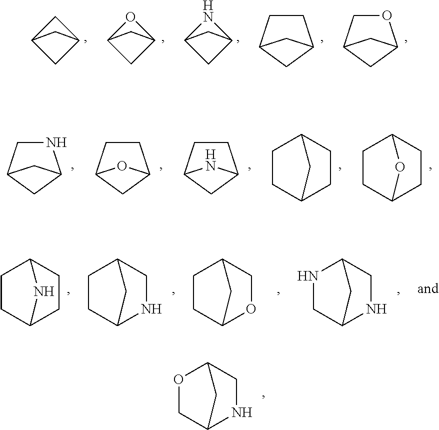

wherein each is substituted with one to three R³. In certain embodiments, ring A is a bridged bicyclic ring selected from:

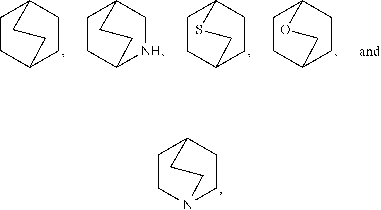

wherein each R³ is attached to a carbon atom on the bridged bicyclic ring.

In certain embodiments, ring A, or the moiety

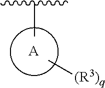

is:

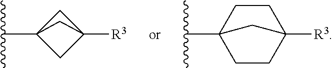

Also provided is a compound of Formula AVIII, or a tautomer, stereoisomer, mixture of stereoisomers, isotopically enriched analog, or pharmaceutically acceptable salt thereof:

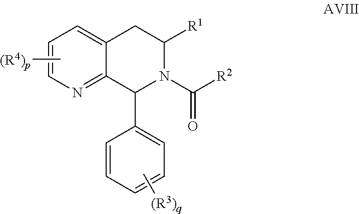

wherein each of R¹, R², R³, R⁴, p, and q are independently as defined herein.

Also provided is a compound of Formula AVIIIA, or a tautomer, stereoisomer, mixture of stereoisomers, isotopically enriched analog, or pharmaceutically acceptable salt thereof:

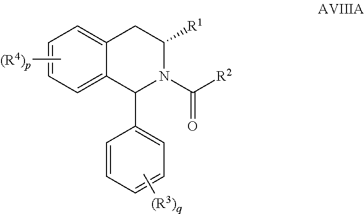

wherein each of R¹, R², R³, R⁴, p, and q are independently as defined herein.

Also provided is a compound of Formula AVIIIB, or a tautomer, stereoisomer, mixture of stereoisomers, isotopically enriched analog, or pharmaceutically acceptable salt thereof:

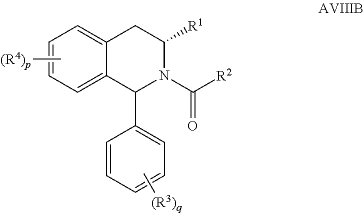

wherein each of R¹, R², R³, R⁴, p, and q are independently as defined herein.

In certain embodiments, R¹ is C₁-C₆alkyl, C₂-C₆alkenyl, C₂-C₆alkynyl, C₁-C₆haloalkyl, C₃-C₁₀cycloalkyl, —CN, —C(O)OR⁶, —C(O)N(R⁷)₂, —N(R⁷)₂, —OR⁷, or —C₁-C₆alkyl-OR⁷.

In certain embodiments, R¹ is —C(O)OR⁶ or —C(O)N(R⁷)₂.

In certain embodiments, R¹ is C₁-C₆alkyl. In certain embodiments, In certain embodiments, R¹ is C₂-C₆alkyl. In certain embodiments, R¹ is C₃-C₆alkyl. In certain embodiments, R¹ is C₅-C₆alkyl. In certain embodiments, R¹ is C₂-C₃alkyl. In certain embodiments, R¹ is C₄-C₆alkyl.

In certain embodiments, R¹ is methyl. In certain embodiments, R¹ is n-butyl.

In certain embodiments, R¹ is —CH₂—R¹⁶, wherein R¹⁶ is C₁-C₅alkyl, C₂-C₅alkenyl, C₂-C₅alkynyl, C₁-C₅haloalkyl, or —C₁-C₅alkyl-OR⁷.

In certain embodiments, R¹ is C₂-C₆alkenyl, C₂-C₆alkynyl, C₁-C₆haloalkyl, C₃-C₁₀cycloalkyl, —CN, —OR⁷, —C(O)N(R⁷)₂, —OC(O)R⁶, —S(O)₂R⁸, —S(O)₂N(R⁷)₂, —S(O)N(R⁷)₂, —S(O)R⁸, —N(R⁷)₂, —NO₂, —CᵣC₆alkyl-OR⁷, or —Si(R⁵)₃.

In certain embodiments, R¹ is other than methyl. In certain embodiments, R¹ is other than n-butyl. In certain embodiments, R¹ is other than —C(O)OR⁶. In certain embodiments, R¹ is other than —C(O)OCH₃.

Also provided is a compound of Formula AIX, or a tautomer, stereoisomer, mixture of stereoisomers, isotopically enriched analog, or pharmaceutically acceptable salt thereof:

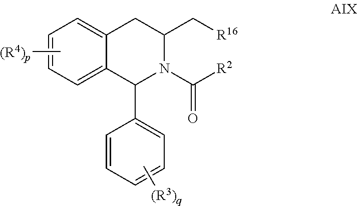

wherein each of R², R³, R⁴, R¹⁶, p, and q are independently as defined herein. In certain embodiments, R¹⁶ is hydrogen or C₂-C₅alkyl.

Also provided is a compound of Formula AIXA, or a tautomer, stereoisomer, mixture of stereoisomers, isotopically enriched analog, or pharmaceutically acceptable salt thereof:

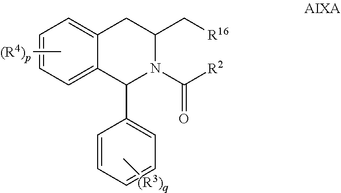

wherein each of R², R³, R⁴, R⁶, p, and q are independently as defined herein. In certain embodiments, R¹⁶ is hydrogen or C₂-C₅alkyl.

Also provided is a compound of Formula AIXB, or a tautomer, stereoisomer, mixture of stereoisomers, isotopically enriched analog, or pharmaceutically acceptable salt thereof:

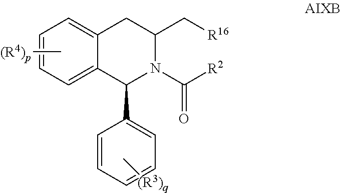

wherein each of R², R³, R⁴, R¹⁶, p, and q are independently as defined herein. In certain embodiments, R¹⁶ is hydrogen or C₂-C₅alkyl.

In certain embodiments, R² is —C₁-C₂haloalkyl, —C₂-C₃alkenyl, —C₂-C₃haloalkenyl, C₂alkynyl, wherein the C₁-C₂haloalkyl and C₂-C₃alkenylhalo are optionally substituted with one or two —CH₃, and the C₂alkynyl is optionally substituted with one —CH₃. In certain embodiments, R² is —C₁-C₂haloalkyl. In certain embodiments, R² is —C₂-C₃alkenyl. In certain embodiments, R² is C₂-C₃haloalkenyl. In certain embodiments, R² is C₂alkynyl.

In certain embodiments, at least one R³ is halo, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R, —NR¹²C(O)OR⁸, —OC(O)R⁸, —C(O)R⁶, or —OC(O)CHR⁸N(R¹²)₂.

In certain embodiments, at least one R³ is halo.

In certain embodiments, at least one R³ is —NHR⁸. In certain embodiments, at least one R³ is —N(R⁸)₂. In certain embodiments, q is 2, and one R³ is halo and the other R³ is —N(R⁸)₂. In certain embodiments, q is 3, and two R³ are independently halo and one R³ is —N(R⁸)₂.

In certain embodiments, at least one R³ is —C(O) OR⁶ or —C(O)R⁶.

In certain embodiments, at least one R³ is —S(O)₂N(R⁷)₂, —S(O)N(R⁷)₂, or —C(O)N(R⁷)₂.

In certain embodiments, at least one R³ is —S(O)₂R, —S(O)R⁸, —NR²C(O)R⁸, —NR¹²C(O)OR⁸, —OC(O)R⁸, or —OC(O)CHRN(R¹²)₂.

In certain embodiments, each R³ is independently halo, —CN, —OR⁸, —NHR⁸, —S(O)₂R⁸, —S(O)₂N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —OC(O) CHR⁸N(R¹²)₂, C₁-C₆alkyl, C₃-C₁₀cycloalkyl, heterocyclyl, heteroaryl, or —C₁-C₆alkylheterocyclyl; wherein each C₁-C₆alkyl, C₃-C₁₀cycloalkyl, heterocyclyl, heteroaryl, or —C₁-C₆alkylheterocyclyl of R³ is independently optionally substituted with one to three R¹⁰.

In certain embodiments, each R³ is independently halo, —CN, —OR⁸, —NHR⁸, —S(O)₂R⁸, —S(O)₂N(R⁷)₂, —NR¹²C(O)R⁸, —NR¹²C(O)OR⁸, —OC(O)R⁸, —OC(O) CHR⁸N(R¹²)₂, C₁-C₆alkyl, C₃-C₁₀cycloalkyl, heterocyclyl, heteroaryl, or —C₁-C₆alkylheterocyclyl; wherein each C₁-C₆alkyl, C₃-C₁₀cycloalkyl, heterocyclyl, heteroaryl, or C₁-C₆alkylheterocyclyl is independently optionally substituted with one to three substituents independently selected from —OR¹², —N(R¹²)₂, —S(O)₂R¹³, —OC(O)CHR¹²N(R¹²)₂, and C₁-C₆alkyl optionally substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein each R¹² is independently hydrogen, C₁-C₆alkyl or C₃-C₁₀cycloalkyl; and each R¹³ is independently C₁-C₆alkyl or C₃-C₁₀cycloalkyl.

In certain embodiments, each R³ is independently —NH₂, fluoro, methyl, pyridine-4-carboxamido, pyridin-3-amino, pentyloxycarbonylamino, N-(3-aminobicyclo[1.1.1]pentan-1-yl)amino, morpholin-4-yl, methoxycarbonyl, dim-ethylcarbamoyl, cyclopropylcarbamoyl, cyclohexyl, cyclobutylcarbamoyl, cyclobutylaminosulfonyl, adamanty-lamino, (adamantan-1-ylamino)methyl, 3-methyl-1,2,4-ox-adiazol-5-yl, 2-methylpyridine-4-carboxamido, (bicyclo[1.1.1]pentan-1-ylamino)methyl, (adamantan-1-yl)carbamoyl, or (2-methoxyethyl)carbamoyl.

In certain embodiments, q is 0 or 1, and R³ is —NH₂, fluoro, methyl, pyridine-4-carboxamido, pyridin-3-amino, pentyloxycarbonylamino, N-(3-aminobicyclo[0.1.1.1]pentan-1-yl)amino, morpholin-4-yl, methoxycarbonyl, dim-ethylcarbamoyl, cyclopropylcarbamoyl, cyclohexyl, cyclobutylcarbamoyl, cyclobutylaminosulfonyl, adamanty-lamino, (adamantan-1-ylamino)methyl, 3-methyl-1,2,4-ox-adiazol-5-yl, 2-methylpyridine-4-carboxamido, (bicyclo[1.1.1]pentan-1-ylamino)methyl, (adamantan-1-yl)carbamoyl, or (2-methoxyethyl)carbamoyl.

In certain embodiments, each R⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R⁵)₃, —C(O)OR⁶, —C(O)N(R⁷)₂, —NR¹²C (O)R⁸, —OC(O)R⁸, —C(O)R⁶, C₁-C₆alkyl, C₂-C₆alkenyl, C₂-C₆alkynyl, or C₃-C₁₀cycloalkyl; wherein each C₁-C₆alkyl, C₂-C₆alkenyl, C₂-C₆alkynyl, or C₃-C₁₀cycloalkyl of R⁴ is independently optionally substituted with one to three R¹⁰.

In certain embodiments, each R⁴ is independently halo, —CN, —OR⁷, C₁-C₆galkyl, C₂-C₆alkynyl, or C₃-C₁₀cycloalkyl; wherein each C₁-C₆alkyl, C₂-C₆alkynyl, or C₃-C₁₀cycloalkyl of R⁴ is independently optionally substituted with one to three R¹⁰.

In certain embodiments, each R⁴ is independently halo, —CN, —OH, C₁-C₆alkyl, C₂-C₆alkynyl, or C₃-C₁₀cycloalkyl.

In certain embodiments, each R⁴ is independently halo, —CN, —OH, —OR⁸, C₁-C₆alkyl, or C₂-C₆alkynyl; wherein the C₁-C₆alkyl of R⁴ is optionally substituted with one to three R¹⁰.

In certain embodiments, each R⁴ is independently halo, —CN, —OH, —OR⁸, C₁-C₆alkyl, C₂-C₆alkynyl; wherein the C₁-C₆alkyl of R⁴ is optionally substituted with one to three substituents independently selected from —OR¹², —N(R¹²)₂, —S(O)₂R¹³, —OC(O)CHR¹²N(R¹²)₂, and C₁-C₆alkyl optionally substituted with one to three halo, —OR¹², —N(R¹²)₂, —Si(R¹²)₃, —C(O)OR¹², —NR¹²C(O) OR¹², —OC(O)CHR¹²N(R¹²)₂, C₁-C₆alkyl, or heterocyclyl; wherein

each R¹² is independently hydrogen, C₁-C₆alkyl or C₃-C₁₀cycloalkyl; and

each R¹³ is independently C₁-C₆alkyl or C₃-C₁₀cycloalkyl.

In certain embodiments, each R⁵ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R⁵)₃, —C(O)OR⁶, —C(O)N(R⁷)₂, —NR¹²C (O)R⁸, —OC(O)R⁸, —C(O)R⁶, C₁-C₆alkyl, C₂-C₆alkenyl, C₂-C₆alkynyl, or C₃-C₁₀cycloalkyl; wherein each C₁-C₆alkyl, C₂-C₆alkenyl, C₂-C₆alkynyl, or C₃-C₁₀cycloalkyl of R⁵ is independently optionally substituted with one to three R¹⁰.

In certain embodiments, each R⁵ is independently halo, —CN, —OR⁷, C₁-C₆alkyl, C₂-C₆alkynyl, or C₃-C₁₀cycloalkyl; wherein each C₁-C₆alkyl, C₂-C₆alkynyl, or C₃-C₁₀cycloalkyl of R⁵ is independently optionally substituted with one to three R¹⁰.

In certain embodiments, each R⁵ is independently halo, —CN, —OH, C₁-C₆alkyl, C₂-C₆alkynyl, or C₃-C₁₀cycloalkyl.

In certain embodiments, each R⁵ is independently halo, —CN, —OH, —OR⁸, C₁-C₆alkyl, or C₂-C₆alkynyl; wherein the C₁-C₆alkyl of R⁵ is optionally substituted with one to three R¹⁰.

In certain embodiments, each R⁵ is independently halo, —CN, —OH, —OR⁸, C₁-C₆alkyl, C₂-C₆alkynyl; wherein the C₁-C₆alkyl of R⁵ is optionally substituted with one to three substituents independently selected from —OR¹², —N(R¹²)₂, —S(O)₂R, —OC(O)CHR¹²N(R¹²)₂, and C₁-C₆alkyl optionally substituted with one to three halo, —OR¹², —N(R¹²)₂, —Si(R¹²)₃, —C(O)OR¹², —NR¹²C(O) OR¹², —OC(O)CHR¹²N(R¹²)₂, C₁-C₆alkyl, or heterocyclyl; wherein

each R¹² is independently hydrogen, C₁-C₆alkyl or C₃-C₁₀cycloalkyl; and

each R¹³ is independently C₁-C₆alkyl or C₃-C₁₀cycloalkyl.

In certain embodiments, each R⁶ is independently hydrogen, C₁-C₆alkyl, C₂-C₆alkenyl, or —C₁-C₆alkylC₃-C₁₀cycloalkyl; wherein each R⁶ is independently further substituted with one to three R¹¹.

In certain embodiments, each R⁶ is independently hydrogen, C₁-C₆alkyl, C₂-C₆alkenyl, or —C₁-C₆alkylC₃-C₁₀cycloalkyl; wherein each R⁶ is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein

each R¹² is independently hydrogen, C₁-C₆alkyl or C₃-C₁₀cycloalkyl.

In certain embodiments, each R⁷ is independently hydrogen, C₁-C₆alkyl, C₃-C₁₀cycloalkyl, heterocyclyl, heteroaryl, —C₁-C₆alkylC₃-C₆cycloalkyl, —C₃-C₆alkylheterocyclyl, or two R⁷ together with the nitrogen atom to which they are attached, form a 4 to 7 membered heterocyclyl; wherein each R⁷ or ring formed thereby is independently further substituted with one to three R¹¹.

In certain embodiments, each R⁷ is independently hydrogen, C₁-C₆alkyl, C₃-C₁₀cycloalkyl, heterocyclyl, heteroaryl, —C₁-C₆alkylC₃-C₆cycloalkyl, —Cj-C₆alkylheterocyclyl, or two R⁷ together with the nitrogen atom to which they are attached, form a 4 to 7 membered heterocyclyl; wherein each R⁷ or ring formed thereby is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein

each R¹² is independently hydrogen, C₁-C₆alkyl or C₃-C₁₀cycloalkyl.

In certain embodiments, each R⁸ is independently C₁-C₆alkyl, C₂-C₆alkynyl, C₃-C₁₀cycloalkyl, —C₁-C₆alkylC₃-C₁₀cycloalkyl, or —C₁-C₆alkylaryl; wherein each R⁸ is independently further substituted with one to three R¹¹.

In certain embodiments, each R⁸ is independently C₁-C₆alkyl, C₂-C₆alkynyl, C₃-C₁₀cycloalkyl, —C₁-C₆alkylC₃-C₁₀cycloalkyl, or —C₁-C₆alkylaryl; wherein each R⁸ is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, Ci-Qalkyl, or heterocyclyl; wherein each R¹² is independently hydrogen, C₁-C₆alkyl or C₃-C₁₀cycloalkyl.

In certain embodiments, each R¹⁰ is independently —OR¹², —N(R¹²)₂, —S(O)₂R¹³, —OC(O)CHR¹²N(R¹²)₂, or C₁-C₆alkyl, wherein the C₁-C₆alkyl, of R¹⁰ is optionally independently substituted with one to three R¹¹;

each R¹¹ is independently halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl;

each R¹² is independently hydrogen, C₁-C₆alkyl or C₃-C₁₀cycloalkyl; and

each R¹³ is independently C₁-C₆alkyl or C₃-C₁₀cycloalkyl.

In certain embodiments, each R⁵ is independently C₁-C₆alkyl.

In certain embodiments, p is 0. In certain embodiments, p is 0 or 1. In certain embodiments, p is 1 or 2. In certain embodiments, p is 1. In certain embodiments, p is 2.

In certain embodiments, q is 0. In certain embodiments, q is 0 or 1. In certain embodiments, q is 1 or 2. In certain embodiments, q is 1. In certain embodiments, q is 2. In certain embodiments, q is 3.

Also provided is a compound, or a tautomer, stereoisomer, mixture of stereoisomers, isotopically enriched analog, or pharmaceutically acceptable salt thereof, selected from Table 3B:

TABLE 3B

| No. | Structure |
|---|---|
| 1 | 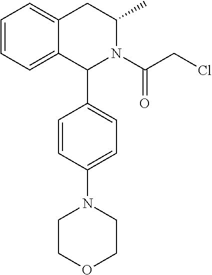 [SMILES: C[C@H]1Cc2ccccc2C(c2ccc(N3CCOCC3)cc2)N1C(=O)CCl] |
| 2 | 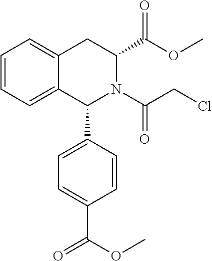 [SMILES: COC(=O)c1ccc([C@@H]2c3ccccc3C[C@H](C(=O)OC)N2C(=O)CCl)cc1] |
| 3 | 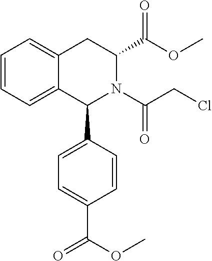 [SMILES: COC(=O)c1ccc([C@H]2c3ccccc3C[C@H](C(=O)OC)N2C(=O)CCl)cc1] |
| 4 | 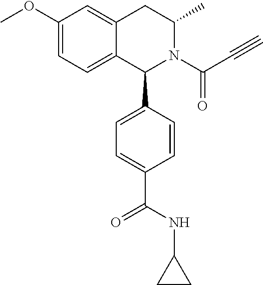 [SMILES: C#CC(=O)N1[C@@H](c2ccc(C(=O)NC3CC3)cc2)c2ccc(OC)cc2C[C@@H]1C] |
| 5 | 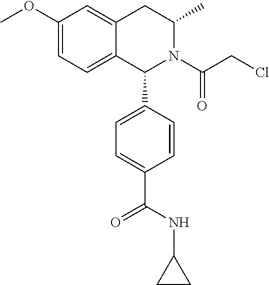 [SMILES: COc1ccc2c(c1)C[C@H](C)N(C(=O)CCl)[C@@H]2c1ccc(C(=O)NC2CC2)cc1] |
| 6 | 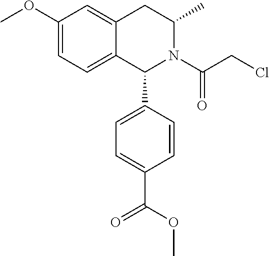 [SMILES: COC(=O)c1ccc([C@@H]2c3ccc(OC)cc3C[C@H](C)N2C(=O)CCl)cc1] |
| 7 | 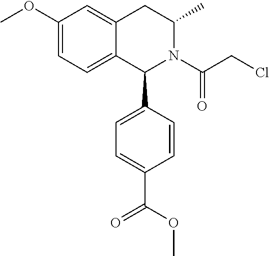 [SMILES: COC(=O)c1ccc([C@H]2c3ccc(OC)cc3C[C@H](C)N2C(=O)CCl)cc1] |
| 8 | 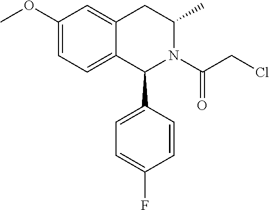 [SMILES: COc1ccc2c(c1)C[C@H](C)N(C(=O)CCl)[C@H]2c1ccc(F)cc1] |
| 9 | 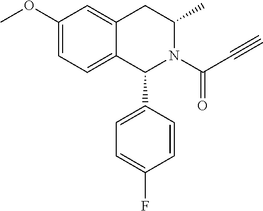 [SMILES: C#CC(=O)N1[C@H](c2ccc(F)cc2)c2ccc(OC)cc2C[C@@H]1C] |
| 10 | 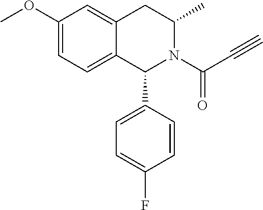 [SMILES: C#CC(=O)N1[C@H](c2ccc(F)cc2)c2ccc(OC)cc2C[C@@H]1C] |
| 11 | 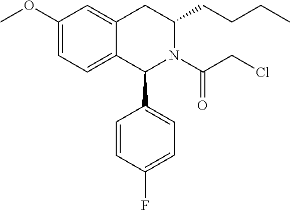 [SMILES: CCCC[C@H]1Cc2cc(OC)ccc2[C@H](c2ccc(F)cc2)N1C(=O)CCl] |
| 12 | 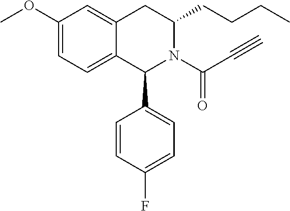 [SMILES: C#CC(=O)N1[C@@H](CCCC)Cc2cc(OC)ccc2[C@@H]1c1ccc(F)cc1] |
| 13 | 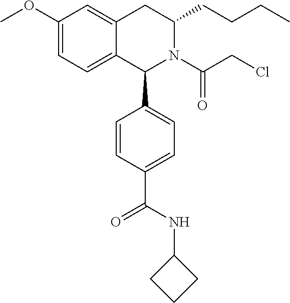 [SMILES: CCCC[C@H]1Cc2cc(OC)ccc2[C@H](c2ccc(C(=O)NC3CCC3)cc2)N1C(=O)CCl] |
| 14 | 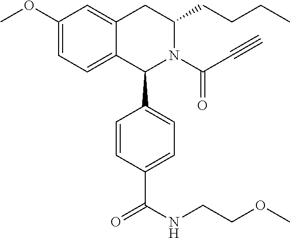 |
| 15 | 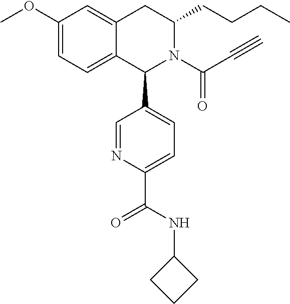 [SMILES: C#CC(=O)N1[C@@H](CCCC)Cc2cc(OC)ccc2[C@@H]1c1ccc(C(=O)NC2CCC2)nc1] |
| 16 | 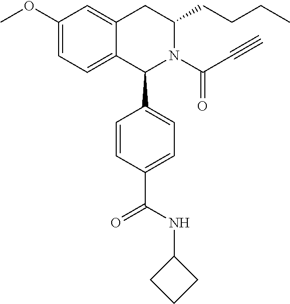 [SMILES: C#CC(=O)N1[C@@H](CCCC)Cc2cc(OC)ccc2[C@@H]1c1ccc(C(=O)NC2CCC2)cc1] |
| 17 | 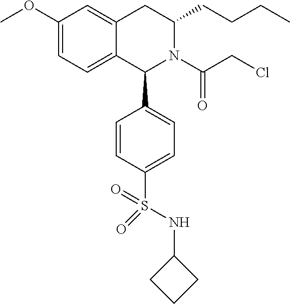 [SMILES: CCCC[C@H]1Cc2cc(OC)ccc2[C@H](c2ccc(S(=O)(=O)NC3CCC3)cc2)N1C(=O)CCl] |
| 18 | 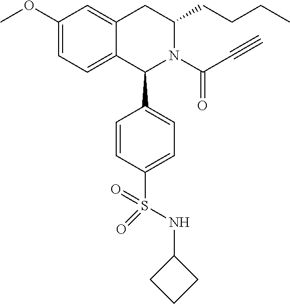 [SMILES: C#CC(=O)N1[C@@H](CCCC)Cc2cc(OC)ccc2[C@@H]1c1ccc(S(=O)(=O)NC2CCC2)cc1] |
| 19 | 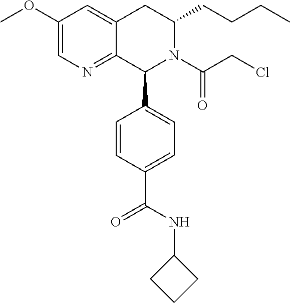 [SMILES: CCCC[C@H]1Cc2cc(OC)cnc2[C@H](c2ccc(C(=O)NC3CCC3)cc2)N1C(=O)CCl] |
| 20 | 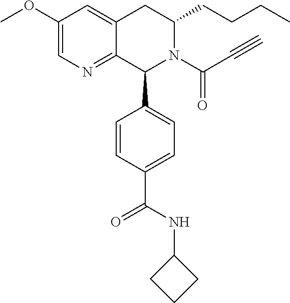 [SMILES: C#CC(=O)N1[C@@H](CCCC)Cc2cc(OC)cnc2[C@@H]1c1ccc(C(=O)NC2CCC2)cc1] |
| 21 | 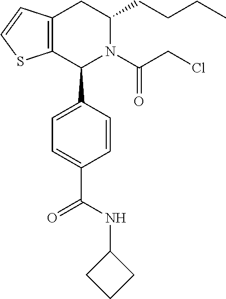 [SMILES: CCCC[C@H]1Cc2ccsc2[C@H](c2ccc(C(=O)NC3CCC3)cc2)N1C(=O)CCl] |
| 22 | 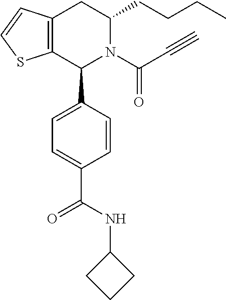 [SMILES: C#CC(=O)N1[C@@H](CCCC)Cc2ccsc2[C@@H]1c1ccc(C(=O)NC2CCC2)cc1] |
| 23 | 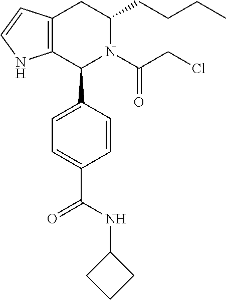 [SMILES: CCCC[C@H]1Cc2cc[nH]c2[C@H](c2ccc(C(=O)NC3CCC3)cc2)N1C(=O)CCl] |
| 24 | 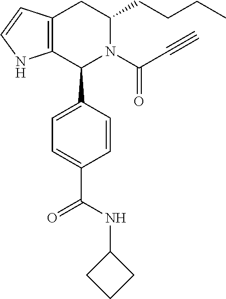 [SMILES: C#CC(=O)N1[C@@H](CCCC)Cc2cc[nH]c2[C@@H]1c1ccc(C(=O)NC2CCC2)cc1] |
| 25 | 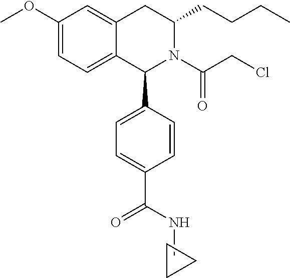 |
| 26 | 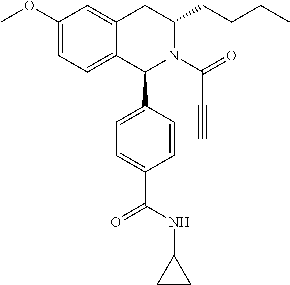 [SMILES: C#CC(=O)N1[C@@H](CCCC)Cc2cc(OC)ccc2[C@@H]1c1ccc(C(=O)NC2CC2)cc1] |
| 27 | 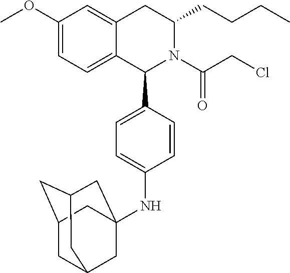 [SMILES: CCCC[C@H]1Cc2cc(OC)ccc2[C@H](c2ccc(NC34CC5CC(CC(C5)C3)C4)cc2)N1C(=O)CCl] |
| 28 | 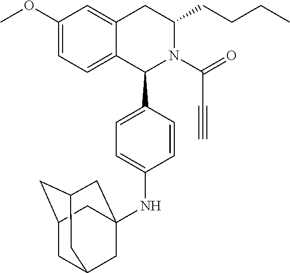 [SMILES: C#CC(=O)N1[C@@H](CCCC)Cc2cc(OC)ccc2[C@@H]1c1ccc(NC23CC4CC(CC(C4)C2)C3)cc1] |
| 29 | 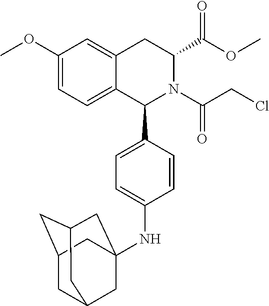 [SMILES: COC(=O)[C@H]1Cc2cc(OC)ccc2[C@H](c2ccc(NC34CC5CC(CC(C5)C3)C4)cc2)N1C(=O)CCl] |
| 30 | 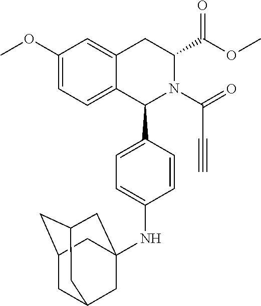 |
| 31 | 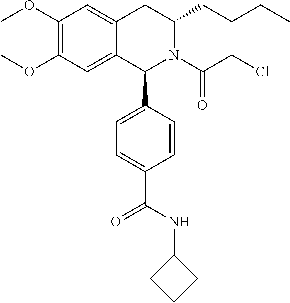 [SMILES: CCCC[C@H]1Cc2cc(OC)c(OC)cc2[C@H](c2ccc(C(=O)NC3CCC3)cc2)N1C(=O)CCl] |
| 32 | 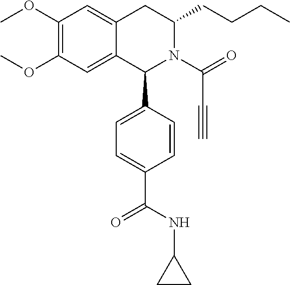 [SMILES: C#CC(=O)N1[C@@H](CCCC)Cc2cc(OC)c(OC)cc2[C@@H]1c1ccc(C(=O)NC2CC2)cc1] |
| 33 | 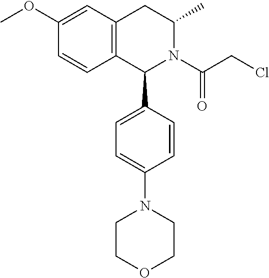 [SMILES: COc1ccc2c(c1)C[C@H](C)N(C(=O)CCl)[C@H]2c1ccc(N2CCOCC2)cc1] |
| 34 | 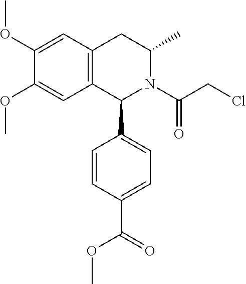 [SMILES: COC(=O)c1ccc([C@H]2c3cc(OC)c(OC)cc3C[C@H](C)N2C(=O)CCl)cc1] |
| 35 | 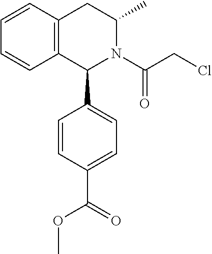 [SMILES: COC(=O)c1ccc([C@H]2c3ccccc3C[C@H](C)N2C(=O)CCl)cc1] |
| 36 | 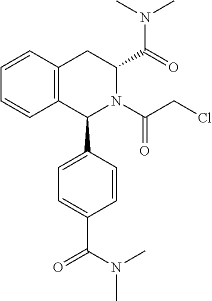 [SMILES: CN(C)C(=O)c1ccc([C@H]2c3ccccc3C[C@H](C(=O)N(C)C)N2C(=O)CCl)cc1] |
| 37 | 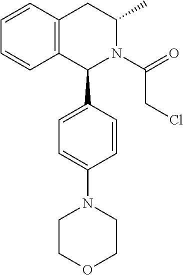 [SMILES: C[C@H]1Cc2ccccc2[C@H](c2ccc(N3CCOCC3)cc2)N1C(=O)CCl] |
| 38 | 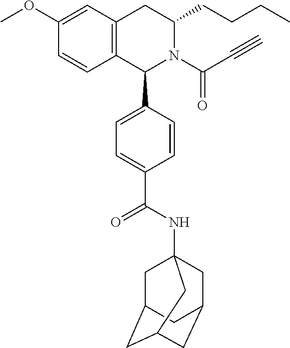 [SMILES: C#CC(=O)N1[C@@H](CCCC)Cc2cc(OC)ccc2[C@@H]1c1ccc(C(=O)NC23CC4CC(CC(C4)C2)C3)cc1] |
| 39 | 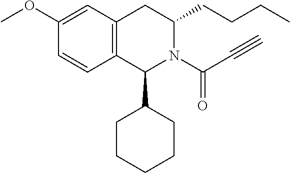 [SMILES: C#CC(=O)N1[C@@H](CCCC)Cc2cc(OC)ccc2[C@@H]1C1CCCCC1] |
| 40 | 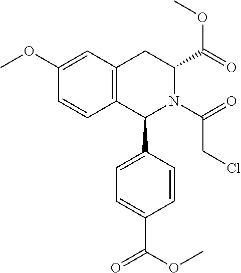 [SMILES: COC(=O)c1ccc([C@H]2c3ccc(OC)cc3C[C@H](C(=O)OC)N2C(=O)CCl)cc1] |
| 41 | 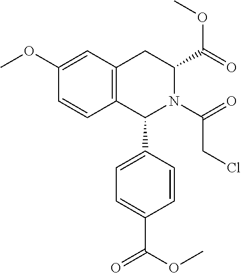 [SMILES: COC(=O)c1ccc([C@@H]2c3ccc(OC)cc3C[C@H](C(=O)OC)N2C(=O)CCl)cc1] |
| 42 | 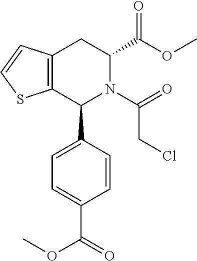 [SMILES: COC(=O)c1ccc([C@H]2c3sccc3C[C@H](C(=O)OC)N2C(=O)CCl)cc1] |
| 43 | 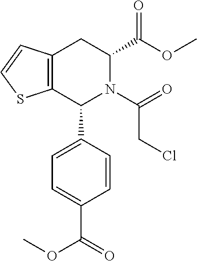 [SMILES: COC(=O)c1ccc([C@@H]2c3sccc3C[C@H](C(=O)OC)N2C(=O)CCl)cc1] |
| 44 | 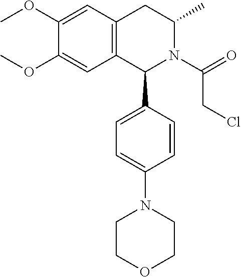 [SMILES: COc1cc2c(cc1OC)[C@H](c1ccc(N3CCOCC3)cc1)N(C(=O)CCl)[C@@H](C)C2] |
| 45 | 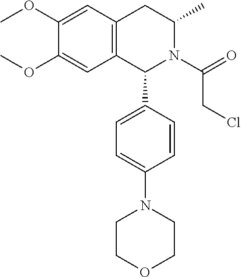 [SMILES: COc1cc2c(cc1OC)[C@@H](c1ccc(N3CCOCC3)cc1)N(C(=O)CCl)[C@@H](C)C2] |
| 46 | 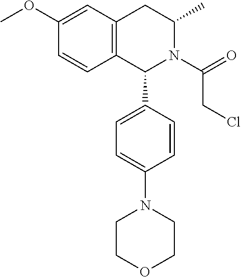 [SMILES: COc1ccc2c(c1)C[C@H](C)N(C(=O)CCl)[C@@H]2c1ccc(N2CCOCC2)cc1] |
| 47 | 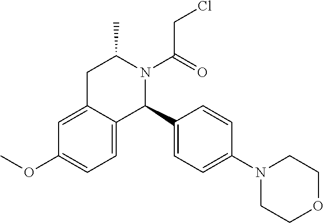 [SMILES: COc1ccc2c(c1)C[C@H](C)N(C(=O)CCl)[C@H]2c1ccc(N2CCOCC2)cc1] |
| 48 | 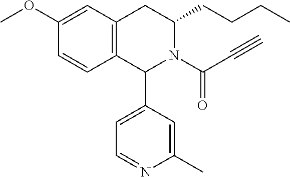 [SMILES: C#CC(=O)N1C(c2ccnc(C)c2)c2ccc(OC)cc2C[C@@H]1CCCC] |
| 49 | 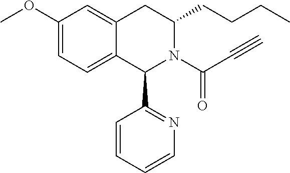 [SMILES: C#CC(=O)N1[C@@H](CCCC)Cc2cc(OC)ccc2[C@@H]1c1ccccn1] |
| 50 | 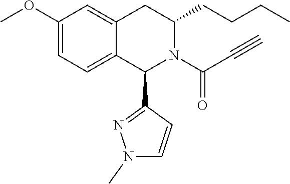 [SMILES: C#CC(=O)N1[C@@H](CCCC)Cc2cc(OC)ccc2[C@@H]1c1ccn(C)n1] |
| 51 | 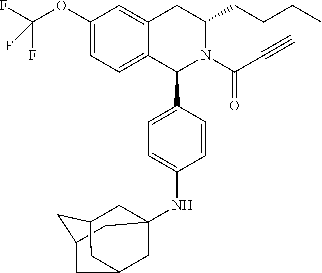 [SMILES: C#CC(=O)N1[C@@H](CCCC)Cc2cc(OC(F)(F)F)ccc2[C@@H]1c1ccc(NC23CC4CC(CC(C4)C2)C3)cc1] |
| 52 | 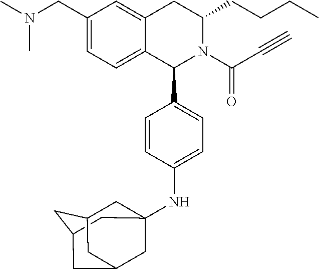 [SMILES: C#CC(=O)N1[C@@H](CCCC)Cc2cc(CN(C)C)ccc2[C@@H]1c1ccc(NC23CC4CC(CC(C4)C2)C3)cc1] |
| 53 | 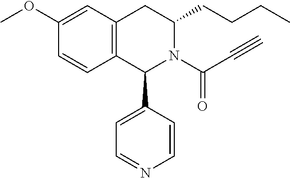 [SMILES: C#CC(=O)N1[C@@H](CCCC)Cc2cc(OC)ccc2[C@@H]1c1ccncc1] |
| 54 | 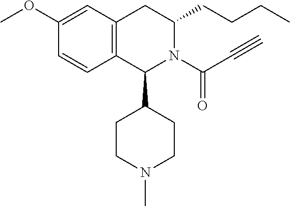 [SMILES: C#CC(=O)N1[C@@H](CCCC)Cc2cc(OC)ccc2[C@@H]1C1CCN(C)CC1] |
| 55 | 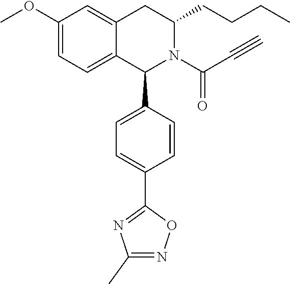 [SMILES: C#CC(=O)N1[C@@H](CCCC)Cc2cc(OC)ccc2[C@@H]1c1ccc(-c2nc(C)no2)cc1] |
| 56 | 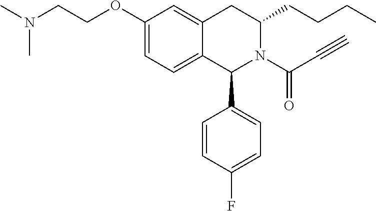 [SMILES: C#CC(=O)N1[C@@H](CCCC)Cc2cc(OCCN(C)C)ccc2[C@@H]1c1ccc(F)cc1] |
| 57 | 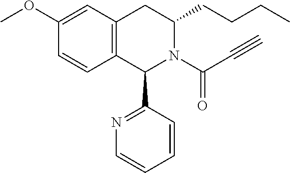 [SMILES: C#CC(=O)N1[C@@H](CCCC)Cc2cc(OC)ccc2[C@@H]1c1ccccn1] |
| 58 | 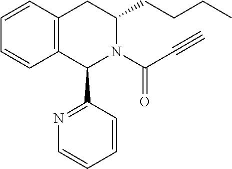 [SMILES: C#CC(=O)N1[C@@H](CCCC)Cc2ccccc2[C@@H]1c1ccccn1] |
| 59 | 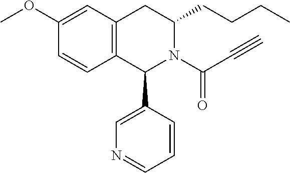 [SMILES: C#CC(=O)N1[C@@H](CCCC)Cc2cc(OC)ccc2[C@@H]1c1cccnc1] |
| 60 | 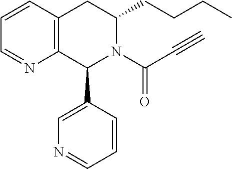 [SMILES: C#CC(=O)N1[C@@H](CCCC)Cc2cccnc2[C@@H]1c1cccnc1] |
| 61 | 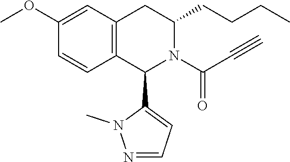 [SMILES: C#CC(=O)N1[C@@H](CCCC)Cc2cc(OC)ccc2[C@@H]1c1ccnn1C] |
| 62 | 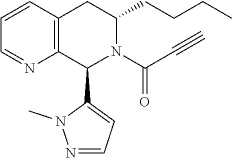 [SMILES: C#CC(=O)N1[C@@H](CCCC)Cc2cccnc2[C@@H]1c1ccnn1C] |
| 63 | 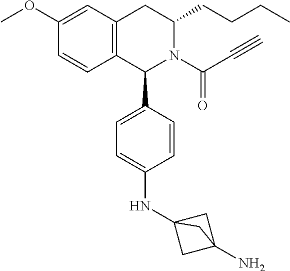 [SMILES: C#CC(=O)N1[C@@H](CCCC)Cc2cc(OC)ccc2[C@@H]1c1ccc(NC23CC(N)(C2)C3)cc1] |
| 64 | 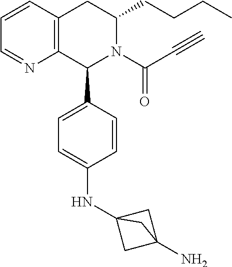 [SMILES: C#CC(=O)N1[C@@H](CCCC)Cc2cccnc2[C@@H]1c1ccc(NC23CC(N)(C2)C3)cc1] |
| 65 | 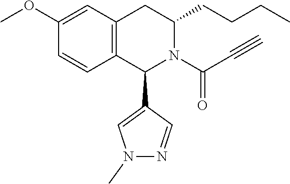 [SMILES: C#CC(=O)N1[C@@H](CCCC)Cc2cc(OC)ccc2[C@@H]1c1cnn(C)c1] |
| 66 | 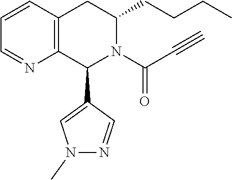 [SMILES: C#CC(=O)N1[C@@H](CCCC)Cc2cccnc2[C@@H]1c1cnn(C)c1] |
| 67 | 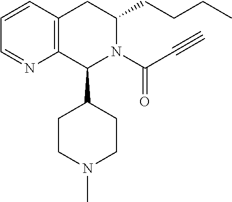 [SMILES: C#CC(=O)N1[C@@H](CCCC)Cc2cccnc2[C@@H]1C1CCN(C)CC1] |
| 68 | 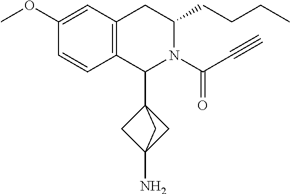 [SMILES: C#CC(=O)N1C(C23CC(N)(C2)C3)c2ccc(OC)cc2C[C@@H]1CCCC] |
| 69 | 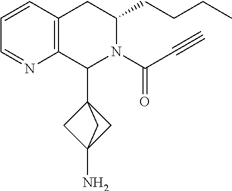 [SMILES: C#CC(=O)N1C(C23CC(N)(C2)C3)c2ncccc2C[C@@H]1CCCC] |
| 70 | 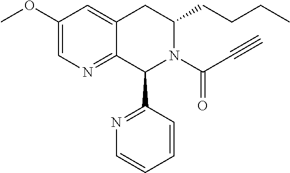 [SMILES: C#CC(=O)N1[C@@H](CCCC)Cc2cc(OC)cnc2[C@@H]1c1ccccn1] |
| 71 | 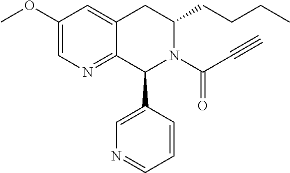 [SMILES: C#CC(=O)N1[C@@H](CCCC)Cc2cc(OC)cnc2[C@@H]1c1cccnc1] |
| 72 | 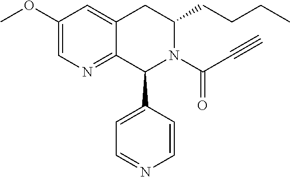 [SMILES: C#CC(=O)N1[C@@H](CCCC)Cc2cc(OC)cnc2[C@@H]1c1ccncc1] |
| 73 | 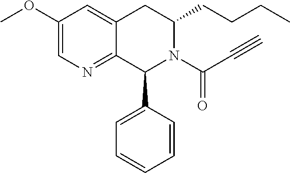 [SMILES: C#CC(=O)N1[C@@H](CCCC)Cc2cc(OC)cnc2[C@@H]1c1ccccc1] |
| 74 | 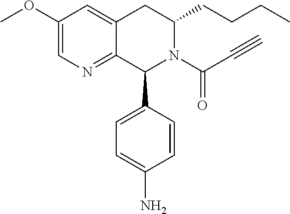 [SMILES: C#CC(=O)N1[C@@H](CCCC)Cc2cc(OC)cnc2[C@@H]1c1ccc(N)cc1] |
| 75 | 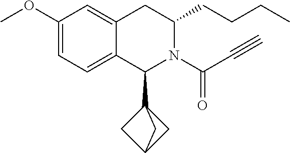 [SMILES: C#CC(=O)N1[C@@H](CCCC)Cc2cc(OC)ccc2[C@@H]1C12CC(C1)C2] |
| 76A | 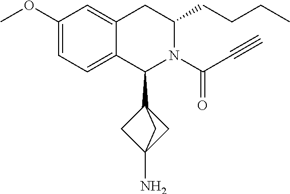 [SMILES: C#CC(=O)N1[C@@H](CCCC)Cc2cc(OC)ccc2[C@@H]1C12CC(N)(C1)C2] |
| 76 | 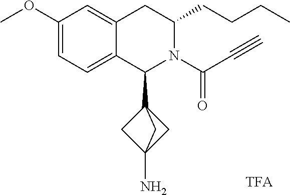 [SMILES: C#CC(=O)N1[C@@H](CCCC)Cc2cc(OC)ccc2[C@@H]1C12CC(N)(C1)C2] |
| 77 | 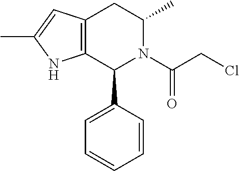 [SMILES: Cc1cc2c([nH]1)[C@H](c1ccccc1)N(C(=O)CCl)[C@@H](C)C2] |
| 78 | 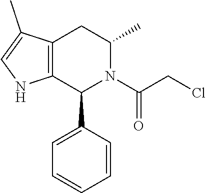 [SMILES: Cc1c[nH]c2c1C[C@H](C)N(C(=O)CCl)[C@H]2c1ccccc1] |
| 79 | 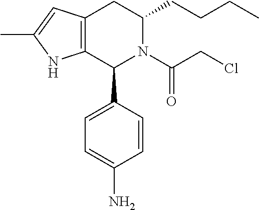 [SMILES: CCCC[C@H]1Cc2cc(C)[nH]c2[C@H](c2ccc(N)cc2)N1C(=O)CCl] |
| 80 | 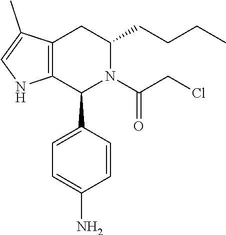 [SMILES: CCCC[C@H]1Cc2c(C)c[nH]c2[C@H](c2ccc(N)cc2)N1C(=O)CCl] |
| 81 | 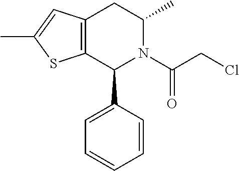 [SMILES: Cc1cc2c(s1)[C@H](c1ccccc1)N(C(=O)CCl)[C@@H](C)C2] |
| 82 | 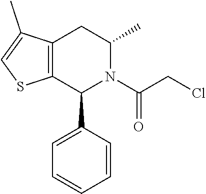 [SMILES: Cc1csc2c1C[C@H](C)N(C(=O)CCl)[C@H]2c1ccccc1] |
| 83 | 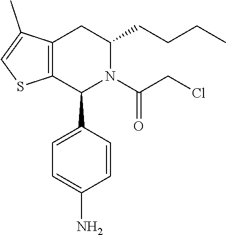 [SMILES: CCCC[C@H]1Cc2c(C)csc2[C@H](c2ccc(N)cc2)N1C(=O)CCl] |
| 84 | 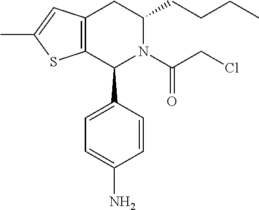 [SMILES: CCCC[C@H]1Cc2cc(C)sc2[C@H](c2ccc(N)cc2)N1C(=O)CCl] |
| 85 | 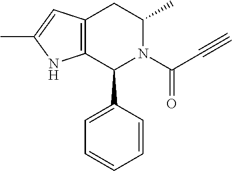 [SMILES: C#CC(=O)N1[C@@H](c2ccccc2)c2[nH]c(C)cc2C[C@@H]1C] |
| 86 | 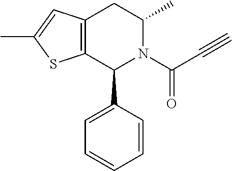 [SMILES: C#CC(=O)N1[C@@H](c2ccccc2)c2sc(C)cc2C[C@@H]1C] |
| 87 | 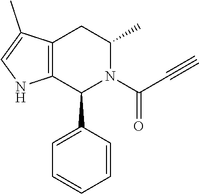 [SMILES: C#CC(=O)N1[C@@H](c2ccccc2)c2[nH]cc(C)c2C[C@@H]1C] |
| 88 | 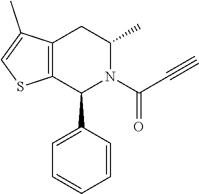 [SMILES: C#CC(=O)N1[C@@H](c2ccccc2)c2scc(C)c2C[C@@H]1C] |
| 89 | 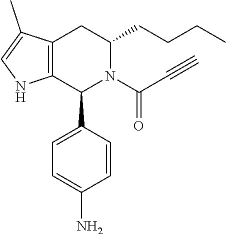 [SMILES: C#CC(=O)N1[C@@H](CCCC)Cc2c(C)c[nH]c2[C@@H]1c1ccc(N)cc1] |
| 90 | 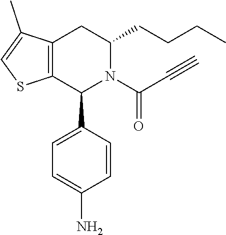 [SMILES: C#CC(=O)N1[C@@H](CCCC)Cc2c(C)csc2[C@@H]1c1ccc(N)cc1] |
| 91 | 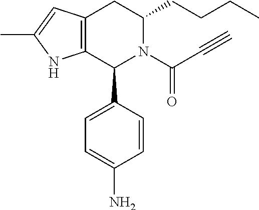 [SMILES: C#CC(=O)N1[C@@H](CCCC)Cc2cc(C)[nH]c2[C@@H]1c1ccc(N)cc1] |
| 92 | 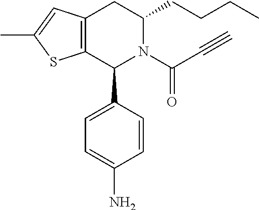 [SMILES: C#CC(=O)N1[C@@H](CCCC)Cc2cc(C)sc2[C@@H]1c1ccc(N)cc1] |
| 93 | 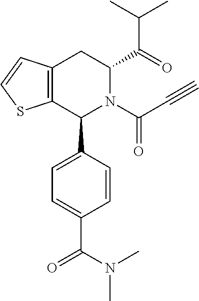 [SMILES: C#CC(=O)N1[C@@H](c2ccc(C(=O)N(C)C)cc2)c2sccc2C[C@@H]1C(=O)C(C)C] |
| 94 | 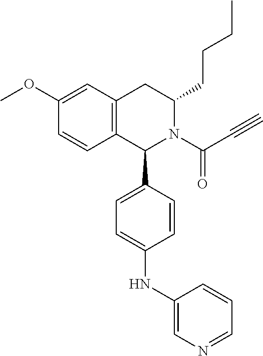 [SMILES: C#CC(=O)N1[C@@H](CCCC)Cc2cc(OC)ccc2[C@@H]1c1ccc(Nc2cccnc2)cc1] |
| 95 | 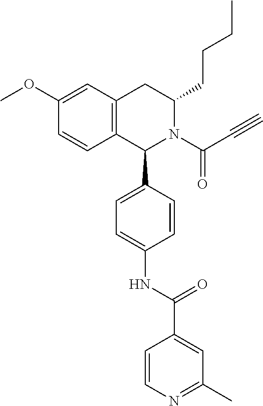 [SMILES: C#CC(=O)N1[C@@H](CCCC)Cc2cc(OC)ccc2[C@@H]1c1ccc(NC(=O)c2ccnc(C)c2)cc1] |
| 96 | 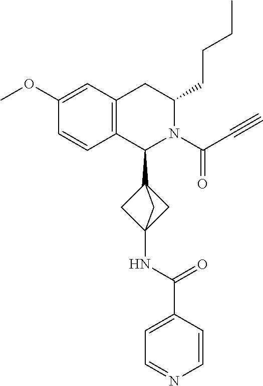 [SMILES: C#CC(=O)N1[C@@H](CCCC)Cc2cc(OC)ccc2[C@@H]1C12CC(NC(=O)c3ccncc3)(C1)C2] |
| 97 | 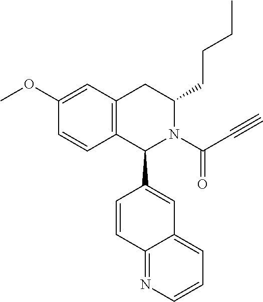 [SMILES: C#CC(=O)N1[C@@H](CCCC)Cc2cc(OC)ccc2[C@@H]1c1ccc2ncccc2c1] |
| 98 | 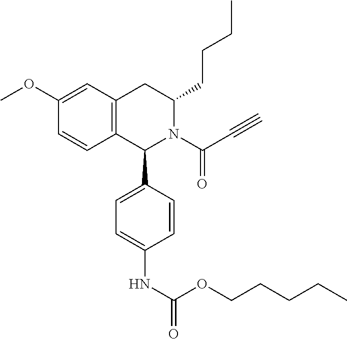 [SMILES: C#CC(=O)N1[C@@H](CCCC)Cc2cc(OC)ccc2[C@@H]1c1ccc(NC(=O)OCCCCC)cc1] |
| 99A | 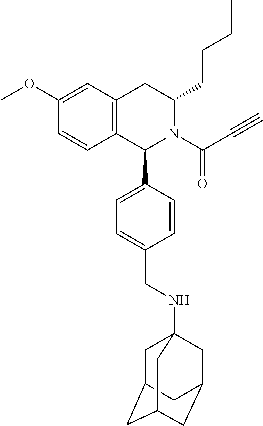 [SMILES: C#CC(=O)N1[C@@H](CCCC)Cc2cc(OC)ccc2[C@@H]1c1ccc(CNC23CC4CC(CC(C4)C2)C3)cc1] |
| 99 | 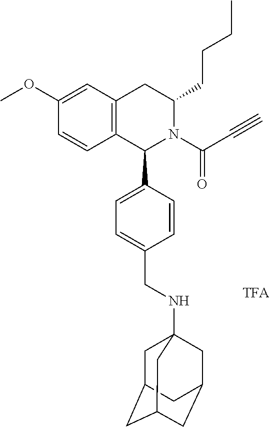 [SMILES: C#CC(=O)N1[C@@H](CCCC)Cc2cc(OC)ccc2[C@@H]1c1ccc(CNC23CC4CC(CC(C4)C2)C3)cc1 ; "TFA"] |
| 100A | 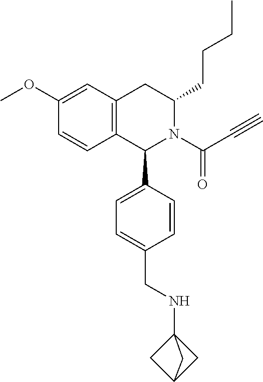 [SMILES: C#CC(=O)N1[C@@H](CCCC)Cc2cc(OC)ccc2[C@@H]1c1ccc(CNC23CC(C2)C3)cc1] |
| 100 | 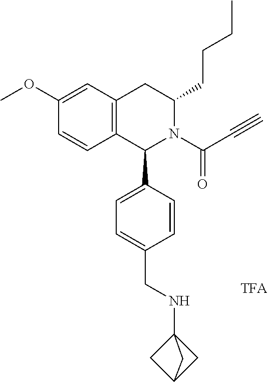 [SMILES: C#CC(=O)N1[C@@H](CCCC)Cc2cc(OC)ccc2[C@@H]1c1ccc(CNC23CC(C2)C3)cc1] |
| 101 | 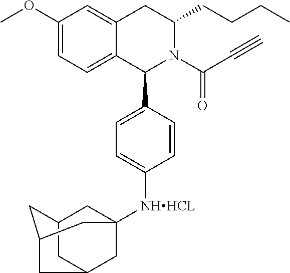 |
| 102 | 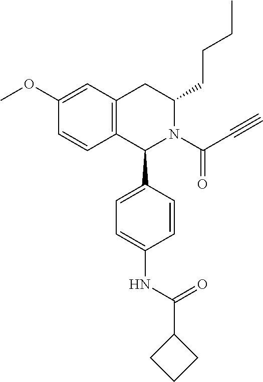 [SMILES: C#CC(=O)N1[C@@H](CCCC)Cc2cc(OC)ccc2[C@@H]1c1ccc(NC(=O)C2CCC2)cc1] |

In some embodiments, the GPX4 inhibitor is

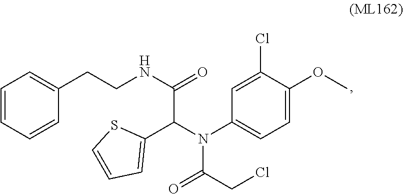 [SMILES: COc1ccc(N(C(=O)CCl)C(C(=O)NCCc2ccccc2)c2cccs2)cc1Cl]

or a pharmaceutically acceptable salt thereof.

ML162 has been identified as a direct inhibitor of GPX4 that induces ferroptosis (see, Dixon et al., 2015, ACS Chem. Bio. 10, 1604-1609).

In some embodiments, the GPX4 inhibitor is a pharmaceutically acceptable form of ML162, including, but not limited to, N-oxides, crystalline form, hydrates, salts, esters, and prodrugs thereof.

In some embodiments, the inhibitor of GPX4 is ML162 or a derivative or analog thereof.

In some embodiments, the GPX4 inhibitor is

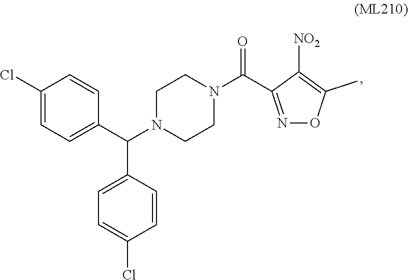 [SMILES: Cc1onc(C(=O)N2CCN(C(c3ccc(Cl)cc3)c3ccc(Cl)cc3)CC2)c1[N+](=O)[O-]]

or a pharmaceutically acceptable salt thereof.

In some embodiments, the GPX4 inhibitor is a pharmaceutically acceptable form of ML210, including, but not limited to, N-oxides, crystalline form, hydrates, salts, esters, and prodrugs thereof.

In some embodiments, the inhibitor of GPX4 is ML210 or a derivative or analog thereof.

In some embodiments, the inhibitor of GPX4 is FIN56 or a derivative or analog thereof.

In one embodiment, FIN56 or a derivative or analog thereof is represented by formula:

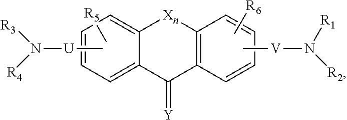

or a pharmaceutically acceptable salt, ester, amide, stereoisomer, geometric isomer, solvate or prodrug thereof, wherein

n=0-2, and wherein when n=1, X is selected from CH₂, O, NR_{A}, CO, and C═NOR_{A} and wherein when n=2, X=CH₂,

Y is O, S, NOR_{A}, or NR_{A},
wherein R_{A} is selected from H, alkyl, heteroalkyl, alkenyl, alkynyl, cycloalkyl, —C(═O)R_{B}, —C(═O)OR_{B}, —C(═O)NR_{B}R_{C}, —C(═NR_{B})R_{C}, —NR_{B}R_{C}, heterocycloalkyl, aryl or polyaromatic, heteroaryl, arylalkyl and alkylaryl,
wherein each of said R_{B} and R_{C} is independently H, alkyl, or heteroalkyl, U and V are each independently selected from C═O, and O═S═O and wherein when U is C═O, V is not C═O,

R₁, R₂, R₃, and R₄ are each independently selected from H, alkyl, heteroalkyl, cycloalkyl, arylcycloalkyl, alkenyl, alkynyl, aryl, heteroaryl, heterocycloalkyl, and each of said NR₁R₂ and NR₃R₄ can independently combine to form a heterocycloalkyl,

R₅ and R₆ are each independently selected from H, OH, SH, alkoxy, thioalkoxy, alkyl, halogen, CN, CF₃, NO₂, COOR_{D}, CONR_{D}R_{E}, NR_{D}R_{E}, NR_{D}COR_{E}, NR_{D}SO₂R_{E}, and NR_{F}CONR_{D}R_{E};
wherein RD, RE and RF are independently H, alkyl, heteroalkyl, aryl, arylalkyl, heteroaryl, heteroarylalkyl, cycloalkyl, or heterocycloalkyl;

provided that if X is O, Y is O and U and V are both O═S═O, then NR₁R₂ and NR₃R⁴ are not identical then R₁ and R₃ are each independently selected from H and lower alkyl, and wherein R₂ and R₄ are each independently selected from lower alkoxy(loweralkyl), di(lower)alkylamino(lower)alkyl, halobenzyl, morpholino(lower)alkyl, or NR₁R₂ and NR₃R⁴ are independently piperidino, morpholino, piperazino, N-phenylpiperazino, ethylamino, or substituted glycine,

and wherein if X is (CH₂)₂, Y is 0 or NOH, and U and V are each O═S═O then none of R₁, R₂, R₃, and R₄ is methyl,

and wherein if n=O, Y is O or NOH, and U and V are each O═S═O, then NR₁R₂ and NR₃R₄ are not identical and R₁, R₂, R₃, and R₄ are each independently selected from C₁-C₅ alkyl, C₁O alkyl, Ciβ alkyl, C₁₇ alkyl, phenyl, benzyl, naphthalenyl, piperizino, pyridinyl, pyrazolyl, benzimidazolyl, triazolyl; or NR₁R₂ and NR₃R₄ are independently piperidino, morpholino, or piperazino,

and wherein if X is CO, Y is O, and U and V are each O═S═O then NR₁R₂ and NR₃R⁴ are not identical, and wherein R₁, R₂, R₃, and R₄ are each independently selected from methyl, ethyl, hydroxy-d-Cralkyl, SH, RO, COOH, SO, NH₂, and phenyl or wherein one or both of non-identical NR₁R₂ and NR₃R₄ is unsubstituted piperidino, N-methylpiperazino or N-methylhomopiperazino,

and wherein when X is C═O or C═NOH, Y is O or NOH, and U and V are each O═S═O and one of R₁ or R₂ and one of R₃ or R₄ is phenyl then the other of R₁ or R₂ and R₃ or R₄ is not H or alkyl.

In one embodiment, the FIN56 derivative or analog thereof is represented by the following formula:

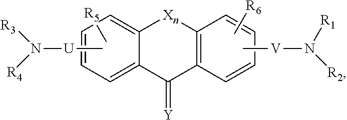

wherein n=1-2 and wherein when n=1, X is selected from CH₂, 0, CO, and C═NOR_{A};

and wherein when n=2, X=CH₂,

Y is O, S, NOR_{A}, or NR_{A},

wherein U and V are each O═S═O,

wherein R_{A} is selected from H, alkyl, heteroalkyl, alkenyl, alkynyl, cycloalkyl, —C(═O)R_{B}, —C(═O)OR_{B}, —C(═O)NR_{B}R_{C}, —C(═NR_{B})R_{C}, —NR_{B}R_{C}, heterocycloalkyl, aryl or polyaromatic, heteroaryl, arylalkyl and alkylaryl,

wherein each of said R_{B} and R_{C} is independently H, alkyl, or heteroalkyl,

R₁, R₂, R₃, and R₄ are each independently selected from H, alkyl, heteroalkyl, cycloalkyl, arylcycloalkyl, alkenyl, alkynyl, aryl, heteroaryl, heterocycloalkyl, and each of said NR₁R₂ and NR₃R₄ can independently combine to form a heterocycloalkyl,

R₅ and R₆ are each independently selected from H, OH, SH, alkoxy, thioalkoxy, alkyl, halogen, CN, CF₃, NO₂, COOR_{D}, CONR_{D}R_{E}, NR_{D}R_{E}, NR_{D}COR_{E}, NR_{D}SO₂R_{E}, and NR_{F}CONR_{D}R_{E};
wherein R_{D}, R_{E} and R_{F} are independently H, alkyl, heteroalkyl, aryl, arylalkyl, heteroaryl, heteroarylalkyl, cycloalkyl, or heterocycloalkyl; provided that is X is O, Y is O and U and V are both O═S═O, then NR₁R₂ and NR₃R₄ are not identical then R₁ and R₃ are each independently selected from H and lower alkyl, and wherein R₂ and R₄ are each independently selected from lower alkoxy(loweralkyl), di(lower)alkylamino(lower)alkyl, halobenzyl, morpholino(lower)alkyl, or NR₁R₂ and NR₃R₄ are independently piperidino, morpholino, piperazino, N-phenylpiperazino, ethylamino, or substituted glycine,

and wherein if X is (CH₂)₂, and Y is O or NOH, then none of R₁, R₂, R₃, and R₄ is methyl,

and wherein if X is CO and Y is O, then NR₁R₂ and NR₃R⁴ are not identical, and wherein R₁, R₂, R₃, and R₄ are each independently selected from methyl, ethyl, hydroxy-C₁-C₃-alkyl, SH, RO, COOH, SO, NH₂, and phenyl or wherein one or both of non-identical NR₁R₂ and NR₃R⁴ is unsubstituted piperidino, N-methylpiperazino or N-methylhomopiperazino, wherein said unsubstituted piperidine, N-methylpiperazino or N-methylhomopiperazino NR₁R₂ and NR₃R⁴ moieties are not identical,

and wherein when X is C═O or C═NOH, Y is O or NOH, and one of R₁ or R₂ and one of R₃ or R₄ is phenyl then the other of R¹ or R₂ and R₃ or R₄ is not H or alkyl,

or a pharmaceutically acceptable salt, ester, amide, stereoisomer or geometric isomer thereof.

In one embodiment, the FIN56 derivative or analog thereof is represented by the following formula:

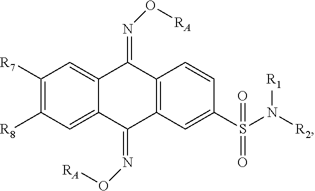

wherein R_{A} is hydrogen, R₇ and R₈ are independently selected from H and SO₂NR₃R₄, wherein one of R₇ and R₈ is hydrogen and wherein NR₁R₂ and NR₃R₄ are independently 6- to 15-membered heterocycloalkyl containing one nitrogen in the ring, or a pharmaceutically acceptable salt, ester, amide, stereoisomer or geometric isomer thereof.

Additional FIN56 derivatives or analogs are described, for example in WO 2008/140792, WO 2010/082912, WO 2017/058716, U.S. Pat. No. 6,693,136, Nature Chemical Biology (2016), 12(7), 497-503 doi: 10.1038/nchembio.2079, ACS Chemical Biology (2015), 10(7), 1604-1609 doi: 10.1021/acschembio.5b00245, Dissertation Abstracts International, (2015) Vol. 76, No. 8B(E). Order No.: AAI3688566. ProQuest Dissertations & Theses. 120 pages, each of which is incorporated by reference herein in its entirety.

In some embodiments, an agent that induces iron-dependent cellular disassembly (e.g., ferroptosis) and is useful in the methods provided herein is a statin. In one embodiment, the statin is selected from the group consisting of atorvastatin, fluvastatin, lovastatin, pitavastatin, pravastatin, rosuvastatin, cerivastatin and simvastatin.

In one embodiment, the agent that induces iron-dependent cellular disassembly (e.g., ferroptosis) and is useful in the methods provided herein is selected from the group consisting of glutamate, BSO, DPI2 (See Yang et al., 2014, Cell 156: 317-331; FIGS. 5 and S5, incorporated in its entirety herein), cisplatin, cysteinase, silica based nanoparticles, CCI4, ferric ammonium citrate, trigonelline and brusatol.

Additional agents that induce iron-dependent cellular disassembly are known in the art and are described, for example in US2020/138829; U.S. Pat. Nos. 8,518,959; 8,535,897; 8,546,421; 9,580,398; 9,695,133; US2010/0081654; US2015/0079035; US2015/0175558; US2016/0229836; US2016/0297748; US2016/0332974; Cell. 2012 May 25; 149(5):1060-72. doi: 10.1016/j.cell.2012.03.042;
Cell. 2014 Jan. 16; 156(1-2):317-331. doi: 10.1016/j.cell.2013.12.010;
J Am Chem Soc. 2014 Mar. 26; 136(12):4551-6. doi: 10.1021/ja411006a;
Elife. 2014 May 20; 3:e02523. doi: 10.7554/eLife.02523;
Proc Natl Acad Sci USA. 2014 Nov. 25; 111(47):16836-41. doi: 10.1073/pnas.1415518111;
Nat Cell Biol. 2014 December; 16(12):1180-91. doi: 10.1038/ncb3064;
ACS Chem Biol. 2015 Jul. 17; 10(7):1604-9. doi: 10.1021/acschembio.5b00245;
Bioorg Med Chem Lett. 2015 Nov. 1; 25(21):4787-92. doi: 10.1016/j.bmcl.2015.07.018;
Nat Rev Drug Discov. 2016 May; 15(5):348-66. doi: 10.1038/nrd.2015.6;
Cell Chem Biol. 2016 Feb. 18; 23(2):225-235. doi:10.1016/j.chembiol.2015.11.016;
Nat Chem Biol. 2016 July; 12(7):497-503. doi: 10.1038/nchembio.2079;
Proc Natl Acad Sci USA. 2016 Aug. 23; 113(34):E4966-75. doi: 10.1073/pnas.1603244113;
ACS Cent Sci. 2016 Sep. 28; 2(9):653-659;
Nat Chem Biol. 2017 January; 13(1):81-90. doi: 10.1038/nchembio.2238;
Biochem Biophys Res Commun. 2017 Jan. 15; 482(3):419-425. doi: 10.1016/j.bbrc.2016.10.086; Nat Chem Biol. 2018 May; 14(5):507-515. doi: 10.1038/s41589-018-0031-6;
Cancer Lett. 2018 Apr. 24. pii: S0304-3835(18)30288-X. doi: 10.1016/j.canlet.2018.04.021;
J Med Chem. 2018 Apr. 24. doi: 10.1021/acs.jmedchem.8b00315;
Eur J Med Chem. 2018 May 10; 151:434-449. doi: 10.1016/j.ejmech.2018.04.005;
Neuropharmacology. 2018 Mar. 15; 135:242-252. doi: 10.1016/j.neuropharm.2018.03.015;
J Am Chem Soc. 2018 Mar. 14; 140(10):3798-3808. doi: 10.1021/jacs.8b00998;
Biochem Biophys Res Commun. 2018 Feb. 26; 497(1):233-240. doi: 10.1016/j.bbrc.2018.02.061;
Org Biomol Chem. 2018 Feb. 28; 16(9):1465-1479. doi: 10.1039/c7ob03086j;
Arch Toxicol. 2018 April; 92(4):1507-1524. doi: 10.1007/s00204-018-2170-7;
Int J Oncol. 2018 March; 52(3):1011-1022. doi: 10.3892/ijo.2018.4259;
Cancer Lett. 2018 Apr. 28; 420:210-227. doi: 10.1016/j.canlet.2018.01.061.Feb 1;
Free Radic Biol Med. 2018 March; 117:45-57. doi: 10.1016/j.freeradbiomed.2018.01.019;
Sci Rep. 2018 Jan. 12; 8(1):574. doi: 10.1038/s41598-017-18935-1;
Cancer Lett. 2018 Mar. 1; 416:124-137. doi: 10.1016/j.canlet.2017.12.025;
Chem Med Chem. 2018 Jan. 22; 13(2):164-177. doi: 10.1002/cmdc.201700629;
Redox Biol. 2018 April; 14:535-548. doi: 10.1016/j.redox.2017.11.001;
Arch Toxicol. 2018 February; 92(2):759-775. doi: 10.1007/s00204-017-2066-y;
Nat Chem. 2017 October; 9(10):1025-1033. doi: 10.1038/nchem.2778;
Free Radic Biol Med. 2017 November; 112:597-607. doi: 10.1016/j.freeradbiomed.2017.09.002;
ACS Chem Biol. 2017 Oct. 20; 12(10):2538-2545. doi: 10.1021/acschembio.7b00730. 20;
PLoS One. 2017 Aug. 21; 12(8):e0182921. doi: 10.1371/journal.pone.0182921. eCollection 2017; Biochem Biophys Res Commun. 2017 Sep. 30; 491(4):919-925. doi: 10.1016/j.bbrc.2017.07.136;
Biochem Pharmacol. 2017 Sep. 15; 140:41-52. doi: 10.1016/j.bcp.2017.06.112. 23;
Cancer Res Treat. 2018 April; 50(2):445-460. doi: 10.4143/crt.2016.572;
Cell Death Discov. 2017 Feb. 27; 3:17013. doi: 10.1038/cddiscovery.2017.13. eCollection 2017; Nat Nanotechnol. 2016 November; 11(11):977-985. doi: 10.1038/nnano.2016.164;
Biochem Biophys Res Commun. 2016 Nov. 25; 480(4):602-607. doi: 10.1016/j.bbrc.2016.10.099; Oncotarget. 2016 Nov. 15; 7(46):74630-74647. doi: 10.18632/oncotarget.11858;
Cancer Lett. 2016 Oct. 10; 381(1):165-75. doi: 10.1016/j.canlet.2016.07.033. 29;
Cell Death Dis. 2016 Jul. 21; 7:e2307. doi:10.1038/cddis.2016.208;
Oncol Rep. 2016 August; 36(2):968-76. doi: 10.3892/or.2016.4867. 31;
Biochem Biophys Res Commun. 2016 May 13; 473(4):775-780. doi: 10.1016/j.bbrc.2016.03.052; Mol Carcinog. 2017 January; 56(1):75-93. doi: 10.1002/mc.22474;
ACS Chem Biol. 2016 May 20; 11(5):1305-12. doi: 10.1021/acschembio.5b00900;
J Med Chem. 2016 Mar. 10; 59(5):2041-53. doi: 10.1021/acs.jmedchem.5b01641;
Phytomedicine. 2015 Oct. 15; 22(11):1045-54. doi: 10.1016/j.phymed.2015.08.002;
Biol Pharm Bull. 2015; 38(8):1234-9. doi: 10.1248/bpb.b15-00048;
Oncoscience. 2015 May 2; 2(5):517-32. eCollection 2015;
Pathol Oncol Res. 2015 September; 21(4):1115-21. doi: 10.1007/s12253-015-9946-3;
Anticancer Res. 2014 November; 34(11):6417-22; and
Int J Cancer. 2013 Oct. 1; 133(7):1732-42. doi: 10.1002/ijc.28159;
   each of which is incorporated by reference herein in its entirety.

In one embodiment, an agent that induces iron-dependent cellular disassembly (e.g., ferroptosis) and is useful in the compositions and methods provided herein induces one or more desirable immune effects in a co-cultured cell, such as an immune cell. For example, in embodiments, the agent that induces iron-dependent cellular disassembly has one or more of the following characteristics:
(a) induces iron-dependent cellular disassembly of a target cell in vitro and activation of an immune response in a co-cultured cell;
(b) induces iron-dependent cellular disassembly of a target cell in vitro and activation of co-cultured macrophages, e.g., RAW264.7 macrophages;
(c) induces iron-dependent cellular disassembly of a target cell in vitro and activation of co-cultured monocytes, e.g., THP-1 monocytes;
(d) induces iron-dependent cellular disassembly of a target cell in vitro and activation of co-cultured bone marrow-derived dendritic cells (BMDCs);
(e) induces iron-dependent cellular disassembly of a target cell in vitro and increases levels or activity of NFkB, IRF and/or STING in a co-cultured cell;
(f) induces iron-dependent cellular disassembly of a target cell in vitro and increases levels or activity of a pro-immune cytokine in a co-cultured cell;
(g) induces iron-dependent cellular disassembly of a target cell in vitro and activation of co-cultured CD4+ cells, CD8+ cells and/or CD3+ cells; and
(h) induces iron-dependent cellular disassembly of a target cell in vitro and increases levels or activity of T cells.
   Numerous methods for determining an agent which, in addition to inducing iron-dependent cellular disassembly of a target cell, induces said immune effects in a co-cultured cell are known in the art and are provided and described in detail herein.

In certain aspects of the invention, it can be desirable to target or direct the delivery of the agent that induces iron-dependent cellular disassembly to a particular target cell, such as a cancer cell. Accordingly, in some embodiments, the agent that induces iron-dependent cellular disassembly is targeted to a cancer cell. Methods of targeting therapeutic agents to cancer cells are known in the art and are described, for example, in US2017/0151345, which is incorporated by reference herein in its entirety. For example, the agent that induces iron-dependent cellular disassembly may be targeted to a cancer cell by combining it, for example in a complex or as a conjugate, with a molecule that specifically binds to a cancer cell marker. As used herein, the term “cancer cell marker” refers to a polypeptide that is present on the surface of a cancer cell. For example, a cancer cell marker may be a cancer cell receptor, e.g., a polypeptide that binds specifically to a molecule in the extracellular environment. A cancer cell marker (e.g., receptor) can be a polypeptide displayed exclusively on cancer cells, a polypeptide displayed at a higher level on cancer cells than normal cells of the same or different tissue types, or a polypeptide displayed on both cancerous and normal cell types. In some embodiments, a cancer cell marker (e.g., receptor) can be a polypeptide that, in cancer cells, has altered (e.g. higher or lower than normal) expression and/or activity. In some embodiments, a cancer cell marker (e.g., receptor) can be a polypeptide that is implicated in the disease process of cancer. In some embodiments, a cancer cell marker (e.g., receptor) can be a polypeptide that is involved in the control of cell death and/or apoptosis. Non-limiting examples of cancer cell markers include, but are not limited to, EGFR, ER, PR, HER2, PDGFR, VEGFR, MET, c-MET, ALK, CD117, RET, DR4, DR5, and FasR. In some embodiments, the molecule that specifically binds to the cancer cell marker (e.g., receptor) is an antibody or cancer cell marker-binding fragment thereof. In some embodiments, the cancer cell marker is a receptor and the molecule that specifically binds to the cancer cell receptor is a ligand or a ligand mimetic of the receptor.

Accordingly, in some embodiments, it is envisaged that a composition of the invention comprises a complex or conjugate comprising the agent that induces iron-dependent cellular disassembly and a molecule that specifically binds to a cancer cell marker (e.g., receptor). In certain embodiments, the complex or conjugate comprises a pharmaceutically acceptable dendrimer, for example, a PAMAM dendrimer. In certain embodiments, the complex comprises a liposome. In certain embodiments, the complex comprises a microparticle or a nanoparticle.

### III. Anti-Neoplastic Agents

In the methods described herein, the agent that induces iron-dependent cellular disassembly is administered in combination with an anti-neoplastic agent.

In certain embodiments, the anti-neoplastic agent is a chemotherapeutic agent, (e.g., alkylating agents, antimetabolites, anti-tumor antibiotics, topoisomerase inhibitors, mitotic inhibitors and corticosteroids). Chemotherapeutic agents include, but are not limited to, alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, mitotic inhibitors, and gonadotropin-releasing hormone analog. Also included are 5-fluorouracil (5-FU), leucovorin (LV), irenotecan, oxaliplatin, capecitabine, paclitaxel and doxetaxel.

Non-limiting examples of chemotherapeutic agents include alkylating agents such as Altretamine, Busulfan, Carboplatin, Carmustine, Chlorambucil, Cisplatin, Cyclophosphamide, Dacarbazine, Lomustine, Melphalan, Oxaliplatin, Temozolomide, and Thiotepa; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; anti-tumor antibiotics such as anthracyclines (e.g., Daunorubicin, Doxorubicin (Adriamycin®), Epirubicin, Idarubicin), Actinomycin-D, Bleomycin, Mitomycin-C, Mitoxantrone (also acts as a topoisomerase II inhibitor), enediyne antibiotics (e.g., calicheamicin, especially calicheamicin gammall and calicheamicin omegall (see, e.g., Agnew, Chem. Intl. Ed Engl. 33:183 1994); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, 5-fluorouracil (5-FU), 6-mercaptopurine (6-MP); Capecitabine (Xeloda®), Cytarabine (Ara-C®), Floxuridine, Fludarabine, Gemcitabine (Gemzar®), Hydroxyurea, Methotrexate, and Pemetrexed (Alimta®); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel; chloranbucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitors, such as Topotecan, Irinotecan (CPT-11), Etoposide (VP-16), Teniposide, Mitoxantrone (also acts as an anti-tumor antibiotic), and RFS 2000; difluoromethylornithine (DMFO); retinoids such as retinoic acid; capecitabine; mitotic inhibitors such as Docetaxel, Estramustine, Ixabepilone, Paclitaxel, Vinblastine, Vincristine, Vinorelbine; corticosteroids such as Prednisone, Methylprednisolone (Solumedrol®), Dexamethasone (Decadron®), and pharmaceutically acceptable salts, acids or derivatives of any of the above. Two or more cytotoxic agents can be used in a cocktail to be administered in combination. Suitable dosing regimens for combinations of cytotoxic agents are known in the art and described in, for example, Saltz et al., Proc ASCO 18:233a, 1999, and Douillard et al., Lancet 355:1041, 2000.

In some embodiments, the anti-neoplastic agent is a biologic agent (e.g. an antibody, cytokine, or enzyme). In some embodiments, the biologic agent is a cytokine (e.g., interferon or an interleukin (e.g., IL-2)) used in cancer treatment. In other embodiments the biologic agent is an anti-angiogenic agent, such as an anti-VEGF agent, e.g., bevacizumab. In some embodiments, the biologic agent is an enzyme such as L-asparaginase, or bortezomib (Velcade®)).

In some embodiments the biologic agent is an immunoglobulin-based biologic, e.g., a monoclonal antibody (e.g., a humanized antibody, a fully human antibody, an Fc fusion protein or a functional fragment thereof). The immunoglobulin-based biologic may agonize a target to stimulate an anti-cancer response, or antagonize an antigen important for cancer. Such agents include anti-TNF antibodies, e.g., adalimumab or infliximab; anti-CD20 antibodies, such as rituximab, anti-VEGF antibodies, such as bevacizumab; anti-HER2 antibodies, such as trastuzumab; and anti-RSV, such as palivizumab. In some embodiments, the immunoglobulin-based biologic is selected from Daclizumab; Basiliximab; Palivizumab; Infliximab; Trastuzumab; Gemtuzumab ozogamicin; Alemtuzumab; Ibritumomab tiuxetan; Adalimumab; Omalizumab; Tositumomab-I-131; Efalizumab; Cetuximab; Bevacizumab; Natalizumab; Tocilizumab; Panitumumab; Ranibizumab; Eculizumab; Certolizumab pegol; Golimumab; Canakinumab; Ustekinumab; Ofatumumab; Denosumab; Motavizumab; Raxibacumab; Belimumab; Ipilimumab; Brentuximab Vedotin; Pertuzumab; Ado-trastuzumab emtansine; and Obinutuzumab. Also included are antibody-drug conjugates. Examples of biologic agents that can be used in the methods described herein are shown in Table 4 below.

TABLE 4
Approved Immunoglobulin-based anti-cancer agents

| Antibody | Antigen | Indication |
|---|---|---|
| ado-trastuzumab | HER2 | Mestastatic breast cancer |
| emtansine | | |
| alemtuzumab | CD52 | B-cell chronic lymphocytic |
| | | leukemia |
| atezolizumab | PD-L1 | Urothelial carcinoma |
| atezolizumab | PD-L1 | Metastatic non-small cell lung |
| | | cancer |
| avelumab | PD-L1 | Metastatic Merkel cell carcinoma |
| bevacizumab | VEGF | Metastatic colorectal cancer |
| blinatumomab | CD19 | Precursor B-cell acute lymphoblastic |
| | | leukemia |
| brentuximab | CD30 | Hodgkin lymphoma |
| vedotin | | Anaplastic large-cell lymphoma |
| cetuximab | EGFR | Metastatic colorectal carcinoma |
| daratumumab | CD38 | Multiple myeloma |
| dinutuximab | GD2 | Pediatric high-risk neuroblastoma |
| durvalumab | PD-L1 | Urothelial carcinoma |
| elotuzumab | SLAMF7 | Multiple myeloma |
| ibritumomab | CD20 | Relapsed or refractory low-grade, |
| tiuxetan | | follicular, or transformed B-cell |
| | | non-Hodgkin's lymphoma |
| ipilimumab | CTLA-4 | Metastatic squamous non-small cell |
| | | lung carcinoma |
| necitumumab | EGFR | Metastatic squamous non-small cell |
| | | lung carcinoma |
| nivolumab | PD-1 | Metastatic melanoma |
| nivolumab | PD-1 | Metastatic squamous non-small cell |
| | | lung carcinoma |
| obinutuzumab | CD20 | Chronic lymphocytic leukemia |
| ofatumumab | CD20 | Chronic lymphocytic leukemia |
| olaratumab | CD20 | Chronic lymphocytic leukemia |
| panitumumab | EGFR | Metastatic colorectal |
| ramucirumab | VEGFR2 | Gastric cancer |
| rituximab | CD20 | B-cell non-Hodgkin's lymphoma |
| trastuzumab | HER2 | Metastatic breast cancer |

In some embodiments, the anti-neoplastic agent is an apoptosis inducer. As used herein, the term “apoptosis inducer” refers to an agent that induces programmed cell death characterized by chromosomal DNA fragmentation. An apoptosis inducer may also induce one or more of blebbing, cell shrinkage, nuclear fragmentation, chromatin condensation, and mRNA degradation. Agents that induce apoptosis are known in the art and are suitable for use in the methods described herein. Non-limiting examples of apoptosis inducers suitable for use in the methods described herein are provided in Table 5 below.

TABLE 5
Apoptosis inducers

| Drug | Target |
|---|---|
| ONY-015 | p53 |
| INGN201 | p53 |
| PS1145 | IKKbeta |
| Bortezomib | 20S proteosome |
| CCI779 | mTOR |
| RAD-001 | mTOR |
| ABT-199 (Venetoclax) | BCL |
| SAR405838 | p53 |
| AMG-232 | p53 |
| PRIMA | p53 |
| APR-246 | p53 |
| CP31398 | p53 |
| JNJ26854165113 | p53 |
| HDM201 | p53 |
| CGM097 | p53 |
| APG-115 | p53 |
| DS-3032b | p53 |
| Idasanutlin | p53 |
| BI 907828 | p53 |
| ALRN-6924 | p53 |
| Navitoclax (ABT-263) | BCL2 inhibitor |
| S55746 | BCL2 inhibitor |
| APG-1252 | BCL2 inhibitor |
| PNT2258 | BCL2 inhibitor |

In some embodiments, the anti-neoplastic agent is a cancer drug (e.g. a targeted therapy) that is known to induce resistance in cancer cells. Examples of targeted anti-neoplastic agents that are known to induce resistance in cancer cells are provided in Table 6 below.

TABLE 6
Targeted anti-neoplastic agents that are known to induce resistance

| | | Diagnostic for |
|---|---|---|
| Drug | Cancer Approved for Therapy | Resistant Cancer |
| Bosutinib | Ph+ chronic myeloid leukemia | BCR-ABL fusion* |
| | (CML) | |
| Dasatinib | Ph+ CML; Ph+ acute | BCR-ABL fusion* |
| | lymphoblastic leukemia (ALL) | |
| Imatinib | Ph+ CML; Ph+ ALL; KIT+ | BCR-ABL fusion* (CML |
| | gastrointestinal stromal tumor | and ALL), KIT protein |
| | (GIST) | expression (GIST) |
| Nilotinib | Ph+ CML | BCR-ABL fusion* |
| Ponatinib | BCR-ABLT315I-mutated CML, | BCR-ABLT315I muta- |
| | or CML with no other TKI | tion*, or ABL mutation* |
| | indicated | and failure of other |
| | | TKIs |
| Cetuximab | KRAS-wild-type colorectal | KRAS mutation, NRAS |
| (epidermal | cancer (CRC) | mutation* |
| growth | | |
| factor | | |
| receptor | | |
| (EGFR) | | |
| inhibitor) | | |
| Panitumu- | KRAS-wild-type CRC | KRAS mutation, NRAS |
| mab | | mutation* |
| Afatinib | EGFR del19 or EGFRL858R | EGFR mutation |
| | non-small cell lung cancer | |
| | (NSCLC) | |
| Erlotinib | EGFR del19 or EGFRL858R | EGFR mutation |
| | NSCLC | |
| Gefitinib | EGFR del19 or EGFRL858R | EGFR mutation |
| | NSCLC | |
| Dabrafenib | BRAFV600E melanoma | BRAF V600 mutation |
| (inhibitor | | |
| of BRAF) | | |
| Vemurafenib | BRAF V600 mutant melanoma | BRAF V600 mutation |
| Ceritinib | ALK+ NSCLC | ALK fusion |
| Crizotinib | ALK+ NSCLC | ALK fusion |
| Trametinib | BRAFV600E/K melanoma | BRAF V600 mutation |
| Olaparib | Ovarian cancer with | BRCA mutation |
| | deleterious germline | |
| | BRCA mutation | |
| Ado- | HER2+ breast cancer | HER2 overexpression |
| trastuzumab | | |
| emtansine | | |
| Lapatinib | HER2+ breast cancer | HER2 overexpression |
| Pertuzumab | HER2+ breast cancer | HER2 overexpression |
| Trastuzumab | HER2+ breast cancer; HER2+ | HER2 |
| | gastric cancer | overexpression*Not |

In some embodiments, the anti-neoplastic agent is a drug. In other embodiments, the anti-neoplastic agent is a therapeutic agent that is a non-drug treatment. For example, in some embodiments the anti-neoplastic agent is radiation therapy, cryotherapy or hyperthermia. In a particular embodiment, the anti-neoplastic agent is radiation therapy. In some embodiments, the anti-neoplastic agent is not a non-drug treatment. For example, in some embodiments the anti-neoplastic agent is not radiation therapy, cryotherapy or hyperthermia. In a particular embodiment, the anti-neoplastic agent is not radiation therapy.

In certain aspects of the invention, it can be desirable to target or direct the delivery of the anti-neoplastic agent to a particular target cell, such as a cancer cell. Accordingly, in some embodiments, the anti-neoplastic agent is targeted to a cancer cell. Methods of targeting therapeutic agents to cancer cells are known in the art and are described, for example, in US2017/0151345, which is incorporated by reference herein in its entirety. For example, the anti-neoplastic agent may be targeted to a cancer cell by combining it, for example in a complex or as a conjugate, with a molecule (e.g. an antibody) that specifically binds to a cancer cell marker. As used herein, the term “cancer cell marker” refers to a polypeptide that is present on the surface of a cancer cell. For example, a cancer cell marker may be a cancer cell receptor, e.g., a polypeptide that binds specifically to a molecule in the extracellular environment. A cancer cell marker (e.g., receptor) can be a polypeptide displayed exclusively on cancer cells, a polypeptide displayed at a higher level on cancer cells than normal cells of the same or different tissue types, or a polypeptide displayed on both cancerous and normal cell types. In some embodiments, a cancer cell marker (e.g., receptor) can be a polypeptide that, in cancer cells, has altered (e.g. higher or lower than normal) expression and/or activity. In some embodiments, a cancer cell marker (e.g., receptor) can be a polypeptide that is implicated in the disease process of cancer. In some embodiments, a cancer cell marker (e.g., receptor) can be a polypeptide that is involved in the control of cell death and/or apoptosis. Non-limiting examples of cancer cell markers include, but are not limited to, EGFR, ER, PR, HER2, PDGFR, VEGFR, MET, c-MET, ALK, CD117, RET, DR4, DR5, and FasR. In some embodiments, the molecule that specifically binds to the cancer cell marker (e.g., receptor) is an antibody or cancer cell marker-binding fragment thereof. In some embodiments, the cancer cell marker is a receptor and the molecule that specifically binds to the cancer cell receptor is a ligand or a ligand mimetic of the receptor.

Accordingly, in some embodiments, it is envisaged that a composition of the invention comprises a complex or conjugate comprising the anti-neoplastic agent and a molecule (e.g. an antibody) that specifically binds to a cancer cell marker (e.g. receptor). In certain embodiments, the complex or conjugate comprises a pharmaceutically acceptable dendrimer, for example, a PAMAM dendrimer. In certain embodiments, the complex comprises a liposome. In certain embodiments, the complex comprises a microparticle or a nanoparticle.

In some embodiments, the anti-neoplastic agent is not an alkylating agent. For example, in some embodiments, the anti-neoplastic agent is not one of the following alkylating agents: Altretamine, Busulfan, Carboplatin, Carmustine, Chlorambucil, Cisplatin, Cyclophosphamide, Dacarbazine, Lomustine, Melphalan, Oxaliplatin, Temozolomide, and Thiotepa. In some embodiments, the anti-neoplastic agent is not an antimetabolite. For example, in some embodiments, the anti-neoplastic agent is not one of the following antimetabolites: 5-fluorouracil (5-FU), 6-mercaptopurine (6-MP); Capecitabine (Xeloda®), Cytarabine (Ara-C®), Floxuridine, Fludarabine, Gemcitabine (Gemzar®), Hydroxyurea, Methotrexate, and Pemetrexed (Alimta®). In some embodiments, the anti-neoplastic agent is not an anti-tumor antibiotic. For example, in some embodiments, the anti-neoplastic agent is not one of the following anti-tumor antibiotics: anthracyclines (e.g., Daunorubicin, Doxorubicin (Adriamycin®), Epirubicin, Idarubicin), Actinomycin-D, Bleomycin, Mitomycin-C, and Mitoxantrone. In some embodiments, the anti-neoplastic agent is not a topoisomerase inhibitor. For example, in some embodiments, the anti-neoplastic agent is not one of the following topoisomerase inhibitors: Topotecan, Irinotecan (CPT-11), Etoposide (VP-16), Teniposide, and Mitoxantrone. In some embodiments, the anti-neoplastic agent is not a mitotic inhibitor. For example, in some embodiments, the anti-neoplastic agent is not one of the following mitotic inhibitors: Docetaxel, Estramustine, Ixabepilone, Paclitaxel, Vinblastine, Vincristine, and Vinorelbine. In some embodiments, the anti-neoplastic agent is not a corticosteroid. For example, in some embodiments the anti-neoplastic agent is not one of the following coritcosteroids: Prednisone, Methylprednisolone (Solumedrol®), and Dexamethasone (Decadron®). In some embodiments the anti-neoplastic agent is not an enzyme. For example, in some embodiments, the anti-neoplastic agents is not one of the following enzymes: L-asparaginase, and bortezomib (Velcade®)). In some embodiments, the anti-neoplastic agent is not a biologic agent. For example, in some embodiments, the anti-neoplastic agent is not an anti-TNF antibody (e.g., adalimumab or infliximab); an anti-CD20 antibody (e.g. rituximab), an anti-VEGF antibody (e.g. bevacizumab); an anti-HER2 antibody (e.g. trastuzumab); or an anti-RSV antibody (e.g. palivizumab).

### IV. Methods of Treating Cancer Using Combination Therapies Comprising Agents that Induce Iron-Dependent Cellular Disassembly and Anti-Neoplastic Agents

Methods are provided for the treatment of cancer by administering an agent that induces iron-dependent cellular disassembly in combination with an anti-neoplastic agent. While not wishing to be bound by theory, it is believed that administration of an agent that induces iron-dependent cellular disassembly to a cancer cell may shift a cancer cell undergoing apoptosis to iron-dependent cellular disassembly (e.g. ferroptosis). This shift from apoptosis to iron-dependent cellular disassembly is expected to result in a greater number of cancer cells undergoing iron-dependent cellular disassembly. An increase in cancer cells undergoing iron-dependent cellular disassembly as a result of the methods provided herein is expected to provide numerous advantages over treatment with either single agent alone, such as a greater extent of cell death, reduced compensatory proliferation of cancer cells, and/or induction of a stronger immune response in immune cells (e.g., immune cells co-cultured with the treated cancer cells or immune cells in a subject treated with the combination of agents).

Accordingly, in certain aspects, the disclosure relates to a method of killing cancer cells in a subject, comprising contacting the cancer cells, or cells adjacent to the cancer cells, with a combination of (a) an anti-neoplastic agent and (b) an agent that induces iron-dependent cellular disassembly. In certain aspects, the disclosure relates to a method of killing cancer cells in a subject, comprising contacting the cancer cells, or cells adjacent to the cancer cells, with a combination of (a) an anti-neoplastic agent and (b) an agent that induces iron-dependent cellular disassembly, wherein the method increases the number of cancer cells undergoing iron-dependent cellular disassembly relative to cancer cells treated with the agent that induces iron-dependent cellular disassembly alone.

In embodiments, the agent that induces iron-dependent cellular disassembly is any one or more of the agents that induce iron-dependent cellular disassembly disclosed herein. In embodiments, the anti-neoplastic agent is any one or more of the anti-neoplastic agents disclosed herein.

In some embodiments, the anti-neoplastic agent is not an immunotherapeutic agent. In some embodiments, the anti-neoplastic agent is not an alkylating agent. For example, in some embodiments, the anti-neoplastic agent is not one of the following alkylating agents: Altretamine, Busulfan, Carboplatin, Carmustine, Chlorambucil, Cisplatin, Cyclophosphamide, Dacarbazine, Lomustine, Melphalan, Oxaliplatin, Temozolomide, and Thiotepa. In some embodiments, the anti-neoplastic agent is not an antimetabolite. For example, in some embodiments, the anti-neoplastic agent is not one of the following antimetabolites: 5-fluorouracil (5-FU), 6-mercaptopurine (6-MP); Capecitabine (Xeloda®), Cytarabine (Ara-C®), Floxuridine, Fludarabine, Gemcitabine (Gemzar®), Hydroxyurea, Methotrexate, and Pemetrexed (Alimta®). In some embodiments, the anti-neoplastic agent is not an anti-tumor antibiotic. For example, in some embodiments, the anti-neoplastic agent is not one of the following anti-tumor antibiotics: anthracyclines (e.g., Daunorubicin, Doxorubicin (Adriamycin®), Epirubicin, Idarubicin), Actinomycin-D, Bleomycin, Mitomycin-C, and Mitoxantrone. In some embodiments, the anti-neoplastic agent is not a topoisomerase inhibitor. For example, in some embodiments, the anti-neoplastic agent is not one of the following topoisomerase inhibitors: Topotecan, Irinotecan (CPT-11), Etoposide (VP-16), Teniposide, and Mitoxantrone. In some embodiments, the anti-neoplastic agent is not a mitotic inhibitor. For example, in some embodiments, the anti-neoplastic agent is not one of the following mitotic inhibitors: Docetaxel, Estramustine, Ixabepilone, Paclitaxel, Vinblastine, Vincristine, and Vinorelbine. In some embodiments, the anti-neoplastic agent is not a corticosteroid. For example, in some embodiments the anti-neoplastic agent is not one of the following coritcosteroids: Prednisone, Methylprednisolone (Solumedrol®), and Dexamethasone (Decadron®). In some embodiments the anti-neoplastic agent is not an enzyme. For example, in some embodiments, the anti-neoplastic agents is not one of the following enzymes: L-asparaginase, and bortezomib (Velcade®)). In some embodiments, the anti-neoplastic agent is not a biologic agent. For example, in some embodiments, the anti-neoplastic agent is not an anti-TNF antibody (e.g., adalimumab or infliximab); an anti-CD20 antibody (e.g. rituximab), an anti-VEGF antibody (e.g. bevacizumab); an anti-HER2 antibody (e.g. trastuzumab); or an anti-RSV antibody (e.g. palivizumab).

As used herein, the terms “administering in combination”, “co-administering” or “co-administration” refer to administration of the agent that induces iron-dependent cellular disassembly prior to, concurrently or substantially concurrently with, subsequently to, or intermittently with the administration of the anti-neoplastic agent. In certain embodiments, that agent that induces iron-dependent cellular disassembly is administered prior to administration of the anti-neoplastic agent. In certain embodiments, the agent that induces iron-dependent cellular disassembly is administered concurrently with the anti-neoplastic agent. In certain embodiments, the agent that induces iron-dependent cellular disassembly is administered after administration of the anti-neoplastic agent.

The agent that induces iron-dependent cellular disassembly and the anti-neoplastic agent can act additively or synergistically. In one embodiment, the agent that induces iron-dependent cellular disassembly and the anti-neoplastic agent act synergistically. In some embodiments the synergistic effects are in the treatment of the cancer. For example, in one embodiment, the combination of the agent that induces iron-dependent cellular disassembly and the anti-neoplastic agent improves the durability, i.e. extends the duration, of the immune response against the cancer that is targeted by the anti-neoplastic agent. In some embodiments, the agent that induces iron-dependent cellular disassembly and the anti-neoplastic agent act additively.

In some embodiments, the combination therapy of the agent that induces iron-dependent cellular disassembly and the anti-neoplastic agent inhibits tumor cell growth. Accordingly, the invention further provides methods of inhibiting tumor cell growth in a subject, comprising administering an agent that induces iron-dependent cellular disassembly and at least one anti-neoplastic agent to the subject, such that tumor cell growth is inhibited. In certain embodiments, treating cancer comprises extending survival or extending time to tumor progression as compared to a control. In some embodiments, the control is a subject that is treated with the anti-neoplastic agent, but is not treated with the agent that induces iron-dependent cellular disassembly. In some embodiments, the control is a subject that is treated with the agent that induces iron-dependent cellular disassembly, but is not treated with the anti-neoplastic agent. In some embodiments, the control is a subject that is not treated with the anti-neoplastic agent or the agent that induces iron-dependent cellular disassembly. In certain embodiments, the subject is a human subject. In preferred embodiments, the subject is identified as having a tumor prior to administration of the first dose of the agent that induces iron-dependent cellular disassembly or the first dose of the anti-neoplastic agent. In certain embodiments, the subject has a tumor at the time of the first administration of the agent that induces iron-dependent cellular disassembly or at the time of first administration of the anti-neoplastic agent.

In certain embodiments, at least 1, 2, 3, 4, or 5 cycles of the combination therapy are administered to the subject. The subject is assessed for response criteria at the end of each cycle. The subject is also monitored throughout each cycle for adverse events (e.g., clotting, anemia, liver and kidney function, etc.) to ensure that the treatment regimen is being sufficiently tolerated.

It should be noted that more than one anti-neoplastic agent e.g., 2, 3, 4, 5, or more anti-neoplastic agents, may be administered in combination with the agent that induces iron-dependent cellular disassembly.

In one embodiment, administration of the agent that induces iron-dependent cellular disassembly and the anti-neoplastic agent as described herein results in one or more of, reducing tumor size, weight or volume, increasing time to progression, inhibiting tumor growth and/or prolonging the survival time of a subject having an oncological disorder. In certain embodiments, administration of the agent that induces iron-dependent cellular disassembly and the anti-neoplastic agent reduces tumor size, weight or volume, increases time to progression, inhibits tumor growth and/or prolongs the survival time of the subject by at least 1%, 2%, 3%, 4%, 5%, 10%, 20%, 30%, 40%, 50%, 60%, 70%, 80%, 90%, 100%, 200%, 300%, 400% or 500% relative to a corresponding control subject that is administered the agent that induces iron-dependent cellular disassembly alone or the anti-neoplastic agent alone. In certain embodiments, administration of the agent that induces iron-dependent cellular disassembly and the anti-neoplastic agent reduces tumor size, weight or volume, increases time to progression, inhibits tumor growth and/or prolongs the survival time of a population of subjects afflicted with an oncological disorder by at least 1%, 2%, 3%, 4%, 5%, 10%, 20%, 30%, 40%, 50%, 60%, 70%, 80%, 90%, 100%, 200%, 300%, 400% or 500% relative to a corresponding population of control subjects afflicted with the oncological disorder that is administered the agent that induces iron-dependent cellular disassembly alone or the anti-neoplastic agent alone. In other embodiments, administration of the agent that induces iron-dependent cellular disassembly and the anti-neoplastic agent stabilizes the oncological disorder in a subject with a progressive oncological disorder prior to treatment.

Cancers for treatment using the methods of the invention include, for example, all types of cancer or neoplasm or malignant tumors found in mammals, including, but not limited to: sarcomas, melanomas, carcinomas, leukemias, and lymphomas.

The term “sarcoma” generally refers to a tumor which is made up of a substance like the embryonic connective tissue and is generally composed of closely packed cells embedded in a fibrillar or homogeneous substance. Examples of sarcomas which can be treated with the methods of the invention include, for example, a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, uterine sarcoma, myxoid liposarcoma, leiomyosarcoma, spindle cell sarcoma, desmoplastic sarcoma, and telangiectaltic sarcoma.

The term “melanoma” is taken to mean a tumor arising from the melanocytic system of the skin and other organs. Melanomas which can be treated with the methods of the invention include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Carcinomas which can be treated with the methods of the invention, as described herein, include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, colon adenocarcinoma of colon, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, merkel cell carcinoma, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, cervical squamous cell carcinoma, tonsil squamous cell carcinoma, and carcinoma villosum. In a particular embodiment, the cancer is renal cell carcinoma.

The term “leukemia” refers to a type of cancer of the blood or bone marrow characterized by an abnormal increase of immature white blood cells called “blasts”. Leukemia is a broad term covering a spectrum of diseases. In turn, it is part of the even broader group of diseases affecting the blood, bone marrow, and lymphoid system, which are all known as hematological neoplasms. Leukemias can be divided into four major classifications, acute lymphocytic (or lymphoblastic) leukemia (ALL), acute myelogenous (or myeloid or non-lymphatic) leukemia (AML), chronic lymphocytic leukemia (CLL), and chronic myelogenous leukemia (CML). Further types of leukemia include Hairy cell leukemia (HCL), T-cell prolymphocytic leukemia (T-PLL), large granular lymphocytic leukemia, and adult T-cell leukemia. In certain embodiments, leukemias include acute leukemias. In certain embodiments, leukemias include chronic leukemias.

The term “lymphoma” refers to a group of blood cell tumors that develop from lymphatic cells. The two main categories of lymphomas are Hodgkin lymphomas (HL) and non-Hodgkin lymphomas (NHL) Lymphomas include any neoplasms of the lymphatic tissues. The main classes are cancers of the lymphocytes, a type of white blood cell that belongs to both the lymph and the blood and pervades both.

In some embodiments, the compositions are used for treatment of various types of solid tumors, for example breast cancer (e.g. triple negative breast cancer), bladder cancer, genitourinary tract cancer, colon cancer, rectal cancer, endometrial cancer, kidney (renal cell) cancer, pancreatic cancer, prostate cancer, thyroid cancer (e.g. papillary thyroid cancer), skin cancer, bone cancer, brain cancer, cervical cancer, liver cancer, stomach cancer, mouth and oral cancers, esophageal cancer, adenoid cystic cancer, neuroblastoma, testicular cancer, uterine cancer, thyroid cancer, head and neck cancer, kidney cancer, lung cancer (e.g. small cell lung cancer, non-small cell lung cancer), mesothelioma, ovarian cancer, sarcoma, stomach cancer, uterine cancer, cervical cancer, medulloblastoma, and vulvar cancer. In certain embodiments, skin cancer includes melanoma, squamous cell carcinoma, and cutaneous T-cell lymphoma (CTCL).

Additional cancers which can be treated with the compositions of the invention include, for example, multiple myeloma, primary thrombocytosis, primary macroglobulinemia, malignant pancreatic insulanoma, malignant carcinoid, malignant hypercalcemia, endometrial cancer, adrenal cortical cancer, and malignant fibrous histiocytoma.

Resistant Cancers

In certain aspects, the disclosure relates to a method of killing a cancer cell in a subject, comprising contacting the cancer cell, or cells adjacent to the cancer cell, with a combination of (a) an anti-neoplastic agent and (b) an agent that induces iron-dependent cellular disassembly, wherein the cancer cell is resistant to the anti-neoplastic agent, thereby killing the cancer cell.

In some aspects, the disclosure relates to a method of treating a cancer in a subject in need thereof, comprising administering to the subject, in combination (a) an anti-neoplastic agent and (b) an agent that induces iron-dependent cellular disassembly, thereby treating the cancer in the subject, wherein the cancer is resistant to the anti-neoplastic agent.

In some embodiments, said contacting or administering induces iron-dependent cellular disassembly of the resistant cancer or cancer cell.

For example, in certain aspects the disclosure relates to a method of treating a cancer in a subject in need thereof, comprising administering to the subject, in combination (a) an anti-neoplastic agent and (b) an agent that induces iron-dependent cellular disassembly, thereby treating the cancer in the subject, wherein the anti-neoplastic agent is known to induce resistance in the cancer. Non-limiting examples of anti-neoplastic agents that are known to induce resistance, and that may be used in the methods of the disclosure, are provided in Table 6.

In some embodiments, the cancer to be treated by the methods disclosed herein is a cancer that is known to develop, or is at high risk for developing, resistance to approved cancer therapies. Examples of cancers that are known to develop resistance to an anti-neoplastic agent, and the approved therapies to which they have been shown to develop resistance, are provided in Table 4 above. For example, in some embodiments, the cancer is selected from the group consisting of chronic myeloid leukemia (CML), acute lymphoblastic leukemia (ALL), gastrointestinal stromal tumor (GIST), colorectal cancer (CRC), non-small cell lung cancer (NSCLC), melanoma, ovarian cancer, breast cancer and gastric cancer.

Accordingly, in some embodiments, the disclosure relates to a method of treating a cancer in a subject in need thereof, comprising administering to the subject, in combination (a) an anti-neoplastic agent and (b) an agent that induces iron-dependent cellular disassembly, thereby treating the cancer in the subject, wherein the anti-neoplastic agent is known to induce resistance in the cancer, wherein the anti-neoplastic agent is an anti-neoplastic agent selected from the agents provided in table 6, and the cancer to be treated in the subject is the corresponding cancer approved for the therapy (i.e., the drug or anti-neoplastic agent) provided in Table 6.

In some embodiments, the cancer comprises cells that are resistant to an anti-neoplastic agent, as well as cells that are susceptible to the anti-neoplastic agent. For example, in certain aspects the disclosure relates to a method of reducing the heterogeneity of a cancer in a subject in need thereof, wherein the cancer comprises cells that are resistant to an anti-neoplastic agent and cells that are sensitive to the anti-neoplastic agent, the method comprising administering to the subject, in combination (a) the anti-neoplastic agent and (b) an agent that induces iron-dependent cellular disassembly, thereby reducing the heterogeneity of the cancer.

In some embodiments, the cells that are resistant to an anti-neoplastic agent comprise or consist of persister cells. The term “persister cell” as used herein refers to a cancer cell that has a resistance to an anti-neoplastic agent that is not dependent on mutation of the anti-neoplastic agent target protein. Persister cells often exhibit gene expression profiles that are typical of mesenchymal cells. Persister cells also often exhibit altered transcriptional states. For example, in some embodiments a resistant cancer cell (e.g. a persister cell) exhibits (i) increased expression of a marker selected from the group consisting of HIF1, CD133, CD24, KDM5A/RBP2/Jarid1A, IGFBP3 (IGF-binding protein 3), Stat3, IRF-1, Interferon gamma, type I interferon, pax6, AKT pathway activation, IGF1, EGF, ANGPTL7, PDGFD, FRA1 (FOSL1), FGFR, KIT, IGF1R and DDR1, relative to a cancer cell that is sensitive to the anti-neoplastic agent; and/or (ii) decreased expression of IGFBP-3 relative to a cancer cell that is sensitive to the anti-neoplastic agent. Examples of markers of drug-resistant cancer cells are provided in Table 7 below.

TABLE 7
Markers of drug-resistant cancer cells

| | Change in | |
|---|---|---|
| | Marker | |
| | Expression | |
| | Relative to | |
| | Drug-Sensitive | |
| Marker | Cancer Cells | Reference |
| CD133 | increased | Sharma S V, et al. A chromatin- |
| CD24 | | mediated reversible drug-tolerant |
| KDM5A/RBP2/ | | state in cancer cell subpopula- |
| Jarid1A | | tions. Cell 2010; 141: 69-80 |
| IGFBP3 | | |
| (IGF-binding | | |
| protein 3) | | |
| Stat3 | increased | Lee H J, et al. Drug resistance |
| IRF-1 | | via feedback activation of Stat3 in |
| Interferon | | oncogene-addicted cancer cells. |
| gamma, type I | | Cancer Cell 2014; 26: 207-21. |
| interferon | | |
| pax6 | | |
| AKT pathway | increased | Obenauf A C, et al. Therapy induced |
| activation | | tumour secretomes promote resistance |
| IGF1 | | and tumour progression. Nature 2015; |
| EGF | | 520: 368-72. |
| ANGPTL7 | | |
| PDGFD | | |
| FRA1 (FOSL1) | | |
| IGFBP-3 | reduced | Obenauf A C, et al. Therapy induced |
| | | tumour secretomes promote resistance |
| | | and tumour progression. Nature 2015; |
| | | 520: 368-72. |
| FGFR | increased | Zawistowski J S, et al. Enhancer |
| KIT | | remodeling during adaptive bypass to |
| IGF1R | | MEK inhibition is attenuated by pharma- |
| DDR1 | | cologic targeting of the P-TEFb complex. |
| | | Cancer Discov 2017; 7: 302-21. |

In some embodiments of the methods described herein, the subject was previously determined to have cancer comprising persister cells or elevated levels of persister cells. Levels of persister cells may be determined in a subject by collecting a sample of cancer cells from the subject after treatment with an anti-neoplastic agent and detecting mutations in the cancer cells collected from the subject. In some embodiments, administration of the combination of the anti-neoplastic agent and the agent that induces iron-dependent cellular disassembly results in reduction of the number of persister cells in the cancer. In some embodiments, administration results in preferential killing of the persister cells in the cancer.

In some embodiments, the cells that are resistant to an anti-neoplastic agent comprise or consist of cancer stem cells (CSCs). The term “cancer stem cell” or “CSC” as used herein refers to a cancer cell that is resistant to an anti-neoplastic agent and is capable of initiating growth of a tumor. In some embodiments the cancer comprises both cancer stem cells (CSCs) and cells that are not cancer stem cells (non-CSCs). In some embodiments, the non-CSCs are sensitive to the anti-neoplastic agent. In some embodiments, the subject was previously determined to have cancer comprising CSCs or elevated levels of CSCs. Levels of CSCs may be determined in a subject by collecting a sample from the subject (e.g. cancer cells, a blood sample, or a urine sample) and measuring expression of CSC markers in the subject. CSC markers are known in the art and are described, for example, in Kim et al., 2017, BMB Reports 50(6):285-298, which is incorporated by reference herein in its entirety. Non-limiting examples of CSC markers are provided in Table 8 below.

TABLE 8
Cancer stem cell (CSC) markers

| Cancer stem cell marker | Type of cancer |
|---|---|
| SSEA3 | Teratocarcinoma, breast |
| SSEA4 | Teratocarcinoma, breast |
| TRA-1-60 | Teratocarcinoma, breast, prostate |
| TRA-1-81 | Teratocarcinoma, breast |
| SSEA1 | Teratocarcinoma,renal, lung |
| CD133 (AC133) | breast, prostrate, colon, glioma, liver, lung, |
| | ovary |
| CD90 (Thy-1) | Brain, liver |
| CD326 (EpCAM) | |
| Cripto-1 (TDGF1) | Breast, colon, lung |
| PODXL-1 (Podocalyxinlike | Leukemia, breast, pancreas, lung |
| protein 1) | |
| ABCG2 ATP-binding | Lung, breast, brain |
| cassette transporter | |
| CD24 | Breast, gastric, pancreas |
| CD49f(Integrin alpha6) | Glioma |
| Notch2 | Pancreas, lung |
| CD146 (MCAM) | Rhabdoid tumor, sarcoma |
| CD10 (Neprilysin) | Breast, head and neck |
| CD117 (c-KIT) | Ovary |
| CD26 (DPP-4) | Colorectal, leukemia |
| CXCR4 | Breast, brain, pancreas |
| CD34 | Leukemia, squamous |
| CD271 | Melanoma, head and neck |
| CD13 (Alanine amino- | Liver |
| peptidase) | |
| CD56 | (NCAM) |
| CD105 (Endoglin) | Renal |
| LGR5 | Intestinal, colorectal |
| CD114 (CSF3R) | Neuroblastoma |
| CD54 (ICAM-1) | Gastric |
| CXCR1, 2 | Breast, pancreas |
| TIM-3 (HAVCR2) | Leukemia |
| CD55 (DAF) | Breast |
| DLL4 (Delta-like ligand | Colorectal, ovarian |
| 4) | |
| CD20 (MS4A1) Melanoma | |
| CD96 | Leukemia |
| CD29 (Integrin alpha1) | Breast, colon |
| CD9 | Leukemia |
| CD166 (ALCAM) | Colorectal, lung |
| CD44 | HNSCC, breast, colon, liver, ovarian, |
| | pancreas, gastric |
| ABCB5 | Melanoma |
| Notch3 CD123 | (IL-3R) Leukemia |

Cancer stem cells may exhibit characteristics of non-cancerous stem cells. e.g. mesenchymal stem cells. In some embodiments, the cancer stem cell is a cell that has undergone an epithelial-mesenchymal transition (EMT). Cells that have undergone an EMT are epithelial cells that have acquired mesenchymal-like properties and show reduced intercellular adhesion and increased motility. The concept that EMT involves the formation of metastatic cancer cells is based on the observation that acquisition of mesenchymal markers such as vimentin or S100A4 (also known as fibroblast-specific protein 1 [FSP1]) by epithelial carcinoma cells is associated with increased metastatic potential, as is nuclear overexpression of β-catenin, and loss of epithelial cell adhesion molecules such as E-cadherin. See Zeisberg et al., 2009, J Clin Invest. 2009 June; 119(6):1429-37.

In some embodiments, the cells that are resistant to an anti-neoplastic agent comprise or consist of EMT cells. In some embodiments, EMT cells exhibit (i) increased expression of a marker selected from the group consisting of Vimentin (S100A4), Beta-catenin, N-cadherin, Beta6 integrin, Alpha4 integrin, DDR2, FSP1, Alpha-SMA, Beta-Catenin, Laminin 5, FTS-1, Twist, FOXX2, OB-cadherin, Alpha5beta1 integrin, alphaVbeta6 integrin, Syndecan-1, Alpha1 (I) collagen, Alpha1 (III) collagen, Snail1, Snail2, ZEB1, CBF-A/KAP-1 complex, LEF-1, Ets-1 and miR-21, relative to an epithelial cell; and/or (ii) decreased expression of a marker selected from the group consisting of E-cadherin, ZO-1, cytokeratin, Alpha1 (IV) collagen and Laminin 1, relative to an epithelial cell. Examples of EMT markers are provided in Table 9 below.

TABLE 9
Epithelial-mesenchymal transition (EMT) markers.

| | Change in Marker | |
|---|---|---|
| | Expression Relative to | |
| Marker | Epithelial Cells | Types of cancer |
| Vimentin or S100A4 | increased | |
| Beta-catenin | increased | |
| E-cadherin | decreased | |
| Switch from E-cadherin | Switch | |
| to N-cadherin | | |
| Beta6 integrin | High expression | Colon carcinoma |
| Alpha4 integrin | High expression | melanoma |
| DDR2 | increased | |
| FSP1 | increased | |
| Alpha-SMA | increased | Breast cancer |
| Beta-Catenin | increased | |
| Laminin 5 | increased | Breast cancer, ductal |
| FTS-1 | increased | |
| Twist | increased | |
| FOXX2 | increased | Breast cancer |
| OB-cadherin | increased | |
| Alpha5beta1 integrin | increased | |
| alphaVbeta6 integrin | increased | |
| Syndecan-1 | increased | |
| Alpha1 (I) collagen | increased | |
| Alpha1 (III) collagen | increased | |
| Snail1 | increased | |
| Snail2 | increased | |
| ZEB1 | increased | |
| CBF-A/KAP-1 complex | increased | |
| LEF-1 | increased | |
| Ets-1 | increased | |
| miR-21 | increased | |
| ZO-1 | decreased | |
| cytokeratin | decreased | |
| Alpha1 (IV) collagen | decreased | |
| Laminin 1 | decreased | |

In some embodiments, the cancer comprises EMT cells and non-CSCs that are epithelial cells. Administration of the combination of the anti-neoplastic agent and the agent that induces iron-dependent cellular disassembly may result in reduction of the number of the CSCs in the cancer and/or preferential killing of the CSCs in the cancer relative to a cancer that is treated with the anti-neoplastic agent alone and/or the agent that induces iron-dependent cellular disassembly alone. In some embodiments, administration increases the number of cells undergoing ferroptosis in the cancer relative to a cancer that is treated with the anti-neoplastic agent alone and/or the agent that induces iron-dependent cellular disassembly alone. In some embodiments, administration reduces proliferation of the cancer. In some embodiments, administration reduces the ratio of CSCs to non-CSCs in the cancer. In some embodiments, administration reduces the number of cancer cells. In some embodiments, administration promotes differentiation of the cancer to a less aggressive cancer. For example, rapidly growing cancer cells may be more dependent on detoxification of radical oxygen species (ROS), and thus may be more susceptible to agents that induce iron dependent cellular disassembly. Eliminating these rapidly growing cells by inducing iron dependent cellular disassembly may select for slower growing cancer cells, resulting in a less aggressive cancer. In some embodiments, administration results in preferential killing of the CSCs in the cancer. In some embodiments, administration reduces risk of relapse of the cancer. In some embodiments, administration reduces risk of metastasis of the cancer.

In some embodiments, the combination therapies described herein may be administered to a subject that has previously failed treatment for a cancer with an anti-neoplastic (e.g. chemotherapeutic) regimen. A “subject who has failed an anti-neoplastic regimen” is a subject with cancer that does not respond, or ceases to respond to treatment with a anti-neoplastic regimen per RECIST 1.1 criteria, i.e., does not achieve a complete response, partial response, or stable disease in the target lesion; or does not achieve complete response or non-CR/non-PD of non-target lesions, either during or after completion of the anti-neoplastic regimen, either alone or in conjunction with surgery and/or radiation therapy which, when possible, are often clinically indicated in conjunction with anti-neoplastic therapy. The RECIST 1.1 criteria are described, for example, in Eisenhauer et al., 2009, Eur. J. Cancer 45:228-24 (which is incorporated herein by reference in its entirety), and discussed in greater detail below. A failed anti-neoplastic regimen results in, e.g., tumor growth, increased tumor burden, and/or tumor metastasis. A failed anti-neoplastic regimen as used herein includes a treatment regimen that was terminated due to a dose limiting toxicity, e.g., a grade III or a grade IV toxicity that cannot be resolved to allow continuation or resumption of treatment with the anti-neoplastic agent or regimen that caused the toxicity. In one embodiment, the subject has failed treatment with a anti-neoplastic regimen comprising administration of one or more anti-angiogenic agents.

A failed anti-neoplastic regimen includes a treatment regimen that does not result in at least stable disease for all target and non-target lesions for an extended period, e.g., at least 1 month, at least 2 months, at least 3 months, at least 4 months, at least 5 months, at least 6 months, at least 12 months, at least 18 months, or any time period less than a clinically defined cure. A failed anti-neoplastic regimen includes a treatment regimen that results in progressive disease of at least one target lesion during treatment with the anti-neoplastic agent, or results in progressive disease less than 2 weeks, less than 1 month, less than two months, less than 3 months, less than 4 months, less than 5 months, less than 6 months, less than 12 months, or less than 18 months after the conclusion of the treatment regimen, or less than any time period less than a clinically defined cure.

A failed anti-neoplastic regimen does not include a treatment regimen wherein the subject treated for a cancer achieves a clinically defined cure, e.g., 5 years of complete response after the end of the treatment regimen, and wherein the subject is subsequently diagnosed with a distinct cancer, e.g., more than 5 years, more than 6 years, more than 7 years, more than 8 years, more than 9 years, more than 10 years, more than 11 years, more than 12 years, more than 13 years, more than 14 years, or more than 15 years after the end of the treatment regimen.

RECIST criteria are clinically accepted assessment criteria used to provide a standard approach to solid tumor measurement and provide definitions for objective assessment of change in tumor size for use in clinical trials. Such criteria can also be used to monitor response of an individual undergoing treatment for a solid tumor. The RECIST 1.1 criteria are discussed in detail in Eisenhauer et al., 2009, Eur. J. Cancer 45:228-24, which is incorporated herein by reference. Response criteria for target lesions include:

Complete Response (CR): Disappearance of all target lesions. Any pathological lymph nodes (whether target or non-target) must have a reduction in short axis to <10 mm.

Partial Response (PR): At least a 30% decrease in the sum of diameters of target lesion, taking as a reference the baseline sum diameters.

Progressive Diseases (PD): At least a 20% increase in the sum of diameters of target lesions, taking as a reference the smallest sum on the study (this includes the baseline sum if that is the smallest on the study). In addition to the relative increase of 20%, the sum must also demonstrate an absolute increase of at least 5 mm. (Note: the appearance of one or more new lesions is also considered progression.)

Stable Disease (SD): Neither sufficient shrinkage to qualify for PR nor sufficient increase to qualify for PD, taking as a reference the smallest sum diameters while on study.

RECIST 1.1 criteria also consider non-target lesions which are defined as lesions that may be measureable, but need not be measured, and should only be assessed qualitatively at the desired time points. Response criteria for non-target lesions include:

Complete Response (CR): Disappearance of all non-target lesions and normalization of tumor marker levels. All lymph nodes must be non-pathological in size (<10 mm short axis).

Non-CR/Non-PD: Persistence of one or more non-target lesion(s) and/or maintenance of tumor marker level above the normal limits.

Progressive Disease (PD): Unequivocal progression of existing non-target lesions. The appearance of one or more new lesions is also considered progression. To achieve “unequivocal progression” on the basis of non-target disease, there must be an overall level of substantial worsening of non-target disease such that, even in the presence of SD or PR in target disease, the overall tumor burden has increased sufficiently to merit discontinuation of therapy. A modest “increase” in the size of one or more non-target lesions is usually not sufficient to qualify for unequivocal progression status. The designation of overall progression solely on the basis of change in non-target disease in the face of SD or PR in target disease will therefore be extremely rare.

In some embodiments, the combination therapies described herein may be administered to a subject having a refractory cancer. A “refractory cancer” is a malignancy for which surgery is ineffective, which is either initially unresponsive to chemo- or radiation therapy, or which becomes unresponsive to chemo- or radiation therapy over time.

Several methods are known in the art and may be employed for identifying cells undergoing iron-dependent cellular disassembly (e.g., ferroptosis) and distinguishing from other types of cellular disassembly and/or cell death through detection of particular markers. (See, for example, Stockwell et al., 2017, Cell 171: 273-285, incorporated by reference herein in its entirety). For example, because iron-dependent cellular disassembly may result from lethal lipid peroxidation, measuring lipid peroxidation provides one method of identifying cells undergoing iron-dependent cellular disassembly. C11-BODIPY and Liperfluo are lipophilic ROS sensors that provide a rapid, indirect means to detect lipid ROS (Dixon et al., 2012, Cell 149: 1060-1072). Liquid chromatography (LC)/tandem mass spectrometry (MS) analysis can also be used to detect specific oxidized lipids directly (Friedmann Angeli et al., 2014, Nat. Cell Biol. 16: 1180-1191; Kagan et al., 2017, Nat. Chem. Biol. 13: 81-90). Isoprostanes and malondialdehyde (MDA) may also be used to measure lipid peroxidation (Milne et al., 2007, Nat. Protoc. 2: 221-226; Wang et al., 2017, Hepatology 66(2): 449-465). Kits for measuring MDA are commercially available (Beyotime, Haimen, China).

Other useful assays for studying iron-dependent cellular disassembly include measuring iron abundance and GPX4 activity. Iron abundance can be measured using inductively coupled plasma-MS or calcein AM quenching, as well as other specific iron probes (Hirayama and Nagasawa, 2017, J. Clin. Biochem. Nutr. 60: 39-48; Spangler et al., 2016, Nat. Chem. Biol. 12: 680-685), while GPX4 activity can be detected using phosphatidylcholine hydroperoxide reduction in cell lysates using LC-MS (Yang et al., 2014, Cell 156: 317-331). In addition, iron-dependent cellular disassembly may be evaluated by measuring glutathione (GSH) content. GSH may be measured, for example, by using the commercially available GSH-Glo Glutathione Assay (Promega, Madison, Wis.).

Iron-dependent cellular disassembly may also be evaluated by measuring the expression of one or more marker proteins. Suitable marker proteins include, but are not limited to, glutathione peroxidase 4 (GPX4), prostaglandin-endoperoxide synthase 2 (PTGS2), and cyclooxygenase-2 (COX-2). The level of expression of the marker protein or a nucleic acid encoding the marker protein may be determined using suitable techniques known in the art including, but not limited to polymerase chain reaction (PCR) amplification reaction, reverse-transcriptase PCR analysis, quantitative real-time PCR, single-strand conformation polymorphism analysis (SSCP), mismatch cleavage detection, heteroduplex analysis, Northern blot analysis, Western blot analysis, in situ hybridization, array analysis, deoxyribonucleic acid sequencing, restriction fragment length polymorphism analysis, and combinations or sub-combinations thereof.

Applicants have surprisingly shown that induction of iron-dependent cellular disassembly (e.g. ferroptosis) in a cell, e.g. a cancer cell, increases immune response as evidenced by increases in NFKB and IRF activity in immune cells. While not wishing to be bound by theory, it is expected that this increased immune response would result in more effective targeting of cancer cells by the subject's immune system. Accordingly, in combination therapies comprising an agent that induces iron-dependent cellular disassembly and an anti-neoplastic agent, a reduced dose of the anti-neoplastic agent may be used and still maintain the same level of cancer control. This approach would be especially suitable for anti-neoplastic agents that exhibit toxicities at the doses required for efficacy.

Accordingly, in certain aspects, the disclosure relates to a method of treating a cancer in a subject in need thereof, comprising administering to the subject, in combination (a) an anti-neoplastic agent and (b) an agent that induces iron-dependent cellular disassembly, wherein the anti-neoplastic agent is administered at a dose that is lower than an effective dose of the anti-neoplastic agent when administered alone to treat the cancer, thereby treating the cancer in the subject. In some embodiments, the anti-neoplastic agent is administered at a dose that has no effect on treating the cancer when not administered in combination with the agent that induces iron-dependent disassembly. In some embodiments, the anti-neoplastic agent is administered at a dose that has a minimal effect in treating the cancer, but is lower than the standard dose for the anti-neoplastic agent. In some embodiments, the anti-neoplastic agent is administered at a dose that is at least 5%, 10%, 20%, 30%, 40%, 50%, 60%, 70%, 80% or 90% less than the effective dose of the anti-neoplastic agent when administered alone to treat the cancer. In some embodiments, the anti-neoplastic agent has a dose limiting effect.

Because a lower dose of the anti-neoplastic agent may be administered while maintaining the same level of efficacy, the combination therapies described herein would be expected to increase the therapeutic index of the anti-neoplastic agent. The “therapeutic index” is the dose ratio between toxic and therapeutic effects, and it can be expressed as the ratio LD₅₀/ED₅₀. LD₅₀ is the dose lethal to 50% of the population. ED₅₀ is the dose therapeutically effective in 50% of the population. Drugs with a higher therapeutic index are desirable. It is expected that the ED₅₀ of the anti-neoplastic agent would be reduced by combination with the agent that induces iron-dependent cellular disassembly, resulting in an increased therapeutic index.

Accordingly, in some aspects, the disclosure relates to a method of increasing the therapeutic index of an anti-neoplastic agent for treating a cancer in a subject in need thereof, comprising administering to the subject, in combination (a) the anti-neoplastic agent and (b) an agent that induces iron-dependent cellular disassembly, thereby increasing the therapeutic index of the anti-neoplastic agent for treating the cancer in the subject.

In any of the methods described herein, administration of the combination of the agent that induces iron-dependent cellular disassembly and the anti-neoplastic agent may be further combined with administration of an additional agent, such as an immunotherapeutic agent, for example, to further increase immune response in a subject. In some embodiments, the immunotherapeutic is selected from the group consisting of an immune checkpoint modulator of an immune checkpoint molecule, a Toll-like receptor (TLR) agonist, a cell-based therapy, a cytokine and a cancer vaccine. Non-limiting examples of immunotherapeutic agents suitable for use in the methods described herein are provided herein and below.

In certain aspects, the disclosure relates to a method for the treatment of cancers by administering an agent that induces iron-dependent cellular disassembly in combination with at least one immune checkpoint modulator to a subject. In certain embodiments, the immune checkpoint modulator stimulates the immune response of the subject. For example, in some embodiments, the immune checkpoint modulator stimulates or increases the expression or activity of a stimulatory immune checkpoint (e.g. CD27, CD28, CD40, CD122, OX40, GITR, ICOS, or 4-1BB). In some embodiments, the immune checkpoint modulator inhibits or decreases the expression or activity of an inhibitory immune checkpoint (e.g. A2A4, B7-H3, B7-H4, BTLA, CTLA-4, IDO, KIR, LAG3, PD-1, PD-L1, PD-L2, TIM-3 or VISTA).

In certain embodiments of the aforementioned methods, the agent that induces iron-dependent disassembly is a compound represented by Structural Formula (VI) as described herein.

In certain aspects, the disclosure relates to a method of killing a cancer cell in a subject, comprising contacting the cancer cell, or cells adjacent to the cancer cell, with a combination of (a) an anti-neoplastic agent and (b) an agent that induces iron-dependent cellular disassembly. In certain embodiments, the anti-neoplastic agent is not an immunotherapeutic agent. In some embodiments, the anti-neoplastic agent is not an alkylating agent. For example, in some embodiments, the anti-neoplastic agent is not one of the following alkylating agents: Altretamine, Busulfan, Carboplatin, Carmustine, Chlorambucil, Cisplatin, Cyclophosphamide, Dacarbazine, Lomustine, Melphalan, Oxaliplatin, Temozolomide, and Thiotepa. In some embodiments, the anti-neoplastic agent is not an antimetabolite. For example, in some embodiments, the anti-neoplastic agent is not one of the following antimetabolites: 5-fluorouracil (5-FU), 6-mercaptopurine (6-MP); Capecitabine (Xeloda®), Cytarabine (Ara-C®), Floxuridine, Fludarabine, Gemcitabine (Gemzar®), Hydroxyurea, Methotrexate, and Pemetrexed (Alimta®). In some embodiments, the anti-neoplastic agent is not an anti-tumor antibiotic. For example, in some embodiments, the anti-neoplastic agent is not one of the following anti-tumor antibiotics: anthracyclines (e.g., Daunorubicin, Doxorubicin (Adriamycin®), Epirubicin, Idarubicin), Actinomycin-D, Bleomycin, Mitomycin-C, and Mitoxantrone. In some embodiments, the anti-neoplastic agent is not a topoisomerase inhibitor. For example, in some embodiments, the anti-neoplastic agent is not one of the following topoisomerase inhibitors: Topotecan, Irinotecan (CPT-11), Etoposide (VP-16), Teniposide, and Mitoxantrone. In some embodiments, the anti-neoplastic agent is not a mitotic inhibitor. For example, in some embodiments, the anti-neoplastic agent is not one of the following mitotic inhibitors: Docetaxel, Estramustine, Ixabepilone, Paclitaxel, Vinblastine, Vincristine, and Vinorelbine. In some embodiments, the anti-neoplastic agent is not a corticosteroid. For example, in some embodiments the anti-neoplastic agent is not one of the following coritcosteroids: Prednisone, Methylprednisolone (Solumedrol®), and Dexamethasone (Decadron®). In some embodiments the anti-neoplastic agent is not an enzyme. For example, in some embodiments, the anti-neoplastic agents is not one of the following enzymes: L-asparaginase, and bortezomib (Velcade®)). In some embodiments, the anti-neoplastic agent is not a biologic agent. For example, in some embodiments, the anti-neoplastic agent is not an anti-TNF antibody (e.g., adalimumab or infliximab); an anti-CD20 antibody (e.g. rituximab), an anti-VEGF antibody (e.g. bevacizumab); an anti-HER2 antibody (e.g. trastuzumab); or an anti-RSV antibody (e.g. palivizumab)

In certain embodiments of the aforementioned methods, the agent that induces iron-dependent disassembly is a compound represented by Structural Formula (VI) as described herein.

### V. Immunotherapeutic Agents

The methods described herein may comprise administration of an immunotherapeutic agent as provided below.

### Immune Checkpoint Modulators

In some embodiments, the immunotherapeutic is an immune checkpoint modulator of an immune checkpoint molecule. Examples include LAG-3 (Triebel et al., 1990, J. Exp. Med. 171: 1393-1405), TIM-3 (Sakuishi et al., 2010, J. Exp. Med. 207: 2187-2194) and VISTA (Wang et al., 2011, J. Exp. Med. 208: 577-592). Examples of co-stimulatory molecules that improve immune responses include ICOS (Fan et al., 2014, J. Exp. Med. 211: 715-725), OX40 (Curti et al., 2013, Cancer Res. 73: 7189-7198) and 4-1BB (Melero et al., 1997, Nat. Med. 3: 682-685).

Immune checkpoints may be stimulatory immune checkpoints (i.e. molecules that stimulate the immune response) or inhibitory immune checkpoints (i.e. molecules that inhibit immune response). In some embodiments, the immune checkpoint modulator is an antagonist of an inhibitory immune checkpoint. In some embodiments, the immune checkpoint modulator is an agonist of a stimulatory immune checkpoint. In some embodiments, the immune checkpoint modulator is an immune checkpoint binding protein (e.g., an antibody, antibody Fab fragment, divalent antibody, antibody drug conjugate, scFv, fusion protein, bivalent antibody, or tetravalent antibody). In certain embodiments, the immune checkpoint modulator is capable of binding to, or modulating the activity of more than one immune checkpoint. Examples of stimulatory and inhibitory immune checkpoints, and molecules that modulate these immune checkpoints that may be used in the methods of the invention, are provided below.

i. Stimulatory Immune Checkpoint Molecules

CD27 supports antigen-specific expansion of naïve T cells and is vital for the generation of T cell memory (see, e.g., Hendriks et al. (2000) Nat. Immunol. 171 (5): 433-40). CD27 is also a memory marker of B cells (see, e.g., Agematsu et al. (2000) Histol. Histopathol. 15 (2): 573-6. CD27 activity is governed by the transient availability of its ligand, CD70, on lymphocytes and dendritic cells (see, e.g., Borst et al. (2005) Curr. Opin. Immunol. 17 (3): 275-81). Multiple immune checkpoint modulators specific for CD27 have been developed and may be used as disclosed herein. In some embodiments, the immune checkpoint modulator is an agent that modulates the activity and/or expression of CD27. In some embodiments, the immune checkpoint modulator is an agent that binds to CD27 (e.g., an anti-CD27 antibody). In some embodiments, the checkpoint modulator is a CD27 agonist. In some embodiments, the checkpoint modulator is a CD27 antagonist. In some embodiments, the immune checkpoint modulator is an CD27-binding protein (e.g., an antibody). In some embodiments, the immune checkpoint modulator is varlilumab (Celldex Therapeutics). Additional CD27-binding proteins (e.g., antibodies) are known in the art and are disclosed, e.g., in U.S. Pat. Nos. 9,248,183, 9,102,737, 9,169,325, 9,023,999, 8,481,029; U.S. Patent Application Publication Nos. 2016/0185870, 2015/0337047, 2015/0299330, 2014/0112942, 2013/0336976, 2013/0243795, 2013/0183316, 2012/0213771, 2012/0093805, 2011/0274685, 2010/0173324; and PCT Publication Nos. WO 2015/016718, WO 2014/140374, WO 2013/138586, WO 2012/004367, WO 2011/130434, WO 2010/001908, and WO 2008/051424, each of which is incorporated by reference herein.

CD28. Cluster of Differentiation 28 (CD28) is one of the proteins expressed on T cells that provide co-stimulatory signals required for T cell activation and survival. T cell stimulation through CD28 in addition to the T-cell receptor (TCR) can provide a potent signal for the production of various interleukins (IL-6 in particular). Binding with its two ligands, CD80 and CD86, expressed on dendritic cells, prompts T cell expansion (see, e.g., Prasad et al. (1994) Proc. Nat'l. Acad. Sci. USA 91(7): 2834-8). Multiple immune checkpoint modulators specific for CD28 have been developed and may be used as disclosed herein. In some embodiments, the immune checkpoint modulator is an agent that modulates the activity and/or expression of CD28. In some embodiments, the immune checkpoint modulator is an agent that binds to CD28 (e.g., an anti-CD28 antibody). In some embodiments, the checkpoint modulator is an CD28 agonist. In some embodiments, the checkpoint modulator is an CD28 antagonist. In some embodiments, the immune checkpoint modulator is an CD28-binding protein (e.g., an antibody). In some embodiments, the immune checkpoint modulator is selected from the group consisting of TAB08 (TheraMab LLC), lulizumab (also known as BMS-931699, Bristol-Myers Squibb), and FR104 (OSE Immunotherapeutics). Additional CD28-binding proteins (e.g., antibodies) are known in the art and are disclosed, e.g., in U.S. Pat. Nos. 9,119,840, 8,709,414, 9,085,629, 8,034,585, 7,939,638, 8,389,016, 7,585,960, 8,454,959, 8,168,759, 8,785,604, 7,723,482; U.S. Patent Application Publication Nos. 2016/0017039, 2015/0299321, 2015/0150968, 2015/0071916, 2015/0376278, 2013/0078257, 2013/0230540, 2013/0078236, 2013/0109846, 2013/0266577, 2012/0201814, 2012/0082683, 2012/0219553, 2011/0189735, 2011/0097339, 2010/0266605, 2010/0168400, 2009/0246204, 2008/0038273; and PCT Publication Nos. WO 2015198147, WO 2016/05421, WO 2014/1209168, WO 2011/101791, WO 2010/007376, WO 2010/009391, WO 2004/004768, WO 2002/030459, WO 2002/051871, and WO 2002/047721, each of which is incorporated by reference herein.

CD40. Cluster of Differentiation 40 (CD40, also known as TNFRSF5) is found on a variety of immune system cells including antigen presenting cells. CD40L, otherwise known as CD154, is the ligand of CD40 and is transiently expressed on the surface of activated CD4⁺ T cells. CD40 signaling is known to ‘license’ dendritic cells to mature and thereby trigger T-cell activation and differentiation (see, e.g., O'Sullivan et al. (2003) Crit. Rev. Immunol. 23 (1): 83-107. Multiple immune checkpoint modulators specific for CD40 have been developed and may be used as disclosed herein. In some embodiments, the immune checkpoint modulator is an agent that modulates the activity and/or expression of CD40. In some embodiments, the immune checkpoint modulator is an agent that binds to CD40 (e.g., an anti-CD40 antibody). In some embodiments, the checkpoint modulator is a CD40 agonist. In some embodiments, the checkpoint modulator is an CD40 antagonist. In some embodiments, the immune checkpoint modulator is a CD40-binding protein selected from the group consisting of dacetuzumab (Genentech/Seattle Genetics), CP-870,893 (Pfizer), bleselumab (Astellas Pharma), lucatumumab (Novartis), CFZ533 (Novartis; see, e.g., Cordoba et al. (2015) Am. J. Transplant. 15(11): 2825-36), RG7876 (Genentech Inc.), FFP104 (PanGenetics, B.V.), APX005 (Apexigen), BI 655064 (Boehringer Ingelheim), Chi Lob 7/4 (Cancer Research UK; see, e.g., Johnson et al. (2015) Clin. Cancer Res. 21(6): 1321-8), ADC-1013 (BioInvent International), SEA-CD40 (Seattle Genetics), XmAb 5485 (Xencor), PG120 (PanGenetics B.V.), teneliximab (Bristol-Myers Squibb; see, e.g., Thompson et al. (2011) Am. J. Transplant. 11(5): 947-57), and AKH3 (Biogen; see, e.g., International Publication No. WO 2016/028810). Additional CD40-binding proteins (e.g., antibodies) are known in the art and are disclosed, e.g., in U.S. Pat. Nos. 9,234,044, 9,266,956, 9,109,011, 9,090,696, 9,023,360, 9,023,361, 9,221,913, 8,945,564, 8,926,979, 8,828,396, 8,637,032, 8,277,810, 8,088,383, 7,820,170, 7,790,166, 7,445,780, 7,361,345, 8,961,991, 8,669,352, 8,957,193, 8,778,345, 8,591,900, 8,551,485, 8,492,531, 8,362,210, 8,388,971; U.S. Patent Application Publication Nos. 2016/0045597, 2016/0152713, 2016/0075792, 2015/0299329, 2015/0057437 2015/0315282, 2015/0307616, 2014/0099317, 2014/0179907, 2014/0349395, 2014/0234344, 2014/0348836, 2014/0193405, 2014/0120103, 2014/0105907, 2014/0248266, 2014/0093497, 2014/0010812, 2013/0024956, 2013/0023047, 2013/0315900, 2012/0087927, 2012/0263732, 2012/0301488, 2011/0027276, 2011/0104182, 2010/0234578, 2009/0304687, 2009/0181015, 2009/0130715, 2009/0311254, 2008/0199471, 2008/0085531, 2016/0152721, 2015/0110783, 2015/0086991, 2015/0086559, 2014/0341898, 2014/0205602, 2014/0004131, 2013/0011405, 2012/0121585, 2011/0033456, 2011/0002934, 2010/0172912, 2009/0081242, 2009/0130095, 2008/0254026, 2008/0075727, 2009/0304706, 2009/0202531, 2009/0117111, 2009/0041773, 2008/0274118, 2008/0057070, 2007/0098717, 2007/0218060, 2007/0098718, 2007/0110754; and PCT Publication Nos. WO 2016/069919, WO 2016/023960, WO 2016/023875, WO 2016/028810, WO 2015/134988, WO 2015/091853, WO 2015/091655, WO 2014/065403, WO 2014/070934, WO 2014/065402, WO 2014/207064, WO 2013/034904, WO 2012/125569, WO 2012/149356, WO 2012/111762, WO 2012/145673, WO 2011/123489, WO 2010/123012, WO 2010/104761, WO 2009/094391, WO 2008/091954, WO 2007/129895, WO 2006/128103, WO 2005/063289, WO 2005/063981, WO 2003/040170, WO 2002/011763, WO 2000/075348, WO 2013/164789, WO 2012/075111, WO 2012/065950, WO 2009/062054, WO 2007/124299, WO 2007/053661, WO 2007/053767, WO 2005/044294, WO 2005/044304, WO 2005/044306, WO 2005/044855, WO 2005/044854, WO 2005/044305, WO 2003/045978, WO 2003/029296, WO 2002/028481, WO 2002/028480, WO 2002/028904, WO 2002/028905, WO 2002/088186, and WO 2001/024823, each of which is incorporated by reference herein.

CD122. CD122 is the Interleukin-2 receptor beta sub-unit and is known to increase proliferation of CD8⁺ effector T cells. See, e.g., Boyman et al. (2012) Nat. Rev. Immunol. 12 (3): 180-190. Multiple immune checkpoint modulators specific for CD122 have been developed and may be used as disclosed herein. In some embodiments, the immune checkpoint modulator is an agent that modulates the activity and/or expression of CD122. In some embodiments, the immune checkpoint modulator is an agent that binds to CD122 (e.g., an anti-CD122 antibody). In some embodiments, the checkpoint modulator is an CD122 agonist. In some embodiments, the checkpoint modulator is an CD22 agonist. In some embodiments, the immune checkpoint modulator is humanized MiK-Beta-1 (Roche; see, e.g., Morris et al. (2006) Proc Nat'l. Acad. Sci. USA 103(2): 401-6, which is incorporated by reference). Additional CD122-binding proteins (e.g., antibodies) are known in the art and are disclosed, e.g., in U.S. Pat. No. 9,028,830, which is incorporated by reference herein.

OX40. The OX40 receptor (also known as CD134) promotes the expansion of effector and memory T cells. OX40 also suppresses the differentiation and activity of T-regulatory cells, and regulates cytokine production (see, e.g., Croft et al. (2009) Immunol. Rev. 229(1): 173-91). Multiple immune checkpoint modulators specific for OX40 have been developed and may be used as disclosed herein. In some embodiments, the immune checkpoint modulator is an agent that modulates the activity and/or expression of OX40. In some embodiments, the immune checkpoint modulator is an agent that binds to OX40 (e.g., an anti-OX40 antibody). In some embodiments, the checkpoint modulator is an OX40 agonist. In some embodiments, the checkpoint modulator is an OX40 antagonist. In some embodiments, the immune checkpoint modulator is a OX40-binding protein (e.g., an antibody) selected from the group consisting of MEDI6469 (AgonOx/Medimmune), pogalizumab (also known as MOXR0916 and RG7888; Genentech, Inc.), tavolixizumab (also known as MEDI0562; Medimmune), and GSK3174998 (GlaxoSmithKline). Additional OX-40-binding proteins (e.g., antibodies) are known in the art and are disclosed, e.g., in U.S. Pat. Nos. 9,163,085, 9,040,048, 9,006,396, 8,748,585, 8,614,295, 8,551,477, 8,283,450, 7,550,140; U.S. Patent Application Publication Nos. 2016/0068604, 2016/0031974, 2015/0315281, 2015/0132288, 2014/0308276, 2014/0377284, 2014/0044703, 2014/0294824, 2013/0330344, 2013/0280275, 2013/0243772, 2013/0183315, 2012/0269825, 2012/0244076, 2011/0008368, 2011/0123552, 2010/0254978, 2010/0196359, 2006/0281072; and PCT Publication Nos. WO 2014/148895, WO 2013/068563, WO 2013/038191, WO 2013/028231, WO 2010/096418, WO 2007/062245, and WO 2003/106498, each of which is incorporated by reference herein.

GITR. Glucocorticoid-induced TNFR family related gene (GITR) is a member of the tumor necrosis factor receptor (TNFR) superfamily that is constitutively or conditionally expressed on Treg, CD4, and CD8 T cells. GITR is rapidly upregulated on effector T cells following TCR ligation and activation. The human GITR ligand (GITRL) is constitutively expressed on APCs in secondary lymphoid organs and some nonlymphoid tissues. The downstream effect of GITR:GITRL interaction induces attenuation of Treg activity and enhances CD4⁺ T cell activity, resulting in a reversal of Treg-mediated immunosuppression and increased immune stimulation. Multiple immune checkpoint modulators specific for GITR have been developed and may be used as disclosed herein. In some embodiments, the immune checkpoint modulator is an agent that modulates the activity and/or expression of GITR. In some embodiments, the immune checkpoint modulator is an agent that binds to GITR (e.g., an anti-GITR antibody). In some embodiments, the checkpoint modulator is an GITR agonist. In some embodiments, the checkpoint modulator is an GITR antagonist. In some embodiments, the immune checkpoint modulator is a GITR-binding protein (e.g., an antibody) selected from the group consisting of TRX518 (Leap Therapeutics), MK-4166 (Merck & Co.), MEDI-1873 (MedImmune), INCAGN1876 (Agenus/Incyte), and FPA154 (Five Prime Therapeutics). Additional GITR-binding proteins (e.g., antibodies) are known in the art and are disclosed, e.g., in U.S. Pat. Nos. 9,309,321, 9,255,152, 9,255,151, 9,228,016, 9,028,823, 8,709,424, 8,388,967; U.S. Patent Application Publication Nos. 2016/0145342, 2015/0353637, 2015/0064204, 2014/0348841, 2014/0065152, 2014/0072566, 2014/0072565, 2013/0183321, 2013/0108641, 2012/0189639; and PCT Publication Nos. WO 2016/054638, WO 2016/057841, WO 2016/057846, WO 2015/187835, WO 2015/184099, WO 2015/031667, WO 2011/028683, and WO 2004/107618, each of which is incorporated by reference herein.

ICOS. Inducible T-cell costimulator (ICOS, also known as CD278) is expressed on activated T cells. Its ligand is ICOSL, which is expressed mainly on B cells and dendritic cells. ICOS is important in T cell effector function. ICOS expression is up-regulated upon T cell activation (see, e.g., Fan et al. (2014) J. Exp. Med. 211(4): 715-25). Multiple immune checkpoint modulators specific for ICOS have been developed and may be used as disclosed herein. In some embodiments, the immune checkpoint modulator is an agent that modulates the activity and/or expression of ICOS. In some embodiments, the immune checkpoint modulator is an agent that binds to ICOS (e.g., an anti-ICOS antibody). In some embodiments, the checkpoint modulator is an ICOS agonist. In some embodiments, the checkpoint modulator is an ICOS antagonist. In some embodiments, the immune checkpoint modulator is a ICOS-binding protein (e.g., an antibody) selected from the group consisting of MEDI-570 (also known as JMab-136, Medimmune), GSK3359609 (GlaxoSmithKline/INSERM), and JTX-2011 (Jounce Therapeutics). Additional ICOS-binding proteins (e.g., antibodies) are known in the art and are disclosed, e.g., in U.S. Pat. Nos. 9,376,493, 7,998,478, 7,465,445, 7,465,444; U.S. Patent Application Publication Nos. 2015/0239978, 2012/0039874, 2008/0199466, 2008/0279851; and PCT Publication No. WO 2001/087981, each of which is incorporated by reference herein.

4-1BB. 4-1BB (also known as CD137) is a member of the tumor necrosis factor (TNF) receptor superfamily. 4-1BB (CD137) is a type II transmembrane glycoprotein that is inducibly expressed on primed CD4+ and CD8⁺ T cells, activated NK cells, DCs, and neutrophils, and acts as a T cell costimulatory molecule when bound to the 4-1BB ligand (4-1BBL) found on activated macrophages, B cells, and DCs. Ligation of the 4-1BB receptor leads to activation of the NF-κB, c-Jun and p38 signaling pathways and has been shown to promote survival of CD8⁺ T cells, specifically, by upregulating expression of the antiapoptotic genes BcL-x(L) and Bfl-1. In this manner, 4-1BB serves to boost or even salvage a suboptimal immune response. Multiple immune checkpoint modulators specific for 4-1BB have been developed and may be used as disclosed herein. In some embodiments, the immune checkpoint modulator is an agent that modulates the activity and/or expression of 4-1BB. In some embodiments, the immune checkpoint modulator is an agent that binds to 4-1BB (e.g., an anti-4-1BB antibody). In some embodiments, the checkpoint modulator is an 4-1BB agonist. In some embodiments, the checkpoint modulator is an 4-1BB antagonist. In some embodiments, the immune checkpoint modulator is a 4-1BB-binding protein is urelumab (also known as BMS-663513; Bristol-Myers Squibb) or utomilumab (Pfizer). In some embodiments, the immune checkpoint modulator is a 4-1BB-binding protein (e.g., an antibody). 4-1BB-binding proteins (e.g., antibodies) are known in the art and are disclosed, e.g., in U.S. Pat. Nos. 9,382,328, 8,716,452, 8,475,790, 8,137,667, 7,829,088, 7,659,384; U.S. Patent Application Publication Nos. 2016/0083474, 2016/0152722, 2014/0193422, 2014/0178368, 2013/0149301, 2012/0237498, 2012/0141494, 2012/0076722, 2011/0177104, 2011/0189189, 2010/0183621, 2009/0068192, 2009/0041763, 2008/0305113, 2008/0008716; and PCT Publication Nos. WO 2016/029073, WO 2015/188047, WO 2015/179236, WO 2015/119923, WO 2012/032433, WO 2012/145183, WO 2011/031063, WO 2010/132389, WO 2010/042433, WO 2006/126835, WO 2005/035584, WO 2004/010947; and Martinez-Forero et al. (2013) J. Immunol. 190(12): 6694-706, and Dubrot et al. (2010) Cancer Immunol. Immunother. 59(8): 1223-33, each of which is incorporated by reference herein.

ii. Inhibitory Immune Checkpoint Molecules

ADORA2A. The adenosine A2A receptor (A2A4) is a member of the G protein-coupled receptor (GPCR) family which possess seven transmembrane alpha helices, and is regarded as an important checkpoint in cancer therapy. A2A receptor can negatively regulate overreactive immune cells (see, e.g., Ohta et al. (2001) Nature 414(6866): 916-20). Multiple immune checkpoint modulators specific for ADORA2A have been developed and may be used as disclosed herein. In some embodiments, the immune checkpoint modulator is an agent that modulates the activity and/or expression of ADORA2A. In some embodiments, the immune checkpoint modulator is an agent that binds to ADORA2A (e.g., an anti-ADORA2A antibody). In some embodiments, the immune checkpoint modulator is a ADORA2A-binding protein (e.g., an antibody). In some embodiments, the checkpoint modulator is an ADORA2A agonist. In some embodiments, the checkpoint modulator is an ADORA2A antagonist. ADORA2A-binding proteins (e.g., antibodies) are known in the art and are disclosed, e.g., in U.S. Patent Application Publication No. 2014/0322236, which is incorporated by reference herein.

B7-H3. B7-H3 (also known as CD276) belongs to the B7 superfamily, a group of molecules that costimulate or down-modulate T-cell responses. B7-H3 potently and consistently down-modulates human T-cell responses (see, e.g., Leitner et al. (2009) Eur. J. Immunol. 39(7): 1754-64). Multiple immune checkpoint modulators specific for B7-H3 have been developed and may be used as disclosed herein. In some embodiments, the immune checkpoint modulator is an agent that modulates the activity and/or expression of B7-H3. In some embodiments, the immune checkpoint modulator is an agent that binds to B7-H3 (e.g., an anti-B7-H3 antibody). In some embodiments, the checkpoint modulator is an B7-H3 agonist. In some embodiments, the checkpoint modulator is an B7-H3 antagonist. In some embodiments, the immune checkpoint modulator is an anti-B7-H3-binding protein selected from the group consisting of DS-5573 (Daiichi Sankyo, Inc.), enoblituzumab (MacroGenics, Inc.), and 8H9 (Sloan Kettering Institute for Cancer Research; see, e.g., Ahmed et al. (2015) J. Biol. Chem. 290(50): 30018-29). In some embodiments, the immune checkpoint modulator is a B7-H3-binding protein (e.g., an antibody). B7-H3-binding proteins (e.g., antibodies) are known in the art and are disclosed, e.g., in U.S. Pat. Nos. 9,371,395, 9,150,656, 9,062,110, 8,802,091, 8,501,471, 8,414,892; U.S. Patent Application Publication Nos. 2015/0352224, 2015/0297748, 2015/0259434, 2015/0274838, 2014/032875, 2014/0161814, 2013/0287798, 2013/0078234, 2013/0149236, 2012/02947960, 2010/0143245, 2002/0102264; PCT Publication Nos. WO 2016/106004, WO 2016/033225, WO 2015/181267, WO 2014/057687, WO 2012/147713, WO 2011/109400, WO 2008/116219, WO 2003/075846, WO 2002/032375; and Shi et al. (2016) Mol. Med. Rep. 14(1): 943-8, each of which is incorporated by reference herein.

B7-H4. B7-H4 (also known as O8E, OV064, and V-set domain-containing T-cell activation inhibitor (VTCN1)), belongs to the B7 superfamily. By arresting cell cycle, B7-H4 ligation of T cells has a profound inhibitory effect on the growth, cytokine secretion, and development of cytotoxicity. Administration of B7-H4Ig into mice impairs antigen-specific T cell responses, whereas blockade of endogenous B7-H4 by specific monoclonal antibody promotes T cell responses (see, e.g., Sica et al. (2003) Immunity 18(6): 849-61). Multiple immune checkpoint modulators specific for B7-H4 have been developed and may be used as disclosed herein. In some embodiments, the immune checkpoint modulator is an agent that modulates the activity and/or expression of B7-H4. In some embodiments, the immune checkpoint modulator is an agent that binds to B7-H4 (e.g., an anti-B7-H4 antibody). In some embodiments, the immune checkpoint modulator is a B7-H4-binding protein (e.g., an antibody). In some embodiments, the checkpoint modulator is an B7-H4 agonist. In some embodiments, the checkpoint modulator is an B7-H4 antagonist. B7-H4-binding proteins (e.g., antibodies) are known in the art and are disclosed, e.g., in U.S. Pat. Nos. 9,296,822, 8,609,816, 8,759,490, 8,323,645; U.S. Patent Application Publication Nos. 2016/0159910, 2016/0017040, 2016/0168249, 2015/0315275, 2014/0134180, 2014/0322129, 2014/0356364, 2014/0328751, 2014/0294861, 2014/0308259, 2013/0058864, 2011/0085970, 2009/0074660, 2009/0208489; and PCT Publication Nos. WO 2016/040724, WO 2016/070001, WO 2014/159835, WO 2014/100483, WO 2014/100439, WO 2013/067492, WO 2013/025779, WO 2009/073533, WO 2007/067991, and WO 2006/104677, each of which is incorporated by reference herein.

BTLA. B and T Lymphocyte Attenuator (BTLA), also known as CD272, has HVEM (Herpesvirus Entry Mediator) as its ligand. Surface expression of BTLA is gradually downregulated during differentiation of human CD8⁺ T cells from the naive to effector cell phenotype, however tumor-specific human CD8⁺ T cells express high levels of BTLA (see, e.g., Derre et al. (2010) J. Clin. Invest. 120 (1): 157-67). Multiple immune checkpoint modulators specific for BTLA have been developed and may be used as disclosed herein. In some embodiments, the immune checkpoint modulator is an agent that modulates the activity and/or expression of BTLA. In some embodiments, the immune checkpoint modulator is an agent that binds to BTLA (e.g., an anti-BTLA antibody). In some embodiments, the immune checkpoint modulator is a BTLA-binding protein (e.g., an antibody). In some embodiments, the checkpoint modulator is an BTLA agonist. In some embodiments, the checkpoint modulator is an BTLA antagonist. BTLA-binding proteins (e.g., antibodies) are known in the art and are disclosed, e.g., in U.S. Pat. Nos. 9,346,882, 8,580,259, 8,563,694, 8,247,537; U.S. Patent Application Publication Nos. 2014/0017255, 2012/0288500, 2012/0183565, 2010/0172900; and PCT Publication Nos. WO 2011/014438, and WO 2008/076560, each of which is incorporated by reference herein.

CTLA-4. Cytotoxic T lymphocyte antigen-4 (CTLA-4) is a member of the immune regulatory CD28-B7 immunoglobulin superfamily and acts on naïve and resting T lymphocytes to promote immunosuppression through both B7-dependent and B7-independent pathways (see, e.g., Kim et al. (2016) J. Immunol. Res., Article ID 4683607, 14 pp.). CTLA-4 is also known as called CD152. CTLA-4 modulates the threshold for T cell activation. See, e.g., Gajewski et al. (2001) J. Immunol. 166(6): 3900-7. Multiple immune checkpoint modulators specific for CTLA-4 have been developed and may be used as disclosed herein. In some embodiments, the immune checkpoint modulator is an agent that modulates the activity and/or expression of CTLA-4. In some embodiments, the immune checkpoint modulator is an agent that binds to CTLA-4 (e.g., an anti-CTLA-4 antibody). In some embodiments, the checkpoint modulator is an CTLA-4 agonist. In some embodiments, the checkpoint modulator is an CTLA-4 antagonist. In some embodiments, the immune checkpoint modulator is a CTLA-4-binding protein (e.g., an antibody) selected from the group consisting of ipilimumab (Yervoy; Medarex/Bristol-Myers Squibb), tremelimumab (formerly ticilimumab; Pfizer/AstraZeneca), JMW-3B3 (University of Aberdeen), and AGEN1884 (Agenus). Additional CTLA-4 binding proteins (e.g., antibodies) are known in the art and are disclosed, e.g., in U.S. Pat. No. 8,697,845; U.S. Patent Application Publication Nos. 2014/0105914, 2013/0267688, 2012/0107320, 2009/0123477; and PCT Publication Nos. WO 2014/207064, WO 2012/120125, WO 2016/015675, WO 2010/097597, WO 2006/066568, and WO 2001/054732, each of which is incorporated by reference herein.

IDO. Indoleamine 2,3-dioxygenase (IDO) is a tryptophan catabolic enzyme with immune-inhibitory properties. Another important molecule is TDO, tryptophan 2,3-dioxygenase. IDO is known to suppress T and NK cells, generate and activate Tregs and myeloid-derived suppressor cells, and promote tumor angiogenesis. Prendergast et al., 2014, Cancer Immunol Immunother. 63 (7): 721-35, which is incorporated by reference herein.

Multiple immune checkpoint modulators specific for IDO have been developed and may be used as disclosed herein. In some embodiments, the immune checkpoint modulator is an agent that modulates the activity and/or expression of IDO. In some embodiments, the immune checkpoint modulator is an agent that binds to IDO (e.g., an IDO binding protein, such as an anti-IDO antibody). In some embodiments, the checkpoint modulator is an IDO agonist. In some embodiments, the checkpoint modulator is an IDO antagonist. In some embodiments, the immune checkpoint modulator is selected from the group consisting of Norharmane, Rosmarinic acid, COX-2 inhibitors, alpha-methyl-tryptophan, and Epacadostat. In one embodiment, the modulator is Epacadostat.

KIR. Killer immunoglobulin-like receptors (KIRs) comprise a diverse repertoire of MHCI binding molecules that negatively regulate natural killer (NK) cell function to protect cells from NK-mediated cell lysis. KIRs are generally expressed on NK cells but have also been detected on tumor specific CTLs. Multiple immune checkpoint modulators specific for KIR have been developed and may be used as disclosed herein. In some embodiments, the immune checkpoint modulator is an agent that modulates the activity and/or expression of KIR. In some embodiments, the immune checkpoint modulator is an agent that binds to KIR (e.g., an anti-KIR antibody). In some embodiments, the immune checkpoint modulator is a KIR-binding protein (e.g., an antibody). In some embodiments, the checkpoint modulator is an KIR agonist. In some embodiments, the checkpoint modulator is an KIR antagonist. In some embodiments the immune checkpoint modulator is lirilumab (also known as BMS-986015; Bristol-Myers Squibb). Additional KIR binding proteins (e.g., antibodies) are known in the art and are disclosed, e.g., in U.S. Pat. Nos. 8,981,065, 9,018,366, 9,067,997, 8,709,411, 8,637,258, 8,614,307, 8,551,483, 8,388,970, 8,119,775; U.S. Patent Application Publication Nos. 2015/0344576, 2015/0376275, 2016/0046712, 2015/0191547, 2015/0290316, 2015/0283234, 2015/0197569, 2014/0193430, 2013/0143269, 2013/0287770, 2012/0208237, 2011/0293627, 2009/0081240, 2010/0189723; and PCT Publication Nos. WO 2016/069589, WO 2015/069785, WO 2014/066532, WO 2014/055648, WO 2012/160448, WO 2012/071411, WO 2010/065939, WO 2008/084106, WO 2006/072625, WO 2006/072626, and WO 2006/003179, each of which is incorporated by reference herein.

LAG-3, Lymphocyte-activation gene 3 (LAG-3, also known as CD223) is a CD4-related transmembrane protein that competitively binds MHC II and acts as a co-inhibitory checkpoint for T cell activation (see, e.g., Goldberg and Drake (2011) Curr. Top. Microbiol. Immunol. 344: 269-78). Multiple immune checkpoint modulators specific for LAG-3 have been developed and may be used as disclosed herein. In some embodiments, the immune checkpoint modulator is an agent that modulates the activity and/or expression of LAG-3. In some embodiments, the immune checkpoint modulator is an agent that binds to LAG-3 (e.g., an anti-PD-1 antibody). In some embodiments, the checkpoint modulator is an LAG-3 agonist. In some embodiments, the checkpoint modulator is an LAG-3 antagonist. In some embodiments, the immune checkpoint modulator is a LAG-3-binding protein (e.g., an antibody) selected from the group consisting of pembrolizumab (Keytruda; formerly lambrolizumab; Merck & Co., Inc.), nivolumab (Opdivo; Bristol-Myers Squibb), pidilizumab (CT-011, CureTech), SHR-1210 (Incyte/Jiangsu Hengrui Medicine Co., Ltd.), MEDI0680 (also known as AMP-514; Amplimmune Inc./Medimmune), PDR001 (Novartis), BGB-A317 (BeiGene Ltd.), TSR-042 (also known as ANB011; AnaptysBio/Tesaro, Inc.), REGN2810 (Regeneron Pharmaceuticals, Inc./Sanofi-Aventis), and PF-06801591 (Pfizer). Additional PD-1-binding proteins (e.g., antibodies) are known in the art and are disclosed, e.g., in U.S. Pat. Nos. 9,181,342, 8,927,697, 7,488,802, 7,029,674; U.S. Patent Application Publication Nos. 2015/0152180, 2011/0171215, 2011/0171220; and PCT Publication Nos. WO 2004/056875, WO 2015/036394, WO 2010/029435, WO 2010/029434, WO 2014/194302, each of which is incorporated by reference herein.

PD-1. Programmed cell death protein 1 (PD-1, also known as CD279 and PDCD1) is an inhibitory receptor that negatively regulates the immune system. In contrast to CTLA-4 which mainly affects naïve T cells, PD-1 is more broadly expressed on immune cells and regulates mature T cell activity in peripheral tissues and in the tumor microenvironment. PD-1 inhibits T cell responses by interfering with T cell receptor signaling. PD-1 has two ligands, PD-L1 and PD-L2. Multiple immune checkpoint modulators specific for PD-1 have been developed and may be used as disclosed herein. In some embodiments, the immune checkpoint modulator is an agent that modulates the activity and/or expression of PD-1. In some embodiments, the immune checkpoint modulator is an agent that binds to PD-1 (e.g., an anti-PD-1 antibody). In some embodiments, the checkpoint modulator is an PD-1 agonist. In some embodiments, the checkpoint modulator is an PD-1 antagonist. In some embodiments, the immune checkpoint modulator is a PD-1-binding protein (e.g., an antibody) selected from the group consisting of pembrolizumab (Keytruda; formerly lambrolizumab; Merck & Co., Inc.), nivolumab (Opdivo; Bristol-Myers Squibb), pidilizumab (CT-011, CureTech), SHR-1210 (Incyte/Jiangsu Hengrui Medicine Co., Ltd.), MEDI0680 (also known as AMP-514; Amplimmune Inc./Medimmune), PDR001 (Novartis), BGB-A317 (BeiGene Ltd.), TSR-042 (also known as ANB011; AnaptysBio/Tesaro, Inc.), REGN2810 (Regeneron Pharmaceuticals, Inc./Sanofi-Aventis), and PF-06801591 (Pfizer). Additional PD-1-binding proteins (e.g., antibodies) are known in the art and are disclosed, e.g., in U.S. Pat. Nos. 9,181,342, 8,927,697, 7,488,802, 7,029,674; U.S. Patent Application Publication Nos. 2015/0152180, 2011/0171215, 2011/0171220; and PCT Publication Nos. WO 2004/056875, WO 2015/036394, WO 2010/029435, WO 2010/029434, WO 2014/194302, each of which is incorporated by reference herein.

PD-L1/PD-L2. PD ligand 1 (PD-L1, also knows as B7-H1) and PD ligand 2 (PD-L2, also known as PDCD1LG2, CD273, and B7-DC) bind to the PD-1 receptor. Both ligands belong to the same B7 family as the B7-1 and B7-2 proteins that interact with CD28 and CTLA-4. PD-L1 can be expressed on many cell types including, for example, epithelial cells, endothelial cells, and immune cells. Ligation of PDL-1 decreases IFNγ, TNFα, and IL-2 production and stimulates production of IL10, an anti-inflammatory cytokine associated with decreased T cell reactivity and proliferation as well as antigen-specific T cell anergy. PDL-2 is predominantly expressed on antigen presenting cells (APCs). PDL2 ligation also results in T cell suppression, but where PDL-1-PD-1 interactions inhibits proliferation via cell cycle arrest in the G1/G2 phase, PDL2-PD-1 engagement has been shown to inhibit TCR-mediated signaling by blocking B7:CD28 signals at low antigen concentrations and reducing cytokine production at high antigen concentrations. Multiple immune checkpoint modulators specific for PD-L1 and PD-L2 have been developed and may be used as disclosed herein.

In some embodiments, the immune checkpoint modulator is an agent that modulates the activity and/or expression of PD-L1. In some embodiments, the immune checkpoint modulator is an agent that binds to PD-L1 (e.g., an anti-PD-L1 antibody). In some embodiments, the checkpoint modulator is an PD-L1 agonist. In some embodiments, the checkpoint modulator is an PD-L1 antagonist. In some embodiments, the immune checkpoint modulator is a PD-L1-binding protein (e.g., an antibody or a Fc-fusion protein) selected from the group consisting of durvalumab (also known as MEDI-4736; AstraZeneca/Celgene Corp./Medimmune), atezolizumab (Tecentriq; also known as MPDL3280A and RG7446; Genetech Inc.), avelumab (also known as MSB0010718C; Merck Serono/AstraZeneca); MDX-1105 (Medarex/Bristol-Meyers Squibb), AMP-224 (Amplimmune, GlaxoSmithKline), LY3300054 (Eli Lilly and Co.). Additional PD-L1-binding proteins are known in the art and are disclosed, e.g., in U.S. Patent Application Publication Nos. 2016/0084839, 2015/0355184, 2016/0175397, and PCT Publication Nos. WO 2014/100079, WO 2016/030350, WO2013181634, each of which is incorporated by reference herein.

In some embodiments, the immune checkpoint modulator is an agent that modulates the activity and/or expression of PD-L2. In some embodiments, the immune checkpoint modulator is an agent that binds to PD-L2 (e.g., an anti-PD-L2 antibody). In some embodiments, the checkpoint modulator is an PD-L2 agonist. In some embodiments, the checkpoint modulator is an PD-L2 antagonist. PD-L2-binding proteins (e.g., antibodies) are known in the art and are disclosed, e.g., in U.S. Pat. Nos. 9,255,147, 8,188,238; U.S. Patent Application Publication Nos. 2016/0122431, 2013/0243752, 2010/0278816, 2016/0137731, 2015/0197571, 2013/0291136, 2011/0271358; and PCT Publication Nos. WO 2014/022758, and WO 2010/036959, each of which is incorporated by reference herein.

TIM-3. T cell immunoglobulin mucin 3 (TIM-3, also known as Hepatitis A virus cellular receptor (HAVCR2)) is a A type I glycoprotein receptor that binds to S-type lectin galectin-9 (Gal-9). TIM-3, is a widely expressed ligand on lymphocytes, liver, small intestine, thymus, kidney, spleen, lung, muscle, reticulocytes, and brain tissue. Tim-3 was originally identified as being selectively expressed on IFN-7-secreting Th1 and Tc1 cells (Monney et al. (2002) Nature 415: 536-41). Binding of Gal-9 by the TIM-3 receptor triggers downstream signaling to negatively regulate T cell survival and function. Multiple immune checkpoint modulators specific for TIM-3 have been developed and may be used as disclosed herein. In some embodiments, the immune checkpoint modulator is an agent that modulates the activity and/or expression of TIM-3. In some embodiments, the immune checkpoint modulator is an agent that binds to TIM-3 (e.g., an anti-TIM-3 antibody). In some embodiments, the checkpoint modulator is an TIM-3 agonist. In some embodiments, the checkpoint modulator is an TIM-3 antagonist. In some embodiments, the immune checkpoint modulator is an anti-TIM-3 antibody selected from the group consisting of TSR-022 (AnaptysBio/Tesaro, Inc.) and MGB453 (Novartis). Additional TIM-3 binding proteins (e.g., antibodies) are known in the art and are disclosed, e.g., in U.S. Pat. Nos. 9,103,832, 8,552,156, 8,647,623, 8,841,418; U.S. Patent Application Publication Nos. 2016/0200815, 2015/0284468, 2014/0134639, 2014/0044728, 2012/0189617, 2015/0086574, 2013/0022623; and PCT Publication Nos. WO 2016/068802, WO 2016/068803, WO 2016/071448, WO 2011/155607, and WO 2013/006490, each of which is incorporated by reference herein.

VISTA. V-domain Ig suppressor of T cell activation (VISTA, also known as Platelet receptor Gi24) is an Ig super-family ligand that negatively regulates T cell responses. See, e.g., Wang et al., 2011, J. Exp. Med. 208: 577-92. VISTA expressed on APCs directly suppresses CD4⁺ and CD8⁺ T cell proliferation and cytokine production (Wang et al. (2010) J Exp Med. 208(3): 577-92). Multiple immune checkpoint modulators specific for VISTA have been developed and may be used as disclosed herein. In some embodiments, the immune checkpoint modulator is an agent that modulates the activity and/or expression of VISTA. In some embodiments, the immune checkpoint modulator is an agent that binds to VISTA (e.g., an anti-VISTA antibody). In some embodiments, the checkpoint modulator is an VISTA agonist. In some embodiments, the checkpoint modulator is an VISTA antagonist. In some embodiments, the immune checkpoint modulator is a VISTA-binding protein (e.g., an antibody) selected from the group consisting of TSR-022 (AnaptysBio/Tesaro, Inc.) and MGB453 (Novartis). VISTA-binding proteins (e.g., antibodies) are known in the art and are disclosed, e.g., in U.S. Patent Application Publication Nos. 2016/0096891, 2016/0096891; and PCT Publication Nos. WO 2014/190356, WO 2014/197849, WO 2014/190356 and WO 2016/094837, each of which is incorporated by reference herein.

In certain embodiments, the immune checkpoint modulator stimulates the immune response of the subject. For example, in some embodiments, the immune checkpoint modulator stimulates or increases the expression or activity of a stimulatory immune checkpoint (e.g. CD27, CD28, CD40, CD122, OX40, GITR, ICOS, or 4-1BB). In some embodiments, the immune checkpoint modulator inhibits or decreases the expression or activity of an inhibitory immune checkpoint (e.g. A2A4, B7-H3, B7-H4, BTLA, CTLA-4, IDO, KIR, LAG3, PD-1, PD-L1, PD-L2, TIM-3 or VISTA).

In certain embodiments the immune checkpoint modulator targets an immune checkpoint molecule selected from the group consisting of CD27, CD28, CD40, CD122, OX40, GITR, ICOS, 4-1BB, A2A4, B7-H3, B7-H4, BTLA, CTLA-4, IDO, KIR, LAG3, PD-1, PD-L1, PD-L2, TIM-3 and VISTA. In certain embodiments the immune checkpoint modulator targets an immune checkpoint molecule selected from the group consisting of CD27, CD28, CD40, CD122, OX40, GITR, ICOS, 4-1BB, A2A4, B7-H3, B7-H4, BTLA, IDO, KIR, LAG3, PD-1, PD-L1, PD-L2, TIM-3 and VISTA. In a particular embodiment, the immune checkpoint modulator targets an immune checkpoint molecule selected from the group consisting of CTLA-4, PD-L1 and PD-1. In a further particular embodiment the immune checkpoint modulator targets an immune checkpoint molecule selected from PD-L1 and PD-1.

In some embodiments, more than one (e.g. 2, 3, 4, 5 or more) immune checkpoint modulator is administered to the subject. Where more than one immune checkpoint modulator is administered, the modulators may each target a stimulatory immune checkpoint molecule, or each target an inhibitory immune checkpoint molecule. In other embodiments, the immune checkpoint modulators include at least one modulator targeting a stimulatory immune checkpoint and at least one immune checkpoint modulator targeting an inhibitory immune checkpoint molecule. In certain embodiments, the immune checkpoint modulator is a binding protein, for example, an antibody. The term “binding protein”, as used herein, refers to a protein or polypeptide that can specifically bind to a target molecule, e.g. an immune checkpoint molecule. In some embodiments the binding protein is an antibody or antigen binding portion thereof, and the target molecule is an immune checkpoint molecule. In some embodiments the binding protein is a protein or polypeptide that specifically binds to a target molecule (e.g., an immune checkpoint molecule). In some embodiments the binding protein is a ligand. In some embodiments, the binding protein is a fusion protein. In some embodiments, the binding protein is a receptor. Examples of binding proteins that may be used in the methods of the invention include, but are not limited to, a humanized antibody, an antibody Fab fragment, a divalent antibody, an antibody drug conjugate, a scFv, a fusion protein, a bivalent antibody, and a tetravalent antibody.

The term “antibody”, as used herein, refers to any immunoglobulin (Ig) molecule comprised of four polypeptide chains, two heavy (H) chains and two light (L) chains, or any functional fragment, mutant, variant, or derivation thereof. Such mutant, variant, or derivative antibody formats are known in the art. In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG 3, IgG4, IgA1 and IgA2) or subclass. In some embodiments, the antibody is a full-length antibody. In some embodiments, the antibody is a murine antibody. In some embodiments, the antibody is a human antibody. In some embodiments, the antibody is a humanized antibody. In other embodiments, the antibody is a chimeric antibody. Chimeric and humanized antibodies may be prepared by methods well known to those of skill in the art including CDR grafting approaches (see, e.g., U.S. Pat. Nos. 5,843,708; 6,180,370; 5,693,762; 5,585,089; and 5,530,101), chain shuffling strategies (see, e.g., U.S. Pat. No. 5,565,332; Rader et al. (1998) PROC. NAT'L. ACAD. SCI. USA 95: 8910-8915), molecular modeling strategies (U.S. Pat. No. 5,639,641), and the like.

The term “antigen-binding portion” of an antibody (or simply “antibody portion”), as used herein, refers to one or more fragments of an antibody that retain the ability to specifically bind to an antigen. It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) NATURE 341: 544-546; and WO 90/05144 A1, the contents of which are herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) SCIENCE 242:423-426; and Huston et al. (1988) PROC. NAT'L. ACAD. SCI. USA 85:5879-5883). Such single chain antibodies are also intended to be encompassed within the term “antigen-binding portion” of an antibody. Other forms of single chain antibodies, such as diabodies are also encompassed. Antigen binding portions can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005).

As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., SEQUENCES OF PROTEINS OF IMMUNOLOGICAL INTEREST (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers found that certain sub-portions within Kabat CDRs adopt nearly identical peptide backbone conformations, despite having great diversity at the level of amino acid sequence (Chothia et al. (1987) J. MOL. BIOL. 196: 901-917, and Chothia et al. (1989) NATURE 342: 877-883). These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan et al. (1995) FASEB J. 9: 133-139, and MacCallum et al. (1996) J. MOL. BIOL. 262(5): 732-45. Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

The term “humanized antibody”, as used herein refers to non-human (e.g., murine) antibodies that are chimeric immunoglobulins, immunoglobulin chains, or fragments thereof (such as Fv, Fab, Fab′, F(ab′)2 or other antigen-binding subsequences of antibodies) which contain minimal sequence derived from a non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al. (1986) NATURE 321: 522-525; Reichmann et al. (1988) NATURE 332: 323-329; and Presta (1992) CURR. OP. STRUCT. BIOL. 2: 593-596, each of which is incorporated by reference herein in its entirety.

The term “immunoconjugate” or “antibody drug conjugate” as used herein refers to the linkage of an antibody or an antigen binding fragment thereof with another agent, such as a chemotherapeutic agent, a toxin, an immunotherapeutic agent, an imaging probe, and the like. The linkage can be covalent bonds, or non-covalent interactions such as through electrostatic forces. Various linkers, known in the art, can be employed in order to form the immunoconjugate. Additionally, the immunoconjugate can be provided in the form of a fusion protein that may be expressed from a polynucleotide encoding the immunoconjugate. As used herein, “fusion protein” refers to proteins created through the joining of two or more genes or gene fragments which originally coded for separate proteins (including peptides and polypeptides). Translation of the fusion gene results in a single protein with functional properties derived from each of the original proteins.

A “bivalent antibody” refers to an antibody or antigen-binding fragment thereof that comprises two antigen-binding sites. The two antigen binding sites may bind to the same antigen, or they may each bind to a different antigen, in which case the antibody or antigen-binding fragment is characterized as “bispecific.” A “tetravalent antibody” refers to an antibody or antigen-binding fragment thereof that comprises four antigen-binding sites. In certain embodiments, the tetravalent antibody is bispecific. In certain embodiments, the tetravalent antibody is multispecific, i.e. binding to more than two different antigens.

Fab (fragment antigen binding) antibody fragments are immunoreactive polypeptides comprising monovalent antigen-binding domains of an antibody composed of a polypeptide consisting of a heavy chain variable region (V_{H}) and heavy chain constant region 1 (C_{H1}) portion and a poly peptide consisting of a light chain variable (V_{L}) and light chain constant (C_{L}) portion, in which the C_{L} and C_{H1} portions are bound together, preferably by a disulfide bond between Cys residues.

Immune checkpoint modulator antibodies include, but are not limited to, at least 4 major categories: i) antibodies that block an inhibitory pathway directly on T cells or natural killer (NK) cells (e.g., PD-1 targeting antibodies such as nivolumab and pembrolizumab, antibodies targeting TIM-3, and antibodies targeting LAG-3, 2B4, CD160, A2aR, BTLA, CGEN-15049, and KIR), ii) antibodies that activate stimulatory pathways directly on T cells or NK cells (e.g., antibodies targeting OX40, GITR, and 4-1BB), iii) antibodies that block a suppressive pathway on immune cells or relies on antibody-dependent cellular cytotoxicity to deplete suppressive populations of immune cells (e.g., CTLA-4 targeting antibodies such as ipilimumab, antibodies targeting VISTA, and antibodies targeting PD-L2, Gr1, and Ly6G), and iv) antibodies that block a suppressive pathway directly on cancer cells or that rely on antibody-dependent cellular cytotoxicity to enhance cytotoxicity to cancer cells (e.g., rituximab, antibodies targeting PD-L1, and antibodies targeting B7-H3, B7-H4, Gal-9, and MUC1). Examples of checkpoint inhibitors include, e.g., an inhibitor of CTLA-4, such as ipilimumab or tremelimumab; an inhibitor of the PD-1 pathway such as an anti-PD-1, anti-PD-L1 or anti-PD-L2 antibody. Exemplary anti-PD-1 antibodies are described in WO 2006/121168, WO 2008/156712, WO 2012/145493, WO 2009/014708 and WO 2009/114335. Exemplary anti-PD-L1 antibodies are described in WO 2007/005874, WO 2010/077634 and WO 2011/066389, and exemplary anti-PD-L2 antibodies are described in WO 2004/007679.

In a particular embodiment, the immune checkpoint modulator is a fusion protein, for example, a fusion protein that modulates the activity of an immune checkpoint modulator.

In one embodiment, the immune checkpoint modulator is a therapeutic nucleic acid molecule, for example a nucleic acid that modulates the expression of an immune checkpoint protein or mRNA. Nucleic acid therapeutics are well known in the art. Nucleic acid therapeutics include both single stranded and double stranded (i.e., nucleic acid therapeutics having a complementary region of at least 15 nucleotides in length) nucleic acids that are complementary to a target sequence in a cell. In certain embodiments, the nucleic acid therapeutic is targeted against a nucleic acid sequence encoding an immune checkpoint protein.

Antisense nucleic acid therapeutic agents are single stranded nucleic acid therapeutics, typically about 16 to 30 nucleotides in length, and are complementary to a target nucleic acid sequence in the target cell, either in culture or in an organism.

In another aspect, the agent is a single-stranded antisense RNA molecule. An antisense RNA molecule is complementary to a sequence within the target mRNA. Antisense RNA can inhibit translation in a stoichiometric manner by base pairing to the mRNA and physically obstructing the translation machinery, see Dias, N. et al., (2002) Mol Cancer Ther 1:347-355. The antisense RNA molecule may have about 15-30 nucleotides that are complementary to the target mRNA. Patents directed to antisense nucleic acids, chemical modifications, and therapeutic uses include, for example: U.S. Pat. No. 5,898,031 related to chemically modified RNA-containing therapeutic compounds; U.S. Pat. No. 6,107,094 related methods of using these compounds as therapeutic agents; U.S. Pat. No. 7,432,250 related to methods of treating patients by administering single-stranded chemically modified RNA-like compounds; and U.S. Pat. No. 7,432,249 related to pharmaceutical compositions containing single-stranded chemically modified RNA-like compounds. U.S. Pat. No. 7,629,321 is related to methods of cleaving target mRNA using a single-stranded oligonucleotide having a plurality of RNA nucleosides and at least one chemical modification. The entire contents of each of the patents listed in this paragraph are incorporated herein by reference.

Nucleic acid therapeutic agents for use in the methods of the invention also include double stranded nucleic acid therapeutics. An “RNAi agent,” “double stranded RNAi agent,” double-stranded RNA (dsRNA) molecule, also referred to as “dsRNA agent,” “dsRNA”, “siRNA”, “iRNA agent,” as used interchangeably herein, refers to a complex of ribonucleic acid molecules, having a duplex structure comprising two anti-parallel and substantially complementary, as defined below, nucleic acid strands. As used herein, an RNAi agent can also include dsiRNA (see, e.g., US Patent publication 20070104688, incorporated herein by reference). In general, the majority of nucleotides of each strand are ribonucleotides, but as described herein, each or both strands can also include one or more non-ribonucleotides, e.g., a deoxyribonucleotide and/or a modified nucleotide. In addition, as used in this specification, an “RNAi agent” may include ribonucleotides with chemical modifications; an RNAi agent may include substantial modifications at multiple nucleotides. Such modifications may include all types of modifications disclosed herein or known in the art. Any such modifications, as used in a siRNA type molecule, are encompassed by “RNAi agent” for the purposes of this specification and claims. The RNAi agents that are used in the methods of the invention include agents with chemical modifications as disclosed, for example, in WO/2012/037254, and WO 2009/073809, the entire contents of each of which are incorporated herein by reference.

Immune checkpoint modulators may be administered at appropriate dosages to treat the oncological disorder, for example, by using standard dosages. One skilled in the art would be able, by routine experimentation, to determine what an effective, non-toxic amount of an immune checkpoint modulator would be for the purpose of treating oncological disorders. Standard dosages of immune checkpoint modulators are known to a person skilled in the art and may be obtained, for example, from the product insert provided by the manufacturer of the immune checkpoint modulator. Examples of standard dosages of immune checkpoint modulators are provided in Table 10 below. In other embodiments, the immune checkpoint modulator is administered at a dosage that is different (e.g. lower) than the standard dosages of the immune checkpoint modulator used to treat the oncological disorder under the standard of care for treatment for a particular oncological disorder.

TABLE 10
Exemplary Standard Dosages of Immune Checkpoint Modulators

| | Immune | |
|---|---|---|
| Immune | Checkpoint | |
| Checkpoint | Molecule | |
| Modulator | Targeted | Exemplary Standard Dosage |
| Ipilimumab | CTLA-4 | 3 mg/kg administered intravenously over |
| (Yervoy ™) | | 90 minutes every 3 weeks for a total of |
| | | 4 doses |
| Pembrolizumab | PD-1 | 2 mg/kg administered as an intravenous |
| (Keytruda ™) | | infusion over 30 minutes every 3 weeks |
| | | until disease progression or unacceptable |
| | | toxicity |
| Atezolizumab | PD-L1 | 1200 mg administered as an intravenous |
| (Tecentriq ™) | | infusion over 60 minutes every 3 weeks |

In certain embodiments, the administered dosage of the immune checkpoint modulator is 5%, 10%, 20%, 30%, 40%, 50%, 60%, 70%, 80%, or 90% lower than the standard dosage of the immune checkpoint modulator for a particular oncological disorder. In certain embodiments, the dosage administered of the immune checkpoint modulator is 95%, 90%, 85%, 80%, 75%, 70%, 65%, 60%, 55%, 50%, 45%, 40%, 35%, 30%, 25%, 20%, 15%, 10% or 5% of the standard dosage of the immune checkpoint modulator for a particular oncological disorder. In one embodiment, where a combination of immune checkpoint modulators are administered, at least one of the immune checkpoint modulators is administered at a dose that is lower than the standard dosage of the immune checkpoint modulator for a particular oncological disorder. In one embodiment, where a combination of immune checkpoint modulators are administered, at least two of the immune checkpoint modulators are administered at a dose that is lower than the standard dosage of the immune checkpoint modulators for a particular oncological disorder. In one embodiment, where a combination of immune checkpoint modulators are administered, at least three of the immune checkpoint modulators are administered at a dose that is lower than the standard dosage of the immune checkpoint modulators for a particular oncological disorder. In one embodiment, where a combination of immune checkpoint modulators are administered, all of the immune checkpoint modulators are administered at a dose that is lower than the standard dosage of the immune checkpoint modulators for a particular oncological disorder.

Additional immunotherapeutics that may be used in the methods disclosed herein include, but are not limited to, Toll-like receptor (TLR) agonists, cell-based therapies, cytokines and cancer vaccines.

TLR Agonists

TLRs are single membrane-spanning non-catalytic receptors that recognize structurally conserved molecules derived from microbes. TLRs together with the Interleukin-1 receptor form a receptor superfamily, known as the “Interleukin-1 Receptor/Toll-Like Receptor Superfamily.” Members of this family are characterized structurally by an extracellular leucine-rich repeat (LRR) domain, a conserved pattern of juxtamembrane cysteine residues, and an intracytoplasmic signaling domain that forms a platform for downstream signaling by recruiting TIR domain-containing adapters including MyD88, TIR domain-containing adaptor (TRAP), and TIR domain-containing adaptor inducing IFNβ (TRIF) (O'Neill et al., 2007, Nat Rev Immunol 7, 353).

The TLRs include TLR1, TLR2, TLR3, TLR4, TLR5, TLR6, TLR7, TLR8, TLR9, and TLR10. TLR2 mediates cellular responses to a large number of microbial products including peptidoglycan, bacterial lipopeptides, lipoteichoic acid, mycobacterial lipoarabinomannan and yeast cell wall components. TLR4 is a transmembrane protein which belongs to the pattern recognition receptor (PRR) family. Its activation leads to an intracellular signaling pathway NF-κB and inflammatory cytokine production which is responsible for activating the innate immune system. TLR5 is known to recognize bacterial flagellin from invading mobile bacteria, and has been shown to be involved in the onset of many diseases, including inflammatory bowel disease.

TLR agonists are known in the art and are described, for example, in US2014/0030294, which is incorporated by reference herein in its entirety. Exemplary TLR2 agonists include mycobacterial cell wall glycolipids, lipoarabinomannan (LAM) and mannosylated phosphatidylinositol (PIIM), MALP-2 and Pam3Cys and synthetic variants thereof. Exemplary TLR4 agonists include lipopolysaccharide or synthetic variants thereof (e.g., MPL and RC529) and lipid A or synthetic variants thereof (e.g., aminoalkyl glucosaminide 4-phosphates). See, e.g., Cluff et al., 2005, Infection and Immunity, p. 3044-3052:73; Lembo et al., 2008, The Journal of Immunology 180, 7574-7581; and Evans et al., 2003, Expert Rev Vaccines 2:219-29. Exemplary TLR5 agonists include flagellin or synthetic variants thereof (e.g., A pharmacologically optimized TLR5 agonist with reduced immunogenicity (such as CBLB502) made by deleting portions of flagellin that are non-essential for TLR5 activation).

Additional TLR agonists include Coley's toxin and Bacille Calmette-Guérin (BCG). Coley's toxin is a mixture consisting of killed bacteria of species Streptococcus pyogenes and Serratia marcescens. See Taniguchi et al., 2006, Anticancer Res. 26 (6A): 3997-4002. BCG is prepared from a strain of the attenuated live bovine tuberculosis bacillus, Mycobacterium bovis. See Venkataswamy et al., 2012, Vaccine. 30 (6): 1038-1049.

Cell Based Therapies

Cell-based therapies for the treatment of cancer include administration of immune cells (e.g. T cells, tumor-infiltrating lymphocytes (TILs), Natural Killer cells, and dendritic cells) to a subject. In autologous cell-based therapy, the immune cells are derived from the same subject to which they are administered. In allogeneic cell-based therapy, the immune cells are derived from one subject and administered to a different subject. The immune cells may be activated, for example, by treatment with a cytokine, before administration to the subject. In some embodiments, the immune cells are genetically modified before administration to the subject, for example, as in chimeric antigen receptor (CAR) T cell immunotherapy.

In some embodiments, the cell-based therapy include an adoptive cell transfer (ACT). ACT typically consists of three parts: lympho-depletion, cell administration, and therapy with high doses of IL-2. Types of cells that may be administered in ACT include tumor infiltrating lymphocytes (TILs), T cell receptor (TCR)-transduced T cells, and chimeric antigen receptor (CAR) T cells.

Tumor-infiltrating lymphocytes are immune cells that have been observed in many solid tumors, including breast cancer. They are a population of cells comprising a mixture of cytotoxic T cells and helper T cells, as well as B cells, macrophages, natural killer cells, and dendritic cells. The general procedure for autologous TIL therapy is as follows: (1) a resected tumor is digested into fragments; (2) each fragment is grown in IL-2 and the lymphocytes proliferate destroying the tumor; (3) after a pure population of lymphocytes exists, these lymphocytes are expanded; and (4) after expansion up to 10¹¹ cells, lymphocytes are infused into the patient. See Rosenberg et al., 2015, Science 348(6230):62-68, which is incorporated by reference herein in its entirety.

TCR-transduced T cells are generated via genetic induction of tumor-specific TCRs. This is often done by cloning the particular antigen-specific TCR into a retroviral backbone. Blood is drawn from patients and peripheral blood mononuclear cells (PBMCs) are extracted. PBMCs are stimulated with CD3 in the presence of IL-2 and then transduced with the retrovirus encoding the antigen-specific TCR. These transduced PBMCs are expanded further in vitro and infused back into patients. See Robbins et al., 2015, Clinical Cancer Research 21(5):1019-1027, which is incorporated by reference herein in its entirety.

Chimeric antigen receptors (CARs) are recombinant receptors containing an extracellular antigen recognition domain, a transmembrane domain, and a cytoplasmic signaling domain (such as CD3ζ, CD28, and 4-1BB). CARs possess both antigen-binding and T-cell-activating functions. Therefore, T cells expressing CARs can recognize a wide range of cell surface antigens, including glycolipids, carbohydrates, and proteins, and can attack malignant cells expressing these antigens through the activation of cytoplasmic costimulation. See Pang et al., 2018, Mol Cancer 17: 91, which is incorporated by reference herein in its entirety.

In some embodiments, the cell-based therapy is a Natural Killer (NK) cell-based therapy. NK cells are large, granular lymphocytes that have the ability to kill tumor cells without any prior sensitization or restriction of major histocompatibility complex (MHC) molecule expression. See Uppendahl et al., 2017, Frontiers in Immunology 8: 1825. Adoptive transfer of autologous lymphokine-activated killer (LAK) cells with high-dose IL-2 therapy have been evaluated in human clinical trials. Similar to LAK immunotherapy, cytokine-induced killer (CIK) cells arise from peripheral blood mononuclear cell cultures with stimulation of anti-CD3 mAb, IFN-γ, and IL-2. CIK cells are characterized by a mixed T-NK phenotype (CD3+CD56+) and demonstrate enhanced cytotoxic activity compared to LAK cells against ovarian and cervical cancer. Human clinical trials investigating adoptive transfer of autologous CIK cells following primary debulking surgery and adjuvant carboplatin/paclitaxel chemotherapy have also been conducted. See Liu et al., 2014, J Immunother 37(2): 116-122.

In some embodiments, the cell-based therapy is a dendritic cell-based immunotherapy. Vaccination with dendritic cells (DC)s treated with tumor lysates has been shown to increase therapeutic antitumor immune responses both in vitro and in vivo. See Jung et al., 2018, Translational Oncology 11(3): 686-690. DCs capture and process antigens, migrate into lymphoid organs, express lymphocyte costimulatory molecules, and secrete cytokines that initiate immune responses. They also stimulate immunological effector cells (T cells) that express receptors specific for tumor-associated antigens and reduce the number of immune repressors such as CD4+CD25+Foxp3+ regulatory T (Treg) cells. For example, a DC vaccination strategy for renal cell carcinoma (RCC), which is based on a tumor cell lysate-DC hybrid, showed therapeutic potential in preclinical and clinical trials. See Lim et al., 2007, Cancer Immunol Immunother 56: 1817-1829.

Cytokines

Several cytokines including IL-2, IL-12, IL-15, IL-18, and IL-21 have been used in the treatment of cancer for activation of immune cells such as NK cells and T cells. IL-2 was one of the first cytokines used clinically, with hopes of inducing antitumor immunity. As a single agent at high dose IL-2 induces remissions in some patients with renal cell carcinoma (RCC) and metastatic melanoma. Low dose IL-2 has also been investigated and aimed at selectively ligating the IL-2 αβγ receptor (IL-2Rαβγ) in an effort to reduce toxicity while maintaining biological activity. See Romee et al., 2014, Scientifica, Volume 2014, Article ID 205796, 18 pages, which is incorporated by reference herein in its entirety.

Interleukin-15 (IL-15) is a cytokine with structural similarity to Interleukin-2 (IL-2). Like IL-2, IL-15 binds to and signals through a complex composed of IL-2/IL-15 receptor beta chain (CD122) and the common gamma chain (gamma-C, CD132). Recombinant IL-15 has been evaluated for treatment of solid tumors (e.g. melanoma, renal cell carcinoma) and to support NK cells after adoptive transfer in cancer patients. See Romee et al., cited above.

IL-12 is a heterodimeric cytokine composed of p35 and p40 subunits (IL-12α and β chains), originally identified as “NK cell stimulatory factor (NKSF)” based on its ability to enhance NK cell cytotoxicity. Upon encounter with pathogens, IL-12 is released by activated dendritic cells and macrophages and binds to its cognate receptor, which is primarily expressed on activated T and NK cells. Numerous preclinical studies have suggested that IL-12 has antitumor potential. See Romee et al., cited above.

IL-18 is a member of the proinflammatory IL-1 family and, like IL-12, is secreted by activated phagocytes. IL-18 has demonstrated significant antitumor activity in preclinical animal models, and has been evaluated in human clinical trials. See Robertson et al., 2006, Clinical Cancer Research 12: 4265-4273.

IL-21 has been used for antitumor immunotherapy due to its ability to stimulate NK cells and CD8+ T cells. For ex vivo NK cell expansion, membrane bound IL-21 has been expressed in K562 stimulator cells, with effective results. See Denman et al., 2012, PLoS One 7(1)e30264. Recombinant human IL-21 was also shown to increase soluble CD25 and induce expression of perforin and granzyme B on CD8+ cells. IL-21 has been evaluated in several clinical trials for treatment of solid tumors. See Romee et al., cited above.

Cancer Vaccines

Therapeutic cancer vaccines eliminate cancer cells by strengthening a patients' own immune responses to the cancer, particularly CD8+ T cell mediated responses, with the assistance of suitable adjuvants. The therapeutic efficacy of cancer vaccines is dependent on the differential expression of tumor associated antigens (TAAs) by tumor cells relative to normal cells. TAAs derive from cellular proteins and should be mainly or selectively expressed on cancer cells to avoid either immune tolerance or autoimmunity effects. See Circelli et al., 2015, Vaccines 3(3): 544-555. Cancer vaccines include, for example, dendritic cell (DC) based vaccines, peptide/protein vaccines, genetic vaccines, and tumor cell vaccines. See Ye et al., 2018, J Cancer 9(2): 263-268.

### VI. Increasing Immune Activity

The combination therapies described herein may be used to increase immune activity in a cell, tissue or in a subject, for example, a subject who would benefit from increased immune activity. As provided herein, an agent that induces iron-dependent cellular disassembly (e.g., ferroptosis) can activate immune cells (e.g., T cells, B cells, NK cells, etc.) and, therefore, can enhance immune cell functions such as, for example, that involved in immunotherapies. The ability of cancer cells to harness a range of complex, overlapping mechanisms to prevent the immune system from distinguishing self from non-self represents the fundamental mechanism of cancers to evade immunesurveillance. Mechanism(s) include disruption of antigen presentation, disruption of regulatory pathways controlling T cell activation or inhibition (immune checkpoint regulation), recruitment of cells that contribute to immune suppression (Tregs, MDSC) or release of factors that influence immune activity (IDO, PGE2). (See Harris et al., 2013, J Immunotherapy Cancer 1:12; Chen et al., 2013, Immunity 39:1; Pardoll, et al., 2012, Nature Reviews: Cancer 12:252; and Sharma et al., 2015, Cell 161:205, each of which is incorporated by reference herein in its entirety.)

Administration of the agent that induces iron-dependent cellular disassembly results in the production of postcellular signaling factors that regulate immune activity. Immune activity may be regulated by interaction of the postcellular signaling factors with a broad range of immune cells, including mast cells, Natural Killer (NK) cells, basophils, neutrophils, monocytes, macrophages, dendritic cells, eosinophils, and lymphocytes (e.g. B-lymphocytes (B-cells)), and T-lymphocytes (T-cells)).

Mast cells are a type of granulocyte containing granules rich in histamine and heparin, an anti-coagulant. When activated, a mast cell releases inflammatory compounds from the granules into the local microenvironment. Mast cells play a role in allergy, anaphylaxis, wound healing, angiogenesis, immune tolerance, defense against pathogens, and blood-brain barrier function.

Natural Killer (NK) cells are cytotoxic lymphocytes that lyse certain tumor and virus infected cells without any prior stimulation or immunization. NK cells are also potent producers of various cytokines, e.g. IFN-gamma (IFNγ), TNF-alpha (TNFα), GM-CSF and IL-3. Therefore, NK cells are also believed to function as regulatory cells in the immune system, influencing other cells and responses. In humans, NK cells are broadly defined as CD56+CD3− lymphocytes. The cytotoxic activity of NK cells is tightly controlled by a balance between the activating and inhibitory signals from receptors on the cell surface. A main group of receptors that inhibits NK cell activation are the inhibitory killer immunoglobulin-like receptors (KIRs). Upon recognition of self MHC class I molecules on the target cells, these receptors deliver an inhibitory signal that stops the activating signaling cascade, keeping cells with normal MHC class I expression from NK cell lysis. Activating receptors include the natural cytotoxicity receptors (NCR) and NKG2D that push the balance towards cytolytic action through engagement with different ligands on the target cell surface. Thus, NK cell recognition of target cells is tightly regulated by processes involving the integration of signals delivered from multiple activating and inhibitory receptors.

Monocytes are bone marrow-derived mononuclear phagocyte cells that circulate in the blood for few hours/days before being recruited into tissues. See Wacleche et al., 2018, Viruses (10)2: 65. The expression of various chemokine receptors and cell adhesion molecules at their surface allows them to exit the bone marrow into the blood and to be subsequently recruited from the blood into tissues. Monocytes belong to the innate arm of the immune system providing responses against viral, bacterial, fungal or parasitic infections. Their functions include the killing of pathogens via phagocytosis, the production of reactive oxygen species (ROS), nitric oxide (NO), myeloperoxidase and inflammatory cytokines. Under specific conditions, monocytes can stimulate or inhibit T-cell responses during cancer as well as infectious and autoimmune diseases. They are also involved in tissue repair and neovascularization.

Macrophages engulf and digest substances such as cellular debris, foreign substances, microbes and cancer cells in a process called phagocytosis. Besides phagocytosis, macrophages play a critical role in nonspecific defense (innate immunity) and also help initiate specific defense mechanisms (adaptive immunity) by recruiting other immune cells such as lymphocytes. For example, macrophages are important as antigen presenters to T cells. Beyond increasing inflammation and stimulating the immune system, macrophages also play an important anti-inflammatory role and can decrease immune reactions through the release of cytokines. Macrophages that encourage inflammation are called M1 macrophages, whereas those that decrease inflammation and encourage tissue repair are called M2 macrophages.

Dendritic cells (DCs) play a critical role in stimulating immune responses against pathogens and maintaining immune homeostasis to harmless antigens. DCs represent a heterogeneous group of specialized antigen-sensing and antigen-presenting cells (APCs) that are essential for the induction and regulation of immune responses. In the peripheral blood, human DCs are characterized as cells lacking the T-cell (CD3, CD4, CD8), the B-cell (CD19, CD20) and the monocyte markers (CD14, CD16) but highly expressing HLA-DR and other DC lineage markers (e.g., CD1a, CD1c). See Murphy et al., Janeway's Immunobiology. 8th ed. Garland Science; New York, N.Y., USA: 2012. 868p.

The term “lymphocyte” refers to a small white blood cell formed in lymphatic tissue throughout the body and in normal adults making up about 22-28% of the total number of leukocytes in the circulating blood that plays a large role in defending the body against disease. Individual lymphocytes are specialized in that they are committed to respond to a limited set of structurally related antigens through recombination of their genetic material (e.g. to create a T cell receptor and a B cell receptor). This commitment, which exists before the first contact of the immune system with a given antigen, is expressed by the presence of receptors specific for determinants (epitopes) on the antigen on the lymphocyte's surface membrane. Each lymphocyte possesses a unique population of receptors, all of which have identical combining sites. One set, or clone, of lymphocytes differs from another clone in the structure of the combining region of its receptors and thus differs in the epitopes that it can recognize. Lymphocytes differ from each other not only in the specificity of their receptors, but also in their functions. (Paul, W. E., “Chapter 1: The immune system: an introduction,” Fundamental Immunology, 4th Edition, Ed. Paul, W. E., Lippicott-Raven Publishers, Philadelphia, (1999), at p. 102).

Lymphocytes include B-lymphocytes (B-cells), which are precursors of antibody-secreting cells, and T-lymphocytes (T-cells).

B-Lymphocytes (B-cells)

B-lymphocytes are derived from hematopoietic cells of the bone marrow. A mature B-cell can be activated with an antigen that expresses epitopes that are recognized by its cell surface. The activation process may be direct, dependent on cross-linkage of membrane Ig molecules by the antigen (cross-linkage-dependent B-cell activation), or indirect, via interaction with a helper T-cell, in a process referred to as cognate help. In many physiological situations, receptor cross-linkage stimuli and cognate help synergize to yield more vigorous B-cell responses (Paul, W. E., “Chapter 1: The immune system: an introduction,” Fundamental Immunology, 4th Edition, Ed. Paul, W. E., Lippicott-Raven Publishers, Philadelphia, (1999)).

Cross-linkage dependent B-cell activation requires that the antigen express multiple copies of the epitope complementary to the binding site of the cell surface receptors, because each B-cell expresses Ig molecules with identical variable regions. Such a requirement is fulfilled by other antigens with repetitive epitopes, such as capsular polysaccharides of microorganisms or viral envelope proteins. Cross-linkage-dependent B-cell activation is a major protective immune response mounted against these microbes (Paul, W. E., “Chapter 1: The immune system: an introduction”, Fundamental Immunology, 4th Edition, Ed. Paul, W. E., Lippicott-Raven Publishers, Philadelphia, (1999)).

Cognate help allows B-cells to mount responses against antigens that cannot cross-link receptors and, at the same time, provides costimulatory signals that rescue B cells from inactivation when they are stimulated by weak cross-linkage events. Cognate help is dependent on the binding of antigen by the B-cell's membrane immunoglobulin (Ig), the endocytosis of the antigen, and its fragmentation into peptides within the endosomal/lysosomal compartment of the cell. Some of the resultant peptides are loaded into a groove in a specialized set of cell surface proteins known as class II major histocompatibility complex (MHC) molecules. The resultant class II/peptide complexes are expressed on the cell surface and act as ligands for the antigen-specific receptors of a set of T-cells designated as CD4⁺ T-cells. The CD4⁺ T-cells bear receptors on their surface specific for the B-cell's class II/peptide complex. B-cell activation depends not only on the binding of the T cell through its T cell receptor (TCR), but this interaction also allows an activation ligand on the T-cell (CD40 ligand) to bind to its receptor on the B-cell (CD40) signaling B-cell activation. In addition, T helper cells secrete several cytokines that regulate the growth and differentiation of the stimulated B-cell by binding to cytokine receptors on the B cell (Paul, W. E., “Chapter 1: The immune system: an introduction, “Fundamental Immunology, 4th Edition, Ed. Paul, W. E., Lippicott-Raven Publishers, Philadelphia, (1999)).

During cognate help for antibody production, the CD40 ligand is transiently expressed on activated CD4⁺ T helper cells, and it binds to CD40 on the antigen-specific B cells, thereby transducing a second costimulatory signal. The latter signal is essential for B cell growth and differentiation and for the generation of memory B cells by preventing apoptosis of germinal center B cells that have encountered antigen. Hyperexpression of the CD40 ligand in both B and T cells is implicated in pathogenic autoantibody production in human SLE patients (Desai-Mehta, A. et al., “Hyperexpression of CD40 ligand by B and T cells in human lupus and its role in pathogenic autoantibody production,” J. Clin. Invest. Vol. 97(9), 2063-2073, (1996)).

T-Lymphocytes (T-cells)

T-lymphocytes derived from precursors in hematopoietic tissue, undergo differentiation in the thymus, and are then seeded to peripheral lymphoid tissue and to the recirculating pool of lymphocytes. T-lymphocytes or T cells mediate a wide range of immunologic functions. These include the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on T cell expression of specific cell surface molecules and the secretion of cytokines (Paul, W. E., “Chapter 1: The immune system: an introduction”, Fundamental Immunology, 4th Edition, Ed. Paul, W. E., Lippicott-Raven Publishers, Philadelphia, (1999)).

T cells differ from B cells in their mechanism of antigen recognition. Immunoglobulin, the B cell's receptor, binds to individual epitopes on soluble molecules or on particulate surfaces. B-cell receptors see epitopes expressed on the surface of native molecules. While antibody and B-cell receptors evolved to bind to and to protect against microorganisms in extracellular fluids, T cells recognize antigens on the surface of other cells and mediate their functions by interacting with, and altering, the behavior of these antigen-presenting cells (APCs). There are three main types of APCs in peripheral lymphoid organs that can activate T cells: dendritic cells, macrophages and B cells. The most potent of these are the dendritic cells, whose only function is to present foreign antigens to T cells. Immature dendritic cells are located in tissues throughout the body, including the skin, gut, and respiratory tract. When they encounter invading microbes at these sites, they endocytose the pathogens and their products, and carry them via the lymph to local lymph nodes or gut associated lymphoid organs. The encounter with a pathogen induces the dendritic cell to mature from an antigen-capturing cell to an APC that can activate T cells. APCs display three types of protein molecules on their surface that have a role in activating a T cell to become an effector cell: (1) MHC proteins, which present foreign antigen to the T cell receptor; (2) costimulatory proteins which bind to complementary receptors on the T cell surface; and (3) cell-cell adhesion molecules, which enable a T cell to bind to the APC for long enough to become activated (“Chapter 24: The adaptive immune system,” Molecular Biology of the Cell, Alberts, B. et al., Garland Science, NY, (2002)).

T-cells are subdivided into two distinct classes based on the cell surface receptors they express. The majority of T cells express T cell receptors (TCR) consisting of α and β-chains. A small group of T cells express receptors made of γ and δ chains. Among the α/β T cells are two sub-lineages: those that express the coreceptor molecule CD4 (CD4⁺ T cells); and those that express CD8 (CD8⁺ T cells). These cells differ in how they recognize antigen and in their effector and regulatory functions.

CD4⁺ T cells are the major regulatory cells of the immune system. Their regulatory function depends both on the expression of their cell-surface molecules, such as CD40 ligand whose expression is induced when the T cells are activated, and the wide array of cytokines they secrete when activated.

T cells also mediate important effector functions, some of which are determined by the patterns of cytokines they secrete. The cytokines can be directly toxic to target cells and can mobilize potent inflammatory mechanisms.

In addition, T cells, particularly CD8⁺ T cells, can develop into cytotoxic T-lymphocytes (CTLs) capable of efficiently lysing target cells that express antigens recognized by the CTLs (Paul, W. E., “Chapter 1: The immune system: an introduction,” Fundamental Immunology, 4th Edition, Ed. Paul, W. E., Lippicott-Raven Publishers, Philadelphia, (1999)).

T cell receptors (TCRs) recognize a complex consisting of a peptide derived by proteolysis of the antigen bound to a specialized groove of a class II or class I MHC protein. CD4⁺ T cells recognize only peptide/class II complexes while CD8⁺ T cells recognize peptide/class I complexes (Paul, W. E., “Chapter 1: The immune system: an introduction,” Fundamental Immunology, 4th Edition, Ed. Paul, W. E., Lippicott-Raven Publishers, Philadelphia, (1999)).

The TCR's ligand (i.e., the peptide/MHC protein complex) is created within APCs. In general, class II MHC molecules bind peptides derived from proteins that have been taken up by the APC through an endocytic process. These peptide-loaded class II molecules are then expressed on the surface of the cell, where they are available to be bound by CD4⁺ T cells with TCRs capable of recognizing the expressed cell surface complex. Thus, CD4⁺ T cells are specialized to react with antigens derived from extracellular sources (Paul, W. E., “Chapter 1: The immune system: an introduction,” Fundamental Immunology, 4th Edition, Ed. Paul, W. E., Lippicott-Raven Publishers, Philadelphia, (1999)).

In contrast, class I MHC molecules are mainly loaded with peptides derived from internally synthesized proteins, such as viral proteins. These peptides are produced from cytosolic proteins by proteolysis by the proteosome and are translocated into the rough endoplasmic reticulum. Such peptides, generally composed of nine amino acids in length, are bound into the class I MHC molecules and are brought to the cell surface, where they can be recognized by CD8⁺ T cells expressing appropriate receptors. This gives the T cell system, particularly CD8⁺ T cells, the ability to detect cells expressing proteins that are different from, or produced in much larger amounts than, those of cells of the remainder of the organism (e.g., viral antigens) or mutant antigens (such as active oncogene products), even if these proteins in their intact form are neither expressed on the cell surface nor secreted (Paul, W. E., “Chapter 1: The immune system: an introduction,” Fundamental Immunology, 4th Edition, Ed. Paul, W. E., Lippicott-Raven Publishers, Philadelphia, (1999)).

T cells can also be classified based on their function as helper T cells; T cells involved in inducing cellular immunity; suppressor T cells; and cytotoxic T cells.

Helper T Cells

Helper T cells are T cells that stimulate B cells to make antibody responses to proteins and other T cell-dependent antigens. T cell-dependent antigens are immunogens in which individual epitopes appear only once or a limited number of times such that they are unable to cross-link the membrane immunoglobulin (Ig) of B cells or do so inefficiently. B cells bind the antigen through their membrane Ig, and the complex undergoes endocytosis. Within the endosomal and lysosomal compartments, the antigen is fragmented into peptides by proteolytic enzymes, and one or more of the generated peptides are loaded into class II MHC molecules, which traffic through this vesicular compartment. The resulting peptide/class II MHC complex is then exported to the B-cell surface membrane. T cells with receptors specific for the peptide/class II molecular complex recognize this complex on the B-cell surface. (Paul, W. E., “Chapter 1: The immune system: an introduction,” Fundamental Immunology, 4th Edition, Ed. Paul, W. E., Lippicott-Raven Publishers, Philadelphia (1999)).

B-cell activation depends both on the binding of the T cell through its TCR and on the interaction of the T-cell CD40 ligand (CD40L) with CD40 on the B cell. T cells do not constitutively express CD40L. Rather, CD40L expression is induced as a result of an interaction with an APC that expresses both a cognate antigen recognized by the TCR of the T cell and CD80 or CD86. CD80/CD86 is generally expressed by activated, but not resting, B cells so that the helper interaction involving an activated B cell and a T cell can lead to efficient antibody production. In many cases, however, the initial induction of CD40L on T cells is dependent on their recognition of antigen on the surface of APCs that constitutively express CD80/86, such as dendritic cells. Such activated helper T cells can then efficiently interact with and help B cells. Cross-linkage of membrane Ig on the B cell, even if inefficient, may synergize with the CD40L/CD40 interaction to yield vigorous B-cell activation. The subsequent events in the B-cell response, including proliferation, Ig secretion, and class switching of the Ig class being expressed, either depend or are enhanced by the actions of T cell-derived cytokines (Paul, W. E., “Chapter 1: The immune system: an introduction,” Fundamental Immunology, 4th Edition, Ed. Paul, W. E., Lippicott-Raven Publishers, Philadelphia, (1999)).

CD4⁺ T cells tend to differentiate into cells that principally secrete the cytokines IL-4, IL-5, IL-6, and IL-10 (T_{H}2 cells) or into cells that mainly produce IL-2, IFN-γ, and lymphotoxin (T_{H}1 cells). The T_{H}2 cells are very effective in helping B-cells develop into antibody-producing cells, whereas the T_{H}1 cells are effective inducers of cellular immune responses, involving enhancement of microbicidal activity of monocytes and macrophages, and consequent increased efficiency in lysing microorganisms in intracellular vesicular compartments. Although CD4⁺ T cells with the phenotype of TH2 cells (i.e., IL-4, IL-5, IL-6 and IL-10) are efficient helper cells, TH1 cells also have the capacity to be helpers (Paul, W. E., “Chapter 1: The immune system: an introduction, “Fundamental Immunology, 4th Edition, Ed. Paul, W. E., Lippicott-Raven Publishers, Philadelphia, (1999)).

T Cell Involvement in Cellular Immunity Induction

T cells also may act to enhance the capacity of monocytes and macrophages to destroy intracellular microorganisms. In particular, interferon-gamma (IFN-γ) produced by helper T cells enhances several mechanisms through which mononuclear phagocytes destroy intracellular bacteria and parasitism including the generation of nitric oxide and induction of tumor necrosis factor (TNF) production. T_{H1} cells are effective in enhancing the microbicidal action, because they produce IFN-γ. In contrast, two of the major cytokines produced by T_{H}2 cells, IL-4 and IL-10, block these activities (Paul, W. E., “Chapter 1: The immune system: an introduction,” Fundamental Immunology, 4th Edition, Ed. Paul, W. E., Lippicott-Raven Publishers, Philadelphia, (1999)).

Regulatory T (Treg) Cells

Immune homeostasis is maintained by a controlled balance between initiation and downregulation of the immune response. The mechanisms of both apoptosis and T cell anergy (a tolerance mechanism in which the T cells are intrinsically functionally inactivated following an antigen encounter (Schwartz, R. H., “T cell anergy”, Annu. Rev. Immunol., Vol. 21: 305-334 (2003)) contribute to the downregulation of the immune response. A third mechanism is provided by active suppression of activated T cells by suppressor or regulatory CD4+T (Treg) cells (Reviewed in Kronenberg, M. et al., “Regulation of immunity by self-reactive T cells”, Nature, Vol. 435: 598-604 (2005)). CD4⁺ Tregs that constitutively express the IL-2 receptor alpha (IL-2Rα) chain (CD4⁺ CD25⁺) are a naturally occurring T cell subset that are anergic and suppressive (Taams, L. S. et al., “Human anergic/suppressive CD4⁺CD25⁺ T cells: a highly differentiated and apoptosis-prone population”, Eur. J. Immunol. Vol. 31: 1122-1131 (2001)). Human CD4⁺CD25⁺ Tregs, similar to their murine counterpart, are generated in the thymus and are characterized by the ability to suppress proliferation of responder T cells through a cell-cell contact-dependent mechanism, the inability to produce IL-2, and the anergic phenotype in vitro. Human CD4⁺CD25⁺ T cells can be split into suppressive (CD25^{high}) and nonsuppressive (CD25^{low}) cells, according to the level of CD25 expression. A member of the forkhead family of transcription factors, FOXP3, has been shown to be expressed in murine and human CD4⁺CD25⁺ Tregs and appears to be a master gene controlling CD4⁺CD25⁺ Treg development (Battaglia, M. et al., “Rapamycin promotes expansion of functional CD4⁺CD25⁺Foxp3⁺ regulator T cells of both healthy subjects and type 1 diabetic patients”, J. Immunol., Vol. 177: 8338-8347, (2006)). Accordingly, in some embodiments, an increase in immune response may be associated with a lack of activation or proliferation of regulatory T cells.

Cytotoxic T Lymphocytes

CD8⁺ T cells that recognize peptides from proteins produced within the target cell have cytotoxic properties in that they lead to lysis of the target cells. The mechanism of CTL-induced lysis involves the production by the CTL of perforin, a molecule that can insert into the membrane of target cells and promote the lysis of that cell. Perforin-mediated lysis is enhanced by granzymes, a series of enzymes produced by activated CTLs. Many active CTLs also express large amounts of fas ligand on their surface. The interaction of fas ligand on the surface of CTL with fas on the surface of the target cell initiates apoptosis in the target cell, leading to the death of these cells. CTL-mediated lysis appears to be a major mechanism for the destruction of virally infected cells.

Lymphocyte Activation

The term “activation” or “lymphocyte activation” refers to stimulation of lymphocytes by specific antigens, nonspecific mitogens, or allogeneic cells resulting in synthesis of RNA, protein and DNA and production of lymphokines; it is followed by proliferation and differentiation of various effector and memory cells. T-cell activation is dependent on the interaction of the TCR/CD3 complex with its cognate ligand, a peptide bound in the groove of a class I or class II MHC molecule. The molecular events set in motion by receptor engagement are complex. Among the earliest steps appears to be the activation of tyrosine kinases leading to the tyrosine phosphorylation of a set of substrates that control several signaling pathways. These include a set of adapter proteins that link the TCR to the ras pathway, phospholipase Cγ1, the tyrosine phosphorylation of which increases its catalytic activity and engages the inositol phospholipid metabolic pathway, leading to elevation of intracellular free calcium concentration and activation of protein kinase C, and a series of other enzymes that control cellular growth and differentiation. Full responsiveness of a T cell requires, in addition to receptor engagement, an accessory cell-delivered costimulatory activity, e.g., engagement of CD28 on the T cell by CD80 and/or CD86 on the APC.

T-memory Cells

Following the recognition and eradication of pathogens through adaptive immune responses, the vast majority (90-95%) of T cells undergo apoptosis with the remaining cells forming a pool of memory T cells, designated central memory T cells (TCM), effector memory T cells (TEM), and resident memory T cells (TRM) (Clark, R. A., “Resident memory T cells in human health and disease”, Sci. Transl. Med., 7, 269rv1, (2015)).

Compared to standard T cells, these memory T cells are long-lived with distinct phenotypes such as expression of specific surface markers, rapid production of different cytokine profiles, capability of direct effector cell function, and unique homing distribution patterns. Memory T cells exhibit quick reactions upon re-exposure to their respective antigens in order to eliminate the reinfection of the offender and thereby restore balance of the immune system rapidly. Increasing evidence substantiates that autoimmune memory T cells hinder most attempts to treat or cure autoimmune diseases (Clark, R. A., “Resident memory T cells in human health and disease”, Sci. Transl. Med., Vol. 7, 269rv1, (2015)).

The agent that induces iron-dependent cellular disassembly may increase immune activity in a tissue or subject by inducing production of postcellular signaling factors that increase the level or activity of immune cells described herein, for example, macrophages, monocytes, dendritic cells, and CD4+, CD8+ or CD3+ cells (e.g. CD4+, CD8+ or CD3+ T cells). For example, in one embodiment, the agent that induces iron-dependent cellular disassembly is administered in an amount sufficient to increase in the tissue or subject one or more of: the level or activity of macrophages, the level or activity of monocytes, the level or activity of dendritic cells, the level or activity of T cells, and the level or activity of CD4+, CD8+ or CD3+ cells (e.g. CD4+, CD8+ or CD3+ T cells).

The agent that induces iron-dependent cellular disassembly may also increase immune activity in a cell, tissue or subject by inducing production of postcellular signaling factors that increase the level or activity of a pro-immune cytokine. For example, in some embodiments, the agent that induces iron-dependent cellular disassembly is administered in an amount sufficient to increase in a cell, tissue or subject the level or activity of a pro-immune cytokine. In one embodiment, the pro-immune cytokine is selected from IFN-α, IL-1, IL-12, IL-18, IL-2, IL-15, IL-4, IL-6, TNF-α, IL-17 and GMCSF.

The agent that induces iron-dependent cellular disassembly may also increase immune activity in a cell, tissue or subject by inducing production of postcellular signaling factors that increase the level or activity of positive regulators of the immune response such as nuclear factor kappa-light-chain-enhancer of activated B cells (NFkB), interferon regulatory factor (IRF), and stimulator of interferon genes (STING). For example, in some embodiments, the agent that induces iron-dependent cellular disassembly is administered in an amount sufficient to increase in a cell, tissue or subject the level or activity of NFkB, IRF and/or STING.

In some embodiments, the immune cell is a macrophage, monocyte, dendritic cell, T cell, CD4+ cell, CD8+ cell, or CD3+ cell. In some embodiments, the immune cell is a THP-1 cell.

In one embodiment, the subject is in need of an increased level or activity of NFkB.

In one embodiment, the level or activity of NFkB is increased by at least 10%, 20%, 30%, 40%, 50%, 60%, 70%, 80%, 90% or 100%, or by at least 2-fold, 4-fold, 6-fold, 8-fold, or 10-fold relative to a cell, tissue or subject that is not treated with the agent that induces iron-dependent cellular disassembly.

In one embodiment, the subject is in need of an increased level or activity of IRF or STING.

In one embodiment, the level or activity of IRF or STING is increased by at least 10%, 20%, 30%, 40%, 50%, 60%, 70%, 80%, 90% or 100%, or by at least 2-fold, 4-fold, 6-fold, 8-fold, or 10-fold relative to a cell, tissue or subject that is not treated with the agent that induces iron-dependent cellular disassembly.

In one embodiment, the subject is in need of an increased level or activity of macrophages, monocytes or dendritic cells.

In one embodiment, the level or activity of macrophages, monocytes, T cells or dendritic cells is increased by at least 10%, 20%, 30%, 40%, 50%, 60%, 70%, 80%, 90% or 100%, or by at least 2-fold, 4-fold, 6-fold, 8-fold, or 10-fold relative to a tissue or subject that is not treated with the agent that induces iron-dependent cellular disassembly.

In one embodiment, the subject is in need of an increased level or activity of CD4+, CD8+, or CD3+ cells.

In one embodiment, the level or activity of CD4+, CD8+, or CD3+ cells is increased by at least 10%, 20%, 30%, 40%, 50%, 60%, 70%, 80%, 90% or 100%, or by at least 2-fold, 4-fold, 6-fold, 8-fold, or 10-fold relative to a tissue or subject that is not treated with the agent that induces iron-dependent cellular disassembly.

In one embodiment, the subject is in need of an increased level or activity of a pro-immune cytokine.

In one embodiment, the level or activity of the pro-immune cytokine is increased by at least 10%, 20%, 30%, 40%, 50%, 60%, 70%, 80%, 90% or 100%, or by at least 2-fold, 4-fold, 6-fold, 8-fold, or 10-fold relative to a cell, tissue or subject that is not treated with the agent that induces iron-dependent cellular disassembly.

In one embodiment, the pro-immune cytokine is selected from IFN-α, IL-1, IL-12, IL-18, IL-2, IL-15, IL-4, IL-6, TNF-α, IL-17 and GMCSF.

Methods of measuring the level or activity of NFkB, IRF or STING; the level or activity of macrophages; the level or activity of monocytes; the level or activity of dendritic cells; the level or activity of CD4+ cells, CD8+ cells or CD3+ cells; the level or activity of T cells; and the level or activity of a pro-immune cytokine are known in the art.

For example, the protein level or activity of NFkB, IRF or STING may be measured by suitable techniques known in the art including ELISA, Western blot or in situ hybridization. The level of a nucleic acid (e.g. an mRNA) encoding NFkB, IRF or STING may be measured using suitable techniques known in the art including polymerase chain reaction (PCR) amplification reaction, reverse-transcriptase PCR analysis, quantitative real-time PCR, single-strand conformation polymorphism analysis (SSCP), mismatch cleavage detection, heteroduplex analysis, Northern blot analysis, in situ hybridization, array analysis, deoxyribonucleic acid sequencing, restriction fragment length polymorphism analysis, and combinations or sub-combinations thereof.

Methods for measuring the level and activity of macrophages are described, for example, in Chitu et al., 2011, Curr Protoc Immunol 14: 1-33. The level and activity of monocytes may be measured by flow cytometry, as described, for example, in Henning et al., 2015, Journal of Immunological Methods 423: 78-84. The level and activity of dendritic cells may be measured by flow cytometry, as described, for example in Dixon et al., 2001, Infect Immun. 69(7): 4351-4357. Each of these references is incorporated by reference herein in its entirety.

The level or activity of T cells may be assessed using a human CD4+ T-cell-based proliferative assay. For example, cells are labeled with the fluorescent dye 5,6-carboxyfluorescein diacetate succinimidyl ester (CFSE). Those cells that proliferate show a reduction in CFSE fluorescence intensity, which is measured directly by flow cytometry. Alternatively, radioactive thymidine incorporation can be used to assess the rate of growth of the T cells.

In some embodiments, an increase in immune response may be associated with reduced activation of regulatory T cells (Tregs). Functional activity T regs may be assessed using an in vitro Treg suppression assay. Such an assay is described in Collinson and Vignali (Methods Mol Biol. 2011; 707: 21-37, incorporated by reference in its entirety herein).

The level or activity of a pro-immune cytokine may be quantified, for example, in CD8+ T cells. In embodiments, the pro-immune cytokine is selected from interferon alpha (IFN-α), interleukin-1 (IL-1), IL-12, IL-18, IL-2, IL-15, IL-4, IL-6, tumor necrosis factor alpha (TNF-α), IL-17, and granulocyte-macrophage colony-stimulating factor (GMCSF). Quantitation can be carried out using the ELISPOT (enzyme-linked immunospot) technique, that detects T cells that secrete a given cytokine (e.g. IFN-α) in response to an antigenic stimulation. T cells are cultured with antigen-presenting cells in wells which have been coated with, e.g., anti-IFN-α antibodies. The secreted IFN-α is captured by the coated antibody and then revealed with a second antibody coupled to a chromogenic substrate. Thus, locally secreted cytokine molecules form spots, with each spot corresponding to one IFN-α-secreting cell. The number of spots allows one to determine the frequency of IFN-α-secreting cells specific for a given antigen in the analyzed sample. The ELISPOT assay has also been described for the detection of TNF-α, interleukin-4 (IL-4), IL-6, IL-12, and GMCSF.

### VII. Pharmaceutical Compositions and Modes of Administration

The therapeutic agents described herein (e.g. anti-neoplastic agents, agents that induce iron-dependent cellular disassembly, and immunotherapeutics) may be administered in one or more pharmaceutical compositions. The pharmaceutical compositions may be administered to a subject in any suitable formulation. These include, for example, liquid, semi-solid, and solid dosage forms, The preferred form depends on the intended mode of administration and therapeutic application.

In certain embodiments the composition is suitable for oral administration. In certain embodiments, the formulation is suitable for parenteral administration, including topical administration and intravenous, intraperitoneal, intramuscular, and subcutaneous, injections. In a particular embodiment, the composition is suitable for intravenous administration.

Pharmaceutical compositions for parenteral administration include aqueous solutions of the active compounds in water-soluble form. For intravenous administration, the formulation may be an aqueous solution. The aqueous solution may include Hank's solution, Ringer's solution, phosphate buffered saline (PBS), physiological saline buffer or other suitable salts or combinations to achieve the appropriate pH and osmolarity for parenterally delivered formulations. Aqueous solutions can be used to dilute the formulations for administration to the desired concentration. The aqueous solution may contain substances which increase the viscosity of the solution, such as sodium carboxymethyl cellulose, sorbitol, or dextran. In some embodiments, the formulation includes a phosphate buffer saline solution which contains sodium phosphate dibasic, potassium phosphate monobasic, potassium chloride, sodium chloride and water for injection.

Formulations suitable for topical administration include liquid or semi-liquid preparations suitable for penetration through the skin, such as liniments, lotions, creams, ointments or pastes, and drops suitable for administration to the eye, ear, or nose. Formulations suitable for oral administration include preparations containing an inert diluent or an assimilable edible carrier. The formulation for oral administration may be enclosed in hard or soft shell gelatin capsule, or it may be compressed into tablets, or it may be incorporated directly with the food of the diet. When the dosage unit form is a capsule, it may contain, in addition to materials of the above type, a liquid carrier. Various other materials may be present as coatings or to otherwise modify the physical form of the dosage unit. Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers including excipients and auxiliaries which facilitate processing of the active compounds into preparations which can be used pharmaceutically.

As will be readily apparent to one skilled in the art, the useful in vivo dosage to be administered and the particular mode of administration will vary depending upon the age, body weight, the severity of the affliction, and mammalian species treated, the particular compounds employed, and the specific use for which these compounds are employed. The determination of effective dosage levels, that is the dosage levels necessary to achieve the desired result, can be accomplished by one skilled in the art using routine methods, for example, human clinical trials, animal models, and in vitro studies.

In certain embodiments, the composition is delivered orally. In certain embodiments, the composition is administered parenterally. In certain embodiments, the compositions is delivered by injection or infusion. In certain embodiments, the composition is delivered topically including transmucosally. In certain embodiments, the composition is delivered by inhalation. In one embodiment, the compositions provided herein may be administered by injecting directly to a tumor. In some embodiments, the compositions may be administered by intravenous injection or intravenous infusion. In certain embodiments, administration is systemic. In certain embodiments, administration is local.

### EXAMPLES

### Example 1: Activation/Stimulation of Human Monocytes by HT1080 Fibrosarcoma Cells Treated with Erastin

Agent/Therapeutic Design:

HT1080 fibrosarcoma cells were treated with either control (DMSO) or various doses of Erastin, piperazine erastin (PE), or imidazole ketoerastin (IKE) for 24 hours prior to co-culture with THP1-Dual cells for an additional 24 hours. Erastin was purchased from Selleckchem (Houston, Tex.) and dissolved in DMSO. Subsequently, the THP1 supernatants were assessed for nuclear factor kappa-light-chain-enhancer of activated B cells (NFKB) or interferon regulatory factor (IRF) reporter activity.

Materials/Methods:

HT1080 cells were acquired from ATCC and THP1-Dual cells were acquired from InvivoGen (San Diego, Calif.). THP1-Dual cells are human monocyte cells that induce reporter proteins upon activation of either NFKB or IRF pathways. Both cell types were cultured in 96-well plates for the duration of the assay. HT1080 cells were cultured in DMEM with 10% FBS, and THP1-Dual cells were cultured in RPMI with 10% FBS. 7,500 HT1080 cells were plated 24 hours prior to dosing with Erastin, PE, or IKE, with a final DMSO concentration of 0.5%. 24 hours post-treatment, THP1-Dual cells (25,000 cells/well) were added to the HT1080 cells. 24 hours later, 30 μl of supernatant was mixed with either 200 μl QuantiBlue (InvivoGen, San Diego, Calif.) (for NFKB reporter activity) or 50 μl QuantiLuc (for IRF reporter activity) and absorbance or luminescence was recorded on a Molecular Devices plate reader.

Conclusion:

As shown in FIG. 1A, Erastin treatment negatively affected the viability of HT1080 cells. FIG. 1B shows that HT1080 cells treated with Erastin, but not the vehicle control DMSO, elicited NFKB signaling in THP1 monocytes. The erastin analogs PE and IKE also negatively affected the viability of HT1080 cells (FIG. 1C) and elicited NFKB signaling in THP1 monocytes (FIG. 1D).

### Example 2: Activation/Stimulation of Human Monocytes by PANC1 Pancreatic Cancer Cells Treated with Erastin

Agent/Therapeutic Design:

PANC1 pancreatic cancer cells were treated with either control (DMSO) or various doses of Erastin, for 24 hours prior to co-culture with THP1-Dual cells for an additional 24 hours. Erastin was purchased from Selleckchem (Houston, Tex.) and dissolved in DMSO. Subsequently, the THP1 supernatants were assessed for NFKB or IRF reporter activity.

Materials/Methods:

PANC1 cells were acquired from ATCC and THP1-Dual cells were acquired from InvivoGen (San Diego, Calif.). Both cell types were cultured in 96-well plates for the duration of the assay. 7,500 PANC-1 cells were plated 24 hours prior to dosing with Erastin, with a final DMSO concentration of 0.5%. 24 hours post-treatment, THP1-Dual cells (25,000 cells/well) were added to the PANC-1 cells. 24 hours later, 30 μl of supernatant was mixed with either 200 μl QuantiBlue (InvivoGen, San Diego, Calif.) or 50 μl QuantiLuc and absorbance or luminescence was recorded on a Molecular Devices plate reader.

Conclusion:

As shown in FIG. 2A, Erastin treatment negatively affected the viability of PANC1 cells. FIG. 1B shows that PANC1 cells treated with Erastin, but not the vehicle control DMSO, elicited NFKB signaling in THP1 monocytes.

### Example 3: Activation/Stimulation of Human Monocytes by Caki-1 Renal Cell Carcinoma Cells Treated with Erastin

Agent/Therapeutic Design:

Caki-1 renal cell carcinoma cells were treated with either control (DMSO) or various doses of Erastin, for 24 hours prior to co-culture with THP1-Dual cells for an additional 24 hours. Erastin was purchased from Selleckchem (Houston, Tex.) and dissolved in DMSO. Subsequently, the THP1 supernatants were assessed for NFKB or IRF reporter activity.

Materials/Methods:

Caki-1 cells were acquired from ATCC and THP1-Dual cells acquired from InvivoGen (San Diego, Calif.). Both cell types were cultured in 96-well plates for the duration of the assay. 7,500 Caki-1 cells were plated 24 hours prior to dosing with Erastin, with a final DMSO concentration of 0.5%. 24 hours post-treatment, THP1-Dual cells (25,000 cells/well) were added to the Caki-1 cells. 24 hours later, 30 μl of supernatant was mixed with either 200 μl QuantiBlue (InvivoGen) or 50 μl QuantiLuc and absorbance or luminescence was recorded on a Molecular Devices plate reader.

Conclusion:

As shown in FIG. 3A, Erastin treatment negatively affected the viability of Caki-1 cells. FIG. 3B shows that Caki-1 cells treated with Erastin, but not the vehicle control DMSO, elicited NFKB signaling in THP1 monocytes.

### Example 4: Activation/Stimulation of Human Monocytes by Caki-1 Renal Cell Carcinoma Cells Treated with RSL3

Agent/Therapeutic Design:

Caki-1 renal cell carcinoma cells were treated with either control (DMSO) or various doses of RSL3, for 24 hours prior to co-culture with THP1-Dual cells for an additional 24 hours. RSL3 was purchased from Selleckchem and dissolved in DMSO. Subsequently, the THP1 supernatants were assessed for NFKB or IRF reporter activity.

Materials/Methods:

Caki-1 cells were acquired from ATCC and THP1-Dual cells acquired from InvivoGen (San Diego, Calif.). Both cell types were cultured in 96-well plates for the duration of the assay. 7,500 Caki-1 cells were plated 24 hours prior to dosing with RSL3, with a final DMSO concentration of 0.5%. 24 hours post-treatment, THP1-Dual cells (25,000 cells/well) were added to the Caki-1 cells. 24 hours later, 30 μl of supernatant was mixed with either 200 μl QuantiBlue (InvivoGen, San Diego, Calif.) or 50 μl QuantiLuc and absorbance or luminescence was recorded on a Molecular Devices plate reader.

Conclusion:

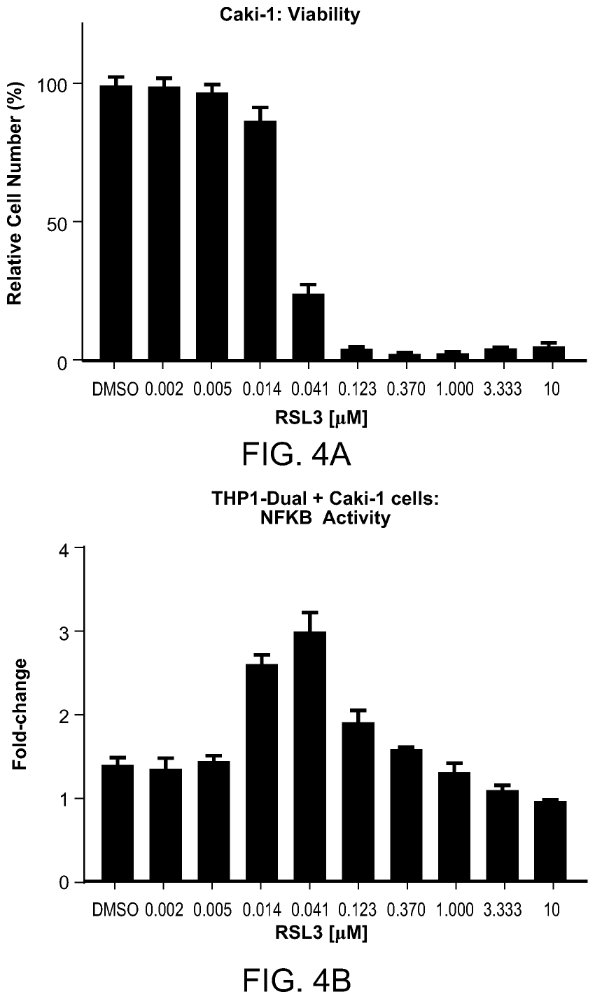

As shown in FIG. 4A, RSL3 treatment negatively affected the viability of Caki-1 cells. FIG. 4B shows that Caki-1 cells treated with RSL3, but not the vehicle control DMSO, elicited NFKB signaling in THP1 monocytes.

### Example 5: Activation/Stimulation of Human Monocytes by Jurkat T Cell Leukemia Cells Treated with RSL3

Agent/Therapeutic Design:

Jurkat T cell leukemia cells were treated with either control (DMSO) or various doses of RSL3, for 24 hours prior to co-culture with THP1-Dual cells for an additional 24 hours. RSL3 was purchased from Selleckchem (Houston, Tex.) and dissolved in DMSO. Subsequently, the THP1 supernatants were assessed for NFKB or IRF reporter activity.

Materials/Methods:

Jurkat cells were acquired from ATCC and THP1-Dual cells acquired from InvivoGen (San Diego, Calif.). Both cell types were cultured in 96-well plates for the duration of the assay. 100,000 Jurkat cells were plated 24 hours prior to dosing with RSL3, with a final DMSO concentration of 0.5%. 24 hours post-treatment, THP1-Dual cells (25,000 cells/well) were added to the Jurkat cells. 24 hours later, 30 μl of supernatant was mixed with either 200 μl QuantiBlue (InvivoGen, San Diego, Calif.) or 50 μl QuantiLuc and absorbance or luminescence was recorded on a Molecular Devices plate reader.

Conclusion:

As shown in FIG. 5A, RSL3 treatment negatively affected the viability of Jurkat T cell leukemia cells. FIG. 5B shows that Jurkat cells treated with RSL3, but not the vehicle control DMSO, elicited NFKB signaling in THP1 monocytes.

### Example 6: Activation/Stimulation of Human Monocytes by A20 B-Cell Leukemia Cells Treated with RSL3

Agent/Therapeutic Design:

A20 B-cell leukemia cells were treated with either control (DMSO) or various doses of RSL3, for 24 hours prior to co-culture with THP1-Dual cells for an additional 24 hours. RSL3 was purchased from Selleckchem (Houston, Tex.) and dissolved in DMSO. Subsequently, the THP1 supernatants were assessed for NFKB or IRF reporter activity.

Materials/Methods:

A20 cells were acquired from ATCC and THP1-Dual cells acquired from InvivoGen (San Diego, Calif.). Both cell types were cultured in 96-well plates for the duration of the assay. 50,000 A20 cells were plated 24 hours prior to dosing with RSL3, with a final DMSO concentration of 0.5%. 24 hours post-treatment, THP1-Dual cells (25,000 cells/well) were added to the A20 cells. 24 hours later, 30 μl of supernatant was mixed with either 200 μl QuantiBlue (InvivoGen, San Diego, Calif.) or 50 μl QuantiLuc and absorbance or luminescence was recorded on a Molecular Devices plate reader.

Conclusion:

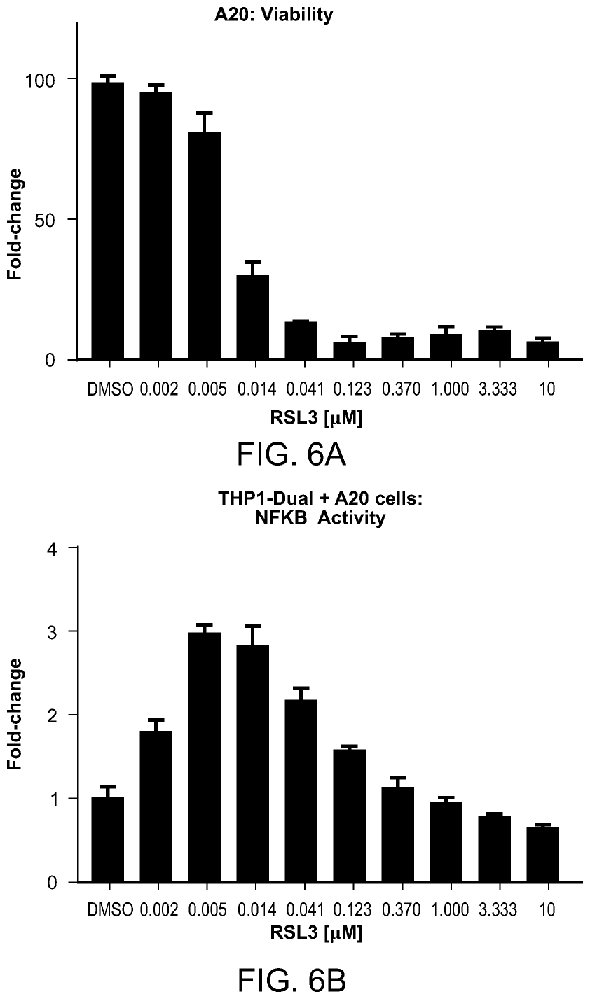

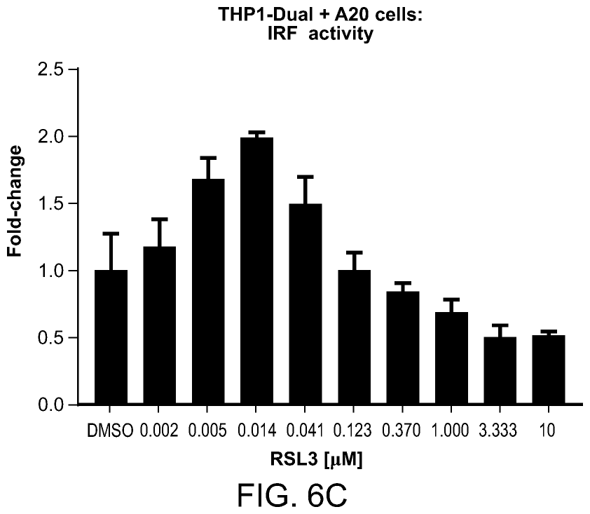

As shown in FIG. 6A, RSL3-treatment negatively affected the viability of A20 B cell leukemia cells. A20 cells treated with RSL3, but not the vehicle control DMSO, elicited NFKB (FIG. 6B) and IRF (FIG. 6C) signaling in THP1 monocytes.

### Example 7: Specificity of Pro-Inflammatory Signaling Elicited by HT1080 Fibrosarcoma Cells Treated with Erastin

Agent/Therapeutic Design:

HT1080 fibrosarcoma cells were treated with various doses of Erastin (e.g. 0.8, 0.16, 0.31, 0.63, 1.25, 2.5, 5, 10 or 20 μM) alone or in combination with a ferroptosis inhibitor (1 μM Ferrostatin-1, 1 μM Liproxstatin-1, 100 μM Trolox, 25 μM β-Mercaptoethanol or 100 μM Deferoxamine) for 24 hours prior to co-culture with THP1-Dual cells for an additional 24 hours. Ferrostatin-1 and Liproxstatin-1 were purchased from Selleckchem (Houston, Tex.) and dissolved in DMSO. Trolox was purchased from Cayman Chemical Company Inc and resuspended in DMSO. Deferoxamine mesylate was purchased from Sigma-Aldrich and resuspended in water. β-Mercaptoethanol was purchased from Life Technologies. Subsequently, the THP1 supernatant was assessed for NFKB activity. HT1080 cells were acquired from ATCC and THP1-Dual cells were acquired from InvivoGen (San Diego, Calif.). Both cell types were cultured in 96-well plates for the duration of the assay.

Results:

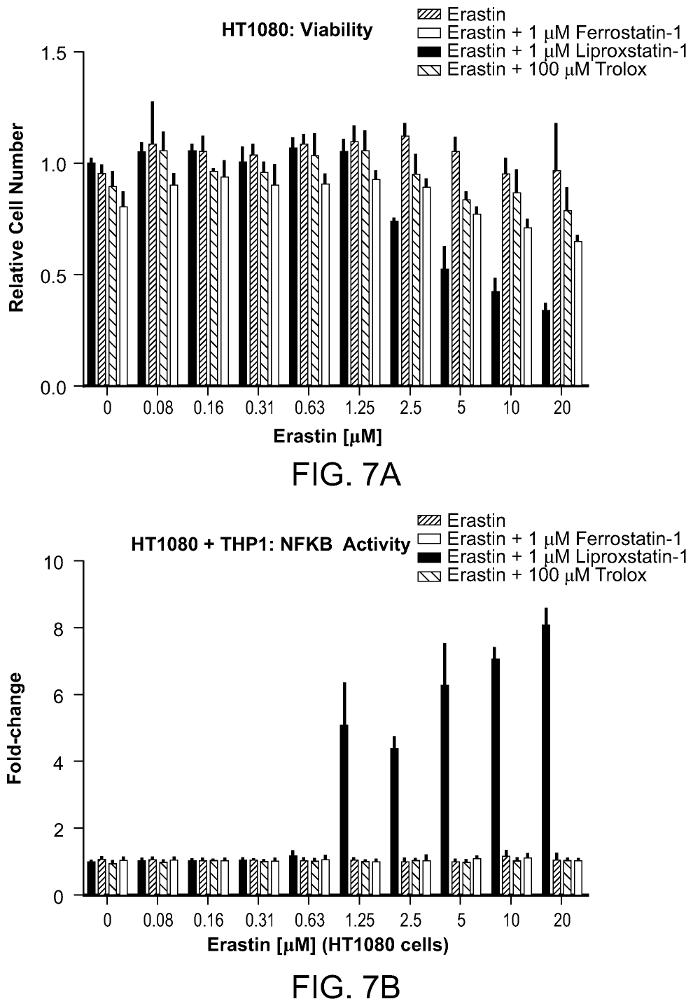

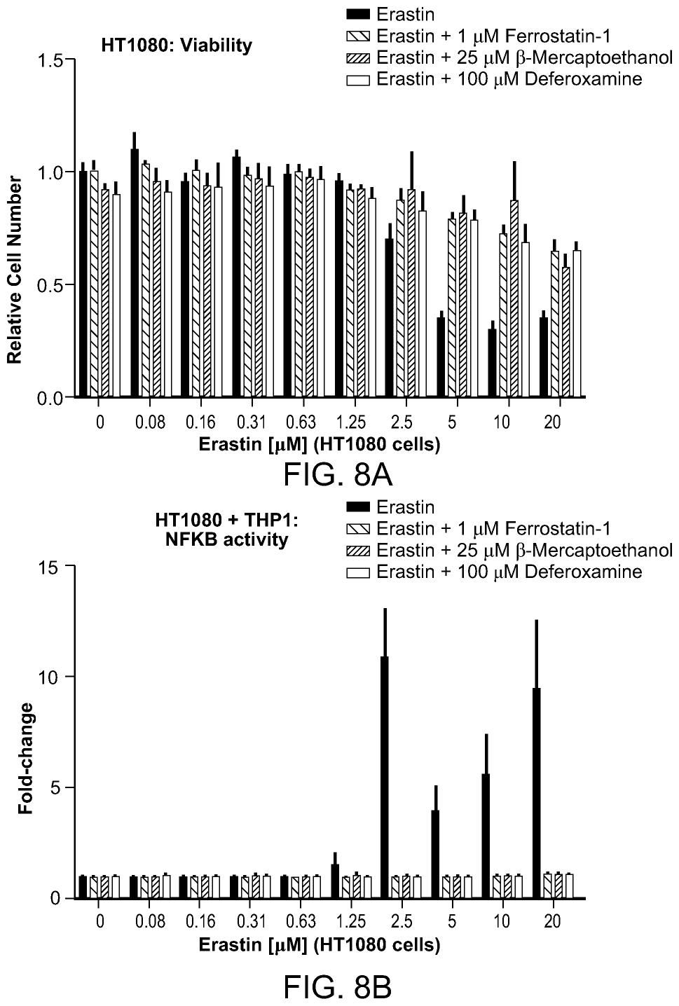

As shown in FIGS. 7A and 8A, Erastin treatment of HT1080 fibrosarcoma cells decreased viability of the cells in a dose dependent manner, and this decreased viability was attenuated by each of the ferroptosis inhibitors. As shown in FIGS. 7B and 8B, Erastin treatment of HT1080 fibrosarcoma cells increased NFKB activity in THP1 cells in a dose dependent manner, and this increased NFKB activity was abrogated by each of the ferroptosis inhibitors. These results demonstrate that cell death plays a role in the induction of NFKB signaling elicited by Erastin-treated HT1080 cells.

### Example 8: Knockdown of ACSL4 and CARS Genes Inhibits Erastin-Mediated Cell Death in HT1080 Fibrosarcoma Cells

Agent/Therapeutic Design:

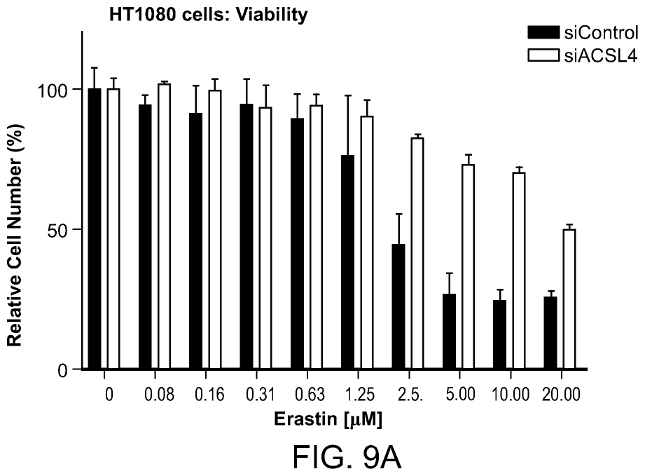

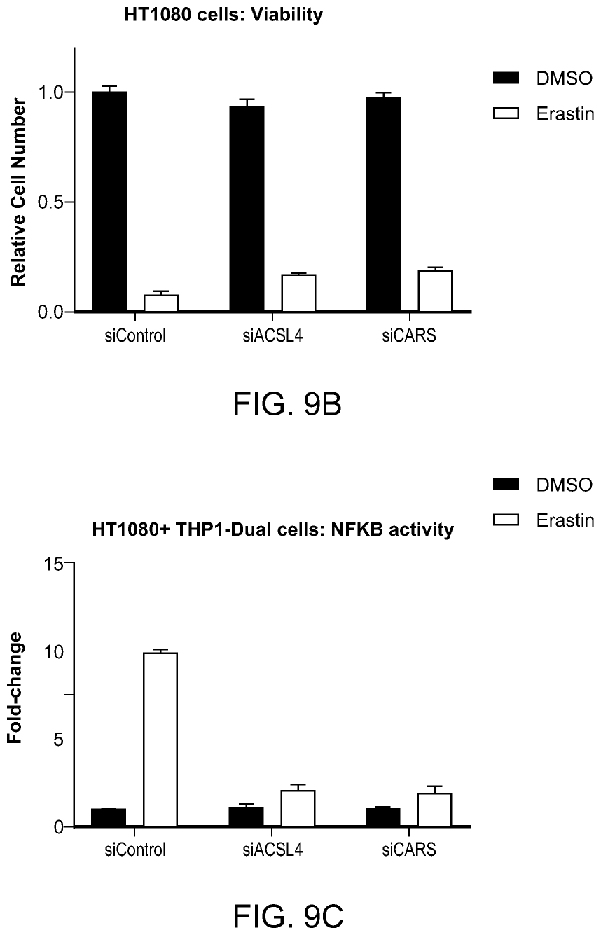

HT1080 cells (5,000 cells/well) were reverse transfected in 96-well format using DharmaFECT I transfection reagent (Catalog #T-2001) and control siRNA (Dharmacon Catalog #D-001810-10-05) or siRNA [37.5 nM] targeting ACSL4 (FIG. 9A, Dharmacon Catalog #L-009364-00-005) or a pool of siRNAs (FIG. 9B/C) against ACSL4 (Thermo Fisher Silencer Select Catalog #'s: s5001, s5001, s5002) or CARS (Thermo Fisher Silencer Select Catalog #'s: S2404, s2405, s2406). 48 hours post-transfection, cell culture medium was replaced by fresh medium containing variours concentrations of Erastin (FIG. 9A) or a fixed concentration of Erastin (10 μM, FIG. 9B/C). In addition, 50,000 reporter THP1-dual cells were added to some plates (FIG. 9C). 24 hours later, HT1080 cell viability was measured (FIG. 9A/B) or THP1 superntatant was assessed for NFKB activity (FIG. 9C). HT1080 cells were acquired from ATCC and THP1-Dual cells were acquired from InvivoGen (San Diego, Calif.).

Results:

The ACSL4 gene encodes Long-chain-fatty-acid-CoA ligase 4, an acyl-CoA synthetase that controls the level of arachidonic acid in cells, and is involved in the regulation of cell death. The CARS gene encodes cysteinyl-tRNA synthetase. Knockdown of CARS has been shown to inhibit erastin-induced ferroptosis by preventing the induction of lipid reactive oxygen species. See Hayano et al., 2016, Cell Death Differ. 23(2): 270-278. As shown in FIGS. 9A and 9B, genetic knockdown of either ACLS4 or CARS in HT1080 cells partially rescues viability of HT1080 cells cultured in the presence of Erastin. In addition, genetic knockdown of either ACLS4 or CARS in HT1080 cells abrogates NFKB activity in monocytes co-cultured with Erastin-treated HT1080 cells (FIG. 9C). These results demonstrate that cell death plays a role in the induction of NFKB signaling elicited by Erastin-treated HT1080 cells and that the absence of specific intracellular proteins reduces the pro-inflammatory nature of ferroptosis.

### Example 9: Specificity of Pro-Inflammatory Signaling Elicited by A20 Cells Treated with a GPX4 Inhibitor (RSL3, ML162 or ML210)

Agent/Therapeutic Design:

A20 lymphoma cells were treated with various doses (e.g. 0.002, 0.005, 0.014, 0.041, 0.123, 0.370, 1.111, 3.333 and 10 μM) of a GPX4 inhibitor (RSL3, ML162 or ML210) in the presence or absence of 1 μM Ferrostatin-1 for 24 hours. ML162 was purchased from Cayman Chemical Company Inc and resuspended in DMSO. ML210 was purchased from Sigma-Aldrich and resuspended in DMSO. A20 lymphoma cells were also treated with DMSO as a negative control. After treatment with DMSO or a GPX4 inhibitor for 24 hours, the A20 lymphoma cells were co-cultured with THP1-Dual cells for an additional 24 hours. Subsequently, the THP1 supernatant was assessed for NFKB reporter activity. A20 cells were acquired from ATCC and THP1-Dual cells were acquired from InvivoGen (San Diego, Calif.). Both cell types were cultured in 96-well plates for the duration of the assay.

Results:

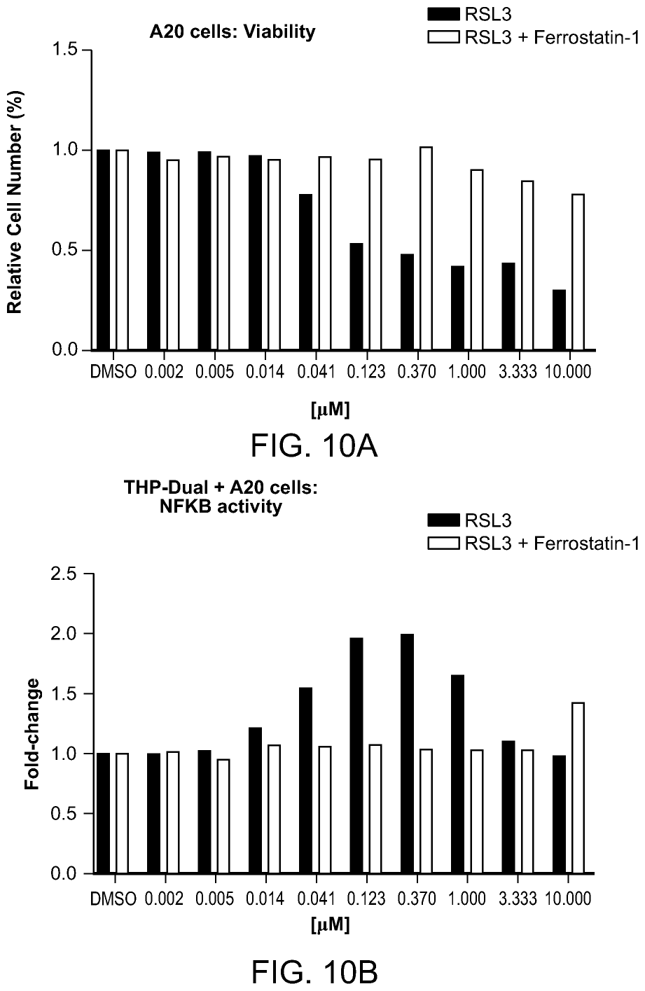

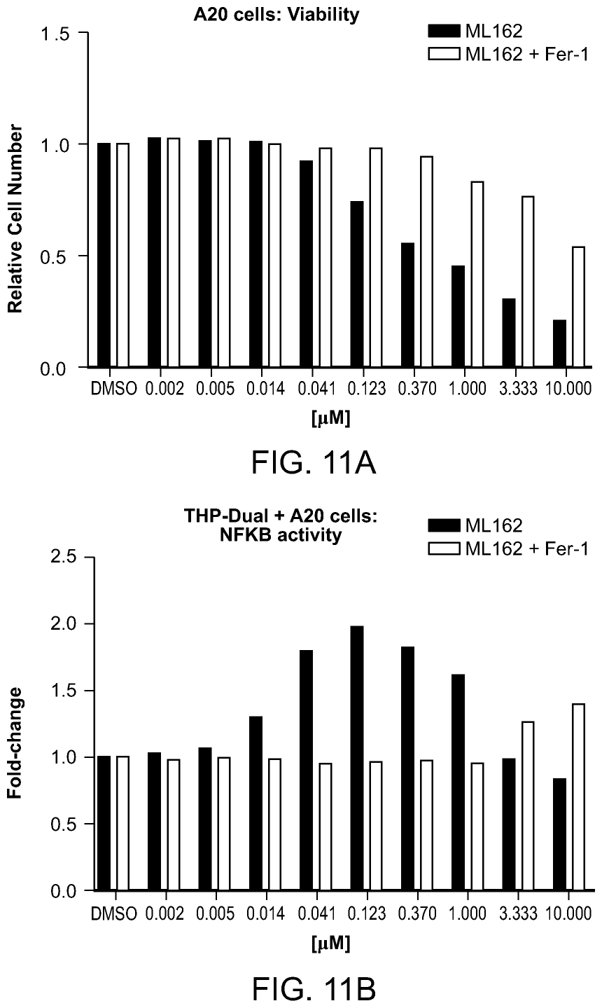

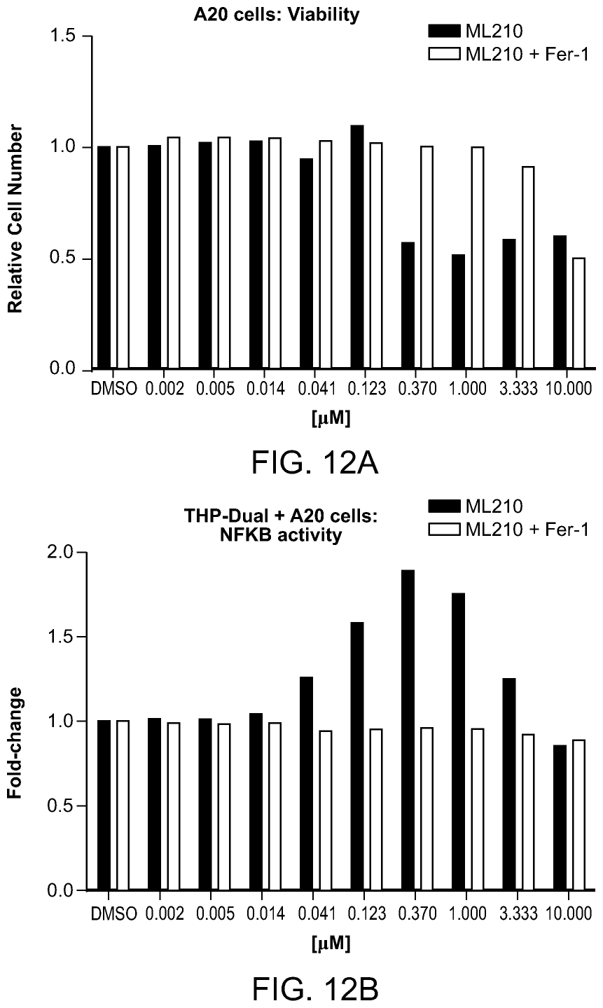

As shown in FIGS. 10A, 11A and 12A, treatment of A20 lymphoma cells with each of the GPX4 inhibitors (RSL3, ML162 or ML210) decreased viability of the cells in a dose dependent manner, and this decreased viability was attenuated by the ferroptosis inhibitor Ferrostatin-1. As shown in FIGS. 10B, 11B and 12B, GPX4 inhibitor treatment of A20 lymphoma cells increased NFKB activity in THP1 cells in a dose dependent manner, and this increased NFKB activity was attenuated by the ferroptosis inhibitor Ferrostatin-1. These results demonstrate that cell death plays a role in the induction of NFKB signaling elicited by GPX-4 inhibitor-treated A20 lymphoma cells.

### Example 10: Specificity of Pro-Inflammatory Signaling Elicited by Caki-1 Renal Carcinoma Cells Treated with a GPX4 Inhibitor (RSL3 or ML162)

Agent/Therapeutic Design:

Caki-1 renal carcinoma cells were treated with either control (DMSO) or various doses (e.g. 0.002, 0.005, 0.014, 0.041, 0.123, 0.370, 1.111, 3.333 and 10 PM) of a GPX4 inhibitor (RSL3 or ML162) in the presence or absence of 1 μM Ferrostatin for 24 hours prior to co-culture with THP1-Dual cells for an additional 24 hours. Subsequently, the THP1 supernatant was assessed for NFKB reporter activity. Caki-1 cells were acquired from ATCC and THP1-Dual cells acquired from InvivoGen (San Diego, Calif.). Both cell types were cultured in 96-well plates for the duration of the assay.

Results:

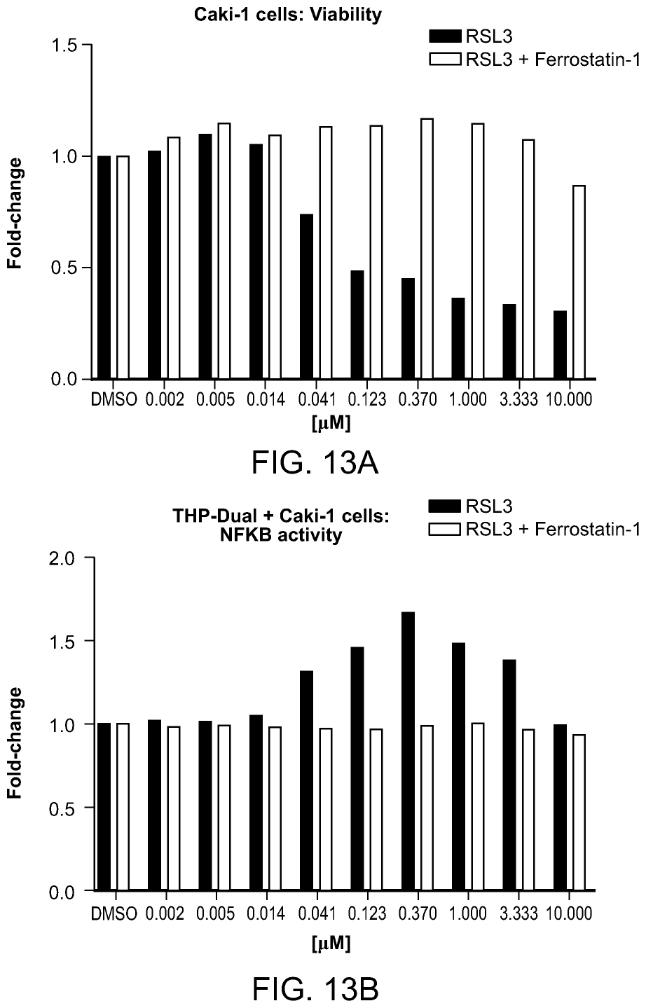

As shown in FIGS. 13A and 14A, treatment of Caki-1 renal carcinoma cells with a GPX4 inhibitor (RSL3 or ML162) decreased viability of the cells in a dose dependent manner, and this decreased viability was attenuated by the ferroptosis inhibitor Ferrostatin-1. As shown in FIGS. 13B and 14B, treatment of Caki-1 renal carcinoma cells with RSL3 or ML162 increased NFKB activity in THP1 cells in a dose dependent manner, and this increased NFKB activity was attenuated by the ferroptosis inhibitor Ferrostatin-1. These results demonstrate that cell death plays a role in the induction of NFKB signaling elicited by GPX-4 inhibitor-treated Caki-1 renal carcinoma cells.

### Example 11: Comparison of Ferroptosis, Apoptosis and Necrosis of HT1080 Fibrosarcoma Cells on NFKB Activity in Human THP1 Monocytes

Agent/Therapeutic Design:

HT1080 fibrosarcoma cells were treated with various doses of Erastin (ferroptosis inducer), docetaxel (apoptosis inducer) or H₂O₂ (necrosis inducer) for 24 hours prior to co-culture with THP1-Dual cells for an additional 24 hours. Erastin was purchased from Selleckchem (Houston, Tex.) and dissolved in DMSO. Subsequently, the THP1 supernatants were assessed for nuclear factor kappa-light-chain-enhancer of activated B cells (NFKB).

Materials/Methods:

HT1080 cells were acquired from ATCC and THP1-Dual cells were acquired from InvivoGen (San Diego, Calif.). THP1-Dual cells are human monocyte cells that induce reporter proteins upon activation of either NFKB or IRF pathways. Both cell types were cultured in 96-well plates for the duration of the assay. HT1080 cells were cultured in DMEM with 10% FBS, and THP1-Dual cells were cultured in RPMI with 10% FBS. 7,500 HT1080 cells were plated 24 hours prior to dosing with Erastin, docetaxel or H₂O₂. 24 hours post-treatment, THP1-Dual cells (25,000 cells/well) were added to the HT1080 cells. 24 hours later, 30 μl of supernatant was mixed with either 200 μl QuantiBlue (InvivoGen, San Diego, Calif.) (for NFKB reporter activity) or 50 μl QuantiLuc (for IRF reporter activity) and absorbance or luminescence was recorded on a Molecular Devices plate reader.

Conclusion:

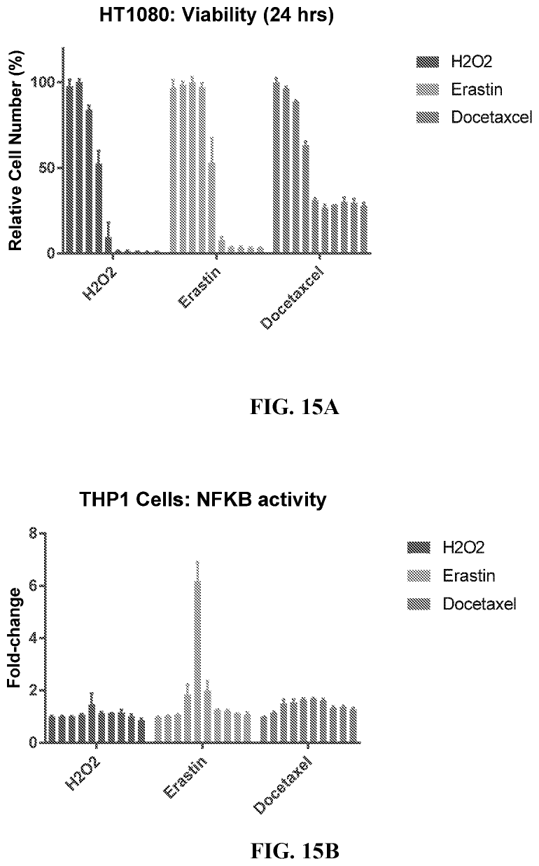

As shown in FIG. 15A, Erastin, docetaxel and H₂O₂ treatment negatively affected the viability of HT1080 cells. FIG. 15B shows that while HT1080 cells treated with Erastin elicited large increases in NFKB signaling in THP1 monocytes, HT1080 cells treated with docetaxel or H₂O₂ did not have this same effect.

### Example 12: Treatment of Cancer Cells In Vitro with Combinations of an Anti-Neoplastic Agent and an Agent that Induces Iron-Dependent Cellular Disassembly

While not wishing to be bound by theory, it is believed that treating a cancer cell with an anti-neoplastic agent that promotes ROS stress makes the cell more dependent upon GPX4 and/or System Xc for survival (i.e. to remove the ROS), thereby making the cell more sensitive to killing by an agent that induces iron-dependent cellular disassembly (e.g an agent that inhibits GPX4 or System Xc). Thus combination of an anti-neoplastic agent and an agent that induces iron-dependent cellular disassembly may kill cancer cells that are not susceptible to either agent alone.

Cancer cell lines are treated with multiple dose levels of an anti-neoplastic agent (e.g. a targeted therapy or an apoptosis inducing agent) alone, an agent that induces iron-dependent cellular disassembly (e.g. a GPX4 inhibitor or a System Xc inhibitors) alone, or with a combination of the anti-neoplastic agent and the agent that induces iron-dependent cellular disassembly. Combination treatments are expected to induce a greater extent of cancer cell death, induce cancer cell death at lower concentrations, reduce compensatory proliferation of the cancer cells, and/or induce stronger immune response in immune cells co-cultured with the treated cancer cells than either single agent alone. For example, cell lines resistant to a ferroptosis inducer are treated with an anti-neoplastic agent with or without the ferroptosis inducer. THP-1 cells with an NFkappaB reporter are added to the culture and assessed for NFKB activity. The level of NFkappaB activity is expected to be greater in the combination of the ferroptosis inducer and the anti-neoplastic agent relative to either single agent alone.

### Example 13: Treatment of Cancer In Vivo with Combinations of an Anti-Neoplastic Agent and an Agent that Induces Iron-Dependent Cellular Disassembly

Syngeneic mouse cancer models are used to explore the effects of combinations of an anti-neoplastic agent and an agent that induces iron-dependent cellular disassembly on cancer development. Mice with syngeneic tumors are treated with an anti-neoplastic agent (e.g. a targeted therapy or an apoptosis inducing agent) alone, an agent that induces iron-dependent cellular disassembly (e.g. a GPX4 inhibitor or a System Xc inhibitors) alone, or with a combination of the anti-neoplastic agent and the agent that induces iron-dependent cellular disassembly. In some experiments, suboptimal doses of the anti-neoplastic agent are used. Treatment with the combination of the anti-neoplastic agent and the agent that induces iron-dependent cellular disassembly is expected to slow tumor growth and/or stimulate immune response in the tumors to a greater extent than either agent alone.

### Example 14: Treatment In Vitro of Cells that have Undergone Epithelial-Mesenchymal Transition (EMT) and Non-Mesenchymal Cancer Cells with an Agent that Induces Iron-Dependent Cellular Disassembly

Cancer cell lines known to show a mesenchymal state (e.g. HT1080 fibrosarcoma, Rh30 and Rh41 rhabdomyosarcoma, and immortalized p53-null fibroblasts MDAH041) and non-mesenchymal cancer cell lines are treated with agents that induce iron-dependent cellular disassembly (e.g. GPX4 inhibitors or System Xc inhibitors). The IC₅₀ for each agent that induces iron-dependent cellular disassembly will be determined for both the cancer cells that show a mesenchymal state and for the non-mesenchymal cancer cell lines. It is expected that the IC₅₀ values will be lower in the cancer cell lines that show a mesenchymal state, indicating a greater sensitivity to the agents that induce iron-dependent cellular disassembly relative to the non-mesenchymal cancer cell lines.

In other experiments, human mammary epithelial cells are neoplastically transformed by stepwise introduction of defined genetic events (activated Ras+c-Myc+p53shRNA and p16shRNA) as described in Taylor et al., 2019, Sci Rep. Apr. 11; 9(1):5926; and Junk et al., 2013, Neoplasia 15, 1100-1109; each of which is incorporated by reference herein in its entirety. The resulting transformed population contains epithelial cells and mesenchymal cells. The transformed cells are treated with an agent that induces iron-dependent cellular disassembly (e.g. a GPX4 inhibitor or a System Xc inhibitor). It is expected that the mesenchymal cells, but not the epithelial cells, will be sensitive to the agents that induce iron-dependent cellular disassembly.

### Example 15: Treatment of Persister Cells In Vitro with Combinations of an Anti-Neoplastic Agent and an Agent that Induces Iron-Dependent Cellular Disassembly

Cancer cells with mutations that make them susceptible to a targeted therapeutic are treated with the targeted therapeutic at the appropriate concentration for at least nine days with fresh drug added every three days. Surviving cells are treated with an agent that induces iron-dependent cellular disassembly (e.g. a GPX4 inhibitor or a System Xc inhibitor) and tested for cell viability. Alternatively, surviving cells are grown for 14-37 days without the targeted therapeutic to increase the number of cells, and then treated with the targeted therapeutic for at least nine days with fresh drug added every three days. Surviving cells from this second treatment of targeted therapy are also treated with the agent that induces iron-dependent cellular disassembly (e.g. a GPX4 inhibitor or a System Xc inhibitor) and tested for cell viability. See Hangauer et al., 2017, Nature, November 9; 551(7679):247-250; and Viswanathan et al., 2017, Nature, July 27; 547(7664):453-457.

In another experiment, cancer cells with mutations that make them susceptible to a targeted therapeutic are treated with the targeted therapeutic alone, with an agent that induces iron-dependent cellular disassembly alone, or with a combination of the targeted agent and the agent that induces iron-dependent cellular disassembly at the appropriate concentration for at least nine days with fresh drug added every three days. Surviving cells are grown for 14-37 days without the targeted therapeutic to increase the number of cells. The number of persister cells in each treatment group is measured. The agent that induces iron-dependent cellular disassembly is expected to reduce the number of persister cells post treatment. In addition, the combination therapy of the targeted agent and the agent that induces iron-dependent cellular disassembly is expected to reduce the number of persister cells to a greater extent than the target agent alone or the agent that induces iron-dependent cellular disassembly alone. See Hangauer et al., 2017; and Viswanathan et al., 2017.

### Example 16: Treatment of EMT Cancers In Vivo with an Agent that Induces Iron-Dependent Cellular Disassembly and an Anti-Neoplastic Agent

Mammary epithelial cells are neoplastically transformed by stepwise introduction of activated Ras+c-Myc+p53shRNA and p16shRNA as described above to produce a transformed population containing epithelial cells and mesenchymal cells. Mesenchymal state cancer cell lines such as HT1080 fibrosarcoma are also analyzed. The cell lines are grown in immunocompromised mice to form tumors, and the animals are treated with vehicle or an agent that induces iron-dependent cellular disassembly (e.g. a GPX4 inhibitor or a System Xc inhibitor). Tumor size is measured daily for 2 months. Treatment with the agent that induces iron-dependent cellular disassembly is expected to slow growth and/or stimulate a stronger immune response in the tumors.

Alternatively, cell lines with known susceptibility to an anti-neoplastic agent (e.g. a targeted therapy) are grown in immune compromised mice. The following experiments are performed.
1) The mice are treated with the anti-neoplastic agent (e.g. the targeted therapy) to shrink tumors to their minimal size. Upon regrowth of the tumor, mice are treated with vehicle, the original anti-neoplastic agent (e.g. the targeted therapy), or an agent that induces iron-dependent cellular disassembly (e.g. a GPX4 inhibitor or a System Xc inhibitor). Treatment with the agent that induces iron-dependent cellular disassembly is expected to slow growth of the tumor more than retreatment with the original anti-neoplastic agent.
2) The mice are treated with the anti-neoplastic agent (e.g. the targeted therapy) alone, or the anti-neoplastic agent and an agent that induces iron-dependent cellular disassembly (e.g. a GPX4 inhibitor or System Xc inhibitor) to shrink tumors to their minimal size. Treatment is then stopped and tumor regrowth is measured. Time to regrowth of the tumor is also measured. The combination treatment of the anti-neoplastic agent and the agent that induces iron-dependent cellular disassembly is expected to slow time to regrowth of the tumor and reduce the amount of tumor growth relative to the mice treated with the anti-neoplastic agent alone.

### Example 17: Treatment of Human Melanoma and Breast Cancer Cell Lines that are Resistant to Anticancer Agents with the Ferroptosis Inducer RSL3 In Vitro

The effects of the ferroptosis inducer RSL3 on cancer cell lines with acquired resistance to standard of care therapeutics was assessed in vitro. Human melanoma cell lines with acquired resistance to BRAF inhibitors (A375-DR or A375-VR) were generated by propagating parental A375 cells in increasing concentrations of dabrafenib or vemurafenib (10 nM-100 μM) over 8 weeks. These resistant melanoma cell lines were further maintained in growth medium containing 5 μM dabrafenib or vemurafenib. BT-474 Clone 5, a human breast cancer cell line that is resistant to trastuzumab, was purchased from American Type Culture Collection (ATCC). 5,000 A375-DR, A375-VR, or BT-474 clone 5 cells were plated per well in flat-bottom 96 well plates and incubated overnight. Cells were then dosed with graded concentrations of RSL3 for 72 hours, and cellular viability was assessed using the CellTiter-Glo 2.0 Cell Viability Assay. The experiments were run in triplicate.

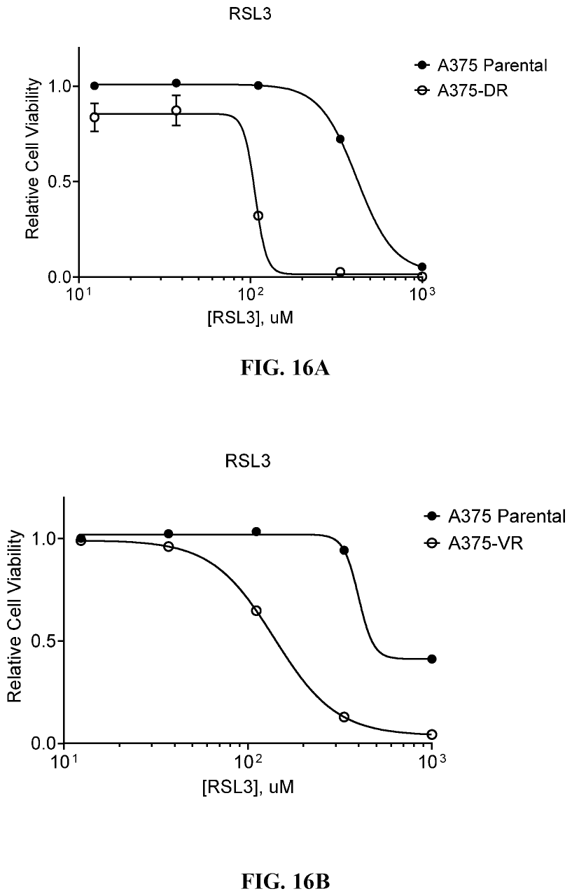

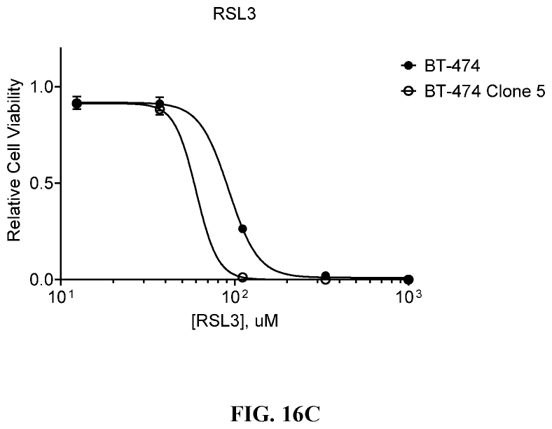

As shown in FIG. 16, BRAF inhibitor-resistant melanoma cell lines A375-DR and A375-VR, and trastuzumab-resistant BT-474 clone 5 cell line, were all more sensitive to RSL3-induced ferroptosis than the corresponding non-resistant parental cell line.

### Example 18: Screen of FDA-Approved Anticancer Drugs and Inducers of Ferroptosis for Induction of Innate Immune Activity as Evidenced by NFkB Activity in THP1 Monocytes In Vitro

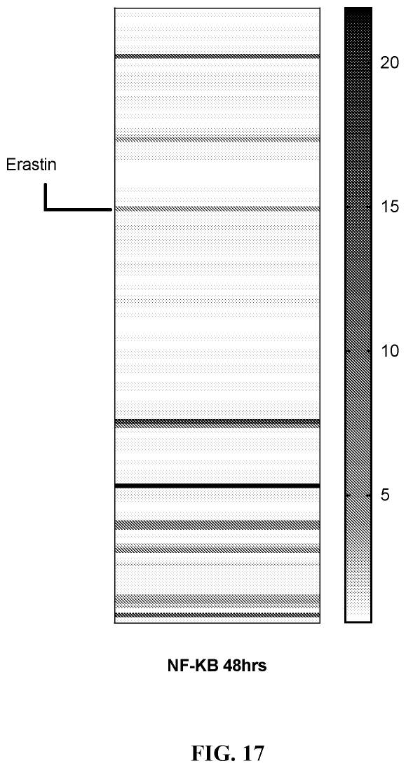

Over 100 FDA-approved anticancer drugs and 6 inducers of ferroptosis were evaluated for their ability to induce innate immune activation, as measured by induction of an NFkB reporter in THP1 monocytes in vitro. The source of the approved anticancer drug compounds was plate Approved Oncology Drug (AOD) IX, available from the National Cancer Institute, Division of Cancer Treatment and Diagnosis (dtp.cancer.gov/organization/dscb/obtaining/available_plates.htm). Briefly, 20,000-30,000 HT-1080 cells were plated per well in 96 well plates, and 10 μl each of compounds from the AOD IX plates were added to each well, for a final compound concentration of 10 PM. The six inducers of ferroptosis listed above were also included in the experiment, as a comparison. After addition of the drug, cells were incubated overnight, and 50,000 THP-1 reporter cells were added to each well on the following day. Reporter induction was measured by colorimetry 48h after reporter cell addition. As shown in FIG. 17, erastin was among the top ten most effective inducers of innate immune signaling of all the compounds tested, exhibiting a 3.2-fold increase in NFkB activity relative to the baseline. IKE also induced innate immune signaling, exhibiting a 2.4-fold increase in NFkB activity relative to the baseline. The other four inducers of ferroptosis could not be evaluated, since they exhibited toxicity to the THP1 monocyte reporter cell line under the conditions in this assay.

In FIG. 17, the intensity of the color of the band indicates the level of NFkB activation, with greater intensity indicating greater activation. The intensity of color corresponding to a particular fold increase in NFkB activity relative to the baseline is indicated in the box on the right, with the color intensity for 5-fold, 10-fold, 15-fold and 20-fold increases in NFkB activity indicated.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

### INCORPORATION BY REFERENCE

Each reference, patent, and patent application referred to in the instant application is hereby incorporated by reference in its entirety as if each reference were noted to be incorporated individually.

1. A method of killing a cancer cell in a subject, comprising contacting the cancer cell, or cells adjacent to the cancer cell, with a combination of (a) an anti-neoplastic agent and (b) an agent that induces iron-dependent cellular disassembly, wherein the cancer cell is resistant to the anti-neoplastic agent, thereby killing the cancer cell.

2. The method of claim 1, wherein the contacting induces iron-dependent cellular disassembly of the resistant cancer cell.

3. The method of claim 1 or 2, wherein the contacting results in an increase in immune response to the resistant cancer cell in the subject.

4. The method of any one of claims 1 to 3, wherein the resistant cancer cell exhibits:
(i) increased expression of a marker selected from the group consisting of HIF1, CD133, CD24, KDM5A/RBP2/Jarid1A, IGFBP3 (IGF-binding protein 3), Stat3, IRF-1, Interferon gamma, type I interferon, pax6, AKT pathway activation, IGF1, EGF, ANGPTL7, PDGFD, FRA1 (FOSL1), FGFR, KIT, IGF1R and DDR1, relative to a cancer cell that is sensitive to the anti-neoplastic agent; or
(ii) decreased expression of IGFBP-3 relative to a cancer cell that is sensitive to the anti-neoplastic agent.

5. The method of claim 1, wherein the antineoplastic agent and the cancer cell are selected from the antineoplastic agent and corresponding cancer cell listed in Table 4.

6. A method of killing cancer cells in a subject, comprising contacting the cancer cells, or cells adjacent to the cancer cells, with a combination of (a) an anti-neoplastic agent and (b) an agent that induces iron-dependent cellular disassembly, wherein the method increases the number of cancer cells undergoing iron-dependent cellular disassembly relative to cancer cells treated with the agent that induces iron-dependent cellular disassembly alone.

7. The method of claim 6, wherein the contacting results in an increase in immune response to the cancer cell in the subject.

8. A method of treating a cancer in a subject in need thereof, comprising administering to the subject, in combination (a) an anti-neoplastic agent and (b) an agent that induces iron-dependent cellular disassembly, thereby treating the cancer in the subject, wherein the cancer is resistant to the anti-neoplastic agent.

9. The method of claim 8, wherein the administration results in resistant cancer cells undergoing iron-dependent cellular disassembly in the subject.

10. The method of claim 8 or 9, wherein the administration results in an increase in immune response to the resistant cancer.

11. The method of any one of claims 8 to 10, wherein the method further comprises administering an immunotherapy to the subject.

12. The method of any one of claims 8 to 11, wherein the resistant cancer exhibits:
(i) increased expression of a marker selected from the group consisting of HIF1, CD133, CD24, KDM5A/RBP2/Jarid1A, IGFBP3 (IGF-binding protein 3), Stat3, IRF-1, Interferon gamma, type I interferon, pax6, AKT pathway activation, IGF1, EGF, ANGPTL7, PDGFD, FRA1 (FOSL1), FGFR, KIT, IGF1R and DDR1, relative to a cancer that is sensitive to the anti-neoplastic agent; or
(ii) decreased expression of IGFBP-3 relative to a cancer that is sensitive to the anti-neoplastic agent.

13. The method of any one of claims 8 to 12, wherein the antineoplastic agent and the cancer are selected from the antineoplastic agent and corresponding cancer listed in Table 4.

14. A method of treating a cancer in a subject in need thereof, comprising administering to the subject, in combination (a) an anti-neoplastic agent and (b) an agent that induces iron-dependent cellular disassembly, thereby treating the cancer in the subject, wherein the anti-neoplastic agent is known to induce resistance in the cancer.

15. A method of reducing the heterogeneity of a cancer in a subject in need thereof, wherein the cancer comprises cells that are resistant to an anti-neoplastic agent and cells that are sensitive to the anti-neoplastic agent, the method comprising administering to the subject, in combination (a) the anti-neoplastic agent and (b) an agent that induces iron-dependent cellular disassembly, thereby reducing the heterogeneity of the cancer.

16. The method of claim 15, wherein the cells that are resistant to the anti-neoplastic agent comprise persister cells.

17. The method of claim 15 or 16, wherein the subject was previously determined to have elevated levels of the persister cells.

18. The method of any one of claims 15 to 17, wherein the administration results in reduction of the number of the persister cells in the cancer.

19. The method of any one of claims 15 to 17, wherein the administration results in preferential killing of the persister cells in the cancer.

20. The method of any one claims 15 to 19, wherein the persister cells exhibit:
(i) increased expression of a marker selected from the group consisting of HIF1, CD133, CD24, KDM5A/RBP2/Jarid1A, IGFBP3 (IGF-binding protein 3), Stat3, IRF-1, Interferon gamma, type I interferon, pax6, AKT pathway activation, IGF1, EGF, ANGPTL7, PDGFD, FRA1 (FOSL1), FGFR, KIT, IGF1R and DDR1, relative to a cancer cell that is sensitive to the anti-neoplastic agent; or
(ii) decreased expression of IGFBP-3 relative to a cancer cell that is sensitive to the anti-neoplastic agent.

21. The method of any one of claims 15 to 20, wherein the cancer is selected from the group consisting of gastrointestinal stromal tumor (GIST), colorectal cancer (CRC), non-small cell lung cancer (NSCLC), melanoma, ovarian cancer, breast cancer and gastric cancer.

22. The method of any one of claims 15 to 21, wherein the cells that are resistant to the anti-neoplastic agent comprise cancer stem cells (CSCs).

23. The method of claim 22, wherein the cancer further comprises non-CSCs.

24. The method of claim 23, wherein the non-CSCs are sensitive to the anti-neoplastic agent.

25. The method of any one of claims 22 to 24, wherein the subject was previously determined to have elevated levels of the CSCs.

26. The method of any one of claims 22 to 25, wherein the CSCs are epithelial-mesenchymal transition (EMT) cells.

27. The method of any one of claims 23 to 25, wherein the non-CSCs are epithelial cells.

28. The method of any one of claims 22 to 27, wherein the administration results in reduction of the number of the CSCs in the cancer.

29. The method of any one of claims 22 to 28, wherein the administration results in preferential killing of the CSCs in the cancer.

30. The method of any one of claims 22 to 29, wherein the CSCs exhibit (i) increased expression of a marker selected from the group consisting of Vimentin (S100A4), Beta-catenin, N-cadherin, Beta6 integrin, Alpha4 integrin, DDR2, FSP1, Alpha-SMA, Beta-Catenin, Laminin 5, FTS-1, Twist, FOXX2, OB-cadherin, Alpha5beta1 integrin, alphaVbeta6 integrin, Syndecan-1, Alpha1 (I) collagen, Alpha1 (III) collagen, Snail1, Snail2, ZEB1, CBF-A/KAP-1 complex, LEF-1, Ets-1 and miR-21, relative to a non-CSC; or (ii) decreased expression of a marker selected from the group consisting of E-cadherin, ZO-1, cytokeratin, Alpha1 (IV) collagen and Laminin 1, relative to a non-CSC.

31. The method of any one of claims 15 to 30, wherein the method reduces risk of relapse of the cancer.

32. The method of any one of claims 15 to 31, wherein the method reduces risk of metastasis of the cancer.

33. A method of increasing the therapeutic index of an anti-neoplastic agent for treating a cancer in a subject in need thereof, comprising administering to the subject, in combination (a) the anti-neoplastic agent and (b) an agent that induces iron-dependent cellular disassembly, thereby increasing the therapeutic index of the anti-neoplastic agent for treating the cancer in the subject.

34. A method of treating a cancer in a subject in need thereof, comprising administering to the subject, in combination (a) an anti-neoplastic agent and (b) an agent that induces iron-dependent cellular disassembly, wherein the anti-neoplastic agent is administered at a dose that is lower than an effective dose of the anti-neoplastic agent when administered alone to treat the cancer, thereby treating the cancer in the subject.

35. The method of claim 34, wherein the anti-neoplastic agent has a dose limiting effect.

36. The method of claim 34 or 35, wherein the anti-neoplastic agent is administered at a dose that is at least 5%, 10%, 20%, 30%, 40%, 50%, or 60% less than the effective dose of the anti-neoplastic agent when administered alone to treat the cancer.

37. The method of any one of claim 1 to 36, wherein the cancer has a mesenchymal phenotype.

38. The method of any one of claims 1 to 36, wherein the cancer exhibits (i) increased expression of a marker selected from the group consisting of Vimentin (S100A4), Beta-catenin, N-cadherin, Beta6 integrin, Alpha4 integrin, DDR2, FSP1, Alpha-SMA, Beta-Catenin, Laminin 5, FTS-1, Twist, FOXX2, OB-cadherin, Alpha5beta1 integrin, alphaVbeta6 integrin, Syndecan-1, Alpha1 (I) collagen, Alpha1 (III) collagen, Snail1, Snail2, ZEB1, CBF-A/KAP-1 complex, LEF-1, Ets-1 and miR-21, relative to a non-CSC; and/or (ii) decreased expression of a marker selected from the group consisting of E-cadherin, ZO-1, cytokeratin, Alpha1 (IV) collagen and Laminin 1, relative to a non-mesenchymal cancer.

39. The method of any one of claims 1 to 36, wherein the cancer is selected from the group consisting of chronic myeloid leukemia (CML), acute lymphoblastic leukemia (ALL), gastrointestinal stromal tumor (GIST), colorectal cancer (CRC), non-small cell lung cancer (NSCLC), melanoma, ovarian cancer, breast cancer and gastric cancer.

40. The method of any one of claims 1 to 39, wherein the anti-neoplastic agent and the agent that induces iron-dependent cellular disassembly are administered to the subject simultaneously.

41. The method of any one of claims 1 to 39, wherein the anti-neoplastic agent and the agent that induces iron-dependent cellular disassembly are administered to the subject sequentially.

42. The method of any one of claims 1 to 41, wherein the anti-neoplastic agent and the agent that induces iron-dependent cellular disassembly are administered in an amount that causes a synergistic effect.

43. The method of any one of claims 1 to 42, wherein the method results in an increased immune response to the cancer.

44. The method of claim 43, wherein the increased immune response comprises activation of one or more cells selected from the group consisting of monocytes, pro-inflammatory macrophages, dendritic cells, neutrophils, NK cells and T cells.

45. The method of claim 43 or 44, wherein the increased immune response comprises an increase in the level or activity of NFκB, IRF or STING in an immune cell.

46. The method of claim 45, wherein the immune cell is a THP-1 cell.

47. The method of any one of claims 1 to 46, wherein the method further comprises administering an immunotherapeutic agent to the subject.

48. The method of claim 47, wherein the anti-neoplastic agent, the agent that induces iron-dependent cellular disassembly, and the immunotherapeutic agent are administered in an amount that causes a synergistic effect.

49. The method of any one of claims 1 to 48, wherein the anti-neoplastic agent is a cytotoxic agent.

50. The method of any one of claims 1 to 48, wherein the anti-neoplastic agent is radiotherapy.

51. The method of any one of claims 1 to 50, wherein the anti-neoplastic agent induces apoptosis in a cancer cell.

52. The method of claim 51, wherein the anti-neoplastic agent induces apoptosis in a cancer cell in the absence of an agent that induces iron-dependent cellular disassembly.

53. The method of claim 51 or 52, wherein the anti-neoplastic agent is selected from the group consisting of ONY-015, INGN201, PS1145, Bortezomib, CCI779, RAD-001 and ABT-199 (Venetoclax).

54. The method of any one of claims 1 to 48, wherein the antineoplastic agent is selected from the group consisting of Bosutinib, Dasatinib, Imatinib, Nilotinib, Ponatinib, Cetuximab, Panitumumab, Afatinib, Erlotinib, Gefitinib, Dabrafenib, Vemurafenib, Ceritinib, Crizotinib Trametinib, Olaparib, Ado-trastuzumab, emtansine, Lapatinib, Pertuzumab and Trastuzumab.

55. The method of any one of claims 1 to 49 and 51 to 54, wherein the antineoplastic agent is unconjugated.

56. The method of any one of claims 1 to 49 and 51 to 54, wherein the antineoplastic agent is conjugated to a targeting moiety.

57. The method of claim 56, wherein the targeting moiety is an antibody or antigen-binding fragment thereof.

58. The method of any one of claims 1, 6, 8, 14, 15, 33 and 34, wherein the agent that induces iron-dependent cellular disassembly is selected from the group consisting of an inhibitor of antiporter system Xc⁻, an inhibitor of GPX4, and a statin.

59. The method of any one of claims 1 to 49 and 55 to 57, wherein the agent that induces iron-dependent cellular disassembly is selected from the group consisting of an inhibitor of antiporter system Xc⁻, an inhibitor of GPX4, and a statin.

60. The method of any one of claims 1 to 59, wherein the iron-dependent cellular disassembly is ferroptosis.

61. The method of any one of claims 58 to 60, wherein the inhibitor of antiporter system Xc⁻ is erastin or a derivative or analog thereof.

62. The method of claim 61 wherein the erastin or derivative or analog thereof has the following formula: 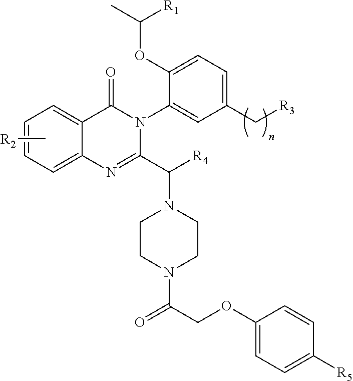
or pharmaceutically acceptable salts or esters thereof, wherein
R₁ is selected from the group consisting of H, C₁₋₄ alkyl, C₁₋₄ alkoxy, hydroxy, and halogen;
R₂ is selected from the group consisting of H, halo, and C₁₋₄ alkyl;
R₃ is selected from the group consisting of H, C₁₋₄ alkyl, C₁₋₄ alkoxy, 5-7 membered heterocycloalkyl, and 5-6 membered heteroaryl;
R₄ is selected from the group consisting of H and C₁₋₄ alkyl;
R₅ is halo; 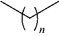
is optionally substituted with ═O; and
n is an integer from 0-4.

63. The method of claim 61, wherein the analog of erastin is PE or IKE.

64. The method of any one of claims 58 to 60, wherein the inhibitor of GPX4 is selected from the group consisting of (1S,3R)-RSL3 or a derivative or analog thereof, ML162, DPI compound 7, DPI compound 10, DPI compound 12, DPI compound 13, DPI compound 17, DPI compound 18, DPI compound 19, FIN56, and FINO2.

65. The method of claim 64, wherein the RSL3 derivative or analog is a compound represented by Structural Formula (I): 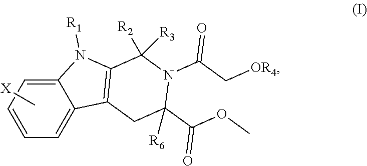
or an enantiomer, optical isomer, diastereomer, N-oxide, crystalline form, hydrate, or pharmaceutically acceptable salt thereof, wherein
R₁, R₂, R₃, and R₆ are independently selected from H, C₁₋₈alkyl, C₁₋₈alkoxy, C₁₋₈aralkyl, 3- to 8-membered carbocyclic, 3- to 8-membered heterocyclic, 3- to 8-membered aryl, or 3- to 8-membered heteroaryl, acyl, alkylsulfonyl, and arylsulfonyl, wherein each alkyl, alkoxy, aralkyl, carbocyclic, heterocyclic, aryl, heteroaryl, acyl, alkylsulfonyl, and arylsulfonyl is optionally substituted with at least one substituent;
R₄ and R₅ are independently selected from H₁ C₁₋₈alkyl, C₁₋₈alkoxy, 3- to 8-membered carbocyclic, 3- to 8-membered heterocyclic, 3- to 8-membered aryl, or 3- to 8-membered heteroaryl, carboxylate, ester, amide, carbohydrate, amino acid, acyl, alkoxy-substituted acyl, alditol, NR⁷R⁸, OC(R⁷)₂COOH, SC(R⁷)₂COOH, NHCHR⁷COOH, COR⁸, CO₂R⁸, sulfate, sulfonamide, sulfoxide, sulfonate, sulfone, thioalkyl, thioester, and thioether, wherein each alkyl, alkoxy, carbocyclic, heterocyclic, aryl, heteroaryl, carboxylate, ester, amide, carbohydrate, amino acid, acyl, alkoxy-substituted acyl, alditol, NR⁷R⁸, OC(R⁷)₂COOH, SC(R⁷)₂COOH, NHCHR⁷COOH, COR⁸, CO₂R⁸, sulfate, sulfonamide, sulfoxide, sulfonate, sulfone, thioalkyl, thioester, and thioether is optionally substituted with at least one substituent;
R⁷ is selected from H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R⁸ is selected from H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent; and
X is 0-4 substituents on the ring to which it is attached.

66. The method of claim 64, wherein the RSL3 derivative or analog is a compound represented by Structural Formula (II): 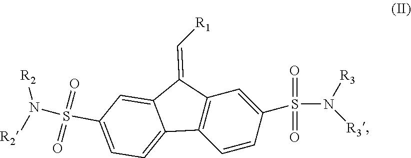
or an N-oxide, crystalline form, hydrate, or pharmaceutically acceptable salt thereof; wherein:
R₁ is selected from the group consisting of H, OH, and —(OCH₂CH₂)ₓOH;
X is an integer from 1 to 6; and
R₂, R₂′, R₃, and R₃′ independently are selected from the group consisting of H, C₃₋₈cycloalkyl, and combinations thereof, or R₂ and R₂′ may be joined together to form a pyridinyl or pyranyl and R₃ and R₃′ may be joined together to form a pyridinyl or pyranyl.

67. The method of claim 64, wherein the RSL3 derivative or analog is a compound represented by Structural Formula (III): 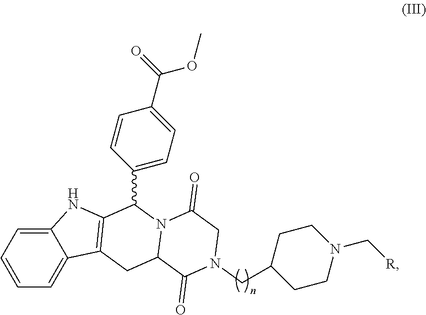
or a stereoisomer thereof, or a pharmaceutically acceptable salt thereof; wherein: n is 2, 3 or 4; and R is a substituted or unsubstituted C₁-C₆ alkyl group, a substituted or unsubstituted C₃-C₁₀ cycloalkyl group, a substituted or unsubstituted C₂-C₈ heterocycloalkyl group, a substituted or unsubstituted C₆-C₁₀ aromatic ring group, or a substituted or unsubstituted C₃-C₈ heteroaryl ring group; wherein the substitution means that one or more hydrogen atoms in each group are substituted by the following groups selected from the group consisting of: halogen, cyano, nitro, hydroxy, C₁-C₆ alkyl, halogenated C₁-C₆ alkyl, C₁-C₆ alkoxy, halogenated C₁-C₆ alkoxy, COOH (carboxy), COOC₁-C₆ alkyl, OCOC₁-C₆ alkyl.

68. The method of claim 64, wherein the RSL3 derivative or analog is a compound represented by Structural Formula (VI): 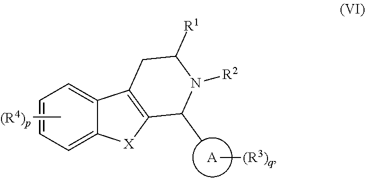
or an enantiomer, optical isomer, diastereomer, N-oxide, crystalline form, hydrate, or pharmaceutically acceptable salt thereof, wherein
ring A is C₄-C₁₀cycloalkyl, heterocyclyl, aryl, or heteroaryl;
X is NR⁵, O or S;
p is 0, 1, 2 or 3;
q is 0, 1, 2 or 3;
R¹ is C₁-C₆alkyl, C₂-C₆alkenyl, C₂-C₆alkynyl, C₁-C₆haloalkyl, C₃-C₁₀cycloalkyl, —CN, —OH, —C(O)OR⁶, —C(O)N(R⁷)₂, —OC(O)R⁶, —S(O)₂R⁸, —S(O)₂N(R⁷)₂, —S(O)N(R⁷)₂, —S(O)R⁸, —NH₂, —NHR⁸, —N(R⁸)₂, —NO₂, —OR⁸, —C₁-C₆alkyl-OH, —C₁-C₆alkyl-OR, or —Si(R¹⁵)₃;
R² is —C(O)R⁹;
each R³ is independently halo, —CN, —OH, —OR, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R, —NR¹²C(O)OR⁸, —OC(O)N(R⁷)₂, —OC(O)R⁸, —C(O)R⁶, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R³ is independently optionally substituted with one to three R¹⁰;
each R⁴ is independently halo, —CN, —OH, —OR, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R⁴ is optionally independently optionally substituted with one to three R¹⁰;
R⁵ is hydrogen or C₁-C₆alkyl;
each R⁶ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each R⁶ is independently further substituted with one to three R¹¹;
each R⁷ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₆cycloalkyl, —C₂-C₆alkenylC₃-C₆cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, —C₂-C₆alkenylheteroaryl, or two R⁷ together with the nitrogen atom to which they are attached, form a 4 to 7 membered heterocyclyl; wherein each R⁷ or ring formed thereby is independently further substituted with one to three R¹¹;
each R⁸ is independently C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or C₂-C₆alkenylheteroaryl; wherein each R⁸ is independently further substituted with one to three R¹¹;
R⁹ is —C₁-C₂haloalkyl, —C₂-C₃alkenyl, —C₂-C₃haloalkenyl, C₂alkynyl, or —CH₂OS(O)₂-phenyl, wherein the C₁-C₂alkylhalo and —C₂-C₃alkenylhalo are optionally substituted with one or two —CH₃, and the C₂alkynyl and phenyl are optionally substituted with one —CH₃;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl, wherein each C₁-C₆alkyl, C₁-C₆haloalkyl, C₂-C₆alkenyl, C₂-C₆alkynyl, C₃-C₁₀cycloalkyl, heterocyclyl, aryl, or heteroaryl of R¹⁰ is optionally independently substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl;
each R¹² is independently hydrogen, C₁-C₆alkyl or C₃-C₁₀cycloalkyl;
each R¹³ is independently C₁-C₆alkyl or C₃-C₁₀cycloalkyl; and
each R¹⁵ is independently C₁-C₆alkyl, C₂-C₆alkenyl, aryl, heteroaryl, —C₁-C₆alkylaryl, —C₂-C₆alkenylaryl, —C₁-C₆alkylheteroaryl, and —C₂-C₆alkenylheteroaryl.

69. The method of claim 68, wherein when X is NR⁵, then R⁹ is C₂alkynyl.

70. The method of claim 68, wherein when X is NR⁵, and R⁹ is —C₁-C₂haloalkyl, —C₂-C₃alkenyl, —C₂-C₃haloalkenyl, or —CH₂OS(O)₂-phenyl, wherein the C₁-C₂alkylhalo and —C₂-C₃alkenylhalo are optionally substituted with one or two —CH₃, and the phenyl is optionally substituted with —CH₃, then R¹ is other than —C(O)OR⁶ and —C(O)N(R⁷)₂.

71. The method of claim 68, wherein when X is NR⁵, then (i) R⁹ is C₂alkynyl; or (ii) R⁹ is —C₁-C₂haloalkyl, —C₂-C₃alkenyl, —C₂-C₃haloalkenyl, or —CH₂OS(O)₂-phenyl, wherein the C₁-C₂alkylhalo and —C₂-C₃alkenylhalo are optionally substituted with one or two —CH₃, and the phenyl is optionally substituted with —CH₃, and R¹ is other than —C(O)OR⁶ and —C(O)N(R⁷)₂.

72. The method of claim 68, wherein when X is NH, R¹ is —C(O)OR⁶, R² is —C(O)CH₂Cl or C(O)CH₂F, q is 1, p is 0, and ring A with the R³ is 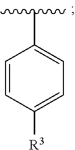
R³; then (i) R³ and R⁶ are not simultaneously —NO₂ and —CH₃, respectively, and (ii) when R⁶ is —CH₃, then R³ is other than H, halo, and —NO₂.

73. The method of claim 68, wherein when X is NH, R¹ is —C(O)OR⁶, R² is —C(O)CH₂Cl or C(O)CH₂F, q is 1, p is 0, ring A with the R³ is 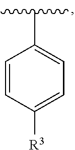
R³, and R³ is —C(O)OR⁶; then both R⁶ are not simultaneously
(i) —CH₃;
(ii) —CH₃ and C₂-C₆alkynyl, respectively; or
(iii) —CH₂CH₃ and —CH₃, respectively.

74. The method of claim 68, wherein when X is NH, R¹ is —C(O)OCH₃, R² is —C(O)CH₂Cl or —C(O)CH₂F, q is 1, p is 0, and R³ is H; then ring A is other than phenyl.

75. The method of claim 68, wherein when X is NH, R¹ is —C(O)N(R⁷)₂, wherein R⁷ are H, R² is —C(O)CH₂Cl or —C(O)CH₂F, q is 0, or 1, p is 0, and ring A is phenyl; then q is not 0, or when q is 1, R³ is other than halo.

76. The method of any one of claims 58 to 60, wherein the statin is selected from the group consisting of atorvastatin, fluvastatin, lovastatin, pitavastatin, pravastatin, rosuvastatin, cerivastatin and simvastatin.

77. The method of any one of claims 1 to 49, wherein the agent that induces iron-dependent cellular disassembly is selected from the group consisting of sorafenib or a derivative or analog thereof, sulfasalazine, glutamate, BSO, DPI2, cisplatin, cysteinase, silica based nanoparticles, CCI4, ferric ammonium citrate, trigonelline and brusatol.

78. The method of any one of claims 1 to 77, wherein the agent that induces iron-dependent cellular disassembly has one or more of the following characteristics:
(a) induces iron-dependent cellular disassembly of a target cell in vitro and subsequent activation of an immune response in a co-cultured cell;
(b) induces iron-dependent cellular disassembly of a target cell in vitro and subsequent activation of co-cultured macrophages, e.g., RAW264.7 macrophages;
(c) induces iron-dependent cellular disassembly of a target cell in vitro and subsequent activation of co-cultured monocytes, e.g., THP-1 monocytes;
(d) induces iron-dependent cellular disassembly of a target cell in vitro and subsequent activation of co-cultured bone marrow-derived dendritic cells (BMDCs);
(e) induces iron-dependent cellular disassembly of a target cell in vitro and subsequent increase in levels or activity of NFkB, IRF and/or STING in a co-cultured cell;
(f) induces iron-dependent cellular disassembly of a target cell in vitro and subsequent increase in levels or activity of a pro-immune cytokine in a co-cultured cell; and
(g) induces iron-dependent cellular disassembly of a target cell in vitro and subsequent activation of co-cultured CD4+ cells, CD8+ cells and/or CD3+ cells.

79. The method of any one of claims 1 to 49, 51 to 54, and 55 to 78, wherein the agent that induces iron-dependent cellular disassembly is targeted to a cancer cell.
